(19) United States
(12) Patent Application Publication
(10) Pub. No.: US 2025/0101500 A1
(43) Pub. Date: Mar. 27, 2025

(54) AMPLIFICATION ASSAYS USING CRISPR-CAS BASED DETECTION

(71) Applicants: The Broad Institute, Inc., Cambridge, MA (US); Massachusetts Institute of Technology, Cambridge, MA (US); THE GENERAL HOSPITAL CORPORATION, Boston, MA (US)

(72) Inventors: Deborah HUNG, Cambridge, MA (US); Gowtham THAKKU, Cambridhge, MA (US); Sharon WONG, Cambridge, MA (US); James GOMEZ, Cambridge, MA (US)

(73) Assignees: The Broad Institute, Inc., Cambridge, MA (US); Massachusetts Institute of Technology, Cambridge, MA (US); THE GENERAL HOSPITAL CORPORATION, Boston, MA (US)

(21) Appl. No.: 18/906,750

(22) Filed: Oct. 4, 2024

Related U.S. Application Data
(63) Continuation of application No. PCT/US2023/065528, filed on Apr. 7, 2023.
(60) Provisional application No. 63/328,881, filed on Apr. 8, 2022.

(51) Int. Cl. C12Q 1/6823; C12N 9/22; C12N 15/11; C12Q 1/34; C12Q 1/6806; C12Q 1/6844; C12Q 1/6888

(52) U.S. Cl. CPC C12Q 1/6823; C12N 9/22; C12N 15/111; C12Q 1/34; C12Q 1/6806; C12Q 1/6844; C12Q 1/6888; C12N 2310/20

(57) ABSTRACT

Described in various embodiments herein are tiled amplification nucleic acid detection systems and uses thereof. In some embodiments, the nucleic acids amplified and detected are cell free DNA (cfDNA).

### CROSS-REFERENCE TO RELATED APPLICATIONS

This application claims the benefit of and priority to U.S. Provisional Patent Application No. 63/328,881, filed on Apr. 8, 2022, the contents of which is incorporated by reference herein in its entirety.

### SEQUENCE LISTING

This application contains a sequence listing filed in electronic form as an xml file entitled “114203-2352_ST26.xml”, created on Oct. 4, 2024, and having a size of 55,847 bytes. The content of the sequence listing is incorporated herein in its entirety.

### TECHNICAL FIELD

The subject matter disclosed herein is generally directed to nucleic acid detection systems and methods of detecting nucleic acid using CRISPR-Cas systems.

### BACKGROUND

In 2017 globally, there were 3.6 million undiagnosed cases of tuberculosis (TB) (World Health, O., Global tuberculosis report 2018. 2018, Geneva: World Health Organization). Diagnosis of TB is challenging, especially among children and individuals with HIV/AIDS and/or extrapulmonary TB. More invasive sampling procedures simply are not an option in many resource-limited settings. Thus, the WHO has prioritized the need for a rapid biomarker-based, non-sputum-based test to detect all forms of TB. Indeed, a urine lipoarabinomannan (LAM) assay is now used for the detection of active TB in HIV-infected patients with CD4<100 but has shown sub-optimal sensitivity for routine clinical use (Lawn, S. D. and A. Gupta-Wright, Detection of lipoarabinomannan (LAM) in urine is indicative of disseminated TB with renal involvement in patients living with HIV and advanced immunodeficiency: evidence and implications. Transactions of the Royal Society of Tropical Medicine and Hygiene, 2016. 110(3): p. 180-185).

More recently, the presence of cell-free DNA (cfDNA) derived from Mycobacterium tuberculosis (Mtb) in the blood and urine of individuals with pulmonary and extrapulmonary TB has been reported, demonstrating the important potential of cfDNA as a viable TB biomarker detectable in easy-to acquire bodily fluids (Cannas, A., et al., Mycobacterium tuberculosis DNA detection in soluble fraction of urine from pulmonary tuberculosis patients. The international journal of tuberculosis and lung disease, 2008. 12(2): p. 146-151; Click, E., et al., Detection of apparent cell-free M. tuberculosis DNA from plasma. Scientific reports, 2018. 8(1): p. 1-6; Labugger, I., et al., Detection of transrenal DNA for the diagnosis of pulmonary tuberculosis and treatment monitoring. Infection, 2017. 45(3): p. 269-276; and Ushio, R., et al., Digital PCR assay detection of circulating Mycobacterium tuberculosis DNA in pulmonary tuberculosis patient plasma. Tuberculosis, 2016. 99: p. 47-53). Indeed, a few pilot studies have used polymerase chain reaction (PCR) to detect Mtb cfDNA in plasma, urine and pleural fluid of TB patients (Cannas et al. 2008., Labugger et al. 2017, and Che, N., et al., Rapid detection of cell-free Mycobacterium tuberculosis DNA in tuberculous pleural effusion. Journal of clinical microbiology, 2017. 55(5): p. 1526-1532). In these reports however, cfDNA PCR assays have suboptimal sensitivity (plasma, 44%-65%; urine, 64%-79%). Thus, current assays lack the sensitivity to adequately detect cfDNA and require expensive instrumentation and reagents (Crowley, E., et al., Liquid biopsy: monitoring cancer-genetics in the blood. Nature reviews Clinical oncology, 2013. 10(8): p. 472 and Fernandez-Carballo, B. L., et al., Toward the development of a circulating free DNA-based in vitro diagnostic test for infectious diseases: a review of evidence for tuberculosis. Journal of clinical microbiology, 2019. 57(4)).

Thus, there is a clear and urgent need for accurate, yet simple tests on field-deployable platforms to diagnose diseases, including but not limited to TB, particularly platforms based on detection of cfDNA or other fragmented nucleic acids.

Citation or identification of any document in this application is not an admission that such a document is available as prior art to the present invention.

### SUMMARY

Described in certain example embodiments herein are nucleic acid detection systems comprising a set of amplification primers configured to amplify a set of target regions in one or more target elements, wherein primers of the set of amplification primers are optionally optimized for pooled amplification; one or more Cas proteins having collateral activity; a set of guide polynucleotides comprising a guide polynucleotide specific for each target region amplified by the set of amplification primers, and wherein each of the guide polynucleotides in the set of guide polynucleotides is capable of forming a CRISPR-Cas complex with the one or more Cas proteins; and an oligonucleotide-based detection construct comprising a non-target sequence, wherein the non-target sequence is configured to be cleaved by the collateral activity of the one or more Cas proteins.

In certain example embodiments, the set of amplification primers optionally optimized for pooled detection are configured to minimize 3′ to 3′ interactions between primers.

In certain example embodiments, the guide polynucleotides of the set of guide polynucleotides are optimized for pooled detection.

In certain example embodiments, the set of amplification primers comprises 2-100 or more amplification primers.

In certain example embodiments, the set of guide polynucleotides comprises 2-50 or more guide polynucleotides.

In certain example embodiments, the system is configured to detect 2-50 or more target elements.

In certain example embodiments, the system is configured to detect 2-50 or more target regions.

In certain example embodiments, the one or more target elements is specific to a microorganism or a virus, optionally a pathogenic microorganism or virus.

In certain example embodiments, the one or more target elements are elements in a genome of a microorganism or virus, optionally a pathogenic microorganism or virus.

In certain example embodiments, wherein the one or more target elements are elements in circulating cell free (ccfDNA).

In certain example embodiments, one or more of the one or more target elements is a repetitive target element.

In certain example embodiments, the set of amplification primers comprises PCR primers, isothermal amplification primers, proximity dependent probes, or any combination thereof. In certain example embodiments, the proximity dependent probes comprise one or more of a forward primer binding site, a reverse primer binding site, and a reverse polymerase binding site.

In certain example embodiments, one or more primers of the set of amplification primers configured to amplify a set of target regions comprise an in vitro transcription promoter sequence, optionally at the 5′end of the one or more primers.

In certain example embodiments, the nucleic acid detection system further comprises a capture agent that binds a capture moiety of a capture moiety modified primer, proximity probe, amplicon, or sequence formed from proximity probe binding a proximity probe target sequence.

In certain example embodiments, the capture agent is streptavidin or streptavidin coated surface and wherein the capture moiety is a biotinylated nucleotides of a capture moiety modified primer, proximity probe, amplicon, or sequence formed from proximity probe binding a proximity probe target sequence.

In certain example embodiments, the proximity dependent probe is a molecular inversion probe (MIP), and wherein the MIP comprises a first target binding sequence and a second target binding sequence linked by a linking region, the linking region comprising one or more of a forward primer binding sequence, a reverse primer binding sequence, an RNA polymerase binding sequence, a guide polynucleotide binding sequence, and a barcode.

In certain example embodiments, the first target binding sequence and the second target binding sequence hybridize on the target sequence directly adjacent to one another.

In certain example embodiments, the first and second target binding sequence hybridize on the target sequence such that there is at least a single nucleotide gap region between the first and second target binding sequence. In certain example embodiments, filling the gap region between the first and second targeting binding sequence generates a guide polynucleotide recognition sequence.

In certain example embodiments, one or more primers of the amplification primer set comprises an origin-specific barcode, a set-specific barcode, a unique molecular identifier (UMI), or any combination thereof.

In certain example embodiments, the amplification primer set is configured to amplify at least one or more target regions of a target element that cover at least 10 percent, at least 20 percent, at least 30 percent, at least 40 percent, at least 50 percent, or at least 60 percent of a target element.

In certain example embodiments, the nucleic acid detection system further comprises amplification reagents for amplifying the set of target regions. In certain example embodiments, the amplification reagents comprise nucleic acid sequence-based amplification (NASBA) reagent(s), recombinase polymerase amplification (RPA) reagent(s), loop-mediated isothermal amplification (LAMP) reagent(s), RT-LAMP reagent(s), strand displacement amplification (SDA) reagent(s), helicase-dependent amplification (HDA) reagent(s), nicking enzyme amplification reaction (NEAR) reagent(s), Polymerase Chain Reaction (PCR) reagent(s), multiple displacement amplification (MDA) reagent(s), rolling circle amplification (RCA) reagent(s), ligase chain reaction (LCR) reagent(s), or ramification amplification method (RAM) reagent(s).

In certain example embodiments, the nucleic acid detection system further comprises a nucleic acid enrichment reagent, optionally a ccfDNA enrichment agent. In certain example embodiments, the nucleic acid enrichment reagent comprises a DNA methylation enrichment agents, size selection reagents to enrich for a nucleic acid, a magnetic or paramagnetic particle configured to bind nucleic acids, or any combination thereof.

In certain example embodiments, the nucleic acid detection system further comprises one or more nuclease inactivation reagents, microorganism or virus inactivation reagents, or both.

In certain example embodiments, the one or more Cas proteins comprise an RNA-targeting protein, a DNA-targeting protein, or a combination thereof.

In certain example embodiments, the one or more Cas proteins comprise Cas13, a Cas 12, or a combination thereof.

In certain example embodiments, the Cas13 is a Cas13a, Cas13b, Cas13c, a Cas13d, or a combination thereof.

In certain example embodiments, the Cas12 is a Cas12a, Cas12b, or Cas12c.

In certain example embodiments, wherein each of the one or more Cas proteins has a different polynucleotide cutting preference and wherein the polynucleotide cutting preference is matched to a guide polynucleotide for a particular amplified target region.

In certain example embodiments, the nucleic acid detection system comprises two or more CRISPR-Cas systems, wherein the two or more CRISPR-Cas systems comprise an RNA-targeting effector protein, a DNA-targeting effector protein, or a combination thereof.

In certain example embodiments, the two or more CRISPR-Cas systems are each a Cas13 system, as Cas12 system, or a combination thereof, optionally wherein the Cas 12 system is a Cpf1 or c2c1 system and/or the Cas 13 system is Cas 13a, Cas 13b, or Cas 13c system.

In certain example embodiments, the one or more guide polynucleotides are each about 28 nucleotides in length and have a mismatch of one or less to the corresponding target sequence.

In certain example embodiments, the nucleic acid detection system comprises two or more oligonucleotide-based detection constructs.

In certain example embodiments, the oligonucleotide-based detection construct comprises a masking construct configured to suppress generation of a detectable positive signal until the non-target sequence is cleaved by the collateral activity of the one or more Cas proteins.

In certain example embodiments, the masking construct suppresses generation of a detectable positive signal by masking the detectable positive signal or generating by a detectable negative signal.

In certain example embodiments, the masking construct comprises a silencing RNA that suppresses generation of a gene product encoded by a reporting construct, wherein the gene product generates the detectable positive signal when expressed.

In certain example embodiments, the masking construct comprises a ribozyme that generates the negative detectable signal, and wherein the positive detectable signal is generated when the ribozyme is deactivated. In certain example embodiments, the ribozyme converts a substrate to a first color and wherein the substrate converts to a second color when the ribozyme is deactivated.

In certain example embodiments, the masking construct is a DNA or RNA aptamer and/or comprises a DNA or RNA-tethered inhibitor.

In certain example embodiments, the aptamer or DNA- or RNA-tethered inhibitor sequesters an enzyme, wherein the enzyme generates a detectable signal upon release from the aptamer or DNA or RNA tethered inhibitor by acting upon a substrate.

In certain example embodiments, the aptamer is an inhibitor aptamer that inhibits an enzyme and prevents the enzyme from catalyzing generation of a detectable signal from a substance or wherein the DNA- or RNA-tethered inhibitor inhibits an enzyme and prevents the enzyme from catalyzing generation of a detectable signal from a substrate.

In certain example embodiments, the enzyme is thrombin and the substrate is para-nitroanilide covalently linked to a peptide substrate for thrombin, or 7-amino-4 methylcoumarin covalently linked to a peptide substrate for thrombin.

In certain example embodiments, the aptamer sequesters a pair of agents that when released from the aptamers combine to generate a detectable signal.

In certain example embodiments, the masking construct comprises a DNA or RNA oligonucleotide to which a detectable ligand and a masking component are attached.

In certain example embodiments, the masking construct comprises DNA or RNA in complex with an intercalating agent, wherein the intercalating agent changes absorbance upon cleavage of the DNA or RNA, optionally wherein the intercalating agent is pyronine-Y or methylene blue, and optionally wherein the detectable ligand is a fluorophore and the masking component is a quencher molecule.

In certain example embodiments, the masking construct comprises a nanoparticle held in aggregate by bridge molecules, wherein at least a portion of the bridge molecules comprises DNA or RNA, and wherein the solution undergoes a color shift when the nanoparticle is disbursed in solution, optionally wherein the nanoparticle is colloidal metal, optionally colloidal gold.

In certain example embodiments, the masking construct comprising a quantum dot linked to one or more quencher molecules by a linking molecule, wherein at least a portion of the linking molecule comprises DNA or RNA.

In certain example embodiments, the nucleic acid detection system comprises two or more CRISPR-Cas systems each comprising one or more Cas proteins having collateral activity and two or more oligonucleotide-based detection constructs each having a masking construct, wherein the masking construct of each of the oligonucleotide-based detection constructs is preferentially cut by one of the two or more Cas proteins having collateral activity.

Described in certain example embodiments herein are methods of detecting one or more nucleic acids in a sample, the method comprising: (a) contacting one or more samples with a set of amplification primers configured to amplify a set of target regions in one or more target elements, wherein primers of the set of amplification primers are optionally optimized for pooled amplification; (b) amplifying, optionally by pooled amplification, two or more target regions in one or more target elements by the set of amplification primers thereby producing one or more amplified target regions; (c) contacting the one or more amplified target regions with one or more Cas proteins having collateral activity, a set of guide polynucleotides comprising a guide polynucleotide specific for each of the amplified target regions, wherein each of the guide polynucleotides in the set of guide polynucleotides is capable of forming a CRISPR-Cas complex with the one or more Cas proteins; (d) contacting the one or more amplified target regions, the one or more Cas proteins having collateral activity, guide polynucleotides, or complexes thereof with one or more oligonucleotide-based detection constructs each comprising a non-target sequence to be cleaved by the collateral activity of the one or more of the one or more Cas proteins upon activation of the Cas proteins; and (e) detecting a signal produced from the one or more oligonucleotide-based detection constructs in response cleavage of one or more of the non-target sequences by collateral activity of an activated CRISPR-Cas protein, thereby detecting one or more target elements in the sample.

In certain example embodiments, amplifying comprises nucleic acid sequence-based amplification (NASBA), recombinase polymerase amplification (RPA), loop-mediated isothermal amplification (LAMP), RT-LAMP, strand displacement amplification (SDA), helicase-dependent amplification (HDA), nicking enzyme amplification reaction (NEAR), polymerase chain reaction (PCR), multiple displacement amplification (MDA), rolling circle amplification (RCA), ligase chain reaction (LCR), or ramification amplification method (RAM), or a combination thereof.

In certain example embodiments, amplifying comprises isothermal amplification.

In certain example embodiments, the method further comprises one or more inactivation steps prior to amplifying.

In certain example embodiments, the one or more inactivation steps comprises heating the sample, optionally to 50 degrees C. or more and/or optionally to about 64 degrees C. or more.

In certain example embodiments, the one or more samples is a biological fluid, optionally plasma, blood, urine, or saliva. In certain example embodiments, the one or more samples is blood or plasma. In certain example embodiments, the method further comprises processing blood to obtain plasma.

In certain example embodiments, the method further comprises extracting cell free DNA, DNA, RNA, or other nucleic acids or any combination thereof from a sample prior to amplification.

In certain example embodiments, the one or more samples is/are obtained from one or more subjects. In certain example embodiments, one or more of the one or more subjects has an active microorganism and/or viral infection, has been infected, or is suspected of being infected with a microorganism and/or virus.

In certain example embodiments, one or more steps of them method are performed using a nucleic acid detection system of the present invention described herein.

A method of detecting and/or monitoring a host response to an infection with a microorganism or virus or treatment thereof comprising performing the method of the present invention on a host sample obtained at a first timepoint; performing the method of the present invention on a host sample obtained at a second timepoint; and detecting the presence of one or more pathogens at the first timepoint and the second timepoint, thereby method of detecting and/or monitoring the host response.

In certain example embodiments, the method further comprises administering a pathogen treatment to the host, subsequent to the first timepoint and prior to the second timepoint and optionally identifying and/or measuring one or more biomarkers associated with immune response, treatment resistance, or both.

In certain example embodiments, the method further comprises repeating the method one or more times over a period of time thereby monitoring the host response and optionally treatment response, immune response, and/or treatment resistance over the period of time.

In certain example embodiments, wherein one or more steps of them method are performed using a nucleic acid detection system of the present invention described herein.

Described in certain example embodiments herein are methods of detecting target cell free nucleic acids in a sample, comprising: performing a method of the present invention described herein.

Described in certain example embodiments herein are devices, optionally a microfluidic devices, configured to detect nucleic acids comprising a sample loading module configured to receive a sample; a sample processing module at least configured to process a sample for optional nucleic acid extraction and/or concentration and nucleic acid detection; an optional nucleic acid extraction and/or concentration module configured to extract and/or concentrate nucleic acids in the sample; and a nucleic acid detection module configured to detect target nucleic acids.

In certain example embodiments, the sample processing module comprises one or more filters, wherein the filters are optionally membranes.

In certain example embodiments, the sample processing module is configured to separate plasma from blood.

In certain example embodiments, the optional nucleic acid extraction and/or concentration module comprises magnetic particles configured to bind, attach, or otherwise associate with nucleic acids.

In certain example embodiments, the device further comprises a movable magnet, wherein the movable magnet is at least operatively coupled to the nucleic acid extraction and/or concentration module.

In certain example embodiments, the device further comprises a power source.

In certain example embodiments, the device further comprises a heating element coupled to the sample processing module.

In certain example embodiments, the sample loading module, sample processing module, optional nucleic acid extraction and/or concentration module comprise one or more reservoirs, vessels, compartments, and/or regions each configured to contain a sample or component thereof.

In certain example embodiments, the nucleic acid detection module is configured to amplify a set of target regions in one or more target elements, and optionally comprises a set of amplification primers configured to amplify the set of target regions in one or more target elements, wherein primers of the set of amplification primers are optionally optimized for pooled amplification.

In certain example embodiments, the nucleic acid detection module comprises a nucleic acid detection system of the present invention described herein.

In certain example embodiments, the nucleic acid detection module comprises one or more flow channels, each flow channel comprising a sample loading region, optionally comprising one or more components of a nucleic acid detection system of the present invention described herein; a detector region comprising a one or more components of a nucleic acid detection system of the present invention described herein; and at least a first capture region and a second capture region, the first capture region comprising a first binding agent and the second capture region comprising a second binding agent.

In certain example embodiments, the nucleic acid detection module is a node, wherein each flow channel is arranged radially from a center node.

In certain example embodiments, the center node comprises transcription and/or amplification reagents.

In certain example embodiments, the device comprises one or more thermally differentiated zones disposed between the sample loading region and the center node.

In certain example embodiments, each flow channel is arranged in parallel.

In certain example embodiments, the detection construct comprises a first molecule on a first end and a second molecule on a second end.

In certain example embodiments, the nucleic acids are RNA, genomic DNA, cell free DNA, and/or circulating cell free DNA.

In certain example embodiments, the device further comprises one or more amplification reagents, optionally in the sample loading region.

In certain example embodiments, the one or more amplification reagents are nucleic acid sequence-based amplification (NASBA) reagent(s), recombinase polymerase amplification (RPA) reagent(s), loop-mediated isothermal amplification (LAMP) reagent(s), RT-LAMP reagent(s), strand displacement amplification (SDA) reagent(s), helicase-dependent amplification (HDA) reagent(s), nicking enzyme amplification reaction (NEAR) reagent(s), PCR reagent(s), multiple displacement amplification (MDA) reagent(s), rolling circle amplification (RCA) reagent(s), ligase chain reaction (LCR) reagent(s), or ramification amplification method (RAM) reagent(s).

In certain example embodiments, one or more of the modules are microfluidic modules.

In certain example embodiments, the device is configured as a point-of-care device.

In certain example embodiments, set of amplification primers of the nucleic acid detection system is contained in the sample loading region or the detector region.

In certain example embodiments, one or more Cas proteins, the set of guide polynucleotides, the oligonucleotide-based detection construct, or any combination thereof, are contained in the sample loading region, the detector region, or both.

Described in certain example embodiments herein are microfluidic devices configured to detect nucleic acids comprising one or more flow channels, each flow channel comprising a sample loading region optionally comprising one or more components of a nucleic acid detection system of the present invention described herein; a detector region comprising one or more components of a nucleic acid detection system of the present invention described herein; and at least a first capture region and a second capture region, the first capture region comprising a first binding agent and the second capture region comprising a second binding agent.

In certain example embodiments, the nucleic acid detection module is a node, wherein each flow channel is arranged radially from a center node.

In certain example embodiments, the center node comprises transcription and/or amplification reagents.

In certain example embodiments, the device further comprises one or more thermally differentiated zones disposed between the sample loading region and the center node.

In certain example embodiments, each flow channel is arranged in parallel.

In certain example embodiments, the detection construct comprises a first molecule on a first end and a second molecule on a second end.

In certain example embodiments, the nucleic acids are RNA, genomic DNA, cell free DNA, and/or circulating cell free DNA.

In certain example embodiments, the device further comprises one or more amplification reagents, optionally in the sample loading region.

In certain example embodiments, the one or more amplification reagents are nucleic acid sequence-based amplification (NASBA) reagent(s), recombinase polymerase amplification (RPA) reagent(s), loop-mediated isothermal amplification (LAMP) reagent(s), RT-LAMP reagent(s), strand displacement amplification (SDA) reagent(s), helicase-dependent amplification (HDA) reagent(s), nicking enzyme amplification reaction (NEAR) reagent(s), PCR reagent(s), multiple displacement amplification (MDA) reagent(s), rolling circle amplification (RCA) reagent(s), ligase chain reaction (LCR) reagent(s), ramification amplification method (RAM) reagent(s), or any combination thereof.

In certain example embodiments, the set of amplification primers of the nucleic acid detection system is contained in the sample loading region or the detector region.

In certain example embodiments, one or more Cas proteins, the set of guide polynucleotides, the oligonucleotide-based detection construct, or any combination thereof, are contained in the sample loading region, the detector region, or both.

In certain example embodiments, the device is configured as a point-of-care device. These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Single-plex PCR-Cas13 and single-plex RPA-Cas13 workflows achieve a limit of detection of 1 genome equivalents per reaction. In these experiments, the RPA and PCR primer pairs were designed to amplify a region of the IS6110 element of M.tb H37Rv. Genomic DNA was enzymatically fragmented to an average fragment size of 180 bp to simulate circulating, bacteria-derived cell-free DNA in blood. Cas13-based fluorescence is measured using a microplate spectrophotometer. (NTC=no template control; +control=10\hat{ }4 genome equivalents per reaction).

FIG. 2—Multiplexed PCR-Cas13 workflow improves limit of detection by an order of magnitude compared to singleplex PCR-Cas13. In the singleplex PCR-Cas13 workflow, a single primer was designed to amplify a region of the IS6110 element of M.tb H37Rv. In the multiplexed PCR-Cas13 workflow, 18 PCR primer pairs were designed to amplify regions in the IS6110 or IS1081 elements of M.tb H37Rv. Six replicates were tested at each dilution. Cas13-based fluorescence is measured using a microplate spectrophotometer. (NTC=no template control).

FIG. 3—Evaluation of specificity of PCR primers and their crRNAs against M. gordonae, M. abscessus, M. fortuitum, M. smegmatis, A. baumannii, E. faecalis, P. aeruginosa, E. coli, K. pneumoniae, S. epidermidis, S. aureus; NTC=no template control.

FIG. 4A-4B—RPA-Cas13 and multiplexed PCR-Cas13 workflows detect signal on 10 uL cell-free DNA extracted from clinical blood samples. (FIG. 4A) Fluorescent-based SHERLOCK assay signal from samples tested using an RPA primer pair targeting IS6110. (FIG. 4B) Heat map of fluorescent signal from samples tested using 18-multiplexed PCR primers that target IS6110 (green shaded primer/crRNA sets) or IS1081 (unshaded primer/crRNA sets). The 18×PCR primer/crRNA set is the experiment where all 18 crRNAs are pooled together. BWH1-BWH12 are the 12 presumed negative samples from patients in Boston in which there was no reason to suspect TB. Samples NG1-NG6 were negative by GeneXpert, culture, and an internal qPCR experiment. Samples PS1-PS6 were confirmed to be TB-positive by GeneXpert or culture and qPCR. NTC=no template control; +control=H37Rv at 100 genome equivalents per reaction.

FIG. 5—Lateral flow strip results of 10 μL of cfDNA extracted from clinical samples. Samples were tested using an RPA primer pair targeting IS6110.

FIG. 6A-6B—Lateral flow strip results from testing 1uL and 100 nL of cell-free DNA extracted from clinical blood samples (FIG. 6A) using single-plex RPA-Cas13 and (FIG. 6B) multiplexed PCR-Cas13 workflows. 100 nL of cell-free DNA was also tested using the multiplexed PCR-Cas13 workflow. (NTC=no template control; +=10 genome equivalents of fragmented DNA).

FIG. 7—Schematic showing the general strategy of target region and optional target element amplification, steps in optimizing, selection, and design amplification primers and amplified target regions and/or elements, and Cas-based detection of amplified target regions (e.g., amplicons produced by primers contained in the set of amplification primers). Target elements amplified can be tiled across a genome to, e.g., increase the assay sensitivity. Further, one or more target regions per target element can be amplified, to e.g., increase the assay sensitivity. As shown in FIG. 8A, amplification can be pooled. Amplification can be paired in the assay with pooled or parallel Cas-based detection (see also, e.g., FIG. 8A).

FIG. 8A-8D—Experimental and computational workflow of a WATSON assay. FIG. 8A. Schematic of singleplex SHERLOCK assay and WATSON assay workflows; in a singleplex SHERLOCK assay, a single genomic target is amplified, and this amplicon is detected by a single crRNA in a single CRISPR/Cas13 reaction; in a WATSON assay, tiled genomic targets are amplified in one pool, and amplicons are detected by CRISPR/Cas13 either in parallel amplicon-specific reactions or in one pooled reaction; FIG. 8B-8C. Computational workflow for identifying unique regions of the pathogen of interest; regions conserved across strains within the species (“In” group) are first identified; then regions present in related species (“Out” group) are excluded; Tiled primers and crRNA are designed to target unique genomic regions FIG. 8D. Heatmap showing individual and pooled crRNA performance (detection; Y axis) with singleplex and 18-plex pooled amplification (amplification; X axis) of Mtb genomic DNA using DropArray at a fixed concentration of 10³ genome equivalents per reaction.

FIG. 9A-9C—Evaluation of the WATSON assay limit of detection (LoD) on engineered samples. FIG. 9A. A WATSON assay and singleplex SHERLOCK assay fluorescence levels across a dilution series of fragmented Mtb gDNA; Heatmap of WATSON assay signals are shown for each individual crRNA tested in parallel and summarized as maximum individual signal (MIS,blue) as well as in a single pool of 18 crRNA (green); singleplex SHERLOCK assay signals are shown in orange (bottom row); FIG. 9B. Fluorescent signals are converted to a binary Test Result (positive=black; negative=white) based on a cut-off of >6 standard deviations above background; FIG. 9C. WATSON assay and singleplex SHERLOCK assay LoD shown for six replicate reactions at each dilution; WATSON assay using parallel detection, summarized as the maximum individual signal (MIS) (top, in blue as represented in greyscale); WATSON assay using pooled detection in a single reaction (middle, in green as represented in greyscale); or singleplex SHERLOCK assay crRNA signal (bottom, in orange as represented in greyscale); variable signals are observed across replicates at the LoD (red dotted line as represented in greyscale); ntc=no template control.

FIG. 10—A table showing clinic samples metadata, tuberculosis (TB) tests performed, and TB diagnosis. HC=Healthy control, SUS=Suspected TB patient who presented with a cough for more than a week but tested negative by culture and GeneXpert, CFM-SA=Confirmed TB patient from South African who tested positive by culture and GeneXpert, +=TB-positive, −=TB-negative. * Sputum-based culture and/or GeneXpert are WHO-recognized TB tests and were used as the reference standard and comparator test, respectively, to determine the TB status of the participants who presented with a respiratory illness. ** A cfDNA-based qPCR assay was performed in this study as a measure of amplifiable cfDNA. Shown are the Ct values. A “Negative” qPCR result reflects a sample where amplification did not cross the assay cut-off threshold.*** WATSON assay using parallel detection, with the fluorescent signal from each of 18 crRNA converted to a binary test result, was performed in this study. Shown are the number of crRNA that produced a signal >6 standard deviations above background. † TB status determined based on the results from culture and GeneXpert testing. †† WATSON assay results based on pooled detection.

FIG. 11A-11C—Evaluation of WATSON assay on clinical samples. cfDNA extracted from the equivalent of 400 μL of patient plasma was amplified by a single pool of 18 primer sets. FIG. 11A. Heatmap of WATSON assay fluorescence for 41 clinical samples for each crRNA tested in parallel and summarized as maximum individual signal (MIS, blue), and for all 18 crRNA pooled together in a single reaction (bottom row labeled “18 pooled guides”). For positive samples, a series of 10-fold dilutions of extracted cfDNA was tested; the equivalent volume of plasma from which the amount of cfDNA in each dilution is extracted is indicated above (HC=Healthy control, SUS=Suspected TB patient who presented with a cough for more than a week but tested negative by culture and GeneXpert, CFM=Confirmed TB patient that tested positive by culture and/or GeneXpert) FIG. 11B. Fluorescent signals converted to binary Test Result based on a cut-off signal (>6 standard deviations above background); positive (black) and negative (white) signals; FIG. 11C. Summary of estimated cfDNA abundance in confirmed tuberculosis samples based on the minimum plasma volume needed to observe a positive signal by any of the eight IS1081 crRNA.

FIG. 12A-12B—A field deployable assay format using RPA and lateral flow readout. FIG. 12A. Singleplex PCR (left graph) and RPA (right graph) amplification using a primer pair targeting IS6110_2 combined with CRISPR-Cas13 detection show similar limits of detection (indicated by dotted red line) based on six replicates at each dilution of fragmented Mtb genomic DNA. FIG. 12B. WATSON assay on cfDNA extracted from the equivalent of 400 μL of patient plasma samples using pooled detection, evaluated using either lateral flow readout from image analysis of lateral flow strip (“lateral flow readout”) or fluorescence readout (“fluorescence readout”); fluorescence (RFU) and Lateral flow readouts are converted to a binary Test Result (positive=black; negative=white) based on a cut-off (>6 standard deviations above background). (HC=Healthy Control, SUS=Suspected TB Patients who presented with a cough for more than a week but tested negative by culture and GeneXpert, CFM=Confirmed TB Patients who tested positive by culture and/or GeneXpert). Representative lateral flow strips from negative and positive clinical samples as well as controls are shown above. More images of lateral flow strips are shown in FIG. 18A-18B.

FIG. 13A-13B—Guide RNA performance. FIG. 13A. Heatmap depicting performance of guides against their corresponding targets; 1-3 crRNA guides were designed for each target, depending on the sequence space between primers; all guides were tested against singleplex amplified targets and the best performing ones were chosen. FIG. 13B. Heatmap depicting single best performing guide for each target.

FIG. 14A-14B—FIG. 14A. Size profiles show the representation of fragment size distribution after NEBNext Fragmentase reaction. Fragmentation profiles are shown for M. tuberculosis H37Rv genomic DNA and fragmented human genomic DNA. FIG. 14B. Heatmap of the WATSON assay using droplet platform over a range of M. tuberculosis genomic DNA concentrations for two different materials. The first is fragmented M. tuberculosis genomic DNA with an average fragment size of 180 bp (shown in FIG. 14A. TB180) with a constant background of 1 genomic equivalent per uL of human fragmented gDNA (shown in FIG. 14A. Human). The second is the same M. tuberculosis genomic DNA without human background.

FIG. 15—Computer simulation of stochastic distribution of cfDNA fragments (assumed to have constant length of 200 bp) when different genome equivalents are considered (genome length assumed to be 4 million bp). Simulations are shown at 1 GE (4 million/200=20,000 total fragments), about 35% of fragments were expected to be absent, about 35% of fragments were expected to be present as 1 copy, about 18% of fragments were expected to be present as 2 copies, and so on.

FIG. 16—Limit of detection of singleplex RPA-Cas13 assay on 4 strains of M. tuberculosis (Erdman, CDC 1551, HN878, and H37Rv, as well as M. bovis Bacille Calmette-Guerin (BCG). The number in parenthesis below each strain indicates the number of copies of IS6110 in that strain. All tests used fragmented gDNA, with an average fragment size of 120-150 bp. Control samples are 3 no template controls (NTC) and one positive control comprising fragmented gDNA from H37Rv.

FIG. 17A-17B—FIG. 17A. Specificity of the WATSON assay and FIG. 17B. singleplex RPA-Cas13 against genomic DNA from other related bacteria: Mycobacterium abscessus (M.a), M. fortuitum (M.f), M. gordonae (M.g) and M. smegmatis (M.s) as well as less closely related species Acinetobacter baumanii (A.b), Pseudomonas aeruginosa (P.a), Escherichia coli (E.c), Klebsiella pneumoniae (K.p), Staphylococcus epidermidis (S.e), and Staphylococcus aureus (S.a). 1e4 genome equivalents per reaction was used for each pathogen shown in FIG. 17A. and 5e3 genome equivalents per reaction for those shown in FIG. 17B. with the exception of H37Rv, which was tested at 1e2 genome equivalents per reaction.

FIG. 18A-18B—Images of lateral flow strips (FIG. 18A) for a dilution series of fragmented Mtb gDNA that were amplified via singleplex PCR (left) and RPA (right) using a primer pair targeting IS6110_2 combined with CRISPR-Cas13 detection. Both methods of amplification, detection and lateral flow readout show a similar limit of detection of 0.1-1 genome equivalents per reaction, which is concordant with the limit of detection using fluorescence readout, as shown in FIG. 12A. Six replicates were run at each gDNA concentration; FIG. 18B. for clinical samples that were amplified and detected by the WATSON assay with a lateral flow readout. cfDNA was extracted from the equivalent of 400 μL, 400 μL, and 400 μL of patient plasma as indicated. HC=Healthy control, SUS=Suspected TB patient who presented with a cough for more than a week but tested negative for culture and GeneXpert, CFM=Confirmed TB patient who tested positive by culture and/or GeneXpert, NTC=no template control, +=fragmented DNA from H37Rv at 1e2 genome equivalents per reaction.

FIG. 19A-19B—Schematic illustration of and exemplary embodiment of detecting Cas13 activity on a lateral flow strip. (FIG. 19A) When target DNA is recognized by the crRNA/Cas13 complex, Cas13 activity is detected as a visible Test line on the lateral flow strip with a weak or no line visible at the Control line. (FIG. 19B) When target DNA is absent and Cas13 is not activated, only a visible line appears at the Control line.

FIG. 20A-20B—Comparison of WATSON assay and singleplex SHERLOCK assay on clinical samples from South Africa. cfDNA extracted from the equivalent of 400 μL of patient plasma was amplified in separate experiments by either a pool of 18 primers (WATSON) or a single primer pair (Singleplex SHERLOCK assay). FIG. 20A Heatmap of WATSON fluorescence signals for 11 clinical samples are shown for each individual crRNA tested in parallel (blue) as well as in a single pool of 18 crRNAs (green, as represented in greyscale); singleplex SHERLOCK assay signal using a crRNA targeting IS6110_9, which coincides with the same region of IS6110 as the cfDNA-based qPCR assay that was also used to evaluate these samples (IS6110_9) (orange, as represented in greyscale). The fluorescent signal corresponding to 6 standard deviations above the average fluorescence of the no template control samples is shown as a visual representation of the analysis performed to convert the fluorescent signals in (FIG. 20A) to the binary test results shown in (FIG. 20B). FIG. 20B Fluorescent signals converted to binary Test Result based on a cut-off signal >6 standard deviations above the no template control. (CFM-SA=Confirmed TB patient from South African cohort that tested positive by culture and GeneXpert; NTC=no template control; TB_e2=positive control where fragmented gDNA from Mtb H37Rv at 102 GE/reaction was used as the template). Source data are provided as a Source Data file of Thakku et al., Nature Communications (2023) 14: Article 1803, which is incorporated by reference herein.

FIG. 21—A table of metadata, tuberculosis (TB) tests performed, and TB diagnosis for clinical samples from Ugandan and American cohorts. HC—Healthy control, SUS Suspected TB patient who presented with a cough for more than a week but tested negative by culture and GeneXpert, CFM-UP Confirmed TB patient from Ugandan or Palo Alto cohort who tested positive by culture and/or GeneXpert, ND Not done, +=TB-positive, −=TB-negative. *Sputum-based culture and/or GeneXpert are WHO-recognized TB tests and were used as the reference standard and comparator test, respectively, to determine the TB status of the participants who presented with a respiratory illness. **A cfDNA-based qPCR assay was performed in this study as a measure of amplifiable cfDNA. Shown are the Ct values. A “Negative” qPCR result reflects a sample where amplification did not cross the assay cut-off threshold. ***WATSON assay using parallel detection, with the fluorescent signal from each of 18 crRNA converted to a binary test result, was performed in this study. Shown are the number of crRNA that produced a signal >6 standard deviations above the no template control sample. †TB status determined based on the results from culture and/or GeneXpert testing.

FIG. 22—Limit of detection of WATSON on 4 strains of M. tuberculosis (H37Rv, Erdman, CDC 1551, and HN878, as well as M. bovis Bacille Calmette-Guerin (BCG)). The number in parenthesis below each strain indicates the number of copies of IS6110 in that strain. All tests used fragmented gDNA, with an average fragment size of 120-150 bp. (ntc=no template control).

FIG. 23A-23B—Schematic illustration of detecting Cas13 activity on a lateral flow strip. FIG. 23A. When nucleic acid target is recognized by the crRNA/Cas13 complex, Cas13 activity is detected as a visible Test line on the lateral flow strip with a weak or no line visible at the Control line. FIG. 23B. When nucleic acid target is absent and Cas13 is not activated, only a visible line appears at the Control line.

FIG. 24—Plot of the maximum number of primers in a pool as a function of the number of 5-mer sequences at the 3′ end that are allowed in the pool as determined by the primer design rules (See Methods). Primers were designed to target the IS6110 and IS1081 regions and minimize 3′-3′ interactions between pairs. The top 100 ranked and filtered 5-mers (as described in the Methods section) were sampled at each number of allowed 5-mer sequences. The maximum number of primers that can be pooled together according to our design rules is 18, where each primer pair has one of 11 5-mer sequences at its 3′ end.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Circulating cell free DNA (cfDNA) are DNA fragments that are released from cells through a variety of mechanisms into surrounding bodily fluids. The blood plasma is an exceptionally rich source of cfDNA, although cfDNA can be found in other bodily fluids as well. Sources of circulating cell-free DNA are varied but include tumors, fetal, mitochondria, and pathogens. As such, cfDNA is a rich source of information regarding the health profile of an individual. Not surprisingly, there has been a significant interest and efforts into developing various non-invasive assays, such as liquid biopsies, for diseases, disorders, and health status based on detection of cfDNA.

Although these efforts have increased the understanding and moved the field forward towards clinically relevant cfDNA assays, there are significant technical and biological challenges that cfDNA presents for conventional nucleic acid detection techniques used to analyze cfDNA such as next generation sequencing and other amplification-based techniques. cfDNA is difficult to detect using sequencing approaches as it is often low in abundance in a sample. Further, patient variability in abundance of cfDNA exists. Additionally, clinically relevant markers are often rare alleles or mutations. Moreover, cfDNA are degraded fragments and markers may be present in their entireties in some fragments but be split amongst others. Tumor sequencing data suggests that clinically relevant mutations are often non-uniformly distributed within a population, thus increasing the input requirements needed for a reliable sequence-based assay. Even if the panel of markers is expanded to reduce the input requirements, the non-uniform distribution of mutation frequencies can mean that dramatically more sequencing depth is required to attain the desired sensitivity to achieve clinical relevancy.

A well understood problem with next-generation sequencing techniques is the relatively high per-base error rate. Although barcoding strategies and computational post-processing has improved this with traditional nucleic acid templates, the result is that the techniques then require higher read depth. In other words, individual molecules are sequenced more than once, with unique molecular identifiers used to group the multiple reads to a single read and correction of errors made by consensus within the grouped reads. This oversampling inflates the total sequencing depth (as opposed to unique sequencing approaches) and ultimately increases the sequencing requirements. Put simply, there would not be enough sample available to attain the coverage needed to for this be feasible. Even if the panel expansion approach were considered to address this challenge, the total amount of sequencing needed to attain the necessary coverage with respect to each target in the panel can result in an exorbitant sequencing cost, making this approach unrealistic. Finally, such approaches are ill-suited for in-field and rapidly deployable methods due to their reliance on sophisticated equipment and processing.

These challenges lead to, inter alia, false negative rates that are unacceptable in the clinical setting.

False positive rates and reliable detection remain a challenge with some conventional approaches used to detect cfDNA. The unacceptable false positive rates observed is inherent to these systems as their sensitivity is increased. The short fragment length and low abundance are major factors in detection reliability. This is further amplified when a target within the cfDNA occurs infrequently. See e.g., Volkmar et al. Genes Crhomosom. Cancer. 2018. 57:123-129.

Thus, there exists an urgent need for improved strategies and techniques for detecting and analyzing cfDNA.

Embodiments disclosed herein provide a diagnostic platform that can rapidly and accurately identify the presence of bacterial or other pathogens and discriminate between strains in clinical samples. Advantageously, the diagnostic platform is rapid, sensitive and can be adapted to any microbe through a bioinformatics pipeline that identifies multiple targets across the entire genome. Methods are also provided for detection of pathogen nucleic acids in a biofluid (e.g., blood or urine) utilizing circulating cell-free nucleic acids and find use in detection of multiple pathogens, identifying markers of antibiotic resistance in pathogens, as well as monitoring host response to infection.

Embodiments disclosed herein provide an assay platform that includes pooled, tiled, and/or multiplexed amplification coupled with tiled and/or multiplexed detection of two or more target regions within one or more target elements. Without being bound by theory, coupling tiled target region amplification with optionally pooled Cas-based detection the assay increases the sensitivity of the assay to detect low abundance nucleic acids, such as cell free DNA, in a sample. Pooling at the amplification stage can allow for detection of low abundance nucleic acids in low volume samples. In some embodiments, one or more of the target elements amplified are repeated elements within the genome of an organism to be detected. Without being bound by theory, amplifying repeated elements within a genome can improve detection of low abundance nucleic acids, particularly in low volume samples.

Amplified target regions of target elements can be detected by any suitable detection system and/or method. In some embodiments, detection utilizes a CRISPR-Cas system, particularly one that includes a Cas having collateral cleavage activity. A Clustered Regularly Interspaced Short Palindromic Repeats (CRISPR) and CRISPR associated enzymes (Cas) are together the hallmark of bacterial defense systems. CRISPR-Cas systems have been shown to be reprogrammable for a variety of genome editing applications, and more recently, for CRISPR-based diagnostics. In particular, CRISPR-Cas system that utilize RNA targeting effectors, which may also be referred to as a SHERLOCK (Specific High-sensitivity Enzymatic Reporter unLOCKing) assay, has been shown to detect a single genomic target at attomolar concentrations. As described herein, CRISPR detection systems, such as a SHERLOCK system, are used to detect the amplified target regions of target elements and to provide a robust CRISPR-based diagnostic.

Without being bound by theory, combination of pooled and tiled, amplification of one or more target region(s) within one or more target elements of nucleic acids in the sample with the CRISPR detection system allows for the detection of even small amounts of fragmented nucleic acids (e.g., cfDNA), optionally of pathogenic organisms, present in samples. Because CRISPR-Cas introduces a second layer of specificity, amplification need not be exclusive to the targets of interest, and non-specific amplification of background may be permissible, with less restrictive initial amplification further increase the system's sensitivity in detecting bacterial DNA. Similarly, the specificity of the guide RNA used in CRISPR-Cas based diagnostic can be adjusted by the design parameters placed on the nucleic acid targets of interest as described herein.

Embodiments herein also provide methods of pooled and tiled amplification of target nucleic acids followed by detecting the amplified target regions within target elements using a CRISPR-Cas based detection method. In some embodiments, the methods are tailored for a simple, easy to use, and/or field deployable point of care device.

Embodiments herein also provide devices configured to perform one or more steps of a method of pooled and tiled multiplexed amplification of target regions in target elements within a genome or other nucleic acid followed by detecting the amplified target regions of target elements using a CRISPR-Cas based detection method. In some embodiments, the device is a field-deployable, point of care device.

### Cell Free Nucleic Acid Detection Systems

Described in several embodiments herein are nucleic acid, particularly cell free nucleic acid detection systems. The systems can be used to detect, for example, fragmented nucleic acids (e.g., fragmented genomes), and more particularly cell free DNA or circulating cell free DNA (ccfDNA). The nucleic acid detection system can include one or more CRISPR-Cas detection systems, where each CRISPR-Cas detection system contains a Cas effector protein and one or more guide RNAs.

As used herein “target element” refers a specific region or sequence of interest within a genome to be amplified. Target elements as used in this context are typically unique to one or more (e.g., a genus, subgenus, and/or the like) organisms, such as microorganisms or viruses, to be detected. Target elements include, without limitation, genes, regulatory sequences, non-coding sequences, and/or the like. Target sequences can be DNA.

The nucleic acid detection systems described herein can be used, e.g., in a method that includes amplification of one or more amplification target regions in one or more target elements followed by detection, such as CRISPR-Cas-based detection, of the amplified target regions. As used herein “amplification target region” is a region composed of a target element or a region within a target element selected to be amplified using a suitable amplification reaction as is further described herein. The systems and methods of the present invention can be configured to amplify one or more amplification target regions in a single target element.

In some embodiments, the nucleic acid system is capable of detecting less than one genome equivalent (GE) of a target, such as target element. In some embodiments, the nucleic acid system is capable of detecting about 0.001 to 1 GE of a target, such as a target element. In some embodiments, the nucleic acid system is capable of detecting about 0.001 to 0.01, 0.01 to 0.1, or 0.1 to 1 GE of a target, such as a target element.

In some embodiments, the nucleic acid system has a sensitivity of detection of about 60, to/or 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or about 100 percent sensitivity. In some embodiments, the nucleic acid system has a sensitivity of detection of at least 60 percent, at least 65 percent, at least 70 percent, at least 75 percent, at least 80 percent, at least 85 percent, at least 90 percent, at least 95 percent, or at least 100 percent.

In some embodiments, the nucleic acid system has a specificity for detecting a specific species or variant of organisms (e.g., a microorganism or virus) of at least 95, 96, 97, 98, 99, 99.1, 99.2, 99.3, 99.4, 99.5, 99.6, 99.7, 99.8, or 99.9%. In some embodiments, the nucleic acid system can have a specificity for detecting a specific species or variant of organisms (e.g., a microorganism or virus) of 100 percent.

The systems can be used to detect the presence of one or more organisms and/or discriminating between one or more organisms in a sample and/or in a subject from which the sample is obtained.

In certain exemplary embodiments, the nucleic acid detection systems are composed of a set of amplification primers configured to amplify a set of target regions in one or more target elements, wherein primers of the set of amplification primers are optionally optimized for pooled amplification, one or more Cas proteins having collateral activity, a set of guide polynucleotides comprising a guide polynucleotide specific for each target region amplified by the set of amplification primers, and wherein each of the guide polynucleotides in the set of guide polynucleotides is capable of forming a CRISPR-Cas complex with the one or more Cas proteins, and an oligonucleotide-based detection construct comprising a non-target sequence, wherein the non-target sequence is configured to be cleaved by the collateral activity of the one or more Cas proteins.

In some embodiments, the one or more guide polynucleotides are optimized for pooled detection. In some embodiments, the one or more guide polynucleotides are optimized for parallel detection.

In some embodiments, the set of amplification primers are optimized for pooled detection by being configured to minimize 3′ to 3′ end interactions between primers within the pool.

In some embodiments, the guide polynucleotides of the set of guide polynucleotides are optimized for pooled detection.

In some embodiments, the set of amplification primers comprises 1-500 or more amplification primers, optionally 1-100 or more 2-100 more. In some embodiments, the set of amplification primers includes 1 to/or 2 or/to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112,113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more amplification primers.

In some embodiments, the set of guide polynucleotides comprises 1-500, optionally 1-50 or more or 2-50 or more guide polynucleotides. In some embodiments, the set of guide polynucleotides includes 1 to/or 2 to/or 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more guide polynucleotides.

In some embodiments, at least two of the two or more guide polynucleotides of the guide polynucleotide set are capable of hybridizing to different target sequences of different amplified target regions of different target elements. In some embodiments, at least two of the two or more guide polynucleotides of the guide polynucleotide set are capable of hybridizing to different target sequences of different amplified target regions of the same target element.

In some embodiments, the system is configured to amplify and/or detect one or more or two or more (e.g., 1-50, 2-50, 1-500, 2-500) target regions. In some embodiments, the system is configured to detect 1 to/or, 2 to/or, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more target regions.

In some embodiments, the system is configured to amplify and/or detect one or more or two or more (e.g., 1-50, 2-50, 1-500, 2-500) target elements. In some embodiments, the system is configured to detect 1 to/or, 2 to/or, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more target elements. The one or more target elements can be elements (e.g., genes, non-transcribed regions, and/or the like) in DNA, such as cell free DNA (cfDNA) and/or elements in circulating cell free DNA (ccfDNA). In some embodiments, at least one of the one or more target elements is a repetitive target element. In some embodiments, the one or more target elements are from or specific to a microorganism or a virus, optionally a pathogenic microorganism or virus.

In some embodiments, the amplification primers in the set of amplification primers are or include PCR primers, isothermal amplification primers, proximity dependent probes, or a combination thereof. The proximity dependent probes can include one or more of a forward primer binding site, a reverse primer binding site, and a reverse polymerase binding site. The proximity dependent probes can be molecular inversion probes (MIPs), padlock probes, or split-ligation probes. The proximity dependent probes can be linked by ligation, splinted ligation, hybridization, or proximity extension. The proximity dependent probes can have a gap region that, upon binding to a target sequence and gap filling, comprises a gap-filled sequence. The gap-filed sequence can include the guide polynucleotide recognition sequence. The gap-filled sequence can include modified nucleotides that have or form a capture moiety. In some embodiments, the nucleic acid detection system further includes a capture agent that binds the capture moiety of the modified nucleotides. In some embodiments, the modified nucleotides are biotinylated nucleotides and the capture agent is streptavidin or a streptavidin coated surface. In some embodiments, the proximity dependent probe is a molecular inversion probe (MIP), and wherein the MIP comprises a first target binding sequence and a second target binding sequence linked by a linking region, the linking region comprising one or more of a forward primer binding sequence, a reverse primer binding sequence, a RNA polymerase binding sequence, a guide polynucleotide binding sequence, and a barcode. In some embodiments, the first target binding sequence and the second target binding sequence hybridize on the target sequence directly adjacent to one another. In some embodiments, the first and second target binding sequence hybridize on the target sequence such that there is at least a single nucleotide gap region between the first and second target binding sequence. In some embodiments, filling the gap region between the first and second targeting binding sequence generates a guide polynucleotide recognition sequence.

In some embodiments, one or more of the plurality of amplification probes comprises an origin-specific barcode, a set-specific barcode, a unique molecular identifier (UMI), or any combination thereof.

In some embodiments, one or more primers of the set of amplification primers configured to amplify a set of target regions comprise an in vitro transcription promoter sequence, optionally at the 5′end of the one or more primers. In some embodiments, the in vitro transcription promoter sequences is for a T7 promoter, SP6 promoter, or a T3 promoter.

In some embodiments, the amplification primer set is configured to amplify at least one or more target regions of a target element that covers at least 10 percent, at least 20 percent, at least 30 percent, at least 40 percent, at least 50 percent, or at least 60 percent of a target element.

In some embodiments, the amplification primer set is configured to amplify at least one or more target regions of a target element that covers at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 percent of a target element. In some embodiments, the amplification primer set is configured to amplify at least one or more target regions of a target element that covers about 10 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 percent of a target element. In some embodiments, the amplification primer set is configured to amplify at least one or more target regions of a target element that covers about 10 to/or 100 percent of a target element. In some embodiments, the amplification primer set is configured to amplify at least one or more target regions of a target element that covers about 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of a target element.

In some embodiments, the nucleic acid detection system further includes amplification reagents for amplifying the set of target regions. In some embodiments, the nucleic acid detection system includes amplification reagents for amplifying amplification target regions of a target element. The amplification reagents can be or include nucleic acid sequence-based amplification (NASBA) reagent(s), recombinase polymerase amplification (RPA) reagent(s), loop-mediated isothermal amplification (LAMP) reagent(s), RT-LAMP reagent(s), strand displacement amplification (SDA) reagent(s), helicase-dependent amplification (HDA) reagent(s), nicking enzyme amplification reaction (NEAR) reagent(s), Polymerase Chain Reaction (PCR) reagent(s), multiple displacement amplification (MDA) reagent(s), rolling circle amplification (RCA) reagent(s), ligase chain reaction (LCR) reagent(s), ramification amplification method (RAM) reagent(s), or any combination thereof.

In some embodiments, the nucleic acid detection system further includes a nucleic acid enrichment reagent. In some embodiments, the nucleic acid enrichment reagent is a cfDNA and/or ccfDNA enrichment agent. The nucleic acid enrichment reagent can include a DNA methylation enrichment agents, size selection reagents to enrich for a nucleic acid, a magnetic or paramagnetic particle configured to bind nucleic acids, or any combination thereof.

In some embodiments, the nucleic acid detection system further includes one or more nuclease inactivation reagents, microorganism or virus inactivation reagents, or both.

### CRISPR-Cas Detection Systems and CRISPR-Cas Proteins

CRISPR Cas based systems that allow for detection down to femtomolar sensitivity can be combined with initial amplification of the target to allow for detectable attomolar concentrations, possibly lower. SHERLOCK assay and DETECTR assay employ preamplification systems with Cas enzymes, for example Cas13a or Cas 12a that target ssRNA and dsDNA respectively. See e.g., Kaminski et al. Nat. Biomed Eng. 5: 643-656 (2021); Mustafa and Makhawi J Clin Microbiol 59(3). 2021. doi:https://doi.org/10.1128/JCM.00745-20; Srivastava et al. Front. Mol. Biosci., 23 Dec. 2020; https://doi.org/10.3389/fmolb.2020.582499; Yan et al., 2019. Cell Biol Toxicol. 35:489-492. In some embodiments, target regions and/or target element(s) can be amplified by a suitable amplification method prior to CRISRP-Cas based detection of target element(s). In some embodiments, the target element(s) and/or target regions therein can be tiled across the genome as is described elsewhere herein. Amplification can be performed using PCR, RPA, and/or the like. Other suitable amplification methods and techniques are described elsewhere herein.

In certain embodiments, the CRISPR-Cas system that can be used for detection includes a Cas polypeptide that has one or more collateral activities, such as collateral nucleic acid cleavage activity. Such activities can be utilized in an assay, such as a detection assay for a target nucleic acid described elsewhere herein. In certain example embodiments, a Cas that has collateral activity (e.g., collateral nucleic acid cleavage activity) that can be included in the CRISPR-Cas system is a Cas13 (e.g., a Cas13a, 13b, Cas13c and/or Cas13d) and/or a Cas12 (e.g., Cas 12a, 12b, 12c, 12c1, 12c2, 12d, 12e, 12a1, 12g1, 12h1, 12i1, 12f (also known as Cas14)). In certain embodiments, the Cas effector can be a Type V-G Cas as in WO 2019/17842, a Type V-I Cas as in WO 2019178427, as Type III Cas as in WO 2019/22555; a Type V Cas or other Cas as in WO2021/050534, a Cas as in WO 2020/1041569, a Cas as in WO 2021/046442, or any combination thereof.

The nucleic acid detection system includes one or more CRISPR-Cas proteins. In some embodiments, the one or more CRISPR-Cas proteins is/are an RNA-targeting protein, a DNA-targeting protein, or any combination thereof. In some embodiments, the one or more CRISPR-Cas protein is a Class 2 CRISPR-Cas protein. In some embodiments, the one or more CRISPR-Cas protein is a Type V or Type VI CRISPR-Cas protein. In some embodiments, the one or more CRISPR-Cas proteins is a Cas13, a Cas 12, or any combination thereof. In some embodiments, the Cas13 is a Cas13a, Cas13b, Cas13c, a Cas13d, or a combination thereof. In some embodiments, the Cas12 is a Cas12a, Cas12b, or Cas12c. In some embodiments, each of the one or more CRISPR-Cas proteins has a different polynucleotide cutting preference and wherein the polynucleotide cutting preference is matched to a guide polynucleotide for a particular amplified target region and/or target element. In some embodiments, the nucleic acid detection system includes a Class 2 CRISPR-Cas protein.

In some embodiments, the nucleic acid detection system includes two or more CRISPR-Cas systems, wherein the two or more CRISPR systems comprise an RNA-targeting effector protein, a DNA-targeting effector protein, or any combination thereof. In some embodiments, the two or more CRISPR-Cas systems are each a Cas13 system, as Cas12 system, or a combination thereof, optionally wherein the Cas 12 system is a Cpf1, c2c1, and/or Cas12f system and/or the Cas 13 system is Cas 13a, Cas 13b, Cas 13c, and/or Cas13d system. The Cas12 and the Cas 13 system can have different cutting preferences which can allow for the use of different detection constructs each incorporating a different detectable label and motif such that they can be specifically cleaved by the different Cas orthologues. In general, Cas 12 systems target dsDNA while Cas 13 systems target ssDNA. Specific target preferences are further discussed elsewhere herein such as with respect to Cas13 and Cas12 polypeptides below.

In certain example embodiments, a Cas is a Cas12 (e.g., Cas 12a, 12b, 12c, 12c1, 12c2, 12d, 12e, 12a1, 12g1, 12h1, 12i1, 12f (also known as Cas14)). In some embodiments, the Cas polypeptide is thermostable. A thermostable protein as used herein comprises a protein that retains catalytic activity at a temperature at or above 32 degrees C., 33 degrees C., 34 degrees C., 35 degrees C., 36 degrees C., 37 degrees C., 38 degrees C., 39 degrees C., 40 degrees C., 41 degrees C., 42 degrees C., 43 degrees C., 44 degrees C., 45 degrees C., 46 degrees C., 47 degrees C., 48 degrees C., 49 degrees C., 50 degrees C., 51 degrees C., 52 degrees C., 53 degrees C., 54 degrees C., 55 degrees C., 56 degrees C., 57 degrees C., 58 degrees C., 59 degrees C., 60 degrees C., 61 degrees C., 62 degrees C., 63 degrees C., 64 degrees C., 65 degrees C., 66 degrees C., 67 degrees C., 68 degrees C., 69 degrees C., 70 degrees C., 71 degrees C., 72 degrees C. In certain example embodiments, the protein is thermostable at or above 55 degrees C.

### Cas3 Polypeptides

In certain example embodiments, the CRISPR-Cas system includes a Cas13 (e.g., a Cas13a, 13b, Cas13c and/or Cas13d). Cas13's non-specific RNase activity can be leveraged to cleave reporters upon target recognition, allowing for the design of sensitive and specific diagnostics using Cas13, including single nucleotide variants, detection based on rRNA sequences, screening for drug resistance, monitoring microbe outbreaks, genetic perturbations, and screening of environmental samples, as described, for example, in PCT/US18/054472 filed Oct. 22, 2018 at [0183]-[0327], incorporated herein by reference; WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR Based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and U.S. Pat. No. 62,767,077 filed Nov. 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed Dec. 21, 2018 entitled “Novel CRISPR Enzymes and Systems”, PCT/US18/67225 filed Dec. 21, 2018 entitled “Novel CRISPR Enzymes and Systems” and PCT/US18/67307 filed Dec. 21, 2018 entitled “Novel CRISPR Enzymes and Systems”, U.S. 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26 2018 entitled “Novel Cas12b Enzymes and Systems”, U.S. 715,640 filed August 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox DBT, et al., RNA editing with CRISPR-Cas13, Science. 2017 Nov. 24; 358(6366):1019-1027; Gootenberg J S, et al., Multiplexed and portable nucleic acid detection platform with Cas13, Cas12a, and Csm6., Science. 2018 Apr. 27; 360(6387):439-444; Gootenberg J S, et al., Nucleic acid detection with CRISPR-Cas13a/C2c2., Science. 2017 Apr. 28; 356(6336):438-442; Abudayyeh 00, et al., RNA targeting with CRISPR-Cas13, Nature. 2017 Oct. 12; 550(7675):280-284; Smargon A A, et al., Cas13b Is a Type VI-B CRISPR-Associated RNA-Guided RNase Differentially Regulated by Accessory Proteins Csx27 and Csx28. Mol Cell. 2017 Feb. 16; 65(4):618-630.e7; Abudayyeh 00, et al., C2c2 is a single-component programmable RNA-guided RNA-targeting CRISPR effector, Science. 2016 Aug. 5; 353(6299):aaf5573; Yang L, et al., Engineering and optimizing deaminase fusions for genome editing. Nat Commun. 2016 Nov. 2; 7:13330, Myrvhold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15(3):169-182, Zhang et al., “Two HPEN domains dictate CRISPR RNA maturation and target cleavage in Cas13d.” Nat. Comm. 10:2544 (2019), Patchsung et al., 2020. Nat. Biomed. Eng. 4:1140-1149; Aquino-Jarquin, G. Drug Discov. Today. 2021. 26(8):2025-2035; Fozouni et al., 2020. Amplification-free detection of SARS-CoV-2 with CRISPR-Cas13a and mobile phone microscopy. Cell. 184:323-333; Lotfi and Rezaei. 2020. CRISPR/Cas13: A potential therapeutic option of COVID-19 Biomedicine & Pharmacotherapy. 131:110738; Khan et al. 2020. CRISPR-Cas13 enzymology rapidly detects SARS-CoV-2 fragments in a clinical setting. medRxiv; doi: https://doi.org/10.1101/2020.12.17.20228593; Schermer et al., Rapid SARS-CoV-2 testing in primary material based on a novel multiplex RT-LAMP assay. PLoS One. https://doi.org/10.1371[journal.pone.0238612; Joung et al., “Detection of SARS-CoV-2 with SHERLOCK One-Pot TestingN Engl J Med 2020; 383:1492-1494” DOI: 10.1056/NEJMc2026172; Joung et al., “Point-of-care testing for COVID-19 using SHERLOCK diagnostics” medRxiv. Preprint. 2020 May 8. doi: 10.1101/2020.05.04.20091231; WO 2017/218573; WO 2019006471; US 20200010878; US 20200010879; US 20190177775; US 20180208977; US 20180208976; US 20190177775; U.S. Pat. No. 10,392,616; U.S. Provisional Application Ser. No. 62/351,172; the disclosure of each can be adapted for use with the present invention in view of the description provided herein and each of which is incorporated herein by reference in its entirety.

In particular embodiments, the Type VI RNA-targeting Cas enzyme is C2c2. In other example embodiments, the Type VI RNA-targeting Cas enzyme is Cas 13b. In particular embodiments, the homologue or orthologue of a Type VI protein such as C2c2 as referred to herein has a sequence homology or identity of at least 30%, or at least 40%, or at least 50%, or at least 60%, or at least 70%, or at least 80%, more preferably at least 85%, even more preferably at least 90%, such as for instance at least 95% with a Type VI protein such as C2c2 (e.g., based on the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2). In further embodiments, the homologue or orthologue of a Type VI protein such as C2c2 as referred to herein has a sequence identity of at least 30%, or at least 40%, or at least 50%, or at least 60%, or at least 70%, or at least 80%, more preferably at least 85%, even more preferably at least 90%, such as for instance at least 95% with the wild type C2c2 (e.g., based on the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2).

In embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In embodiments, the effector protein comprises dual HEPN domains. The HPEN domains can play a role in target cleavage in Cas13d (Zhang et al., Nat. comm. 2019. 10:2544) In certain embodiments, the effector protein lacks a counterpart to the Helical-1 domain of Cas13a. The effector protein can be a Cas13d protein, which has a median size of 928 aa. This median size is 190 aa (17%) less than that of Cas13c, more than 200 aa (18%) less than that of Cas13b, and more than 300 aa (26%) less than that of Cas13a. In embodiments, the effector protein has no requirement for a flanking sequence (e.g., PFS, PAM).

The Cas13d effector protein locus structures can include a WYL domain containing accessory protein (so denoted after three amino acids that were conserved in the originally identified group of these domains; see, e.g., WYL domain IPR026881. In embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. The WYL domain containing accessory protein can comprise an N-terminal RHH domain, as well as a pattern of primarily hydrophobic conserved residues, including an invariant tyrosine-leucine doublet corresponding to the original WYL motif. In certain embodiments, the WYL domain containing accessory protein is WYL1. WYL1 is a single WYL-domain protein associated primarily with Ruminococcus. In example embodiments, the Type VI RNA-targeting Cas enzyme is Cas 13d. In certain embodiments, Cas13d is Eubacterium siraeum DSM 15702 (EsCas13d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018) 70(2):P327-339, doi.org/10.1016[j.molcel.2018.02.028). RspCas13d and EsCas13d have no flanking sequence requirements (e.g., PFS, PAM). In certain example embodiments, the Cas 13d is as in U.S. Pat. No. 10,666,592, which is incorporated by reference as if expressed in its entirety herein.

In other example embodiments, the Type VI RNA-targeting Cas enzyme is Cas 13d. In certain embodiments, Cas13d is Eubacterium siraeum DSM 15702 (EsCas13d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016[j.molcel.2018.02.028). RspCas13d and EsCas13d have no flanking sequence requirements (e.g., PFS, PAM).

In certain example embodiments, the Cas 13d is as in U.S. Pat. No. 10,666,592, which is incorporated by reference as if expressed in its entirety herein.

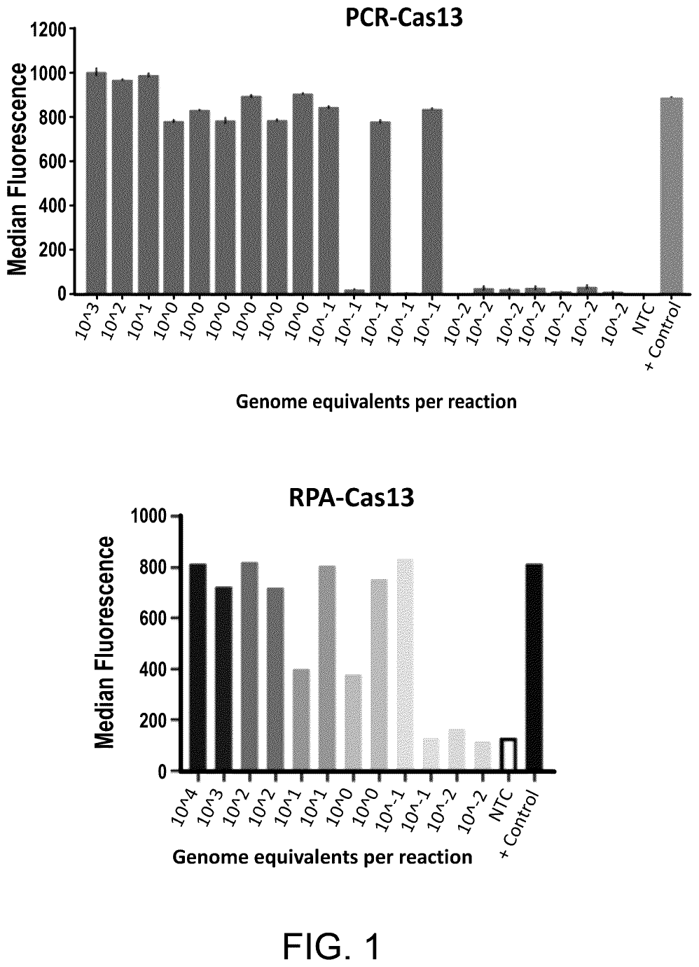

In some embodiments, the Cas13 is a thermostable Cas13. In certain embodiments, the thermostable CRISPR-Cas protein is a Cas13a. In an aspect, the Cas13a thermostable protein is from FIG. 1A of U.S. Provisional Application 62/967,408, filed Jan. 29, 2020, entitled “Novel CRISPR Enzymes and Systems” which were identified from stable anaerobic thermophilic methanogenic microbiomes fermenting switchgrass, supporting their thermostability. See, Liang et al., Biotechnol Biofuels 2018; 11: 243 doi: 10.1186/s13068-018-1238-1. Similarly, the 0J26742_10014101 clusters with the verified thermophilic sourced Cas13a sequences detailed in FIG. 1A of U.S. Provisional Application 62/967,408, filed Jan. 29, 2020, entitled “Novel CRISPR Enzymes and Systems”. The nucleic acid identified at loci 123519_10037894 was identified from a study focusing on 70° C. organism. In certain example embodiments, the Cas13 orthologue has at least two HEPN domains and at least 80% identity to a polypeptide encoded by the nucleic acid sequence 0123519_10037894 or 0J26742_10014101.

In certain example embodiments, the effector protein may be a Leptotrichia sp., Leptotrichia wadei F0279, or a Listeria sp., preferably Listeria newyorkensis FSL M6-0635.

In certain example embodiments, the C2c2 effector proteins of the invention include, without limitation, the following 21 ortholog species (including multiple CRISPR loci: Leptotrichia shahii; Leptotrichia wadei (Lw2); Listeria seeligeri; Lachnospiraceae bacterium MA2020; Lachnospiraceae bacterium NK4A179; [Clostridium]aminophilum DSM 10710; Carnobacterium gallinarum DSM 4847; Carnobacterium gallinarum DSM 4847 (second CRISPR Loci); Paludibacter propionicigenes WB4; Listeria weihenstephanensis FSL R9-0317; Listeriaceae bacterium FSL M6-0635; Leptotrichia wadei F0279; Rhodobacter capsulatus SB 1003; Rhodobacter capsulatus R121; Rhodobacter capsulatus DE442; Leptotrichia buccalis C-1013-b; Herbinix hemicellulosilytica; [Eubacterium]rectale; Eubacteriaceae bacterium CHKCI004; Blautia sp. Marseille-P2398; and Leptotrichia sp. oral taxon 879 str. F0557. Twelve (12) further non-limiting examples are: Lachnospiraceae bacterium NK4A144; Chloroflexus aggregans; Demequina aurantiaca; Thalassospira sp. TSL5-1; Pseudobutyrivibrio sp. OR37; Butyrivibrio sp. YAB3001; Blautia sp. Marseille-P2398; Leptotrichia sp. Marseille-P3007; Bacteroides ihuae; Porphyromonadaceae bacterium KH3CP3RA; Listeria riparia; and Insolitispirillum peregrinum.

In certain embodiments, the C2c2 protein according to the invention is or is derived from one of the orthologues as described in the table below, or is a chimeric protein of two or more of the orthologues as described in the table below, or is a mutant or variant of one of the orthologues as described in the table below (or a chimeric mutant or variant), including dead C2c2, split C2c2, destabilized C2c2, etc. as defined herein elsewhere, with or without fusion with a heterologous/functional domain.

In certain example embodiments, the C2c2 effector protein is selected from Table 1.

TABLE 1
Exemplary C2c2 effectors

| C2c2 orthologue | Code | Multi Letter |
|---|---|---|
| Leptotrichia shahii | C2-2 | Lsh |
| L. wadei F0279 (Lw2) | C2-3 | Lw2 |
| Listeria seeligeri | C2-4 | Lse |
| Lachnospiraceae bacterium MA2020 | C2-5 | LbM |
| Lachnospiraceae bacterium NK4A179 | C2-6 | LbNK179 |
| Clostridium aminophilum DSM 10710 | C2-7 | Ca |
| Carnobacterium gallinarum DSM | C2-8 | Cg |
| Carnobacterium gallinarum DSM 4847 | C2-9 | Cg2 |
| Paludibacter propionicigenes WB4 | C2-10 | Pp |
| Listeria weihenstephanensis FSL R9-0317 | C2-11 | Lwei |
| Listeriaceae bacterium FSL M6-0635 | C2-12 | LbFSL |
| Leptotrichia wadei F0279 | C2-13 | Lw |
| Rhodobacter capsulatus SB 1003 | C2-14 | Rc |
| Rhodobacter capsulatus R121 | C2-15 | Rc |
| Rhodobacter capsulatus DE442 | C2-16 | Rc |
| Leptotrichia buccalis C-1013-b | C2-17 | LbuC2c2 |
| Herbinix hemicellulosilytics | C2-18 | HheC2c2 |
| Eubacterium rectale | C2-19 | EreC2c2 |
| Eubacteriaceae bacterium CHKC1004 | C2-20 | EbaC2c2 |
| Blautia sp. Marseille-P2398 | C2-21 | BsmC2c2 |
| Leptotrichia sp. oral taxon 879 str. F0557 | C2-22 | LspC2c2 |
| Lachnospiraceae bacterium NK4a144 | | |
| Chloroflexus aggregans | | |
| Demequina aurantiaca | | |
| Thalassospira sp. TSL5-1 | | |
| Pseudobutyrivibrio sp. OR37 | | |
| Butyrivibrio sp. YAB3001 | | |
| Blautia sp. Marseille-P2398 | | |
| Leptotrichia sp. Marseille-P300 | | |
| Bacteroides ihuae | | |
| Porphyromonadaceae bacterium KH3CP3RA | | |
| Listeria riparia | | |
| Insolitispirillum peregrinum | | |

The wild-type protein sequences of the above species are listed in Table 2.

TABLE 2
Wild-Type C2c2 Effectors

| | | |
|---|---|---|
| C2c2-2 | | L. shahii (Lsh) |
| c2c2-3 | | L. wadei (Lw2) |
| c2c2-4 | | Listeria seeligeri |
| c2c2-5 | 1 | Lachnospiraceae bacterium MA2020 |
| c2c2-6 | 2 | Lachnospiraceae bacterium NK4A179 |
| c2c2-7 | 3 | Clostridium aminophilum DSM 10710 |
| c2c2-8 | 5 | Carnobacterium gallinarum DSM 4847 |
| c2c2-9 | 6 | Carnobacterium gallinarum DSM 4847 |
| c2c2-10 | 7 | Paludibacter propionicigenes WB4 |
| c2c2-11 | 9 | Listeria weihenstephanensis FSL R9-0317 |
| c2c2-12 | 10 | Listeriaceae bacterium FSL M6-0635 = |
| | | Listeria newyorkensis FSL M6-0635 |
| c2c2-13 | 12 | Leptotrichia wadei F0279 |
| c2c2-14 | 15 | Rhodobacter capsulatus SB 1003 |
| c2c2-15 | 16 | Rhodobacter capsulatus R121 |
| c2c2-16 | 17 | Rhodobacter capsulatus DE442 |
| LbuC2c2 | | Leptorichia buccalis C-1013-b |
| HheC2c2 | | Herbinix hemicellulosilytica |
| EreC2c2 | | Eubacterium rectale |
| EbaC2C2 | | Eubacteriaceae bacterium CHKCI004 |
| C2c2 NK4A144 | | Lachnospiraceae bacterium NK4A144 |
| C2c2 Chloro_agg | | RNA-binding protein S1 Chloroflexus aggregans |
| C2c2 Dem_Aur | | Demequina aurantiaca |
| C2c2 | | Thalassospira sp. TSL5-1 |
| Thal_Sp_TSL5 | | |
| C2c2 Pseudo_sp | | Pseudobutyrivibrio sp. OR37 |
| C2c2_Buty_sp | | Butyrivibrio sp. YAB3001 |
| C2c2_Blautia_sp | | Blautia sp. Marseille-P2398 |
| C2c2_Lepto_sp_ | | Leptotrichia sp. Marseille-P3007 |
| Marseille | | |
| C2c2_Bacteroides_ | | Bacteroides ihuae |
| ihuae | | |
| C2c2_Porph_ | | Porphyromonadaceae bacterium KH3CP3RA |
| bacterium | | |
| C2c2_Listeria_ | | Listeria riparia |
| riparia | | |
| C2c2_insolitis_ | | Insolitispirillum peregrinum |
| peregrinum | | |

A consensus sequence can be generated from multiple C2c2 orthologs, which can assist in locating conserved amino acid residues, and motifs, including but not limited to catalytic residues and HEPN motifs in C2c2 orthologs that mediate C2c2 function. One such consensus sequence, generated from the 33 orthologs mentioned above using Geneious alignment is SEQ ID NO: 1.

(SEQ ID NO: 1)
MKISKVXXXVXKKXXXGKLXKXVNERNRXAKRLSNXLBKYIXXIDKIXK
KEXXKKFXAXEEITLKLNQXXXBXLXKAXXDLRKDNXYSXJKKILHNED
INXEEXELLINDXLEKLXKIESXKYSYQKXXXNYXMSVQEHSKKSIXRI
XESAKRNKEALDKFLKEYAXLDPRMEXLAKLRKLLELYFYFKNDXIXXE
EEXNVXXHKXLKENHPDFVEXXXNKENAELNXYAIEXKKJLKYYFPXKX
AKNSNDKIFEKQELKKXWIHQJENAVERILLXXGKVXYKLQXGYLAELW
KIRINEIFIKYIXVGKAVAXFALRNXXKBENDILGGKIXKKLNGITSFX
YEKIKAEEILQREXAVEVAFAANXLYAXDLXXIRXSILQFFGGASNWDX
FLFFHFATSXISDKKWNAELIXXKKJGLVIREKLYSNNVAMFYSKDDLE
KLLNXLXXFXLRASQVPSFKKVYVRXBFPQNLLKKENDEKDDEAYSAXY
YLLKEIYYNXFLPYFSANNXFFFXVKNLVLKANKDKFXXAFXDIREMNX
GSPIEYLXXTQXNXXNEGRKKEEKEXDFIKFLLQIFXKGFDDYLKNNXX
FILKFIPEPTEXIEIXXELQAWYIVGKFLNARKXNLLGXFXSYLKLLDD
IELRALRNENIKYQSSNXEKEVLEXCLELIGLLSLDLNDYFBDEXDFAX
YJGKXLDFEKKXMKDLAELXPYDQNDGENPIVNRNIXLAKKYGTLNLLE
KJXDKVSEKEIKEYYELKKEIEEYXXKGEELHEEWXQXKNRVEXRDILE
YXEELXGQIINYNXLXNKVLLYFQLGLHYLLLDILGRLVGYTGIWERDA
XLYQIAAMYXNGLPEYIXXKKNDKYKDGQIVGXKINXFKXDKKXLYNAG
LELFENXNEHKNIXIRNYIAHFNYLSKAESSLLXYSENLRXLFSYDRKL
KNAVXKSLINILLRHGMVLKFKFGTDKKSVXIRSXKKIXHLKSIAKKLY
YPEVXVSKEYCKLVKXLLKYK

In another non-limiting example, a sequence alignment tool to assist generation of a consensus sequence and identification of conserved residues is the MUSCLE alignment tool (www.ebi.ac.uk/Tools/msa/muscle/). For example, using MUSCLE, the following amino acid locations conserved among C2c2 orthologs can be identified in Leptotrichia wadei C2c2:K2; K5; V6; E301; L331; I335; N341; G351; K352; E375; L392; L396; D403; F446; I466; I470; R474 (HEPN); H475; H479 (HEPN), E508; P556; L561; I595; Y596; F600; Y669; I673; F681; L685; Y761; L676; L779; Y782; L836; D847; Y863; L869; I872; K879; I933; L954; I958; R961; Y965; E970; R971; D972; R1046 (HEPN), H1051 (HEPN), Y1075; D1076; K1078; K1080; I1083; I1090.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In particular embodiments, the Cas13b enzyme is derived from Bergeyella zoohelcum. In certain other example embodiments, the effector protein is, or comprises an amino acid sequence having at least 80% sequence homology to any of the sequences listed in Table 3.

TABLE 3

| | |
|---|---|
| B-01 | Bergeyella zoohelcum |
| B-02 | Prevotella intermedia |
| B-03 | Prevotella buccae |
| B-04 | Alistipes sp. ZOR0009 |
| B-05 | Prevotella sp. MA2016 |
| B-06 | Riemerella anatipestifer |
| B-07 | Prevotella aurantiaca |
| B-08 | Prevotella saccharolytica |
| B-09 | Prevotella intermedia |
| B-10 | Capnocytophaga canimorsus |
| B-11 | Porphyromonas gulae |
| B-12 | Prevotella sp. P5-125 |
| B-13 | Flavobacterium branchiophilum |
| B-14 | Porphyromonas gingivalis |
| B-15 | Prevotella intermedia |

In certain example embodiments, the wild type sequence of the Cas13b orthologue is found in Table 4 or 5 below.

TABLE 4

| | |
|---|---|
| Bergeyella zoohelcum (SEQ. I.D. No. 326) | 1 |
| Prevotella intermedia (SEQ. I.D. No. 327) | 2 |
| Prevotella buccae (SEQ. I.D. No. 328) | 3 |
| Porphyromonas gingivalis (SEQ. I.D. No. 329) | 4 |
| Bacteroides pyogenes (SEQ. I.D. No. 330) | 5 |
| Alistipes sp. ZOR0009 (SEQ. I.D. No. 331) | 6 |
| Prevotella sp. MA2016 (SEQ. I.D. No. 332) | 7a |
| Prevotella sp. MA2016 (SEQ. I.D. No. 333) | 7b |
| Riemerella anatipestifer (SEQ. I.D. No. 334) | 8 |
| Prevotella aurantiaca (SEQ. I.D. No. 335) | 9 |
| Prevotella saccharolytica (SEQ. I.D. No. 336) | 10 |
| HMPREF9712_03108 [Myroides odoratimimus | 11 |
| CCUG 10230] (SEQ. I.D. No. 337) | |
| Prevotella intermedia (SEQ. I.D. No. 338) | 12 |
| Capnocytophaga canimorsus (SEQ. I.D. No. 339) | 13 |
| Porphyromonas gulae (SEQ. I.D. No. 340) | 14 |
| Prevotella sp. P5-125 (SEQ. I.D. No. 341) | 15 |
| Flavobacterium branchiophilum (SEQ. I.D. No. 342) | 16 |
| Myroides odoratimimus (SEQ. I.D. No. 343) | 17 |
| Flavobacterium columnare (SEQ. I.D. No. 344) | 18 |
| Porphyromonas gingivalis (SEQ. I.D. No. 345) | 19 |
| Porphyromonas sp. COT-052 OH4946 | 20 |
| (SEQ. I.D. No. 346) | |
| Prevotella intermedia (SEQ. I.D. No. 347) | 21 |
| PIN17 0200 [Prevotella intermedia 17] | AFJ07523 |
| (SEQ. I.D. No. 348) | |
| Prevotella intermedia (SEQ. I.D. No. 349) | BAU18623 |
| HMPREF6485 0083 [Prevotella buccae ATCC | EFU31981 |
| 33574] (SEQ. I.D. No. 350) | |
| HMPREF9144_1146 [Prevotella pallens ATCC | EGQ18444 |
| 700821] (SEQ. I.D. No. 351) | |
| HMPREF9714_02132 [Myroides odoratimimus | EHO08761 |
| CCUG 12901] (SEQ. I.D. No. 352) | |
| HMPREF9711_00870 [Myroides odoratimimus | EKB06014 |
| CCUG 3837] (SEQ. I.D. No. 353) | |
| HMPREF9699_02005 [Bergeyella zoohelcum ATCC | EKB54193 |
| 43767] (SEQ. I.D. No. 354) | |
| HMPREF9151_01387 [Prevotella saccharolytica | EKY00089 |
| F0055] (SEQ. I.D. No. 355) | |
| A343_1752 [Porphyromonas gingivalis JCVI | EOA10535 |
| SC001] (SEQ. I.D. No. 356) | |
| HMPREF1981_03090 [Bacteroides pyogenes | ERI81700 |
| F0041] (SEQ. I.D. No. 357) | |
| HMPREF1553_02065 [Porphyromonas gingivalis | ERJ65637 |
| F0568] (SEQ. I.D. No. 358) | |
| HMPREF1988_01768 [Porphyromonas gingivalis | ERJ81987 |
| F0185] (SEQ. I.D. No. 359) | |
| HMPREF1990_01800 [Porphyromonas gingivalis | ERJ87335 |
| W4087] (SEQ. I.D. No. 360) | |
| M573_117042 [Prevotella intermedia ZT] | KJJ86756 |
| (SEQ. I.D. No. 361) | |
| A2033_10205 [Bacteroidetes bacterium | OFX18020.1 |
| GWA2_31_9] (SEQ. I.D. No. 362) | |
| SAMN05421542_0666 [Chryseobacterium jejuense] | SDI27289.1 |
| (SEQ. I.D. No. 363) | |
| SAMN05444360_11366 [Chryseobacterium | SHM52812.1 |
| carnipullorum] (SEQ. I.D. No. 364) | |
| SAMN05421786_1011119 [Chryseobacterium | SIS70481.1 |
| ureilyticum] (SEQ. I.D. No. 365) | |
| Prevotella buccae (SEQ. I.D. No. 366) | WP_004343581 |
| Porphyromonas gingivalis (SEQ. I.D. No. 367) | WP_005873511 |
| Porphyromonas gingivalis (SEQ. I.D. No. 368) | WP_005874195 |
| Prevotella pallens (SEQ. I.D. No. 369) | WP_006044833 |
| Myroides odoratimimus (SEQ. I.D. No. 370) | WP_006261414 |
| Myroides odoratimimus (SEQ. I.D. No. 371) | WP_006265509 |
| Prevotella sp. MSX73 (SEQ. I.D. No. 372) | WP_007412163 |
| Porphyromonas gingivalis (SEQ. I.D. No. 373) | WP_012458414 |
| Paludibacter propionicigenes (SEQ. I.D. No. 374) | WP_013446107 |
| Porphyromonas gingivalis (SEQ. I.D. No. 375) | WP_013816155 |
| Flavobacterium columnare (SEQ. I.D. No. 376) | WP_014165541 |
| Psychroflexus torquis (SEQ. I.D. No. 377) | WP_015024765 |
| Riemerella anatipestifer (SEQ. I.D. No. 378) | WP_015345620 |
| Prevotella pleuritidis (SEQ. I.D. No. 379) | WP_021584635 |
| Porphyromonas gingivalis (SEQ. I.D. No. 380) | WP_021663197 |
| Porphyromonas gingivalis (SEQ. I.D. No. 381) | WP_021665475 |
| Porphyromonas gingivalis (SEQ. I.D. No. 382) | WP_021677657 |
| Porphyromonas gingivalis (SEQ. I.D. No. 383) | WP_021680012 |
| Porphyromonas gingivalis (SEQ. I.D. No. 384) | WP_023846767 |
| Prevotella falsenii (SEQ. I.D. No. 385) | WP_036884929 |
| Prevotella pleuritidis (SEQ. I.D. No. 386) | WP_036931485 |
| [Porphyromonas gingivalis (SEQ. I.D. No. 387) | WP_039417390 |
| Porphyromonas gulae (SEQ. I.D. No. 388) | WP_039418912 |
| Porphyromonas gulae (SEQ. I.D. No. 389) | WP_039419792 |
| Porphyromonas gulae (SEQ. I.D. No. 390) | WP_039426176 |
| Porphyromonas gulae (SEQ. I.D. No. 391) | WP_039431778 |
| Porphyromonas gulae (SEQ. I.D. No. 392) | WP_039437199 |
| Porphyromonas gulae (SEQ. I.D. No. 393) | WP_039442171 |
| Porphyromonas gulae (SEQ. I.D. No. 394) | WP_039445055 |
| Capnocytophaga cynodegmi (SEQ. I.D. No. 395) | WP_041989581 |
| Prevotella sp. P5-119 (SEQ. I.D. No. 396) | WP_042518169 |
| Prevotella sp. P4-76 (SEQ. I.D. No. 397) | WP_044072147 |
| Prevotella sp. P5-60 (SEQ. I.D. No. 398) | WP_044074780 |
| Phaeodactylibacter xiamenensis (SEQ. I.D. No. 399) | WP_044218239 |
| Flavobacterium sp. 316 (SEQ. I.D. No. 400) | WP_045968377 |
| Porphyromonas gulae (SEQ. I.D. No. 401) | WP_046201018 |
| WP_047431796 (SEQ. I.D. No. 402) | Chryseobacterium |
| | sp. YR477 |
| Riemerella anatipestifer (SEQ. I.D. No. 403) | WP_049354263 |
| Porphyromonas gingivalis (SEQ. I.D. No. 404) | WP_052912312 |
| Porphyromonas gingivalis (SEQ. I.D. No. 405) | WP_058019250 |
| Flavobacterium columnare (SEQ. I.D. No. 406) | WP_060381855 |
| Porphyromonas gingivalis (SEQ. I.D. No. 407) | WP_061156470 |
| Porphyromonas gingivalis (SEQ. I.D. No. 408) | WP_061156637 |
| Riemerella anatipestifer (SEQ. I.D. No. 409) | WP_061710138 |
| Flavobacterium columnare (SEQ. I.D. No. 410) | WP_063744070 |
| Riemerella anatipestifer (SEQ. I.D. No. 411) | WP_064970887 |
| Sinomicrobium oceani (SEQ. I.D. No. 412) | WP_072319476.1 |
| Reichenbachiella agariperforans (SEQ. I.D. No. 413) | WP_073124441.1 |

The SEQ ID NO: in this table are SEQ ID NO: from Table 4a of WO2018/107129, which is herein incorporated by reference

TABLE 5
Name or Accession No.

WP_015345620 (SEQ. I.D. No. 479)
WP_049354263 (SEQ. I.D. No. 480)
WP_061710138 (SEQ. I.D. No. 481)
6 (SEQ. I.D. No. 482) Alistipes sp. ZOR0009
SIS70481.1
15 Prevotella sp. (SEQ. I.D. No. 484)
WP_042518169 (SEQ. I.D. No. 485)
WP_044072147 (SEQ. I.D. No. 486)
WP_044074780 (SEQ. I.D. No. 487)
8 (modified) (SEQ. I.D. No. 488)
WP_064970887 (SEQ. I.D. No. 489)
5 (SEQ. I.D. No. 490)
ERI81700 (SEQ. I.D. No. 491)
WP_036931485 (SEQ. I.D. No. 492)
19 (SEQ. I.D. No. 493)
WP_012458414 (SEQ. I.D. No. 494)
WP_013816155 (SEQ. I.D. No. 495)
WP_039417390 (SEQ. I.D. No. 496)
WP_039419792 (SEQ. I.D. No. 497)
WP_039426176 (SEQ. I.D. No. 498)
WP_039437199 (SEQ. I.D. No. 499)
WP_061156470 (SEQ. I.D. No. 500)
12 (SEQ. I.D. No. 501)
9 (SEQ. I.D. No. 502)
EGQ18444 (SEQ. I.D. No. 503)
KJJ86756 (SEQ. I.D. No. 504)
WP_006044833 (SEQ. I.D. No. 505)
2 (SEQ. I.D. No. 506)
3 (SEQ. I.D. No. 507)
EFU31981 (SEQ. I.D. No. 508)
WP_004343581 (SEQ. I.D. No. 509)
WP_007412163 (SEQ. I.D. No. 510)
WP_044218239 (SEQ. I.D. No. 511)
21 (SEQ. I.D. No. 512)
BAU18623 (SEQ. I.D. No. 513)
WP_036884929 (SEQ. I.D. No. 514)
WP_073124441.1 (SEQ. I.D. No. 515)
AFJ07523 (SEQ. I.D. No. 516)
4 (SEQ. I.D. No. 517)
ERJ65637 (SEQ. I.D. No. 518)
ERJ81987 (SEQ. I.D. No. 519)
ERJ87335 (SEQ. I.D. No. 520)
WP_005873511 (SEQ. I.D. No. 521)
WP_021663197 (SEQ. I.D. No. 522)
WP_021665475 (SEQ. I.D. No. 523)
WP_021677657 (SEQ. I.D. No. 524)
WP_021680012 (SEQ. I.D. No. 525)
WP_023846767 (SEQ. I.D. No. 526)
WP_039445055 (SEQ. I.D. No. 527)
WP_061156637 (SEQ. I.D. No. 528)
WP_021584635 (SEQ. I.D. No. 529)
WP_015024765 (SEQ. I.D. No. 530)
WP_047431796 (SEQ. I.D. No. 531)
WP_072319476.1 (SEQ. I.D. No. 532)
16 (SEQ. I.D. No. 533)
EKY00089 (SEQ. I.D. No. 534)
10 (SEQ. I.D. No. 535)
WP_013446107 (SEQ. I.D. No. 536)
WP_045968377 (SEQ. I.D. No. 537)
SHM52812.1 (SEQ. I.D. No. 538)
EHO08761 (SEQ. I.D. No. 539)
EKB06014 (SEQ. I.D. No. 540)
WP_006261414 (SEQ. I.D. No. 541)
WP_006265509 (SEQ. I.D. No. 542)
11 (SEQ. I.D. No. 543)
17 (SEQ. I.D. No. 544)
OFX18020.1 (SEQ. I.D. No. 545)
SDI27289.1 (SEQ. I.D. No. 546)
WP_039442171 (SEQ. I.D. No. 547)
14 (SEQ. I.D. No. 548)
20 (SEQ. I.D. No. 549)
EOA10535 (SEQ. I.D. No. 550)
WP_005874195 (SEQ. I.D. No. 551)
WP_039418912 (SEQ. I.D. No. 552)
WP_039431778 (SEQ. I.D. No. 553)
WP_046201018 (SEQ. I.D. No. 554)
WP_052912312 (SEQ. I.D. No. 555)
WP_058019250 (SEQ. I.D. No. 556)
WP_014165541 (SEQ. I.D. No. 557)
13 (SEQ. I.D. No. 558)
WP_060381855 (SEQ. I.D. No. 559)
WP_063744070 (SEQ. I.D. No. 560)
18 (SEQ. I.D. No. 561)
WP_041989581 (SEQ. I.D. No. 562)
1 (SEQ. I.D. No. 563)
EKB54193 (SEQ. I.D. No. 564)
7 (modified) (SEQ. I.D. No. 565)
7 (modified)—residues only (SEQ. I.D. No. 566)
The SEQ ID NO: in this table are SEQ ID NO: from Table 4b of WO2018/107129, which is herein incorporated by reference

In certain example embodiments, the RNA-targeting effector protein is a Cas13c effector protein as disclosed in U.S. Provisional Patent Application No. 62/525,165 filed Jun. 26, 2017, and PCT Application No. US 2017/047193 filed Aug. 16, 2017. Example wildtype orthologue sequences of Cas13c are provided in Table 6 below.

TABLE 6
Name

EHO19081 (SEQ. I.D. No. 567)
WP_094899336 (SEQ. I.D. No. 568)
WP_040490876 (SEQ. I.D. No. 569)
WP_047396607 (SEQ. I.D. No. 570)
WP_035935671 (SEQ. I.D. No. 571)
WP_035906563 (SEQ. I.D. No. 572)
WP_042678931 (SEQ. I.D. No. 573)
WP_062627846 (SEQ. I.D. No. 574)
WP_005959231 (SEQ. I.D. No. 575)
WP_027128616 (SEQ. I.D. N. 576)
WP_062624740 (SEQ. I.D. No. 577)
WP_096402050 (SEQ. I.D. No. 578)
The SEQ ID NO: in this table are SEQ ID NO: from Table 5 of WO2018/107129, which is herein incorporated by reference

In certain example embodiments, the Cas13 protein may be selected from any of the following.

TABLE 7

| | | Seq. |
|---|---|---|
| | | ID. |
| ID | Species | No: |
| Cas13a1 | Leptotrichia shahii | 580 |
| Cas13a2 | Leptotrichia wadei (Lw2) | 581 |
| Cas13a3 | Listeria seeligeri | 582 |
| Cas13a4 | Lachnospiraceae bacterium MA2020 | 583 |
| Cas13a5 | Lachnospiraceae bacterium NK4A179 | 584 |
| Cas13a6 | [Clostridium] aminophilum DSM 10710 | 585 |
| Cas13a7 | Carnobacterium gallinarum DSM 4847 | 586 |
| Cas13a8 | Carnobacterium gallinarum DSM 4847 | 587 |
| Cas13a9 | Paludibacter propionicigenes WB4 | 588 |
| Cas13a10 | Listeria weihenstephanensis FSL R9-0317 | 589 |
| Cas13a11 | Listeriaceae bacterium FSL M6-0635 | 590 |
| Cas13a12 | Leptotrichia wadei F0279 | 591 |
| Cas13a13 | Rhodobacter capsulatus SB 1003 | 592 |
| Cas13a14 | Rhodobacter capsulatus R121 | 593 |
| Cas13a15 | Rhodobacter capsulatus DE442 | 594 |
| Cas13a16 | Leptotrichia buccalis C-1013-b | 595 |
| Cas13a17 | Herbinix hemicellulosilytica | 596 |
| Cas13a18 | [Eubacterium] rectale | 597 |
| Cas13a19 | Eubacteriaceae bacterium CHKCI004 | 598 |
| Cas13a20 | Blautia sp. Marseille-P2398 | 599 |
| Cas13a21 | Leptotrichia sp. oral taxon 879 str. F0557 | 600 |
| Cas13b1 | Bergeyella zoohelcum | 601 |
| Cas13b2 | Prevotella intermedia | 602 |
| Cas13b3 | Prevotella buccae | 603 |
| Cas13b4 | Alistipes sp. ZOR0009 | 604 |
| Cas13b5 | Prevotella sp. MA2016 | 605 |
| Cas13b6 | Riemerella anatipestifer | 606 |
| Cas13b7 | Prevotella aurantiaca | 607 |
| Cas13b8 | Prevotella saccharolytica | 608 |
| Cas13b9 | Prevotella intermedia | 609 |
| Cas13b10 | Capnocytophaga canimorsus | 610 |
| Cas13b11 | Porphyromonas gulae | 611 |
| Cas13b12 | Prevotella sp. P5-125 | 612 |
| Cas13b13 | Flavobacterium branchiophilum | 613 |
| Cas13b14 | Porphyromonas gingivalis | 614 |
| Cas13b15 | Prevotella intermedia | 615 |
| Cas13cl | Fusobacterium necrophorum subsp. | 616 |
| | funduliforme ATCC 51357 contig00003 | |
| Cas13c2 | Fusobacterium necrophorum DJ-2 contig0065, | 617 |
| | whole genome shotgun sequence | |
| Cas13c3 | Fusobacterium necrophorum BFTR-1 | 618 |
| | contig0068 | |
| Ca13c4 | Fusobacterium necrophorum subsp. | 619 |
| | funduliforme 1_1_36S cont1.14 | |
| Cas13c5 | Fusobacterium perfoetens ATCC 29250 | 620 |
| | T364DRAFT scaffold00009.9 C | |
| Cas13c6 | Fusobacterium ulcerans ATCC 49185 cont2.38 | 621 |
| Cas13c7 | Anaerosalibacter sp. ND1 genome assembly | 622 |
| | Anaerosalibacter massiliensis ND1 | |

The SEQ ID NO: in this table are SEQ ID NO: from Table 6 of WO2018/107129, which is herein incorporated by reference

### Cas12 Polypeptides

In certain example embodiments, a Cas that has collateral activity that can be included in the CRISPR-Cas system is or includes one or more Cas12 polypeptides (e.g., Cas 12a (also known as Cpf1), 12b (also known as C2c1), 12c, 12c1, 12c2, 12d, 12e, 12a1, 12g1, 12h1, 12i1, 12f (also known as Cas14) See e.g., Kaminski et al., Nat. Biomed. Eng. 5:643-656 (2021)). In some embodiments, the Cas12 protein can have trans-cleavage activity (also referred to as collateral cleavage), which cleaves ssDNA indiscriminately. In some embodiments, the Cas12 has multiple-turnover nuclease activity, which can be harnessed in the context of an assay described herein for amplified detection of targets.

Cas12's non-specific cleavage can be leveraged to cleave reporters upon target recognition, allowing for the design of sensitive and specific diagnostics using Cas12, including single nucleotide variants, detection based on rRNA sequences, screening for drug resistance, monitoring microbe outbreaks, genetic perturbations, and screening of environmental samples, as described, for example, in Broughton et al. 2020. CRISPR-Cas12-based detection of SARS-CoV-2. Nat. Biotech. 38:870-874, https://doi.org/10.1038/s41587-020-0513-4; Leung et al. 2021. CRISPR-Cas12-based nucleic acids detection systems. Methods.; S1046-2023(21)00063-3.doi: 10.1016/j.ymeth.2021.02.018; Mahas et al., Viruses. 2021. 13:466, https://doi.org/10.3390/v13030466; Ali et al., 2020. iSCAN: An RT-LAMP-coupled CRISPR-Cas12 module for rapid, sensitive detection of SARS-CoV-2Vir. Res. 288:198129. https://doi.org/10.1016[j.virusres.2020.198129; Ramachandran et al., 2020. Electric field-driven microfluidics for rapid CRISPR-based diagnostics and its application to detection of SARS-CoV-2. PNAS Nov. 24, 2020 117 (47) 29518-29525; Mukama et al., An ultrasensitive and specific point-of care CRISPR-Cas12 based lateral flow biosensor for the rapid detection of nucleic acids. Biosens Bioelectron. 2020 Jul. 1; 159:112143. doi: 10.1016/j.bios.2020.1 12143; Chen et al., 2018. CRISPR-Cas12a target binding unleashes indiscriminate single-stranded DNase activity. Science. April 27; 360(6387):436-439. doi: 10.1126/science.aar6245; Kellner et al., 2019. Nat Protoc. 2019 October; 14(10):2986-3012. doi: 10.1038/s41596-019-0210-2; Broughton et al., 2020. Rapid Detection of 2019 Novel Coronavirus SARS-CoV-2 Using a CRISPR-based DETECTR Lateral Flow Assay. 2020. medRxiv. March 27; 2020.03.06.20032334. doi: 10.1101/2020.03.06.20032334; Wu et al. 2021. CRISPR-Cas12-Based Rapid Authentication of Halal Food. J Agric Food Chem. 2021 Aug. 26. doi: 10.1021/acs.jafc.1c03078; Long et al. 2021. CRISPR/Cas12-Based Ultra-Sensitive and Specific Point-of-Care Detection of HBV. Int J Mol Sci. 2021 May 3; 22(9):4842. doi: 10.3390/ijms22094842; Curti et al., Viruses. 2021 Mar. 5; 13(3):420. doi: 10.3390/v13030420; Li et al., Cell Discovery (2018)4:20. DOI 10.1038/s41421-018-0028-z; Lucia et al. 2020. An ultrasensitive, rapid, and portable coronavirus SARS-Cov-2 sequence detection method based on CRISPR-Cas12. bioRxiv preprint doi: https/doi.org/10.1101/2020.02.29.971127; MammothBiosciences. 2020. Broughton et al., available at https://mammoth.bio/wp-content/uploads/2020/04/200423-A-protocol-for-rapid-detection-of-SARS-CoV-2-using-CRISPR-diagnostics_3.pdf, East-Seletsky et al., Nat. 538:270, doi:10.1038/nature19802; International Pat. Pub. WO2019/233358; WO2019/011022; U.S. Pat. Nos. 10,337,051; 10,449,4664, 10,253,365; 10,392,616, WO2019006471, US 2020/0299768; US 2020/0399697; US 2019/0241954; the disclosure of each can be adapted for use with the present invention in view of the description provided herein and each of which is incorporated herein by reference in its entirety.

### Cpf1 Orthologs

The present invention encompasses the use of a Cpf1 effector protein, derived from a Cpf1 locus denoted as subtype V-A. Herein such effector proteins are also referred to as “Cpf1p”, e.g., a Cpf1 protein (and such effector protein or Cpf1 protein or protein derived from a Cpf1 locus is also called “CRISPR enzyme”). Presently, the subtype V-A loci encompasses cas1, cas2, a distinct gene denoted cpf1 and a CRISPR array. Cpf1(CRISPR-associated protein Cpf1, subtype PREFRAN) is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in all Cas9 proteins, and the RuvC-like domain is contiguous in the Cpf1 sequence, in contrast to Cas9 where it contains long inserts including the HNH domain. Accordingly, in particular embodiments, the CRISPR-Cas enzyme comprises only a RuvC-like nuclease domain.

The programmability, specificity, and collateral activity of the RNA-guided Cpf1 also make it an ideal switchable nuclease for non-specific cleavage of nucleic acids. In one embodiment, a Cpf1 system is engineered to provide and take advantage of collateral non-specific cleavage of RNA. In another embodiment, a Cpf1 system is engineered to provide and take advantage of collateral non-specific cleavage of ssDNA. Accordingly, engineered Cpf1 systems provide platforms for nucleic acid detection and transcriptome manipulation. Cpf1 is developed for use as a mammalian transcript knockdown and binding tool. Cpf1 is capable of robust collateral cleavage of RNA and ssDNA when activated by sequence-specific targeted DNA binding.

The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related or are only partially structurally related. An “orthologue” of a protein as used herein is a protein of a different species which performs the same or a similar function as the protein it is an orthologue of. Orthologous proteins may but need not be structurally related or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22(4):359-66. doi: 10.1002/pro.2225.). See also Shmakov et al. (2015) for application in the field of CRISPR-Cas loci. Homologous proteins may but need not be structurally related, or are only partially structurally related.

The Cpf1 gene is found in several diverse bacterial genomes, typically in the same locus with cas1, cas2, and cas4 genes and a CRISPR cassette (for example, FNFX1_1431-FNFX1_1428 of Francisella cf. novicida Fx1). In particular embodiments, the effector protein is a Cpf1 effector protein from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus.

In further particular embodiments, the Cpf1 effector protein is from an organism selected from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii.

The effector protein may comprise a chimeric effector protein comprising a first fragment from a first effector protein (e.g., a Cpf1) ortholog and a second fragment from a second effector (e.g., a Cpf1) protein ortholog, and wherein the first and second effector protein orthologs are different. At least one of the first and second effector protein (e.g., a Cpf1) orthologs may comprise an effector protein (e.g., a Cpf1) from an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus; e.g., a chimeric effector protein comprising a first fragment and a second fragment wherein each of the first and second fragments is selected from a Cpf1 of an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus wherein the first and second fragments are not from the same bacteria; for instance a chimeric effector protein comprising a first fragment and a second fragment wherein each of the first and second fragments is selected from a Cpf1 of S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae, wherein the first and second fragments are not from the same bacteria.

In a more preferred embodiment, the Cpf1p is derived from a bacterial species selected from Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In certain embodiments, the Cpf1p is derived from a bacterial species selected from Acidaminococcus sp. BV3L6, Lachnospiraceae bacterium MA2020. In certain embodiments, the effector protein is derived from a subspecies of Francisella tularensis 1, including but not limited to Francisella tularensis subsp. Novicida.

In some embodiments, the Cpf1p is derived from an organism from the genus of Eubacterium. In some embodiments, the CRISPR effector protein is a Cpf1 protein derived from an organism from the bacterial species of Eubacterium rectale. In some embodiments, the amino acid sequence of the Cpf1 effector protein corresponds to NCBI Reference Sequence WP_055225123.1, NCBI Reference Sequence WP_055237260.1, NCBI Reference Sequence WP_055272206.1, or GenBank ID OLA16049.1. In some embodiments, the Cpf1 effector protein has a sequence homology or sequence identity of at least 60%, more particularly at least 70, such as at least 80%, more preferably at least 85%, even more preferably at least 90%, such as for instance at least 95%, with NCBI Reference Sequence WP_055225123.1, NCBI Reference Sequence WP_055237260.1, NCBI Reference Sequence WP_055272206.1, or GenBank ID OLA16049.1. The skilled person will understand that this includes truncated forms of the Cpf1 protein whereby the sequence identity is determined over the length of the truncated form. In some embodiments, the Cpf1 effector recognizes the PAM sequence of TTTN or CTTN.

In particular embodiments, the homologue or orthologue of Cpf1 as referred to herein has a sequence homology or identity of at least 80%, more preferably at least 85%, even more preferably at least 90%, such as for instance at least 95% with Cpf1. In further embodiments, the homologue or orthologue of Cpf1 as referred to herein has a sequence identity of at least 80%, more preferably at least 85%, even more preferably at least 90%, such as for instance at least 95% with the wild type Cpf1. Where the Cpf1 has one or more mutations (mutated), the homologue or orthologue of said Cpf1 as referred to herein has a sequence identity of at least 80%, more preferably at least 85%, even more preferably at least 90%, such as for instance at least 95% with the mutated Cpf1.

In an embodiment, the Cpf1 protein may be an ortholog of an organism of a genus which includes, but is not limited to Acidaminococcus sp, Lachnospiraceae bacterium or Moraxella bovoculi; in particular embodiments, the type V Cas protein may be an ortholog of an organism of a species which includes, but is not limited to, Acidaminococcus sp. BV3L6; Lachnospiraceae bacterium ND2006 (LbCpf1) or Moraxella bovoculi 237. In particular embodiments, the homologue or orthologue of Cpf1 as referred to herein has a sequence homology or identity of at least 80%, more preferably at least 85%, even more preferably at least 90%, such as for instance at least 95% with one or more of the Cpf1 sequences disclosed herein. In further embodiments, the homologue or orthologue of Cpf as referred to herein has a sequence identity of at least 80%, more preferably at least 85%, even more preferably at least 90%, such as for instance at least 95% with the wild type FnCpf1, AsCpf1 or LbCpf1. The skilled person will understand that this includes truncated forms of the Cpf1 protein whereby the sequence identity is determined over the length of the truncated form. In certain of the following, Cpf1 amino acids are followed by nuclear localization signals (NLS) (italics), a glycine-serine (GS) linker, and 3×HA tag.

In particular embodiments, the Cpf1 protein of the invention has a sequence homology or identity of at least 60%, more particularly at least 70, such as at least 80%, more preferably at least 85%, even more preferably at least 90%, such as for instance at least 95% with FnCpf1, AsCpf1 or LbCpf1. In further embodiments, the Cpf1 protein as referred to herein has a sequence identity of at least 60%, such as at least 70%, more particularly at least 80%, more preferably at least 85%, even more preferably at least 90%, such as for instance at least 95% with the wild type AsCpf1 or LbCpf1. In particular embodiments, the Cpf1 protein of the present invention has less than 60% sequence identity with FnCpf1. The skilled person will understand that this includes truncated forms of the Cpf1 protein whereby the sequence identity is determined over the length of the truncated form.

In certain of the following, Cpf1 amino acids are followed by nuclear localization signals (NLS) (italics), a glycine-serine (GS) linker, and 3×HA tag. 1—Franscisella tularensis subsp. novicida U112 (FnCpf1) (SEQ ID NO: 281 of WO2019/126577); 3—Lachnospiraceae bacterium MC2017 (Lb3Cpf1) (SEQ ID NO: 282 of WO2019/126577); 4—Butyrivibrio proteoclasticus (BpCpf1) (SEQ ID NO: 283 of WO2019/126577); 5—Peregrinibacteria bacterium GW2011_GWA_33_10 (PeCpf1) (SEQ ID NO: 284 of WO2019/126577); 6—Parcubacteria bacterium GWC2011_GWC2_44_17 (PbCpf1) (SEQ ID NO: 285 of WO2019/126577); 7—Smithella sp. SC_K08D17 (SsCpf1) (SEQ ID NO: 286 of WO2019/126577); 8—Acidaminococcus sp. BV3L6 (AsCpf1) (SEQ ID NO: 287 of WO2019/126577); 9—Lachnospiraceae bacterium MA2020 (Lb2Cpf1) (SEQ ID NO: 288 of WO2019/126577); 10—Candidatus Methanoplasma termitum (CMtCpf1) (SEQ ID NO: 289 of WO2019/126577); 11—Eubacterium eligens (EeCpf1)(SEQ ID NO: 290 of WO2019/126577); 12—Moraxella bovoculi 237 (MbCpf1) (SEQ ID NO: 291 of WO2019/126577); 13—Leptospira inadai (LiCpf1) (SEQ ID NO: 292 of WO2019/126577); 14—Lachnospiraceae bacterium ND2006 (LbCpf1) (SEQ ID NO: 293 of WO2019/126577); 15—Porphyromonas crevioricanis (PcCpf1) (SEQ ID NO: 294 of WO2019/126577); 16—Prevotella disiens (PdCpf1) (SEQ ID NO: 295 of WO2019/126577); 17—Porphyromonas macacae (PmCpf1) (SEQ ID NO: 296 of WO2019/126577); 18—Thiomicrospira sp. XS5 (TsCpf1) (SEQ ID NO: 297 of WO2019/126577); 19—Moraxella bovoculi AAX08_00205 (Mb2Cpf1) (SEQ ID NO: 298 of WO2019/126577); 20—Moraxella bovoculi AAX11_00205 (Mb3Cpf1) (SEQ ID NO: 299 of WO2019/126577); and 21—Butyrivibrio sp. NC3005 (BsCpf1) (SEQ ID NO: 300 of WO2019/126577). WO2019/126577 is incorporated by reference as if expressed in its entirety herein.

Further Cpf1 orthologs include NCBI WP_055225123.1, NCBI WP_055237260.1, NCBI WP_055272206.1, and GenBank OLA16049.1.

In some embodiments the Cas 12a is a variant as set forth in Kleinstriver et al., Nat Biotechnol. 2019 March; 37(3):276-282. doi: 10.1038/s41587-018-0011-0.

### C2c1 Orthologs

The present invention encompasses the use of a C2c1 effector proteins, derived from a C2c1 locus denoted as subtype V-B and orthologuyes and homologues thereof. Herein such effector proteins are also referred to as “C2c1p”, e.g., a C2c1 protein (and such effector protein or C2c1 protein or protein derived from a C2c1 locus is also called “CRISPR enzyme”). The C2c1 gene is found in several diverse bacterial genomes, typically in the same locus with cas1, cas2, and cas4 genes and a CRISPR cassette. Thus, the layout of this putative novel CRISPR-Cas system appears to be similar to that of type II-B. Furthermore, similar to Cas9, the C2c1 protein contains an active RuvC-like nuclease, an arginine-rich region, and a Zn finger (absent in Cas9).

Presently, the subtype V-B loci encompasses cas1-Cas4 fusion, cas2, a distinct gene denoted C2c1 and a CRISPR array. C2c1 (CRISPR-associated protein C2c1) is a large protein (about 1100-1300 amino acids) that contains a RuvC-like nuclease domain homologous to the corresponding domain of Cas9 along with a counterpart to the characteristic arginine-rich cluster of Cas9. However, C2c1 lacks the HNH nuclease domain that is present in all Cas9 proteins, and the RuvC-like domain is contiguous in the C2c1 sequence, in contrast to Cas9 where it contains long inserts including the HNH domain. Accordingly, in particular embodiments, the CRISPR-Cas enzyme comprises only a RuvC-like nuclease domain.

The programmability, specificity, and collateral activity of the RNA-guided C2c1 also make it an ideal switchable nuclease for non-specific cleavage of nucleic acids. In one embodiment, a C2c1 system is engineered to provide and take advantage of collateral non-specific cleavage of RNA. In another embodiment, a C2c1 system is engineered to provide and take advantage of collateral non-specific cleavage of ssDNA. Accordingly, engineered C2c1 systems provide platforms for nucleic acid detection and transcriptome manipulation, and inducing cell death. C2c1 is developed for use as a mammalian transcript knockdown and binding tool. C2c1 is capable of robust collateral cleavage of RNA and ssDNA when activated by sequence-specific targeted DNA binding.

In certain embodiments, C2c1 is provided or expressed in an in vitro system or in a cell, transiently or stably, and targeted or triggered to non-specifically cleave cellular nucleic acids. In one embodiment, C2c1 is engineered to knock down ssDNA, for example viral ssDNA. In another embodiment, C2c1 is engineered to knock down RNA. The system can be devised such that the knockdown is dependent on a target DNA present in the cell or in vitro system, or triggered by the addition of a target nucleic acid to the system or cell.

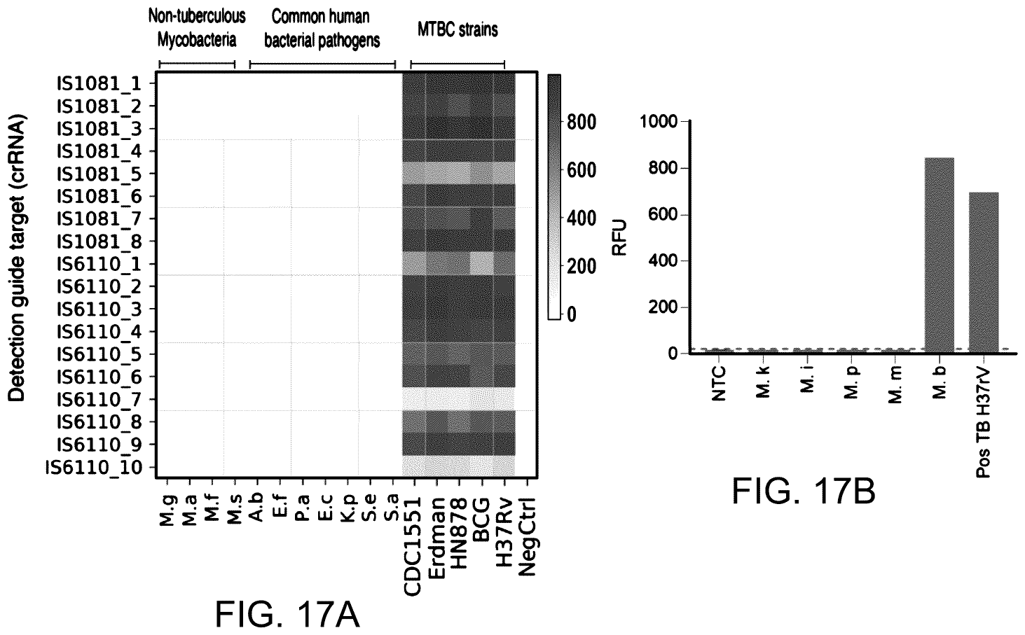

C2c1 (also known as Cas12b) proteins are RNA guided nucleases. In certain embodiments, the Cas protein may comprise at least 80% sequence identity to a polypeptide as described in International Patent Publication WO 2016/205749 at FIG. 17-21, FIG. 41A-41M, 44A-44E, incorporated herein by reference. Its cleavage relies on a tracr RNA to recruit a guide RNA comprising a guide sequence and a direct repeat, where the guide sequence hybridizes with the target nucleotide sequence to form a DNA/RNA heteroduplex. Based on current studies, C2c1 nuclease activity also requires relies on recognition of PAM sequence. C2c1 PAM sequences are T-rich sequences. In some embodiments, the PAM sequence is 5′ TTN 3′ or 5′ ATTN 3′, wherein N is any nucleotide. In a particular embodiment, the PAM sequence is 5′ TTC 3′. In a particular embodiment, the PAM is in the sequence of Plasmodium falciparum. C2c1 creates a staggered cut at the target locus, with a 5′ overhang, or a “sticky end” at the PAM distal side of the target sequence. In some embodiments, the 5′ overhang is 7 nt. See Lewis and Ke, Mol Cell. 2017 Feb. 2; 65(3):377-379.

In particular embodiments, the effector protein is a C2c1 effector protein from an organism from a genus comprising Alicyclobacillus, Desulfovibrio, Desulfonatronum, Opitutaceae, Tuberibacillus, Bacillus, Brevibacillus, Candidatus, Desulfatirhabdium, Citrobacter, Elusimicrobia, Methylobacterium, Omnitrophica, Phycisphaerae, Planctomycetes, Spirochaetes, and Verrucomicrobiaceae.

In further particular embodiments, the C2c1 effector protein is from a species selected from Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060).

In certain example embodiments, the Cas protein may be a Type V CRISPR-Cas, a Type VI CRISPR-Cas, or combination thereof. In certain example embodiments, the Type V or Type VI Cas protein is a thermostable case protein with a nuclease activity above at least 500 C. In certain example embodiments, the Cas protein is a Cas12b protein. In certain other example embodiments, the Cas12b is Alicyclobacillus acidiphilus (AapCas12b). In certain other example embodiments, the Cas12b protein is Brevibacillus sp. SYSU G02855 (BrCas12b). In certain example embodiments, the Cas12 protein is any described in International Patent Application Publication No. WO2021/163584, which is incorporated by reference as if expressed in its entirety herein, particularly any Cas protein set forth in Table 2A, 2B or both therein. In certain embodiments, the Cas 12 protein has at least 80% identity (e.g., 80-100% identity) to a polypeptide from Table 2A or 2B of International Patent Application Publication No. WO2021/163584. In some embodiments, the Cas 12 protein has at least 80% identity (e.g., 80-100% identity) to any one or more of SEQ ID NOS: 61644-61990 of International Patent Application Publication No. WO2021/163584..

In certain embodiments, the CRISPR-Cas protein is a Cas12b from a thermostable species, for example Alicyclobacillus acidiphilus (Aap). Cas 12a proteins can be identified from similar organisms as identified in any of BROD_5090P4_Cas12b_sequences.txt (see U.S. Application No. 63/019,406). In certain embodiments, the AapCas12b protein comprises a sequence with 80%, 85%, 90%, 95% identity to, or consisting of a sequence according to SEQ ID NO: 61995 of International Patent Application Publication No. WO2021/163584. In certain embodiments, the CRISPR-Cas protein is a BrCas12b. In certain embodiments, the BrCas12b protein comprises a sequence with 80%, 85%, 90°/, 95% identity to, or consisting of a sequence according to SEQ ID NO: 61995 of International Patent Application Publication No. WO2021/163584.

The effector protein may comprise a chimeric effector protein comprising a first fragment from a first effector protein (e.g., a C2c1) ortholog and a second fragment from a second effector (e.g., a C2c1) protein ortholog, and wherein the first and second effector protein orthologs are different. At least one of the first and second effector protein (e.g., a C2c1) orthologs may comprise an effector protein (e.g., a C2c1) from an organism comprising Alicyclobacillus, Desulfovibrio, Desulfonatronum, Opitutaceae, Tuberibacillus, Bacillus, Brevibacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae; e.g., a chimeric effector protein comprising a first fragment and a second fragment wherein each of the first and second fragments is selected from a C2c1 of an organism comprising Alicyclobacillus, Desulfovibrio, Desulfonatronum, Opitutaceae, Tuberibacillus, Bacillus, Brevibacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae wherein the first and second fragments are not from the same bacteria; for instance a chimeric effector protein comprising a first fragment and a second fragment wherein each of the first and second fragments is selected from a C2c1 of Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060), wherein the first and second fragments are not from the same bacteria.

In a more preferred embodiment, the C2c1p is derived from a bacterial species selected from Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060). In certain embodiments, the C2c1p is derived from a bacterial species selected from Alicyclobacillus acidoterrestris (e.g., ATCC 49025), Alicyclobacillus contaminans (e.g., DSM 17975).

In particular embodiments, the homologue or orthologue of C2c1 as referred to herein has a sequence homology or identity of at least 80%, more preferably at least 85%, even more preferably at least 90%, such as for instance at least 95% with C2c1. In further embodiments, the homologue or orthologue of C2c1 as referred to herein has a sequence identity of at least 80%, more preferably at least 85%, even more preferably at least 90%, such as for instance at least 95% with the wild type C2c1. Where the C2c1 has one or more mutations (mutated), the homologue or orthologue of said C2c1 as referred to herein has a sequence identity of at least 80%, more preferably at least 85%, even more preferably at least 90%, such as for instance at least 95% with the mutated C2c1.

In an embodiment, the C2c1 protein may be an ortholog of an organism of a genus which includes, but is not limited to Alicyclobacillus, Desulfovibrio, Desulfonatronum, Opitutaceae, Tuberibacillus, Bacillus, Brevibacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae; in particular embodiments, the type V Cas protein may be an ortholog of an organism of a species which includes, but is not limited to Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060).In particular embodiments, the homologue or orthologue of C2c1 as referred to herein has a sequence homology or identity of at least 80%, more preferably at least 85%, even more preferably at least 90%, such as for instance at least 95% with one or more of the C2c1 sequences disclosed herein. In further embodiments, the homologue or orthologue of C2c1 as referred to herein has a sequence identity of at least 80%, more preferably at least 85%, even more preferably at least 90%, such as for instance at least 95% with the wild type AacC2c1 or BthC2c1.

In particular embodiments, the C2c1 protein of the invention has a sequence homology or identity of at least 60%, more particularly at least 70, such as at least 80%, more preferably at least 85%, even more preferably at least 90%, such as for instance at least 95% with AacC2c1 or BthC2c1. In further embodiments, the C2c1 protein as referred to herein has a sequence identity of at least 60%, such as at least 70%, more particularly at least 80%, more preferably at least 85%, even more preferably at least 90%, such as for instance at least 95% with the wild type AacC2c1. In particular embodiments, the C2c1 protein of the present invention has less than 60% sequence identity with AacC2c1. The skilled person will understand that this includes truncated forms of the C2c1 protein whereby the sequence identity is determined over the length of the truncated form.

In certain methods according to the present invention, the CRISPR-Cas protein is preferably mutated with respect to a corresponding wild-type enzyme such that the mutated CRISPR-Cas protein lacks the ability to cleave one or both DNA strands of a target locus containing a target sequence. In particular embodiments, one or more catalytic domains of the C2c1 protein are mutated to produce a mutated Cas protein which cleaves only one DNA strand of a target sequence.

In particular embodiments, the CRISPR-Cas protein may be mutated with respect to a corresponding wild-type enzyme such that the mutated CRISPR-Cas protein lacks substantially all DNA cleavage activity. In some embodiments, a CRISPR-Cas protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form.

In certain embodiments of the methods provided herein the CRISPR-Cas protein is a mutated CRISPR-Cas protein which cleaves only one DNA strand, i.e., a nickase. More particularly, in the context of the present invention, the nickase ensures cleavage within the non-target sequence, i.e., the sequence which is on the opposite DNA strand of the target sequence and which is 3′ of the PAM sequence. By means of further guidance, and without limitation, an arginine-to-alanine substitution (R911A) in the Nuc domain of C2c1 from Alicyclobacillus acidoterrestris converts C2c1 from a nuclease that cleaves both strands to a nickase (cleaves a single strand). It will be understood by the skilled person that where the enzyme is not AacC2c1, a mutation may be made at a residue in a corresponding position.

In certain embodiments of the methods provided herein the CRISPR-Cas protein is a mutated CRISPR-Cas protein which cleaves only one DNA strand, i.e., a nickase. More particularly, in the context of the present invention, the nickase ensures cleavage within the non-target sequence, i.e., the sequence which is on the opposite DNA strand of the target sequence and which is 3′ of the PAM sequence. By means of further guidance, and without limitation, an arginine-to-alanine substitution (R911A) in the Nuc domain of C2c1 from Alicyclobacillus acidoterrestris converts C2c1 from a nuclease that cleaves both strands to a nickase (cleaves a single strand). It will be understood by the skilled person that where the enzyme is not AacC2c1, a mutation may be made at a residue in a corresponding position.

In certain embodiments, the C2c1 protein is a catalytically inactive C2c1 which comprises a mutation in the RuvC domain. In some embodiments, the catalytically inactive C2c1 protein comprises a mutation corresponding to amino acid positions D570, E848, or D977 in Alicyclobacillus acidoterrestris C2c1. In some embodiments, the catalytically inactive C2c1 protein comprises a mutation corresponding to D570A, E848A, or D977A in Alicyclobacillus acidoterrestris C2c1.

The programmability, specificity, and collateral activity of the RNA-guided C2c1 also make it an ideal switchable nuclease for non-specific cleavage of nucleic acids. In one embodiment, a C2c1 system is engineered to provide and take advantage of collateral non-specific cleavage of RNA. In another embodiment, a C2c1 system is engineered to provide and take advantage of collateral non-specific cleavage of ssDNA. Accordingly, engineered C2c1 systems provide platforms for nucleic acid detection and transcriptome manipulation, and inducing cell death. C2c1 is developed for use as a mammalian transcript knockdown and binding tool. C2c1 is capable of robust collateral cleavage of RNA and ssDNA when activated by sequence-specific targeted DNA binding.

In certain embodiments, C2c1 is provided or expressed in an in vitro system or in a cell, transiently or stably, and targeted or triggered to non-specifically cleave cellular nucleic acids. In one embodiment, C2c1 is engineered to knock down ssDNA, for example viral ssDNA. In another embodiment, C2c1 is engineered to knock down RNA. The system can be devised such that the knockdown is dependent on a target DNA present in the cell or in vitro system, or triggered by the addition of a target nucleic acid to the system or cell.

In an embodiment, the C2c1 system is engineered to non-specifically cleave RNA in a subset of cells distinguishable by the presence of an aberrant DNA sequence, for instance where cleavage of the aberrant DNA might be incomplete or ineffectual. In one non-limiting example, a DNA translocation that is present in a cancer cell and drives cell transformation is targeted. Whereas a subpopulation of cells that undergoes chromosomal DNA and repair may survive, non-specific collateral ribonuclease activity advantageously leads to cell death of potential survivors.

Collateral activity was recently leveraged for a highly sensitive and specific nucleic acid detection platform termed SHERLOCK assay that is useful for many clinical diagnoses (Gootenberg, J. S. et al. Nucleic acid detection with CRISPR-Cas13a/C2c2. Science 356, 438-442 (2017)).

### Cas 12c Orthologs

In certain embodiments, the effector protein, particularly a Type V loci effector protein, more particularly a Type V-C loci effector protein, even more particularly a C2c3p, may originate, may be isolated or may be derived from a bacterial metagenome selected from the group consisting of the bacterial metagenomes listed in the Table in FIG. 43A-43B of PCT/US2016/038238, specifically incorporated by reference, which presents analysis of the Type-V-C Cas12c loci.

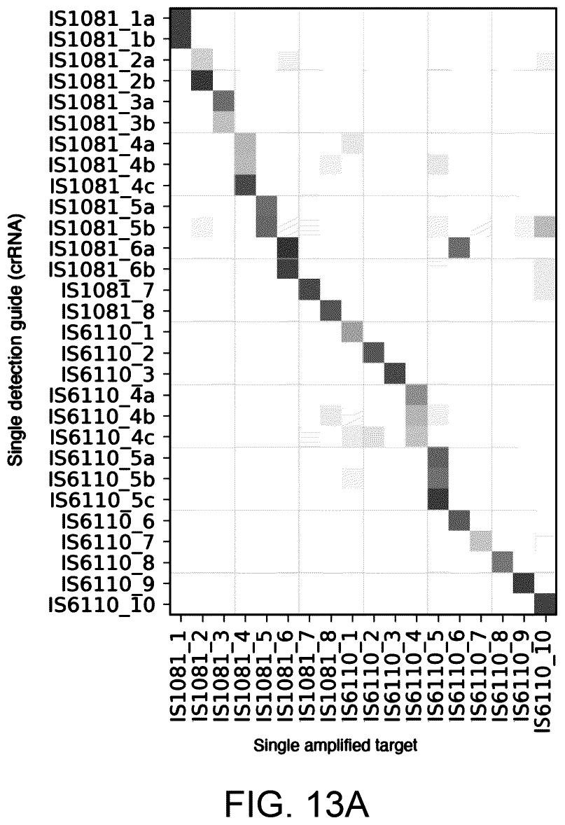

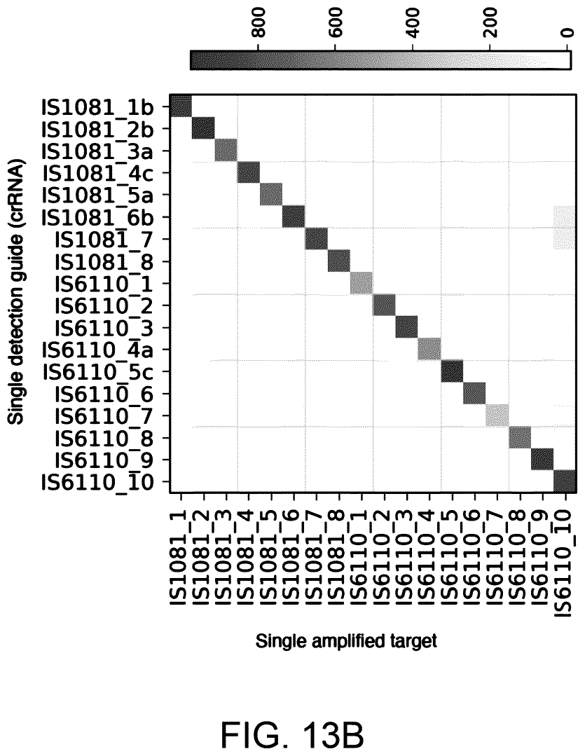

In certain embodiments, the effector protein, particularly a Type V loci effector protein, more particularly a Type V-C loci effector protein, even more particularly a C2c3p, may comprise, consist essentially of or consist of an amino acid sequence selected from the group consisting of amino acid sequences shown in the multiple sequence alignment in FIG. 13I of PCT/US2016/038238, specifically incorporated by reference.

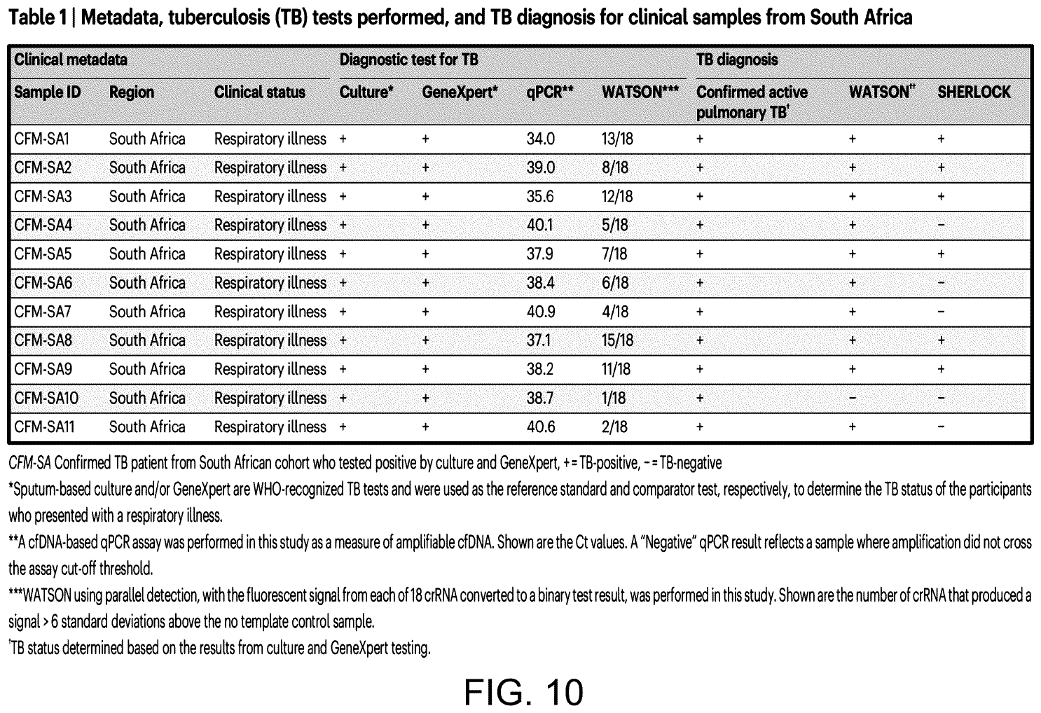

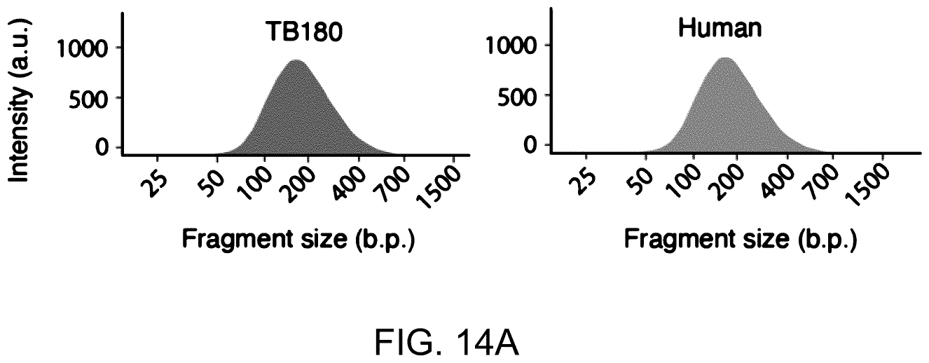

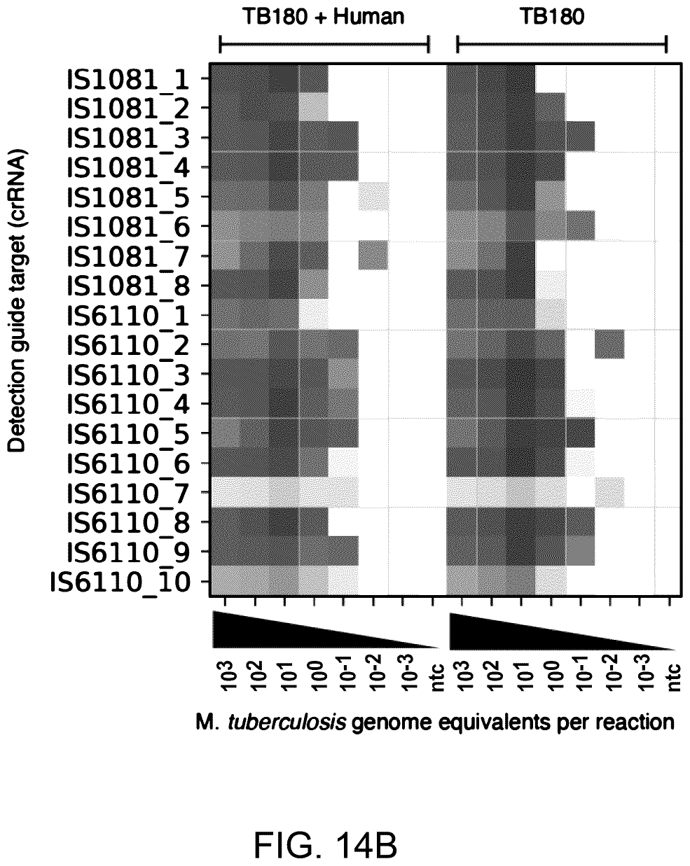

In certain embodiments, a Type V-C locus as intended herein may encode Cas1 and the C2c3p effector protein. See FIG. 14 of PCT/US2016/038238, specifically incorporated by reference, depicting the genomic architecture of the Cas12c CRISPR-Cas loci. In certain embodiments, a Cas1 protein encoded by a Type V-C locus as intended herein may cluster with Type I-B system. See FIG. 10A and 10B and FIG. 10C-V of PCT/US2016/038238, specifically incorporated by reference, illustrating a Cas1 tree including Cas1 encoded by representative Type V-C loci.

In certain embodiments, the effector protein, particularly a Type V loci effector protein, more particularly a Type V-C loci effector protein, even more particularly a C2c3p, such as a native C2c3p, may be about 1100 to about 1500 amino acids long, e.g., about 1100 to about 1200 amino acids long, or about 1200 to about 1300 amino acids long, or about 1300 to about 1400 amino acids long, or about 1400 to about 1500 amino acids long, e.g., about 1100, about 1200, about 1300, about 1400 or about 1500 amino acids long, or at least about 1100, at least about 1200, at least about 1300, at least about 1400 or at least about 1500 amino acids long.

In certain embodiments, the effector protein, particularly a Type V loci effector protein, more particularly a Type V-C loci effector protein, even more particularly a C2c3p, and preferably the C-terminal portion of said effector protein, comprises the three catalytic motifs of the RuvC-like nuclease (i.e., RuvCI, RuvCII and RuvCIII). In certain embodiments, said effector protein, and preferably the C-terminal portion of said effector protein, may further comprise a region corresponding to the bridge helix (also known as arginine-rich cluster) that in Cas9 protein is involved in crRNA-binding. In certain embodiments, said effector protein, and preferably the C-terminal portion of said effector protein, may further comprise a Zn finger region. Preferably, the Zn-binding cysteine residue(s) may be conserved in C2c3p. In certain embodiments, said effector protein, and preferably the C-terminal portion of said effector protein, may comprise the three catalytic motifs of the RuvC-like nuclease (i.e., RuvCI, RuvCII and RuvCIII), the region corresponding to the bridge helix, and the Zn finger region, preferably in the following order, from N to C terminus: RuvCI-bridge helix-RuvCII-Zinc finger-RuvCIII. See FIGS. 13A and 13C of PCT/US2016/038238, specifically incorporated by reference, for illustration of representative Type V-C effector proteins domain architecture.

In certain embodiments, Type V-C loci as intended herein may comprise CRISPR repeats between 20 and 30 bp long, more typically between 22 and 27 bp long, yet more typically 25 bp long, e.g., 20, 21, 22, 23, 24, 25, 26, 27, 28, 29, or 30 bp long.

Orthologous proteins may but need not be structurally related, or are only partially structurally related. In particular embodiments, the homologue or orthologue of a Type V protein such as Cas12c as referred to herein has a sequence homology or identity of at least 80%, more preferably at least 85%, even more preferably at least 90%, such as for instance at least 95% with a Cas12c. In further embodiments, the homologue or orthologue of a Type V Cas12c as referred to herein has a sequence identity of at least 80%, more preferably at least 85%, even more preferably at least 90%, such as for instance at least 95% with the wild type Cas12c.

In an embodiment, the Type V RNA-targeting Cas protein may be a Cas12c ortholog of an organism of a genus which includes but is not limited to Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter.

In an embodiment, the Cas12c or an ortholog or homolog thereof, may comprise one or more mutations (and hence nucleic acid molecule(s) coding for same may have mutation(s). The mutations may be artificially introduced mutations and may include but are not limited to one or more mutations in a catalytic domain. Examples of catalytic domains with reference to a Cas9 enzyme may include but are not limited to RuvC I, RuvC II, RuvC III and HNH domains. In an embodiment, the Cas12c or an ortholog or homolog thereof, may comprise one or more mutations. The mutations may be artificially introduced mutations and may include but are not limited to one or more mutations in a catalytic domain. Examples of catalytic domains with reference to a Cas enzyme may include but are not limited to RuvC I, RuvC IL, RuvC III, HNH domains, and HEPN domains.

### Cas12f Orthologs

In some embodiments, the Cas12 is Cas12f (also known as Cas14). In general, Cas12f is smaller in size than other Cas12 proteins or Cas9, which can be advantageous for in-cell detection assays. Cas12f can also have increased specificity towards ssDNA than Cas12a, making it advantageously suitable for use in assays that are configured to detect single nucleotide differences at certain protospacer sites (see e.g., Harrington et al. Science. 2018. 362:839-842).

In some embodiments, the Cas12f is any one set forth and described in Karvelis et al., 2020. Nucleic Acids Res. 48(9):5016-5023; Harrington et al. Science 2018. 362(6416):839-842; Savag, D. F. 2019. Biochemistry. 58(8):1024-1025. Aquino-Jarquin G. Nanomedicine. 2019.18:428-431; and/or Takeda et al., Mol. Cell. 2021. 81(3):558-570. In some embodiments, the Cas12f proteins are about 400 to about 700 amino acids in size.

### Detection Constructs

The nucleic acid detection system can include one or more oligonucleotide-based detection constructs. Generally, a detection construct is a molecule that can be cleaved or otherwise deactivated by an activated CRISPR system effector protein described herein. The detection construct can be capable of producing a detection signal, such as a positive detection signal, in response to a CRISPR system described herein specifically acting on or otherwise specifically interacting with a target polynucleotide. In some embodiments, the nucleic acid detection system includes two or more oligonucleotide-based detection constructs. In some embodiments the collateral activity of the effector molecule(s) in the CRISPR system can modify the detection construct. In some embodiments, the collateral activity is DNA or RNA collateral activity. In some embodiments the modification resulting from the collateral activity of the CRISPR system effector molecule(s) is cleavage of DNA or RNA. In some embodiments the modification can directly or indirectly generate a detection signal, such as a positive detection signal.

In some embodiments, the detection construct is or includes an RNA or DNA oligonucleotide that includes a first molecule on a first end and a second molecule on a second end. The first and second molecules can be part of a binding pair as is discussed in greater detail below. In some embodiments, the first molecule can quench the second molecule when the detection construct is in the unmodified state. In other words, the first molecule can prevent the second molecule from producing a detectable positive signal when the detection construct is in the unmodified state. When the detection construct is modified, the second molecule can be released, have a conformational change, or be otherwise positioned in relation to the first molecule such that the second molecule can produce a detectable signal. The oligonucleotide (DNA or RNA) component can be modified by an activated CRISPR system effector(s). Modification can be cleavage, sequence modification, base editing, methylation, demethylation, or other modification. In some embodiments, the modification is cleavage of the oligonucleotide component.

In some embodiments, the composition of the oligonucleotide component may be generic i.e., not the same as target molecule. In some embodiments, the detector construct is configured so that it prevents or masks generation of a detectable positive signal when in the unmodified (e.g., uncleaved) configuration, but allows or facilitates generation of a positive detectable signal when cleaved or otherwise modified. In the context of the present invention, detection constructs can be composed of or include a first molecule and a second molecule connected by an RNA or DNA nucleic acid linker. Use of an RNA or DNA linker will depend on whether the CRISPR effector protein(s) used have RNA or DNA collateral activity. The first and/or second molecule can be part of a binding pair. In some embodiments the binding partners for each of the first and second can be affixed to a substrate that can be part of a container and/or device (including, but not limited to, a lateral flow substrate) or be free in solution as is described in greater detail elsewhere herein. As a reaction control, in some embodiments, in the unmodified state only detection of binding between e.g., the first molecule and its binding partner will be detected via a positive signal. In some embodiments, after modification of the detection construct by the activated CRISPR effector(s), the second molecule can be released from the detector construct and/or bind its binding partner and produce a detectable positive signal indicating the presence of the target molecule. Where a reaction control positive detectable signal is included in the reaction, the detectable positive signal of the reaction control and the detectable positive signal indicating presence of the target molecule are distinguishable from one another by signal type (e.g., different labels, dyes, reactions etc.) and/or by position on a substrate.

A positive detectable signal may be any signal that can be detected using optical, fluorescent, chemiluminescent, electrochemical or other detection methods known in the art. The term “positive detectable signal” is used to differentiate from other detectable signals that may be detectable in the presence of the detection construct. For example, in certain embodiments a first signal may be detected when a detection agent is present (i.e., a negative detectable signal), which then converts to a second signal (e.g., the positive detectable signal) upon detection of the target molecules and cleavage or deactivation of the masking agent by the activated CRISPR effector protein.

A detection agent can be included in some embodiments, that specifically binds the one of the molecules that is not bound to a binding partner. For example, the detection agent can be or include a detectable label.

In some embodiments, the detection construct can be configured for a CRISPR-Cas based nucleic acid detection reaction, such as a SHERLOCK (Specific High Sensitivity Enzymatic Reporter UnLOCKing) reaction or DETECTR reaction. Exemplary SHERLOCK systems and the reactions they facilitate are described in e.g., Kellner et al. Nat. Protoc. 2019. 14(10):2986-3012, International Patent Publications WO 2018/07129, WO 2018/180340, WO 2019/051318, WO 2019/071051, WO 2019/126577; WO 2019/148206, WO 2020/0060067, WO 2020/006049, WO 2020/006036, US Pubs. 2018/0298445, US 2019-0144929, 2018/0305773, Gootenberg et al. 2017, Science. 356:438-442, Gootenberg et al., 2018. Science. 360:439-444, Myhrvold et al. Science. 360:444-448, Jong et al. Point-of-care testing for COVID-19 using SHERLOCK diagnostics. medRxiv 2020.05.04.20091231; doi: https://doi.org/10.1101/2020.05.04.20091231, Abudayyeh et al., CRISPRJ. 2019. 2(3):165-171; Broughten et al., 2020. CRISPR-Cas12-based detection of SARS-CoV2. Nat. Biotech. 38:870-874; Mustafa and Makhawi et al. 2021. Biotechnol. https://doi.org/10.1128/JCM.00745-20 and others herein, which are each incorporated by reference as if expressed in their entirety herein. If a target molecule is present in a sample, the corresponding guide molecule will guide the CRSIPR Cas/guide complex to the target molecule by hybridizing with the target molecule, thereby triggering the CRISPR effector protein's nuclease activity. This activated CRISPR effector protein will cleave both the target molecule and then non-specifically cleave the linker portion of the detection construct, resulting in a detectable signal.

In some embodiments, the detection construct comprises a detection construct configured to suppress generation of a detectable positive signal until the non-target sequence is cleaved by exhibited collateral activity of the one or more CRISPR-Cas proteins. The detection construct can suppress generation of a detectable positive signal by detection the detectable positive signal or generating by a detectable negative signal. The detection construct can include a silencing RNA that suppresses generation of a gene product encoded by a reporting construct, wherein the gene product generates the detectable positive signal when expressed. The detection construct can include a ribozyme that generates the negative detectable signal, and wherein the positive detectable signal is generated when the ribozyme is deactivated. In some embodiments, the ribozyme converts a substrate to a first color and wherein the substrate converts to a second color when the ribozyme is deactivated.

In some embodiments, the detection construct is a DNA or RNA aptamer and/or includes a DNA or RNA-tethered inhibitor. In some embodiments, the aptamer or DNA- or RNA-tethered inhibitor sequesters an enzyme, wherein the enzyme generates a detectable signal upon release from the aptamer or DNA or RNA tethered inhibitor by acting upon a substrate. In some embodiments, the aptamer is an inhibitor aptamer that inhibits an enzyme and prevents the enzyme from catalyzing generation of a detectable signal from a substance or wherein the DNA- or RNA-tethered inhibitor inhibits an enzyme and prevents the enzyme from catalyzing generation of a detectable signal from a substrate. In some embodiments, the enzyme is thrombin and the substrate is para-nitroanilide covalently linked to a peptide substrate for thrombin, or 7-amino-4 methylcoumarin covalently linked to a peptide substrate for thrombin. In some embodiments, the aptamer sequesters a pair of agents that when released from the aptamers combine to generate a detectable signal.

In some embodiments, the detection construct comprises a DNA or RNA oligonucleotide to which a detectable ligand and a detection component are attached. In some embodiments, the detection construct comprises DNA or RNA in complex with an intercalating agent, wherein the intercalating agent changes absorbance upon cleavage of the DNA or RNA, optionally wherein the intercalating agent is pyronine-Y or methylene blue, optionally wherein the detectable ligand is a fluorophore and the detection component is a quencher molecule. In some embodiments, the detection construct comprises a nanoparticle held in aggregate by bridge molecules, wherein at least a portion of the bridge molecules comprises DNA or RNA, and wherein the solution undergoes a color shift when the nanoparticle is disbursed in solution, optionally wherein the nanoparticle is colloidal metal, optionally colloidal gold.

In some embodiments, the detection construct comprising a quantum dot linked to one or more quencher molecules by a linking molecule, wherein at least a portion of the linking molecule comprises DNA or RNA.

In some embodiments, the detection construct comprises DNA or RNA in complex with an intercalating agent, wherein the intercalating agent changes absorbance upon cleavage of the DNA or RNA, optionally wherein the intercalating agent is pyronine-Y or methylene blue, optionally wherein the detectable ligand is a fluorophore and the detection component is a quencher molecule.

In certain example embodiments, the detection construct may suppress generation of a gene product. The gene product may be encoded by a reporter construct that is added to the sample. The detection construct may be an interfering RNA involved in a RNA interference pathway, such as a short hairpin RNA (shRNA) or small interfering RNA (siRNA). The detection construct may also comprise microRNA (miRNA). While present, the detection construct suppresses expression of the gene product. The gene product may be a fluorescent protein or other RNA transcript or proteins that would otherwise be detectable by a labeled probe, aptamer, or antibody but for the presence of the masking construct. Upon activation of the effector protein the detection construct is cleaved or otherwise silenced allowing for expression and detection of the gene product as the positive detectable signal.

In certain example embodiments, the detection construct may sequester one or more reagents needed to generate a detectable positive signal such that release of the one or more reagents from the detection construct results in generation of the detectable positive signal. The one or more reagents may combine to produce a colorimetric signal, a chemiluminescent signal, a fluorescent signal, or any other detectable signal and may comprise any reagents known to be suitable for such purposes. In certain example embodiments, the one or more reagents are sequestered by RNA aptamers that bind the one or more reagents. The one or more reagents are released when the effector protein is activated upon detection of a target molecule and the RNA aptamers are degraded.

In certain example embodiments, the detection construct may be immobilized on a solid substrate in an individual discrete volume (defined further elsewhere herein) and sequesters a single reagent involved in producing a detectable signal.

In certain example embodiments, the detection construct may comprise a ribozyme. Ribozymes are RNA molecules having catalytic properties. Ribozymes, both naturally and engineered, comprise or consist of RNA that may be targeted by the effector proteins disclosed herein. The ribozyme may be selected or engineered to catalyze a reaction that either generates a negative detectable signal or prevents generation of a positive control signal. Upon deactivation of the ribozyme by the activated effector protein the reaction generating a negative control signal, or preventing generation of a positive detectable signal, is removed thereby allowing a positive detectable signal to be generated. Alternatively, ribozymes, when present can generate cleavage products of, for example, RNA transcripts. Thus, detection of a positive detectable signal may comprise detection of non-cleaved RNA transcripts that are only generated in the absence of the ribozyme.

In certain embodiments, RNAse activity is detected via cleavage of enzyme-inhibiting aptamers, which can result in signal amplification.

In certain example embodiments, the detection construct may comprise an initiator for an HCR reaction. See e.g., Dirks and Pierce. PNAS 101, 15275-15728 (2004), results of which can be detected by any suitable method such as colometrically.

In some embodiments, the nucleic acid detection system includes two or more CRISPR-Cas systems each comprising one or more CRISPR-Cas proteins capable of exhibiting collateral activity and two or more oligonucleotide-based detection constructs each having a detection construct, wherein the detection construct of each of the oligonucleotide-based detection constructs is preferentially cut by one of the two or more CRISPR-Cas proteins exhibiting collateral activity. Utilizing two or more oligonucleotide probes allows for the ability to simultaneously detect multiple sample inputs, also allowing for multiplexed detection panels or for in sample controls. Orthogonal base preferences of the Cas13 enzymes as described herein offer the opportunity to have multiplexed detection systems. In some embodiments, the collateral activity of different Cas13 and/or Cas12 enzymes in the same reaction via fluorescent homopolymer sensors of different base identities and fluorophore colors, enabling multiple targets to be simultaneously measured.

### Multiplexed Guides and Tiled Guide Sets for CRISPR-Cas Based Detection

In general, guide design and selection for CRISPR-Cas systems is known. See e.g., WO 2020/236972, particularly at para. [0139]-[0357], which can be adapted for use with the present invention.. Other guide design and selection approaches are described elsewhere herein. Target selection for CRISPR-Cas systems is also generally known See e.g., WO 2020/236972, particularly at para. [0358]-[0372], which can be adapted for use with the present invention. Other target selection considerations are discussed elsewhere herein.

### Devices

The nucleic acid detection systems or one or more components thereof described herein can be embodied on diagnostic devices. The devices can be, for example, point-of-care (POC) devices, microfluidic devices, and/or the like that are configured to perform one or more steps of the methods described herein.

A number of substrates and configurations of devices capable of defining multiple individual discrete volumes within the device may be used. As used herein “individual discrete volume” refers to a discrete space, such as a container, receptacle, or other arbitrary defined volume or space that can be defined by properties that prevent and/or inhibit migration of target molecules, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof that can contain a target molecule and a indexable nucleic acid identifier (for example nucleic acid barcode). By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the use of non-walled, or semipermeable discrete volumes is that some reagents, such as buffers, chemical activators, or other agents may be passed through the discrete volume, while other materials, such as target molecules, may be maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain embodiments, the compartment is an aqueous droplet in a water-in-oil emulsion. In specific embodiments, any of the applications, methods, or systems described herein requiring exact or uniform volumes may employ the use of an acoustic liquid dispenser.

In certain example embodiments, the device comprises a flexible material substrate on which a number of spots may be defined. Flexible substrate materials suitable for use in diagnostics and biosensing are known within the art. The flexible substrate materials may be made of plant derived fibers, such as cellulosic fibers, or may be made from flexible polymers such as flexible polyester films and other polymer types. Within each defined spot, reagents of the system described herein are applied to the individual spots. Each spot may contain the same reagents except for a different guide RNA or set of guide RNAs, or where applicable, a different detection aptamer to screen for multiple targets at once. Thus, the systems and devices herein may be able to screen samples from multiple sources (e.g., multiple clinical samples from different individuals) for the presence of the same target, or a limited number of target, or aliquots of a single sample (or multiple samples from the same source) for the presence of multiple different targets in the sample. In certain example embodiments, the elements of the systems described herein are freeze dried onto the paper or cloth substrate. Example flexible material based substrates that may be used in certain example devices are disclosed in Pardee et al. Cell. 2016, 165(5):1255-66 and Pardee et al. Cell. 2014, 159(4):950-54. Suitable flexible material-based substrates for use with biological fluids, including blood are disclosed in International Patent Application Publication No. WO/2013/071301 entitled “Paper based diagnostic test” to Shevkoplyas et al. U.S. Patent Application Publication No. 2011/0111517 entitled “Paper-based microfluidic systems” to Siegel et al. and Shafiee et al. “Paper and Flexible Substrates as Materials for Biosensing Platforms to Detect Multiple Biotargets” Scientific Reports 5:8719 (2015). Further flexible based materials, including those suitable for use in wearable diagnostic devices are disclosed in Wang et al. “Flexible Substrate-Based Devices for Point-of-Care Diagnostics” Cell 34(11):909-21 (2016). Further flexible based materials may include nitrocellulose, polycarbonate, methylethyl cellulose, polyvinylidene fluoride (PVDF), polystyrene, or glass (see e.g., US20120238008). In certain embodiments, discrete volumes are separated by a hydrophobic surface, such as but not limited to wax, photoresist, or solid ink.

In some embodiments, a dosimeter or badge may be provided that serves as a sensor or indicator such that the wearer is notified of exposure to certain microbes or other agents. For example, the systems described herein may be used to detect a particular pathogen. Likewise, aptamer based embodiments disclosed above may be used to detect both polypeptide as well as other agents, such as chemical agents, to which a specific aptamer may bind. Such a device may be useful for surveillance of soldiers or other military personnel, as well as clinicians, researchers, hospital staff, and the like, in order to provide information relating to exposure to potentially dangerous microbes as quickly as possible, for example for biological or chemical warfare agent detection. In other embodiments, such a surveillance badge may be used for preventing exposure to dangerous microbes or pathogens in immunocompromised patients, burn patients, patients undergoing chemotherapy, children, or elderly individuals.

Samples sources that may be analyzed using the systems and devices described herein include biological samples of a subject or environmental samples. Environmental samples may include surfaces or fluids. The biological samples may include, but are not limited to, saliva, blood, plasma, sera, stool, urine, sputum, mucous, lymph, synovial fluid, spinal fluid, cerebrospinal fluid, a swab from skin or a mucosal membrane, or combination thereof. In an example embodiment, the environmental sample is taken from a solid surface, such as a surface used in the preparation of food or other sensitive compositions and materials.

In other example embodiments, the elements of the systems described herein may be place on a single use substrate, such as swab or cloth that is used to swab a surface or sample fluid. For example, the system could be used to test for the presence of a pathogen on a food by swabbing the surface of a food product, such as a fruit or vegetable. Similarly, the single use substrate may be used to swab other surfaces for detection of certain microbes or agents, such as for use in security screening. Single use substrates may also have applications in forensics, where the CRISPR systems are designed to detect, for example identifying DNA SNPs that may be used to identify a suspect, or certain tissue or cell markers to determine the type of biological matter present in a sample. Likewise, the single use substrate could be used to collect a sample from a patient—such as a saliva sample from the mouth—or a swab of the skin. In other embodiments, a sample or swab may be taken of a meat product on order to detect the presence of absence of contaminants on or within the meat product.

Near-real-time microbial diagnostics are needed for food, clinical, industrial, and other environmental settings (see e.g., Lu T K, Bowers J, and Koeris M S., Trends Biotechnol. 2013 June; 31(6):325-7). In certain embodiments, the present invention is used for rapid detection of foodborne pathogens using guide RNAs specific to a pathogen (e.g., Campylobacter jejuni, Clostridium perfringens, Salmonella spp., Escherichia coli, Bacillus cereus, Listeria monocytogenes, Shigella spp., Staphylococcus aureus, Staphylococcal enteritis, Streptococcus, Vibrio cholerae, Vibrio parahaemolyticus, Vibrio vulnificus, Yersinia enterocolitica and Yersinia pseudotuberculosis, Brucella spp., Corynebacterium ulcerans, Coxiella burnetii, or Plesiomonas shigelloides).

In certain embodiments, the device is or comprises a flow strip. For instance, a lateral flow strip allows for RNAse (e.g., C2c2) detection by color. The RNA reporter is modified to have a first molecule (such as for instance FITC) attached to the 5′ end and a second molecule (such as for instance biotin) attached to the 3′ end (or vice versa). The lateral flow strip is designed to have two capture lines with anti-first molecule (e.g., anti-FITC) antibodies hybridized at the first line and anti-second molecule (e.g., anti-biotin) antibodies at the second downstream line. As the SHERLOCK reaction flows down the strip, uncleaved reporter will bind to anti-first molecule antibodies at the first capture line, while cleaved reporters will liberate the second molecule and allow second molecule binding at the second capture line. Second molecule sandwich antibodies, for instance conjugated to nanoparticles, such as gold nanoparticles, will bind any second molecule at the first or second line and result in a strong readout/signal (e.g., color). As more reporter is cleaved, more signal will accumulate at the second capture line and less signal will appear at the first line. In certain aspects, the invention relates to the use of a follow strip as described herein for detecting nucleic acids or polypeptides. In certain aspects, the invention relates to a method for detecting nucleic acids or polypeptides with a flow strip as defined herein, e.g., (lateral) flow tests or (lateral) flow immunochromatographic assays.

In certain example embodiments, the device is a microfluidic device that generates and/or merges different droplets (i.e., individual discrete volumes). For example, a first set of droplets may be formed containing samples to be screened and a second set of droplets formed containing the elements of the systems described herein. The first and second set of droplets are then merged and then diagnostic methods as described herein are carried out on the merged droplet set. Microfluidic devices disclosed herein may be silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methylacrylate) (PMMA). In one embodiment, soft lithography in PDMS may be used to prepare the microfluidic devices. For example, a mold may be made using photolithography which defines the location of flow channels, valves, and filters within a substrate. The substrate material is poured into a mold and allowed to set to create a stamp. The stamp is then sealed to a solid support, such as but not limited to, glass. Due to the hydrophobic nature of some polymers, such as PDMS, which absorbs some proteins and may inhibit certain biological processes, a passivating agent may be necessary (Schoffner et al. Nucleic Acids Research, 1996, 24:375-379). Suitable passivating agents are known in the art and include, but are not limited to, silanes, parylene, n-Dodecyl-b-D-matoside (DDM), pluronic, Tween-20, other similar surfactants, polyethylene glycol (PEG), albumin, collagen, and other similar proteins and peptides.

In certain example embodiments, the system and/or device may be adapted for conversion to a flow-cytometry readout in or allow to all of sensitive and quantitative measurements of millions of cells in a single experiment and improve upon existing flow-based methods, such as the PrimeFlow assay. In certain example embodiments, cells may be cast in droplets containing unpolymerized gel monomer, which can then be cast into single-cell droplets suitable for analysis by flow cytometry. A detection construct comprising a fluorescent detectable label may be cast into the droplet comprising unpolymerized gel monomer. Upon polymerization of the gel monomer to form a bead within a droplet. Because gel polymerization is through free-radical formation, the fluorescent reporter becomes covalently bound to the gel. The detection construct may be further modified to comprise a linker, such as an amine. A quencher may be added post-gel formation and will bind via the linker to the reporter construct. Thus, the quencher is not bound to the gel and is free to diffuse away when the reporter is cleaved by the CRISPR effector protein. Amplification of signal in droplet may be achieved by coupling the detection construct to a hybridization chain reaction (HCR initiators) amplification. DNA/RNA hybrid hairpins may be incorporated into the gel which may comprise a hairpin loop that has a RNase sensitive domain. By protecting a strand displacement toehold within a hairpin loop that has a Rnase sensitive domain, HCR initiators may be selectively deprotected following cleavage of the hairpin loop by the CRISPR effector protein. Following deprotection of HCR initiators via toehold mediated strand displacement, fluorescent HCR monomers may be washed into the gel to enable signal amplification where the initiators are deprotected.

An example of microfluidic device that may be used in the context of the invention is described in Hou et al. “Direct Detection and drug-resistance profiling of bacteremias using inertial microfluidics” Lap Chip. 15(10):2297-2307 (2016).

In systems described herein, may further be incorporated into wearable medical devices that assess biological samples, such as biological fluids, of a subject outside the clinic setting and report the outcome of the assay remotely to a central server accessible by a medical care professional. The device may include the ability to self-sample blood, such as the devices disclosed in U.S. Patent Application Publication No. 2015/0342509 entitled “Needle-free Blood Draw to Peeters et al., U.S. Patent Application Publication No. 2015/0065821 entitled “Nanoparticle Phoresies” to Andrew Conrad.

In certain example embodiments, the device may comprise individual wells, such as microplate wells. The size of the microplate wells may be the size of standard 6, 24, 96, 384, 1536, 3456, or 9600 sized wells. In certain example embodiments, the elements of the systems described herein may be freeze dried and applied to the surface of the well prior to distribution and use.

Utilizing microfluidic devices in exemplary embodiments can include mixing each component with a unique combination of three encoding dyes. Droplets are then generated separately for each component, pooled together, and loaded onto a microwell array with tens of thousands of wells, each designed to hold exactly two droplets. Droplets settle into wells in a stochastic fashion, giving rise to all possible pair-wise combination of components. Fluorescence microscopy is then used to determine droplet identity based on encoding dye signal. The droplets are then merged using a corona generator (which destabilizes droplet surfaces) and a fourth fluorescence channel is used to readout an assay score after incubation, and is amenable to CRISPR-diagnostics, as disclosed herein.

Microfluidic devices comprise an array of microwells with at least one flow channel beneath the microwells. In certain example embodiments, the device is a microfluidic device that generates and/or merges different droplets (i.e., individual discrete volumes). For example, a first set of droplets may be formed containing samples to be screened and a second set of droplets formed containing the elements of the systems described herein. The first and second set of droplets are then merged and then diagnostic methods as described herein are carried out on the merged droplet set.

Microfluidic devices disclosed herein may be silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methylacrylate) (PMMA). In one embodiment, soft lithography in PDMS may be used to prepare the microfluidic devices. For example, a mold may be made using photolithography which defines the location of flow channels, valves, and filters within a substrate. The substrate material is poured into a mold and allowed to set to create a stamp. The stamp is then sealed to a solid support, such as but not limited to, glass. Due to the hydrophobic nature of some polymers, such as PDMS, which absorbs some proteins and may inhibit certain biological processes, a passivating agent may be necessary (Schoffner et al. Nucleic Acids Research, 1996, 24:375-379). Suitable passivating agents are known in the art and include, but are not limited to, silanes, parylene, n-Dodecyl-b-D-matoside (DDM), pluronic, Tween-20, other similar surfactants, polyethylene glycol (PEG), albumin, collagen, and other similar proteins and peptides.

An example of microfluidic device that may be used in the context of the invention is described in Kulesa, et al. PNAS, 115, 6685-6690, incorporated herein by reference.

In certain example embodiments, the device may comprise individual wells, such as microplate wells. The size of the microplate wells may be the size of standard 6, 24, 96, 384, 1536, 3456, or 9600 sized wells. In certain embodiments, the microwells can number at more than 40,0000 or more than 190,000. In certain example embodiments, the elements of the systems described herein may be freeze dried and applied to the surface of the well prior to distribution and use.

Microwell chips can be designed as disclosed in U.S. Provisional Application Attorney Docket No. 52199-505P03US. In one embodiment, the microwell chip can be designed in a format measuring around 6.2×7.2 cm, containing 49200 microwells, or a larger format, measuring 7.4×10 cm, containing 97, 194 microwells. The array of microwells can be shaped, for example, as two circles of a diameter of about 50-300 μm, in particular embodiments at 150 μm diameter set at 10% overlap. The array of microwells can be arranged in a hexagonal lattice at 50 μm inter-well spacing. In some instances, the microwells can be arranged in other shapes, spacing and sizes in order to hold a varying number of droplets. The microwell chips are advantageously, in some embodiments, sized for use with standard laboratory equipment, including imaging equipment such as microscopes. In some embodiments, the microwell chips can be configured to be utilized with standard microscopes as well as microscopes that include means for incubation. In instances where imaging and incubation can be coupled, assays can enable fluorescence kinetics and quantitation.

In an exemplary method, compounds can be mixed with a unique ratio of fluorescent dyes (e.g., Alexa Fluor 555, 594, 647). Each mixture of target molecule with a dye mixture can be emulsified into droplets. Similarly, each detection CRISPR-Cas system with optical barcode can be emulsified into droplets. In some embodiments, the droplets are approximately 1 nL each. The CRISPR-Cas detection system droplets and target molecule droplets can then be combined and applied to the microwell chip. The droplets can be combined by simple mixing or other methods of combination. In one exemplary embodiment, the microwell chip is suspended on a platform such as a hydrophobic glass slide with removable spacers that can be clamped from above and below by clamps or other securing means, which can be, for example, neodymium magnets. The gap between the chip and the glass created by the spacers can be loaded with oil, and the pool of droplets injected into the chip, continuing to flow the droplets by injecting more oil and draining excess droplets. After loading is completed, the chip can be washed with oil, and spacers can be removed to seal microwells against the glass slide and clamp closed. The chip can be imaged, for example with an epifluorescence microscope, droplets merged to mix the compounds in each microwell by applying an AC electric field, for example, supplied by a corona treater, and subsequently treated according to desired protocols. In one embodiment, the microwell can be incubated at 37° C. with measurement of fluorescence using 81pifluorescence microscope. Following manipulation of the droplets, the droplets can be eluted off of the microwell as described herein for additional analyses, processing and/or manipulations.

The devices disclosed herein may further comprise inlet and outlet ports, or openings, which in turn may be connected to valves, tubes, channels, chambers, and syringes and/or pumps for the introduction and extraction of fluids into and from the device. The devices may be connected to fluid flow actuators that allow directional movement of fluids within the microfluidic device. Example actuators include, but are not limited to, syringe pumps, mechanically actuated recirculating pumps, electroosmotic pumps, bulbs, bellows, diaphragms, or bubbles intended to force movement of fluids. In certain example embodiments, the devices are connected to controllers with programmable valves that work together to move fluids through the device. In certain example embodiments, the devices are connected to the controllers discussed in further detail below. The devices may be connected to flow actuators, controllers, and sample loading devices by tubing that terminates in metal pins for insertion into inlet ports on the device.

As shown herein the elements of the system are stable when freeze dried, therefore embodiments that do not require a supporting device are also contemplated, i.e., the system may be applied to any surface or fluid that will support the reactions disclosed herein and allow for detection of a positive detectable signal from that surface or solution. In addition to freeze-drying, the systems may also be stably stored and utilized in a pelletized form. Polymers useful in forming suitable pelletized forms are known in the art.

In certain embodiments, the CRISPR effector protein (e.g., Cas) is bound to each discrete volume in the device. Each discrete volume may comprise a different guide RNA specific for a different target molecule. In certain embodiments, a sample is exposed to a solid substrate comprising more than one discrete volume each comprising a guide RNA specific for a target molecule. Not being bound by a theory, each guide RNA will capture its target molecule from the sample and the sample does not need to be divided into separate assays. Thus, a valuable sample may be preserved. The effector protein may be a fusion protein comprising an affinity tag. Affinity tags are well known in the art (e.g., HA tag, Myc tag, Flag tag, His tag, biotin). The effector protein may be linked to a biotin molecule and the discrete volumes may comprise streptavidin. In other embodiments, the CRISPR effector protein is bound by an antibody specific for the effector protein. Methods of binding a CRISPR enzyme has been described previously (see, e.g., US20140356867A1).

The devices disclosed herein may also include elements of point of care (POC) devices known in the art for analyzing samples by other methods. See, for example St John and Price, “Existing and Emerging Technologies for Point-of-Care Testing” (Clin Biochem Rev. 2014 August; 35(3): 155-167).

Digital microfluidics can also be utilized with the methods and systems disclosed herein. The present invention may be used with a wireless lab-on-chip (LOC) diagnostic sensor system (see e.g., U.S. Pat. No. 9,470,699 “Diagnostic radio frequency identification sensors and applications thereof”). In certain embodiments, the present invention is performed in a LOC controlled by a wireless device (e.g., a cell phone, a personal digital assistant (PDA), a tablet) and results are reported to said device.

The present invention may be used with a wireless lab-on-chip (LOC) diagnostic sensor system (see e.g., U.S. Pat. No. 9,470,699 “Diagnostic radio frequency identification sensors and applications thereof”). In certain embodiments, the present invention is performed in a LOC controlled by a wireless device (e.g., a cell phone, a personal digital assistant (PDA), a tablet) and results are reported to said device.

Radio frequency identification (RFID) tag systems include an RFID tag that transmits data for reception by an RFID reader (also referred to as an interrogator). In a typical RFID system, individual objects (e.g., store merchandise) are equipped with a relatively small tag that contains a transponder. The transponder has a memory chip that is given a unique electronic product code. The RFID reader emits a signal activating the transponder within the tag through the use of a communication protocol. Accordingly, the RFID reader is capable of reading and writing data to the tag. Additionally, the RFID tag reader processes the data according to the RFID tag system application. Currently, there are passive and active type RFID tags. The passive type RFID tag does not contain an internal power source, but is powered by radio frequency signals received from the RFID reader. Alternatively, the active type RFID tag contains an internal power source that enables the active type RFID tag to possess greater transmission ranges and memory capacity. The use of a passive versus an active tag is dependent upon the particular application.

Lab-on-the chip technology is well described in the scientific literature and consists of multiple microfluidic channels, input or chemical wells. Reactions in wells can be measured using radio frequency identification (RFID) tag technology since conductive leads from RFID electronic chip can be linked directly to each of the test wells. An antenna can be printed or mounted in another layer of the electronic chip or directly on the back of the device. Furthermore, the leads, the antenna and the electronic chip can be embedded into the LOC chip, thereby preventing shorting of the electrodes or electronics. Since LOC allows complex sample separation and analyses, this technology allows LOC tests to be done independently of a complex or expensive reader. Rather a simple wireless device such as a cell phone or a PDA can be used. In one embodiment, the wireless device also controls the separation and control of the microfluidics channels for more complex LOC analyses. In one embodiment, a LED and other electronic measuring or sensing devices are included in the LOC-RFID chip. Not being bound by a theory, this technology is disposable and allows complex tests that require separation and mixing to be performed outside of a laboratory.

In preferred embodiments, the LOC may be a microfluidic device. The LOC may be a passive chip, wherein the chip is powered and controlled through a wireless device. In certain embodiments, the LOC includes a microfluidic channel for holding reagents and a channel for introducing a sample. In certain embodiments, a signal from the wireless device delivers power to the LOC and activates mixing of the sample and assay reagents. Specifically, in the case of the present invention, the system may include a masking agent, CRISPR effector protein, and guide RNAs specific for a target molecule. Upon activation of the LOC, the microfluidic device may mix the sample and assay reagents. Upon mixing, a sensor detects a signal and transmits the results to the wireless device. In certain embodiments, the unmasking agent is a conductive RNA molecule. The conductive RNA molecule may be attached to the conductive material. Conductive molecules can be conductive nanoparticles, conductive proteins, metal particles that are attached to the protein or latex or other beads that are conductive. In certain embodiments, if DNA or RNA is used then the conductive molecules can be attached directly to the matching DNA or RNA strands. The release of the conductive molecules may be detected across a sensor. The assay may be a one step process.

Since the electrical conductivity of the surface area can be measured precisely quantitative results are possible on the disposable wireless RFID electro-assays. Furthermore, the test area can be very small allowing for more tests to be done in a given area and therefore resulting in cost savings. In certain embodiments, separate sensors each associated with a different CRISPR effector protein and guide RNA immobilized to a sensor are used to detect multiple target molecules. Not being bound by a theory, activation of different sensors may be distinguished by the wireless device.

In addition to the conductive methods described herein, other methods may be used that rely on RFID or Bluetooth as the basic low cost communication and power platform for a disposable RFID assay. For example, optical means may be used to assess the presence and level of a given target molecule. In certain embodiments, an optical sensor detects unmasking of a fluorescent masking agent.

In certain embodiments, the device of the present invention may include handheld portable devices for diagnostic reading of an assay (see e.g., Vashist et al., Commercial Smartphone-Based Devices and Smart Applications for Personalized Healthcare Monitoring and Management, Diagnostics 2014, 4(3), 104-128; mReader from Mobile Assay; and Holomic Rapid Diagnostic Test Reader).

As noted herein, certain embodiments allow detection via colorimetric change which has certain attendant benefits when embodiments are utilized in POC situations and or in resource poor environments where access to more complex detection equipment to readout the signal may be limited. However, portable embodiments disclosed herein may also be coupled with hand-held spectrophotometers that enable detection of signals outside the visible range. An example of a hand-held spectrophotometer device that may be used in combination with the present invention is described in Das et al. “Ultra-portable, wireless smartphone spectrophotometer for rapid, non-destructive testing of fruit ripeness.” Nature Scientific Reports. 2016, 6:32504, DOI: 10.1038/srep32504. Finally, in certain embodiments utilizing quantum dot-based masking constructs, use of a handheld UV light, or other suitable device, may be successfully used to detect a signal owing to the near complete quantum yield provided by quantum dots.

In some embodiments, the device is a lateral flow device. In some embodiments, the lateral flow device can be composed of a CRISPR system and detection construct described elsewhere herein and a lateral flow substrate for carrying out the detection reaction and/or nucleic acid release from the sample.

In one aspect, the embodiments disclosed herein are directed to a nucleic acid detection system comprising a CRISPR-Cas system, one or more guide RNAs designed to bind to corresponding target molecules, a reporter construct (also referred to herein as a detection construct in this context), and optional amplification reagents (discussed in greater detail elsewhere herein) to amplify target nucleic acid molecules and/or detectable signals in a sample. The reporter construct is a molecule that comprises an oligonucleotide component (DNA or RNA) that can be cleaved by an activated CRISPR effector protein. The composition of the oligonucleotide component may be generic i.e., not the same as a target molecule. The reporter construct is configured so that it prevents or masks generation of a detectable positive signal when in the uncleaved configuration but allows or facilitates generation of a positive detectable signal when cleaved. In the context of the present invention, reporting constructs comprising a first molecule and a second molecule connected by an RNA or DNA nucleic acid linker. Use of an RNA or DNA linker will depend on whether the CRISPR effector protein(s) used have RNA or DNA collateral activity. The first and second molecule are generally part of a binding pair, where the other binding partner is affixed to the lateral flow substrate as described in further detail below. The systems further comprise a detection agent that specifically binds the second molecule and further comprises a detectable label. For ease of reference, these systems may be referred to herein as SHERLOCK systems and the reactions they facilitate as SHERLOCK reactions. If a target molecule is present in a sample, the corresponding guide molecule will guide the CRISPR Cas/guide complex to the target molecule by hybridizing with the target molecule, thereby triggering the CRISPR effector protein's nuclease activity. This activated CRISPR effector protein will cleave both the target molecule and then non-specifically cleave the linker portion of the RNA construct.

In some embodiments, the device can include a lateral flow substrate for detecting a SHERLOCK reaction. Substrates suitable for use in lateral flow assays are known in the art. These may include, but are not necessarily limited to, membranes or pads made of cellulose and/or glass fiber, polyesters, nitrocellulose, or absorbent pads (J Saudi Chem Soc 19(6):689-705; 2015). The SHERLOCK system, i.e., one or more CRISPR systems and corresponding reporter constructs are added to the lateral flow substrate at a defined reagent portion of the lateral flow substrate, typically on one end of the lateral flow substrate. Reporting constructs used within the context of the present invention comprise a first molecule and a second molecule linked by an RNA or DNA linker. The lateral flow substrate further comprises a sample portion. The sample portion may be equivalent to, continuous with, or adjacent to the reagent portion. The lateral flow strip further comprises a first capture line, typically a horizontal line running across the device, but other configurations are possible. The first capture region is proximate to and on the same end of the lateral flow substrate as the sample loading portion. A first binding agent that specifically binds the first molecule of the reporter construct is fixed or otherwise immobilized to the first capture region. The second capture region is located towards the opposite end of the lateral flow substrate from the first binding region. A second binding agent is fixed or otherwise immobilized at the second capture region. The second binding agent specifically binds the second molecule of the reporter construct, or the second binding agent may bind a detectable ligand. For example, the detectable ligand may be a particle, such as a colloidal particle, that when it aggregates can be detected visually. The particle may be modified with an antibody that specifically binds the second molecule on the reporter construct. If the reporter construct is not cleaved it will facilitate accumulation of the detectable ligand at the first binding region. If the reporter construct is cleaved the detectable ligand is released to flow to the second binding region. In such an embodiment, the second binding agent is an agent capable of specifically or non-specifically binding the detectable ligand on the antibody on the detectable ligand. Examples of suitable binding agents for such an embodiment include, but are not limited to, protein A and protein G.

Lateral support substrates may be located within a housing (see for example, “Rapid Lateral Flow Test Strips” Merck Millipore 2013). The housing may comprise at least one opening for loading samples and a second single opening or separate openings that allow for reading of detectable signal generated at the first and second capture regions.

The SHERLOCK system may be freeze-dried to the lateral flow substrate and packaged as a ready to use device, or the SHERLOCK system may be added to the reagent portion of the lateral flow substrate at the time of using the device. Samples to be screened are loaded at the sample loading portion of the lateral flow substrate. The samples must be liquid samples or samples dissolved in an appropriate solvent, usually aqueous. The liquid sample reconstitutes the SHERLOCK reagents such that a SHERLOCK reaction can occur. The liquid sample begins to flow from the sample portion of the substrate towards the first and second capture regions. Intact reporter construct is bound at the first capture region by binding between the first binding agent and the first molecule. Likewise, the detection agent will begin to collect at the first binding region by binding to the second molecule on the intact reporter construct. If target molecule(s) are present in the sample, the CRISPR effector protein collateral effect is activated. As activated CRISPR effector protein comes into contact with the bound reporter construct, the reporter constructs are cleaved, releasing the second molecule to flow further down the lateral flow substrate towards the second binding region. The released second molecule is then captured at the second capture region by binding to the second binding agent, where additional detection agent may also accumulate by binding to the second molecule. Accordingly, if the target molecule(s) is not present in the sample, a detectable signal will appear at the first capture region, and if the target molecule(s) is present in the sample, a detectable signal will appear at the location of the second capture region.

Specific binding-integrating molecules comprise any members of binding pairs that can be used in the present invention. Such binding pairs are known to those skilled in the art and include, but are not limited to, antibody-antigen pairs, enzyme-substrate pairs, receptor-ligand pairs, and streptavidin-biotin. In addition to such known binding pairs, novel binding pairs may be specifically designed. A characteristic of binding pairs is the binding between the two members of the binding pair.

Oligonucleotide Linkers having molecules on either end may comprise DNA if the CRISPR effector protein has DNA collateral activity (Cpf1 and C2c1) or RNA if the CRISPR effector protein has RNA collateral activity. Oligonucleotide linkers may be single stranded or double stranded, and in certain embodiments, they could contain both RNA and DNA regions. Oligonucleotide linkers may be of varying lengths, such as 5-10 nucleotides, 10-20 nucleotides, 20-50 nucleotides, or more.

In some embodiments, the polypeptide identifier elements include affinity tags, such as hemagglutinin (HA) tags, Myc tags, FLAG tags, V5 tags, chitin binding protein (CBP) tags, maltose-binding protein (MBP) tags, GST tags, poly-His tags, and fluorescent proteins (for example, green fluorescent protein (GFP), yellow fluorescent protein (YFP), cyan fluorescent protein (CFP), dsRed, mCherry, Kaede, Kindling, and derivatives thereof, FLAG tags, Myc tags, AU1 tags, T7 tags, OLLAS tags, Glu-Glu tags, VSV tags, or a combination thereof. Other Affinity tags are well known in the art. Such labels can be detected and/or isolated using methods known in the art (for example, by using specific binding agents, such as antibodies, that recognize a particular affinity tag). Such specific binding agents (for example, antibodies) can further contain, for example, detectable labels, such as isotope labels and/or nucleic acid barcodes such as those described herein.

For instance, a lateral flow strip allows for RNAse (e.g., Cas13a) detection by color. The RNA reporter is modified to have a first molecule (such as for instance FITC) attached to the 5′ end and a second molecule (such as for instance biotin) attached to the 3′ end (or vice versa). The lateral flow strip is designed to have two capture lines with anti-first molecule (e.g., anti-FITC) antibodies hybridized at the first line and anti-second molecule (e.g., anti-biotin) antibodies at the second downstream line. As the SHERLOCK reaction flows down the strip, uncleaved reporter will bind to anti-first molecule antibodies at the first capture line, while cleaved reporters will liberate the second molecule and allow second molecule binding at the second capture line. Second molecule sandwich antibodies, for instance conjugated to nanoparticles, such as gold nanoparticles, will bind any second molecule at the first or second line and result in a strong readout/signal (e.g., color). As more reporter is cleaved, more signal will accumulate at the second capture line and less signal will appear at the first line. In certain aspects, the invention relates to the use of a follow strip as described herein for detecting nucleic acids or polypeptides. In certain aspects, the invention relates to a method for detecting nucleic acids or polypeptides with a flow strip as defined herein, e.g., (lateral) flow tests or (lateral) flow immunochromatographic assays.

In certain example embodiments, a lateral flow device comprises a lateral flow substrate comprising a first end for application of a sample. The first region is loaded with a detectable ligand, such as those disclosed herein, for example a gold nanoparticle. The gold nanoparticle may be modified with a first antibody, such as an anti-FITC antibody. The first region also comprises a detection construct. In one example embodiment, an RNA detection construct and a CRISPR effector system (a CRISPR effector protein and one or more guide sequences configured to bind to one or more target sequences) as disclosed herein. In one example embodiment, and for purposes of further illustration, the RNA construct may comprise a FAM molecule on a first end of the detection construction and a biotin on a second end of the detection construct. Upstream of the flow of solution from the first end of the lateral flow substrate is a first test band. The test band may comprise a biotin ligand. Accordingly, when the RNA detection construct is present it its initial state, i.e., in the absence of target, the FAM molecule on the first end will bind the anti-FITC antibody on the gold nanoparticle, and the biotin on the second end of the RNA construct will bind the biotin ligand allowing for the detectable ligand to accumulate at the first test, generating a detectable signal. Generation of a detectable signal at the first band indicate the absence of the target ligand. In the presence of target, the CRISPR effector complex forms and the CRISPR effector protein is activated resulting in cleavage of the RNA detection construct. In the absence of intact RNA detection construct the colloidal gold will flow past the second strip. The lateral flow device may comprise a second band, upstream of the first band. The second band may comprise a molecule capable of binding the antibody-labeled colloidal gold molecule, for example an anti-rabbit antibody capable of binding a rabbit anti-FTIC antibody on the colloidal gold. Therefore, in the presence of one or more targets, the detectable ligand will accumulate at the second band, indicating the presence of the one or more targets in the sample. See also WO 2019/071051, which is incorporated by reference herein.

### Droplets

In some embodiments, the device can generate and/or manipulate individual discrete volumes, such as droplets. As described elsewhere herein one or more reactions can take place in a droplet. The droplets as provided herein are typically water-in-oil microemulsions formed with an oil input channel and an aqueous input channel. The droplets can be formed by a variety of dispersion methods known in the art. In one particular embodiment, a large number of uniform droplets in oil phase can be made by microemulsion. Exemplary methods can include, for example, R-junction geometry where an aqueous phase is sheared by oil and thereby generates droplets; flow-focusing geometry where droplets are produced by shearing the aqueous stream from two directions; or co-flow geometry where an aqueous phase is ejected through a thin capillary, placed coaxially inside a bigger capillary through which oil is pumped.

The use of monodisperse aqueous droplets can be generated by a microfluidic device as a water-in-oil emulsion. In one embodiment, the droplets are carried in a flowing oil phase and stabilized by a surfactant. In one aspect single cells or single organelles or single molecules (proteins, RNA, DNA) are encapsulated into uniform droplets from an aqueous solution/dispersion. In a related aspect, multiple cells or multiple molecules may take the place of single cells or single molecules.

The aqueous droplets of volume ranging from 1 μL to 10 nL work as individual reactors. 10⁴ to 10⁵ single cells in droplets may be processed and analyzed in a single run. To utilize microdroplets for rapid large-scale chemical screening or complex biological library identification, different species of microdroplets, each containing the specific chemical compounds or biological probes cells or molecular barcodes of interest, have to be generated and combined at the preferred conditions, e.g., mixing ratio, concentration, and order of combination. Each species of droplet is introduced at a confluence point in a main microfluidic channel from separate inlet microfluidic channels. Preferably, droplet volumes are chosen by design such that one species is larger than others and moves at a different speed, usually slower than the other species, in the carrier fluid, as disclosed in U.S. Publication No. US 2007/0195127 and International Publication No. WO 2007/089541, each of which are incorporated herein by reference in their entirety. The channel width and length is selected such that faster species of droplets catch up to the slowest species. Size constraints of the channel prevent the faster moving droplets from passing the slower moving droplets resulting in a train of droplets entering a merge zone. Multi-step chemical reactions, biochemical reactions, or assay detection chemistries often require a fixed reaction time before species of different type are added to a reaction. Multi-step reactions are achieved by repeating the process multiple times with a second, third or more confluence points each with a separate merge point. Highly efficient and precise reactions and analysis of reactions are achieved when the frequencies of droplets from the inlet channels are matched to an optimized ratio and the volumes of the species are matched to provide optimized reaction conditions in the combined droplets. Fluidic droplets may be screened or sorted within a fluidic system of the invention by altering the flow of the liquid containing the droplets. For instance, in one set of embodiments, a fluidic droplet may be steered or sorted by directing the liquid surrounding the fluidic droplet into a first channel, a second channel, etc. In another set of embodiments, pressure within a fluidic system, for example, within different channels or within different portions of a channel, can be controlled to direct the flow of fluidic droplets. For example, a droplet can be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled.

The invention can thus involve forming sample droplets. The droplets are aqueous droplets that are surrounded by an immiscible carrier fluid. Methods of forming such droplets are shown for example in Link et al. (U.S. patent application numbers 2008/0014589, 2008/0003142, and 2010/0137163), Stone et al. (U.S. Pat. No. 7,708,949 and U.S. patent application number 2010/0172803), Anderson et al. (U.S. Pat. No. 7,041,481 and which reissued as RE41,780) and European publication number EP2047910 to Raindance Technologies Inc. The content of each of which is incorporated by reference herein in its entirety. The present invention may relate to systems and methods for manipulating droplets within a high throughput microfluidic system.

From this disclosure and herein cited documents and knowledge in the art, it is within the ambit of the skilled person to develop flow rates, channel lengths, and channel geometries; and establish droplets containing random or specified reagent combinations can be generated on demand and merged with the “reaction chamber” droplets containing the samples/cells/substrates of interest. By incorporating a plurality of unique tags into the additional droplets and joining the tags to a solid support designed to be specific to the primary droplet, the conditions that the primary droplet is exposed to may be encoded and recorded. For example, nucleic acid tags can be sequentially ligated to create a sequence reflecting conditions and order of same. Alternatively, the tags can be added independently appended to solid support. Non-limiting examples of a dynamic labeling system that may be used to bioinformatically record information can be found at US Provisional Patent Application entitled “Compositions and Methods for Unique Labeling of Agents” filed Sep. 21, 2012 and Nov. 29, 2012. In this way, two or more droplets may be exposed to a variety of different conditions, where each time a droplet is exposed to a condition, a nucleic acid encoding the condition is added to the droplet each ligated together or to a unique solid support associated with the droplet such that, even if the droplets with different histories are later combined, the conditions of each of the droplets are remain available through the different nucleic acids. Non-limiting examples of methods to evaluate response to exposure to a plurality of conditions can be found at US Provisional Patent Application filed Sep. 21, 2012, and U.S. patent application Ser. No. 15/303,874 filed Apr. 17, 2015 entitled “Systems and Methods for Droplet Tagging.” Accordingly, in or as to the invention it is envisioned that there can be the dynamic generation of molecular barcodes (e.g., DNA oligonucleotides, fluorophores, etc.) either independent from or in concert with the controlled delivery of various compounds of interest (siRNA, CRISPR guide RNAs, reagents, etc.). For example, unique molecular barcodes can be created in one array of nozzles while individual compounds or combinations of compounds can be generated by another nozzle array. Barcodes/compounds of interest can then be merged with CRISPR detection system-containing droplets. An electronic record in the form of a computer log file is kept to associate the barcode delivered with the downstream reagent(s) delivered. This methodology makes it possible to efficiently screen a large population of samples according to the methods disclosed herein. The device and techniques of the disclosed invention facilitate efforts to perform studies that require data resolution at the single cell (or single molecule) level and in a cost-effective manner. A high throughput and high-resolution delivery of reagents to individual emulsion droplets that may contain samples of target molecules for further evaluation through the use of monodisperse aqueous droplets that are generated one by one in a microfluidic chip as a water-in-oil emulsion.

### Exemplary Devices

In some embodiments, the device, optionally a microfluidic device, configured to detect nucleic acids includes a sample loading module configured to receive a sample; a sample processing module at least configured to process a sample for optional nucleic acid extraction and/or concentration and nucleic acid detection; an optional nucleic acid extraction and/or concentration module configured to extract and/or concentrate nucleic acids in the sample; and a nucleic acid detection module configured to detect target nucleic acids. In some embodiments, the sample processing module comprises one or more filters, wherein the filters are optionally membranes. In some embodiments, the sample processing module is configured to separate plasma from blood. In some embodiments, the optional nucleic acid extraction and/or concentration module comprises magnetic particles configured to bind, attach, or otherwise associate with nucleic acids. In some embodiments, the movable magnet is at least operatively coupled to the nucleic acid extraction and/or concentration module. In some embodiments, the device further reincludes a power source. In some embodiments, the device further includes a heating element coupled to the sample processing module. In some embodiments, the sample loading module, sample processing module, optional nucleic acid extraction and/or concentration module comprise one or more reservoirs, vessels, compartments, and/or regions each configured to contain a sample or component thereof. In some embodiments, the nucleic acid detection module is configured to amplify one or more target regions of one or more target elements.

In some embodiments, the nucleic acid detection module is configured to amplify a set of target regions in one or more target elements, and optionally comprises a set of amplification primers configured to amplify the set of target regions in one or more target elements, wherein primers of the set of amplification primers are optionally optimized for pooled amplification. In some embodiments, the nucleic acid detection module comprises one or more flow channels, each flow channel comprising a sample loading region; a detector region comprising an oligonucleotide-based detection construct and one or more nucleic acid detection systems; and at least a first capture region and a second capture region, the first capture region comprising a first binding agent and the second capture region comprising a second binding agent. In some embodiments, the nucleic acid detection module is a node, wherein each flow channel is arranged radially from a center node. In some embodiments, the center node comprises transcription and/or amplification reagents. In some embodiments, the device further includes one or more thermally differentiated zones disposed between the sample loading region and the center node. In some embodiments, each flow channel is arranged in parallel. In some embodiments the detection construct comprises a first molecule on a first end and a second molecule on a second end. In some embodiments, one or more nucleic acid detection systems each comprise one or more Cas protein, and one or more target-specific guide polynucleotides, wherein the target for each of the one or more target-specific guide polynucleotides is a region of a target element of a nucleic acid.

In some embodiments, the nucleic acid detection module comprises a nucleic acid detection system of the present invention or one or more components thereof.

In some embodiments, the nucleic acid detection module comprises one or more flow channels, each flow channel containing a sample loading region, optionally comprising one or more components of a nucleic acid detection system of the present invention or one or more components thereof, a detector region comprising a one or more components of a nucleic acid detection system of the present invention or one or more components thereof, and at least a first capture region and a second capture region, the first capture region comprising a first binding agent and the second capture region comprising a second binding agent.

In some embodiments, the target region(s) and/or target element(s) is/are or includes one or more target regions and/or elements specific to one or more microorganisms or viruses, optionally one or more pathogenic microorganisms or viruses. In some embodiments, the target regions and/or target element(s) is/are or include target region(s) and/or target element(s) specific to one or more diseased or abnormal cells or tissues.

In some embodiments, the nucleic acid is RNA, genomic DNA, cell free DNA, and/or circulating cell free DNA.

In some embodiments, the device further includes one or more amplification reagents, optionally in the sample loading region. In some embodiments, the one or more amplification reagents are nucleic acid sequence-based amplification (NASBA) reagent(s), recombinase polymerase amplification (RPA) reagent(s), loop-mediated isothermal amplification (LAMP) reagent(s), RT-LAMP reagent(s), strand displacement amplification (SDA) reagent(s), helicase-dependent amplification (HDA) reagent(s), nicking enzyme amplification reaction (NEAR) reagent(s), PCR reagent(s), multiple displacement amplification (MDA) reagent(s), rolling circle amplification (RCA) reagent(s), ligase chain reaction (LCR) reagent(s), or ramification amplification method (RAM) reagent(s).

In some embodiments, the nucleic acid detection module comprises a set of amplification primers, optionally contained in the sample loading region or detector region of the device.

In some embodiments, the device is a microfluidic device. In some embodiments, one or more of the modules a microfluidic modules. In some embodiments, the device is configured as a point-of-care and/or field deployable device.

In certain exemplary embodiments the microfluidic devices are configured to detect nucleic acids include one or more flow channels, each flow channel comprising: a sample loading region optionally containing one or more nucleic acid detection systems of the present invention or one or more components thereof, a detector region comprising an oligonucleotide-based detection construct and one or more nucleic acid detection systems of the present invention or one or more components thereof, and at least a first capture region and a second capture region, the first capture region comprising a first binding agent and the second capture region comprising a second binding agent. In some embodiments, such as those where the nucleic acid detection module is a node, wherein each flow channel is arranged radially from a center node. In some embodiments, the center node comprises transcription and/or amplification reagents. In some embodiments, the method further includes one or more thermally differentiated zones disposed between the sample loading region and the center node. In some embodiments, each flow channel is arranged in parallel to one another.

In some embodiments, the detection construct comprises a first molecule on a first end and a second molecule on a second end.

In some embodiments, one or more nucleic acid detection systems each comprise one or more Cas protein, and one or more target-specific guide polynucleotides, wherein the target for each of the one or more target-specific guide polynucleotides is a region of a target element of a nucleic acid.

In some embodiments, the target element are one or more elements specific to one or more microorganisms, viruses, diseased cells and/or diseased tissue.

In some embodiments, the nucleic acid is RNA, genomic DNA, cell free DNA, and/or circulating cell free DNA.

In some embodiments, the device further includes one or more amplification reagents, optionally in the sample loading region.

In some embodiments, the one or more amplification reagents are nucleic acid sequence-based amplification (NASBA) reagent(s), recombinase polymerase amplification (RPA) reagent(s), loop-mediated isothermal amplification (LAMP) reagent(s), RT-LAMP reagent(s), strand displacement amplification (SDA) reagent(s), helicase-dependent amplification (HDA) reagent(s), nicking enzyme amplification reaction (NEAR) reagent(s), PCR reagent(s), multiple displacement amplification (MDA) reagent(s), rolling circle amplification (RCA) reagent(s), ligase chain reaction (LCR) reagent(s), ramification amplification method (RAM) reagent(s), or any combination thereof.

In some embodiments, the device comprises one or more target amplification probe sets, optionally contained in the sample loading region.

In some embodiments, the device can be configured as a fully integrated, sample in-answer out diagnostic devices that can process a blood (or other fluid) sample from collection (e.g., a fingerstick) to an output reading (e.g., on a lateral flow strip). In some embodiments, the devices is composed of three modules: (1) a sample processing step and/or component that is configured to separate out specific components of the fluid (e.g., separate plasma from blood) sample and subsequently isolate nucleic acids (e.g., cfDNA and/or ccfDNA) present in the sample; (2) an amplification step and/or component (e.g., an isothermal amplification step/component); and (3) a detection component (e.g., visual detection on a lateral flow strip).

### Sample Processing Module

The sample processing module can be configured to process a sample, such as a fluid sample (e.g., blood), to optionally separate one or more components within the sample from each other and isolate nucleic acids (e.g., cfDNA and/or ccfDNA) present in the sample for downstream processing and/or analysis. In some embodiments, the sample processing module can contain one or more point-of-care consumables, including without limitation blister packs and syringes. Using blood as a sample example, plasma separation can be accomplished by incorporating a membrane downstream of the sample input in the device. Such plasma-separating membranes are generally known in the art and have been field tested for POC diagnostics, particularly those for low-resource settings (see e.g., Pollock, N. R., et al., A paper-based multiplexed transaminase test for low-cost, point-of-care liver function testing. Science translational medicine, 2012. 4(152): p. 152ra129-152ra129). In some embodiments, syringe-driven flow can be used to drive the sample through the sample processing module. In some embodiments, plastic-embossed, one way valves can be used to prevent backflow. In some embodiments, the blister pack can be coupled to vacuum reservoirs to drive flow into the device and across the membrane and into the initial sample chamber similar to the approach utilized with Vacutainers.

cfDNA/ccfDNA Extraction

Different approaches to extracting the cfDNA and/or the ccfDNA from the separated plasma can be used. In some embodiments, a strategy similar to the Promega Maxwell magnetic particle technology is used. In some embodiments, a commercially available ccf or cfDNA kit (e.g., Promega Maxwell RSC ccfDNA plasma kit) can be employed with blister packs configured to work with the device described herein to release beads (e.g., magnetic beads) into the mixing chamber(s) along with one or more buffers necessary for extraction. Movement of the magnetic particles in and out of the pre-filled chambers in the blister pack can be achieved via corresponding movement of an external magnet operated by an e.g., battery-powered micro-controller. Another embodiment of the device can employ the same blister packs loaded into but the movement of the external magnet can be automated in similar fashion to the Promega Maxwell device.

In some embodiments, an adaptation of the HUDSON method (see e.g., Myrvhold et al. Science. 360:444-448 (2018) and International Pat. Pub. WO 2018170340) and/or the SHINE method (see e.g., and U.S. Provisional Application No. 63/074,307 and Arizti-Sanz et al. 2020. BioRxiv. https://doi.org/10.1101/2020.05.28.119131) can be utilized. HUDSON is simpler than the Promega Maxwell magnetic-based method previously described as it only needs the addition of an inactivation solution and heating from about 50 degrees C. and about 64 degrees C. SHINE is likewise simpler with low or no heating requirements. A the both require only low-power, which can be provided by e.g., solar-charged batteries which are suitable for field-deployment devices. When combined with the tiling and optional multiplexed amplification and/or detection approaches described herein, the sensitivity of the approach can be sufficiently increased such that detection of low concentrations of nucleic acids (e.g., cfDNA, ccfDNA, and cell free nucleic acids).

### Amplification and CRISPR-Cas System Detection Module

In some embodiments, the second module is configured to amplify target elements and target regions and optionally detect amplified target elements and amplified target regions via a CRISPR-Cas system (e.g., a CRISPR-Cas system having collateral activity, which are described in greater detail elsewhere herein).

For embodiments utilizing the magnetic bead-based processing module/method previously described once the ccfDNA and/or cfDNA has been captured, they can be eluted off the magnetic beads using any suitable technique and/or reagents into an amplification chamber (e.g., an RPA or isothermal amplification chamber) where amplification can occur. After amplification, amplified products can be moved into a CRISPR-Cas system (e.g., a Cas13 (e.g., Cas 13a) containing CRISPR-Cas system) detection chamber where CRISPR-Cas detection of the target(s) can occur. After CRISPR-Cas detection, detection complexes can be moved onto a lateral flow strip for visual detection of CRISPR-Cas detection. Prior reports have demonstrated the stability of lyophilized and subsequently rehydrated amplification (e.g., RPA) reagents) and CRISPR-Cas system components (e.g., Cas13 reagents) (see e.g., World Health, O., Global tuberculosis report 2018. 2018, Geneva: World Health Organization and Gootenberg, J. S., et al., Nucleic acid detection with CRISPR-Cas13a/C2c2. Science, 2017. 356(6336): p. 438-442).

For embodiments utilizing the HUDSON and/or SHINE approach in the first module, the heat-inactivated plasma sample can be flowed directly into the amplification (e.g., RPA) chamber, the CRISPR-Cas detection chamber, and onto a LFS as described in connection with the magnetic bead-based approach.

### Lateral Flow Detection of CRISPR-Cas Activity Module

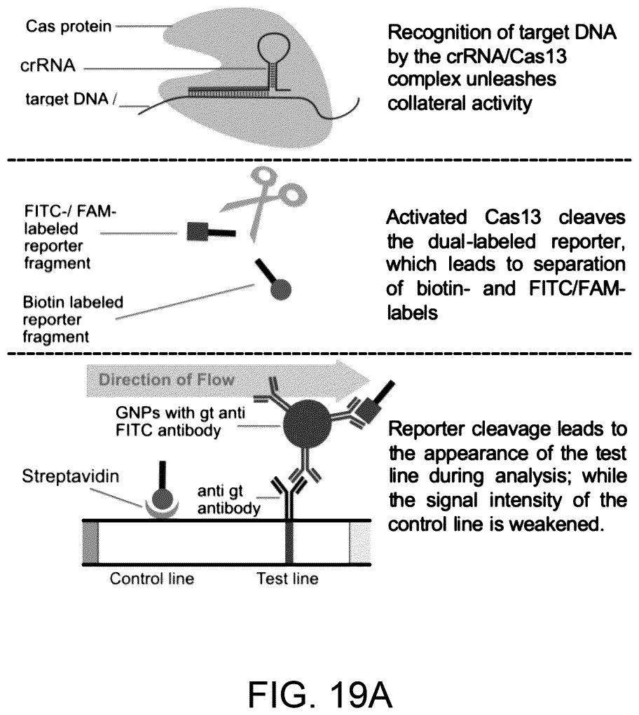

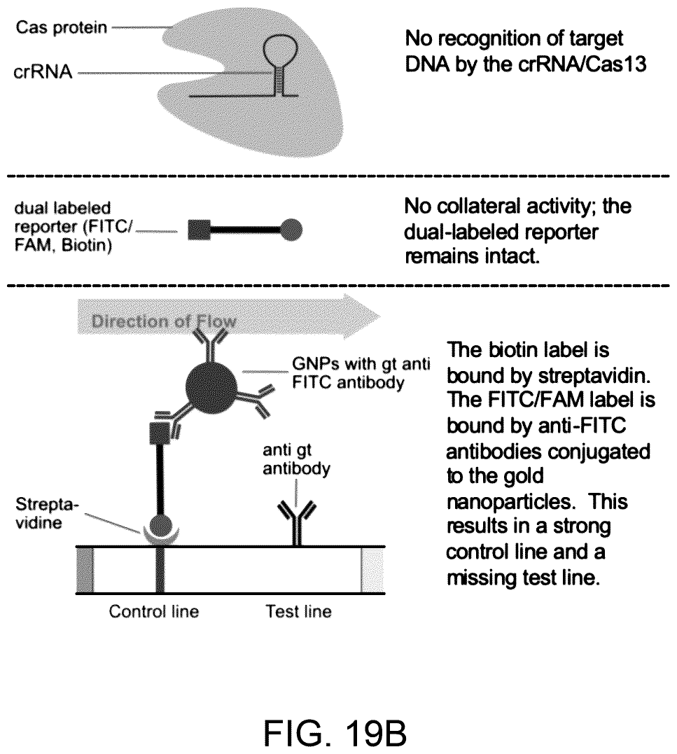

Collateral Cas (e.g., a Cas13) activity in response to binding a target polynucleotide can be detected on commercially available lateral flow strips and those described elsewhere herein (see e.g., Myhrvold, C., et al., Field-deployable viral diagnostics using CRISPR-Cas13. Science, 2018.360(6387): p. 444-448 and Gootenberg, J. S., et al., Nucleic acid detection with CRISPR-Cas13a/C2c2. Science, 2017. 356(6336): p. 438-442). In some embodiments, when Cas (e.g., a Cas 13) detects its target—as mediated by the guide RNA—it becomes activated and cleaves a labeled reporter (e.g., a dual-labeled, biotin- and FITC/FAM-labeled reporters) present in the detection reaction. Upon incubating the Cas (e.g., a Cas13) reaction with a LFS buffer, which can contain gold nanoparticles labeled with anti-FITC antibodies, the cleaved reporter is then detected on the LFS where the anti-rabbit antibody containing control line on the LFS captures the anti-FITC antibodies on the gold nanoparticles. A streptavidin containing test line captures the biotin. Thus, when a target is present, the Cas (e.g., Cas 13) is activated, the reporter is cleaved, and the gold-nanoparticle-induced line at the “test line” and a weak to no line visible at the “control line”. When Cas (e.g., Cas 13) is not activated due to an absence of the target or amounts below the LOD, the gold nanoparticle-induced line is visible at the “control line” only. See e.g., FIG. 19A-19B.

### Kits

Any of the nucleic acid detection systems and/or components thereof, compositions, formulations, devices, or any combination thereof described herein can be presented as a combination kit. As used herein, the terms “combination kit” or “kit of parts” refers to the nucleic acid detection systems and/or components thereof, compositions, formulations, devices, or any combination thereof described herein and any additional components that are used to package, sell, market, deliver, and/or administer the combination of elements or a single element, such as the active ingredient, contained therein. Such additional components include, but are not limited to, packaging, syringes, blister packages, dipsticks, substrates, bottles, tubes, sampling devices, and the like. The separate kit components can be contained in a single package or in separate packages within the kit.

In some embodiments, the combination kit also includes instructions printed on or otherwise contained in a tangible medium of expression. The instructions can provide information regarding the content of the nucleic acid detection systems and/or components thereof, compositions, formulations, devices, or any combination thereof described herein, safety information regarding the content of the nucleic acid detection systems and/or components thereof, compositions, formulations, devices, or any combination thereof described herein, information regarding the dosages, working amounts, indications for use, and/or recommended methods, protocols, sampling protocols, sample types, for the nucleic acid detection systems and/or components thereof, compositions, formulations, devices, or any combination thereof described herein contained in the kit. In some embodiments, the instructions can be specific to the target(s) being detected by a CRISPR effector detection system. In some embodiments, the instructions are specific to detecting cfDNA from an organism of interest, such as a microbe (e.g., a pathogenic microbe) or diseased cell or tissue. Exemplary microbes and diseases that can be detected by the kits described herein are described elsewhere herein. In some embodiments, the target is TB.

### Methods of Detecting Nucleic Acids

Described in various exemplary embodiments herein are tiled amplification and detection assays that can be capable of detecting target nucleic acids, particularly cfDNA. The nucleic acid detection systems described herein can be used to detect one or more nucleic acids, such as those present in a sample. In some embodiments, the nucleic acids that are detected are cfDNA or other cell free nucleic acid. The methods described herein couple pooled and tiled amplification with optionally pooled CRISPR-Cas detection of amplified target regions. In some embodiments, amplification of the amplification target regions can be pooled in a single reaction. this can provide an added advantage for small volume samples and/or detection of low abundance nucleic acids as the sample is not diluted or extended across multiple reactions, which reduces the likelihood of target detection. Further, amplification can be tiled to amplify multiple target elements across a genome, which can further increase the sensitivity of the method. Cas-based detection of amplified target regions can also be pooled (i.e., multiple amplified target regions are detected per detection reaction) or run in parallel (i.e., single target amplified regions are detected per detection reaction run in parallel). Without being bound by theory, such strategies can increase the sensitivity of the assay such that low abundance, small, and/or fragmented nucleic acids, such as ccfDNA, can be detected.

Described in certain exemplary embodiments herein are methods of detecting one or more nucleic acids in a sample that include contacting one or more samples with a set of amplification primers configured to amplify a set of target regions in one or more target elements, wherein primers of the set of amplification primers are optionally optimized for pooled amplification; amplifying, optionally by pooled amplification, two or more target regions in one or more target elements by the set of amplification primers thereby producing one or more amplified target regions; contacting the one or more amplified target regions with one or more Cas proteins having collateral activity, a set of guide polynucleotides comprising a guide polynucleotide specific for each of the amplified target regions, wherein each of the guide polynucleotides in the set of guide polynucleotides is capable of forming a CRISPR-Cas complex with the one or more Cas proteins; and detecting a signal produced from the one or more oligonucleotide-based detection constructs in response cleavage of the non-target sequence by collateral activity of an activated CRISPR-Cas protein, thereby detecting one or more target elements in the sample.

In some embodiments, the amplification primer set comprises a plurality of amplification primers configured to amplify two or more amplification target regions in the same target element. In some embodiments, the amplification primer set comprises a plurality of amplification primers configured to amplify two or more amplification target regions in one or more different target elements. In some embodiments the amplification primer set comprises a plurality of amplification primers configured to amplify two or more amplification target regions in the same target element and a plurality of amplification primers configured to amplify two or more amplification target regions in one or more different target elements.

In some embodiments, the number of target elements amplified ranges from 1 to 500 or more, such as 1 or/to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309,310,311,312,313,314,315,316,317,318,319,320,321,322,323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422,423, 424,425, 426,427, 428,429, 430,431, 432,433, 434,435, 436, 437,438, 439, 440, 441, 442, 443, 444, 445, 446, 447, 448, 449, 450, 451, 452, 453, 454, 455, 456, 457, 458, 459, 460,461, 462,463, 464,465, 466,467, 468,469, 470,471, 472,473, 474, 475,476, 477, 478, 479,480, 481,482, 483, 484, 485, 486, 487,488, 489,490, 491,492, 493, 494,495, 496, 497, 498, 499, 500, or more.

In some embodiments the total number of amplification target regions amplified ranges from 1 to 500 or more, such as 1 or/to 2 or/to 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304,305,306,307,308,309,310,311,312,313,314,315,316,317,318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400,401, 402,403, 404,405, 406,407, 408,409, 410,411, 412, 413,414, 415, 416, 417,418, 419,420, 421,422, 423,424, 425,426, 427,428, 429,430, 431, 432,433, 434, 435, 436, 437, 438, 439, 440, 441, 442, 443, 444, 445, 446, 447, 448, 449, 450, 451, 452, 453, 454, 455, 456, 457,458, 459,460, 461,462, 463,464, 465,466, 467,468, 469, 470,471, 472, 473, 474, 475, 476, 477, 478, 479, 480, 481, 482, 483, 484, 485, 486, 487, 488, 489, 490, 491, 492, 493, 494, 495, 496, 497, 498, 499, 500, or more.

In some embodiments, the number of amplification target regions amplified for each target element independently ranges from 1-100 or more, such as such as 1 or/to 2, 3, 4, 5,6,7,8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more.

In some embodiments, the method is capable of detecting less than one genome equivalent (GE) of a target, such as target element. In some embodiments, the method is capable of detecting about 0.001 to 1 GE of a target, such as a target element. In some embodiments, the nucleic acid system is capable of detecting about 0.001 to 0.01, 0.01 to 0.1, or 0.1 to 1 GE of a target, such as a target element.

In some embodiments, the method has a sensitivity of detection of about 60, to/or 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or about 100 percent sensitivity. In some embodiments, the method has a sensitivity of detection of at least 60 percent, at least 65 percent, at least 70 percent, at least 75 percent, at least 80 percent, at least 85 percent, at least 90 percent, at least 95 percent, or at least 100 percent.

In some embodiments, the method has a specificity for detecting a specific species or variant of organisms (e.g., a microorganism or virus) of at least 95, 96, 97, 98, 99, 99.1, 99.2, 99.3, 99.4, 99.5, 99.6, 99.7, 99.8, or 99.9%. In some embodiments, the nucleic acid system can have a specificity for detecting a specific species or variant of organisms (e.g., a microorganism or virus) of 100 percent.

In some embodiments, amplification comprises nucleic acid sequence-based amplification (NASBA), recombinase polymerase amplification (RPA), loop-mediated isothermal amplification (LAMP), RT-LAMP, strand displacement amplification (SDA), helicase-dependent amplification (HDA), nicking enzyme amplification reaction (NEAR), polymerase chain reaction (PCR), multiple displacement amplification (MDA), rolling circle amplification (RCA), ligase chain reaction (LCR), or ramification amplification method (RAM), or a combination thereof. In some embodiments, amplification comprises isothermal amplification.

In some embodiments the sample can contain one or more components that can interfere with downstream processing and/or analysis. In some embodiments, the method further includes one or more inactivation steps prior to amplification. In some embodiments, the one or more inactivation steps comprises heating the sample, optionally to 50 degrees C. or more and/or optionally to about 64 degrees C. or more.

In some embodiments, the sample is a bodily fluid. In some embodiments, the sample is plasma, blood, urine or saliva. In some embodiments the sample is blood or plasma. Other exemplary sample types are described elsewhere herein. In some embodiments, the method further includes processing blood to obtain plasma.

In some embodiments, the method further includes extracting cell free DNA, DNA, RNA, or other nucleic acids or any combination thereof from a sample or a processed sample prior to amplification. In some embodiments, the nucleic acid is cell free nucleic acid.

In some embodiments, the sample is obtained from a subject. In some embodiments, the subject has an active microorganism and/or viral infection, has been infected, or is suspected of being infected with a microorganism and/or virus. In some embodiments, the subject has a disease or is suspected of having a disease. In some embodiments, the disease is a cancer.

In some embodiments, one or more steps of the method are performed using a nucleic acid detection system of the present invention as described in greater detail elsewhere herein.

The amplification and detection assays described herein can be used to detect and/or monitor a host response to an infection with a pathogen or response to disease, response to a disease treatment, and/or disease progression. In some embodiments, the method includes performing a method of detecting one or more nucleic acids in a sample as described elsewhere herein on a host sample obtained at a first time; performing the method of detecting one or more nucleic acids in a sample as described elsewhere herein on a host sample obtained at a second time; and detecting the presence of one or more pathogens or diseased cells and/or tissues at the first time and the second time. A change in the detection of nucleic acids (e.g., amount, type, target etc.) from the first time sample to the second time sample can indicate a host response to an infection with a pathogen, a host response to a disease, a host response to a disease treatment, and/or disease or infection progression. In some embodiments, the method further includes administering a pathogen or disease treatment to the host, subsequent to the first time and prior to the second time and optionally identifying and/or measuring one or more biomarkers associated with immune response, treatment resistance, or both. In some embodiments, the method includes repeating the method one or more times over a period of time thereby monitoring the host response and optionally treatment response, immune response, and/or treatment resistance over the period of time. In some embodiments, one or more steps of them method are performed using a nucleic acid detection system as described in greater detail elsewhere herein.

As previously described any of the methods described herein can be utilized for detection of cfDNA.

The tiled amplification assay performance can be optimized and benchmarked for detecting infection from blood samples. Clinical samples can contain a variety of inhibitors that may impede the performance of the assay. In some instances, assays can be optimized utilizing blood samples collected from patients with active infections using protocols to preserve cfDNA and/or ccfDNA, Collection of clinical samples in specialized tubes that stabilize cell membranes in whole blood to minimize cell lysis can aid in stabilizing the sample prior to testing but may introduce agents that hinder molecular analysis of cfDNA and/or ccfDNA. The sensitivity and specificity of the tiled amplification assay can be evaluated against alternative NATs, such as real-time PCR, and digital droplet PCR.

In one aspect, the embodiments disclosed herein are directed to a nucleic acid detection system comprising two or more CRISPR systems one or more guide RNAs designed to bind to corresponding target molecules, a masking construct, and optional amplification reagents to amplify target nucleic acid molecules in a sample. In certain example embodiments, the system may further comprise one or more detection aptamers. The one or more detection aptamers may comprise an RNA polymerase site or primer binding site. The one or more detection aptamers specifically bind one or more target polypeptides and are configured such that the RNA polymerase site or primer binding site is exposed only upon binding of the detection aptamer to a target peptide. Exposure of the RNA polymerase site facilitates generation of a trigger RNA oligonucleotide using the aptamer sequence as a template. Accordingly, in such embodiments the one or more guide RNAs are configured to bind to a trigger RNA.

In another aspect, the embodiments disclosed herein are directed to a diagnostic device comprising a plurality of individual discrete volumes. Each individual discrete volume comprises a CRISPR effector protein, one or more guide RNAs designed to bind to a corresponding target molecule, and a masking construct. In certain example embodiments, RNA amplification reagents may be pre-loaded into the individual discrete volumes or be added to the individual discrete volumes concurrently with or subsequent to addition of a sample to each individual discrete volume. The device may be a microfluidic based device, a wearable device, or device comprising a flexible material substrate on which the individual discrete volumes are defined.

In another aspect, the embodiments disclosed herein are directed to a method for detecting target nucleic acids in a sample comprising distributing a sample or set of samples into a set of individual discrete volumes, each individual discrete volume comprising a CRISPR effector protein, one or more guide RNAs designed to bind to one target oligonucleotides, and a masking construct. The set of samples are then maintained under conditions sufficient to allow binding of the one or more guide RNAs to one or more target molecules. Binding of the one or more guide RNAs to a target nucleic acid in turn activates the CRISPR effector protein. Once activated, the CRISPR effector protein then deactivates the masking construct, for example, by cleaving the masking construct such that a detectable positive signal is unmasked, released, or generated. Detection of the positive detectable signal in an individual discrete volume indicates the presence of the target molecules.

In yet another aspect, the embodiments disclosed herein are directed to a method for detecting polypeptides. The method for detecting polypeptides is similar to the method for detecting target nucleic acids described above. However, a peptide detection aptamer is also included. The peptide detection aptamers function as described above and facilitate generation of a trigger oligonucleotide upon binding to a target polypeptide. The guide RNAs are designed to recognize the trigger oligonucleotides thereby activating the CRISPR effector protein. Deactivation of the masking construct by the activated CRISPR effector protein leads to unmasking, release, or generation of a detectable positive signal.

In certain example embodiments, a single guide RNA specific to a single target is placed in separate volumes. Each volume may then receive a different sample or aliquot of the same sample. In certain example embodiments, multiple guide RNA each to separate target may be placed in a single well such that multiple targets may be screened in a different well. In order to detect multiple guide RNAs in a single volume, in certain example embodiments, multiple effector proteins with different specificities may be used. For example, different orthologs with different sequence specificities may be used. For example, one orthologue may preferentially cut A, while others preferentially cut C, U, or T. Accordingly, guide RNAs that are all, or comprise a substantial portion, of a single nucleotide may be generated, each with a different fluorophore. In this way up to four different targets may be screened in a single individual discrete volume.

Generally, the nucleic acid amplification and detection method described herein can be composed of two parts: 1) sample preparation (which can include sample collection and/or processing and e.g., pooled and/or tiled amplification) and 2) CRISPR-Cas system detection of one or more targets present in the sample. These steps are described in greater detail below and elsewhere herein. In some embodiments, one or more of the steps within each of the portions of the method are performed in the same reaction vessel, reaction area/location, and/or device. In some embodiments all of the steps of the method are performed in the same reaction vessel, same reaction vessel, reaction area/location, and/or device.

In some embodiments, the nucleic acid detection systems and methods herein are capable of detecting down to at least attomolar concentrations of target molecules, such as viral polynucleotides. In some embodiments, the nucleic acid detection systems and methods herein are capable of detecting down to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 copies of genomic DNA or RNA per microliter (cp/μL). In some embodiments, the nucleic acid detection systems and methods herein are capable of detecting down to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 copies of genomic DNA or RNA per microliter (cp/μL) using a fluorescent or colorimetric readout.

In some embodiments, the polynucleotides are released from cells in the sample and the nucleic acid detection system detection can occur on the released polynucleotides without extracting the sample polynucleotides from other components in the sample. In some embodiments, the nucleic acids are already outside of the cell, such as in the case of ccfDNA and cfDNA. This can allow for the sample preparation and CRISRP-Cas system detection reaction to be performed in the same reaction vessel.

In some embodiments, one or more or all of the steps included in the nucleic acid detection system amplification and detection reaction can occur at about 22 to about 64 degrees C., about 50 to about 64 degrees C., or about 22-55 degrees C. (including any target and/or signal amplification). In some embodiments, one or more or all of the steps included in the nucleic acid detection system amplification and detection reaction occur at about 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, to/or 64 degrees C. In some embodiments, one or more or all of the steps included in the nucleic acid detection system amplification and detection reaction occur at about 55, 56, 57, 58, 59, 60, 61, 62, 63, to/or 64 degrees C. In some embodiments, one or more or all of the steps included in the nucleic acid detection system amplification and detection reaction occur at about 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, to/or 55 degrees C., 22, 23, 24, 25, 26, 27, 28, 29, 30, 31, 32, 33, 34, 35, 36, to/or 37 degrees C., about 22, 23, 24, 25, 26, 27, 28, 29, 30, 31, 32, or/to 33 degrees C., about 22, 23, 24, 25, 26, or/to 27 degrees C., or about 22, 23, 24, to/or 25 degrees C. In some embodiments, one or more or all of the steps included in the nucleic acid detection system amplification and detection reaction can occur at about room temperature (about 22-25 degrees C.).

In some embodiments, the nucleic acid detection system amplification and detection reaction can occur as a two-step reaction in which amplification of target(s) and target detection via the CRISPR effector system occur in separate reactions. In some embodiments, The CRISPR-effector system detection reaction (including any target and/or signal amplification) can occur as a single, one-pot reaction. In some embodiments where the nucleic acid detection system amplification and detection reaction is a one-pot reaction, target amplification is achieved using LAMP or RPA. In some embodiments where the CRISRP-effector system detection reaction is a one-pot reaction the CRISPR-effector system includes a Cas 12 (such as a Cas12b) or a Cas13 (such as a cas13a). In some embodiments where the CRISRP-effector system detection reaction is a one-pot reaction and target amplification is achieved using LAMP, the CRISPR-effector system includes a Cas12, such as a Cas12b. In some embodiments where the CRISRP-effector system detection reaction is a one-pot reaction and tiled amplification is achieved using RPA, the CRISPR-Cas system includes a Cas13, such as a Cas13a. In some embodiments, sample preparation and a single, one-pot nucleic acid detection system amplification and detection reaction can occur in the same reaction vessel, thus eliminating the need to move potentially hazardous samples from one reaction vessel to another.

In some embodiments, the total time to perform the nucleic acid detection system amplification and detection method (from sample preparation to CRISPR-Cas based detection) can be greater than 0 hours but less than about 8, 7, 6, 5, 4, 3.5, 3, 2.5, 2, 1.5, 1, or 0.5 hours. In some embodiments, the total time to perform the nucleic acid detection system amplification and detection method (from sample preparation to detection) can occur within about 20 to 120 minutes, such as within about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, to/or 120 minutes. In some embodiments, the total time to perform the nucleic acid detection system amplification and detection method (from sample preparation to detection) can occur within about 20 to about 60 minutes, e.g., within about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or/to 60 minutes. In some embodiments, the total time to perform the nucleic acid detection system amplification and detection method (from sample preparation to detection) can occur within about 20 to about 45 minutes, e.g., within about 20, 21, 22, 23, 24, 25, 26, 27, 28, 29, 30, 31, 32, 33, 34, 35, 36, 37, 38, 39, 40, 41, 42, 43, 44, and/or 45 minutes. In some embodiments, the total time to perform the nucleic acid detection system amplification and detection method (from sample preparation to detection) can occur within about 20 to about 30 minutes, e.g., within about 20, 21, 22, 23, 24, 25, 26, 27, 28, 29, and/or 30 minutes.

In some embodiments, the nucleic acid detection system amplification and detection reaction can occur within about 1 to about 60 minutes, e.g. within about 1, 2, 3, 4, 5, 6, 7,8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to/or about 60 minutes. In some embodiments, the nucleic acid detection system amplification and detection reaction can occur within about 1 to about 45 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or about 45 minutes. In some embodiments, the nucleic acid detection system amplification and detection can occur within about 1 to about 30 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, to/or about 30 minutes. In some embodiments, the nucleic acid detection system amplification and detection reaction can occur within about 1 to about 25 minutes, e.g., within about 1, 2, 3, 4, 5, 6, 7, 8, 9, 10, 11, 12, 13, 14, 15, 16, 17, 18, 19, 20, 21, 22, 23, 24, to/or about 25 minutes. In some embodiments, the nucleic acid detection system amplification and detection reaction can occur within about 1 to about 20 minutes, e.g., within about 1, 2, 3, 4, 5, 6, 7, 8, 9, 10, 11, 12, 13, 14, 15, 16, 17, 18, 19, to/or about 20 minutes. In some embodiments, nucleic acid detection system amplification and detection reaction can occur within about 1 to about 15 minutes, e.g., within about 1, 2, 3, 4, 5, 6, 7, 8, 9, 10, 11, 12, 13, to/or about 15 minutes. In some embodiments, the nucleic acid detection system amplification and detection reaction can occur within about 1 to about 10 minutes, e.g., within about 1, 2, 3, 4, 5, 6, 7, 8, 9, to/or about 10 minutes. In some embodiments, the nucleic acid detection system amplification and detection reaction can occur within about 1 to about 5 minutes, e.g., within about 1, 2, 3, 4, to/or about 5 minutes.

### Sample Preparation

In some embodiments, the sample preparation can include extraction-free release of polynucleotides (e.g., DNA and/or RNA) from cells and/or microorganisms, such as viruses, present in the sample. In some embodiments, the sample preparation can include virus inactivation and/or nuclease inactivation. In some embodiments sample preparation is composed of inactivating nucleases present in a sample followed by virus inactivation. The step of sample preparation can occur prior to any target amplification and/or CRISPR-effector system detection. In some embodiments, sample preparation can include nuclease inactivation and/or viral inactivation by 1, 2, 3, 4 or more thermal (heat or cold) inactivation steps, chemical inactivation steps, biologic inactivation, physiologic inactivation, physical inactivation steps, or any combination thereof.

In some embodiments, sample preparation can include one or more thermal steps. In some embodiments, nuclease inactivation can include one or more thermal steps. In some embodiments, microbe inactivation can include one or more thermal steps. Thermal steps can be heating, cooling, cycles of heating and cooling at one or more rates of temperature change. Without being bound by theory, in some embodiments, heating and/or cooling, and/or one or more heating/cooling cycles as described herein can disrupt the integrity, function, and/or activity of biological molecules and structures (such as enzymes, membranes, viral capsids, and the like). In some embodiments, the sample presentation can be composed of or include 1, 2, 3, 4, or more heating steps at one or more different temperatures. In some embodiments, the sample presentation can be composed of or include 1, 2, 3, 4, or more cooling steps at one or more different temperatures. The duration of thermal each step can be independently selected from about 0.5 to about 60 minutes or more, such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60 minutes or more.

In some embodiments, one or more or all of the sample preparation steps can occur at about 15-95 degrees C. In some embodiments, one or more or all of the sample preparation steps can occur at about 15-30 degrees C., about 20-25 degrees C., or about 22-25 degrees C. In some embodiments, one or more or all of the sample preparations steps can occur at about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, to/or 95 degrees C. In some embodiments, the one or more or all of the sample preparations steps can occur at about 15, 16, 17, 18, 19, 20, 21, 22, 23, 24, 25, 26, 27, 28, 29, 30, 31, 32, 33, 34, 35, 36, or/to 37 degrees C., about 15, 16, 17, 18, 19, 20, 21, 22, 23, 24, 25, 26, 27, 28, 29, 30, 31, 32, or/to 33 degrees C., about 22, 23, 24, 25, 26, or/to 27 degrees C., about 15, 16, 17, 18, 19, 20, 21, 22, 23, 24, 25, 26, 27, 28, 29, to/or 30 degrees C., about 15, 16, 17, 18, 19, 20, 21, 22, 23, 24, to/or 25 degrees C., about 20, 21, 22, 23, 24, 25, 26, 27, 28, 29, to/or 30 degrees C., about 20, 21, 22, 23, 24, or/to 25 degrees C., or about 22, 23, 24, or/to 25 degrees C. In some embodiments, one or more or all of the sample preparation steps reaction can be performed at about room temperature (about 15-30 degrees C.). In some embodiments, one or more or all of the sample preparations steps can be carried out at 37° C. to 50° C., such as about 37, 38, 39, 40, 41, 42, 43, 44, 45, 46, 47, 48, 49, to/or about 50 degrees C. In some embodiments, one or more or all of the sample preparation steps can be carried out at about 64-95 degrees C., such as 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, to/or 95 degrees C.

In some embodiments, nuclease inactivation can occur at about 15-50 degrees C. In some embodiments, one or more or all of the sample preparations steps can occur at about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, to/or 50 degrees C. In some embodiments, nuclease inactivation can occur at about 15, 16, 17, 18, 19, 20, 21, 22, 23, 24, 25, 26, 27, 28, 29, 30, 31, 32, 33, 34, 35, 36, or/to 37 degrees C., about 15, 16, 17, 18, 19, 20, 21, 22, 23, 24, 25, 26, 27, 28, 29, 30, 31, 32, or/to 33 degrees C., about 15, 16, 17, 18, 19, 20, 21, 22, 23, 24, 25, 26, or/to 27 degrees C., about 15, 16, 17, 18, 19, 20, 21, 22, 23, 24, to/or 25 degrees C., about 15, 16, 17, 18, 19, 20, 21, 22, 23, 24, 25, 26, 27, 28, 29, to/or 30 degrees C., about 15, 16, 17, 18, 19, 20, 21, 22, 23, 24, to/or 25 degrees C., about 20, 21, 22, 23, 24, 25, 26, 27, 28, 29, to/or 30 degrees C., about 20, 21, 22, 23, 24, or/to 25 degrees C., or about 22, 23, 24, or/to 25 degrees C. In some embodiments, nuclease inactivation can occur at about room temperature (about 15-30 degrees C.). In some embodiments, nuclease inactivation can occur at about 37° C. to 50° C., such as about 37, 38, 39, 40, 41, 42, 43, 44, 45, 46, 47, 48, 49, to/or about 50 degrees C.

In some embodiments, microbe inactivation can occur at about 15 to about 95 degrees C., such as about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, to/or 95 degrees C. In some embodiments, microbe inactivation can occur at about 15-37 degrees C., e.g., 15, 16, 17, 18, 19, 20, 21, 22, 23, 24, 25, 26, 27, 28, 29, 30, 31, 32, 33, 34, 35, 36, and/or 37 degrees C. In some embodiments, microbe inactivation can occur at about 15 to about 33 degrees C., such as, 15, 16, 17, 18, 19, 20, 21, 22, 23, 24, 25, 26, 27, 28, 29, 30, 31, 32, to/or 33 degrees C. In some embodiments, microbe inactivation can occur at about 15 to about 30 degrees C., such as, 15, 16, 17, 18, 19, 20, 21, 22, 23, 24, 25, 26, 27, 28, 29, to/or 30 degrees C. In some embodiments, microbe inactivation can occur at about 15 to about 25 degrees C., such as, 15, 16, 17, 18, 19, 20, 21, 22, 23, 24, to/or 25 degrees C. In some embodiments, microbe inactivation can occur at about 22 to about 25 degrees C., such as, 22, 23, 24, to/or 25 degrees C. In some embodiments, the microbe inactivation step is carried out at a temperature ranging from 64° C. to 95° C., such as 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, to/or 95 degrees C.

In some embodiments, the sample preparation can occur within about 1 to about 60 minutes, e.g. within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to/or about 60 minutes. In some embodiments, the sample preparation can occur within about 1 to about 45 minutes, e.g., within about 1, 2, 3, 4,5,6,7,8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or about 45 minutes. In some embodiments, the sample preparation can occur within about 1 to about 60 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, to/or about 30 minutes. In some embodiments, the sample preparation can occur within about 1 to about 25 minutes, e.g., within about 1, 2, 3, 4, 5, 6, 7, 8, 9, 10, 11, 12, 13, 14, 15, 16, 17, 18, 19, 20, 21, 22, 23, 24, to/or about 25 minutes. In some embodiments, the sample preparation can occur within about 1 to about 20 minutes, e.g., within about 1, 2, 3, 4, 5, 6, 7, 8, 9, 10, 11, 12, 13, 14, 15, 16, 17, 18, 19, to/or about 20 minutes. In some embodiments, the sample preparation can occur within about 1 to about 15 minutes, e.g., within about 1, 2, 3, 4, 5, 6, 7, 8, 9, 10, 11, 12, 13, to/or about 15 minutes. In some embodiments, the sample preparation can occur within about 1 to about 10 minutes, e.g., within about 1, 2, 3, 4, 5, 6, 7, 8, 9, to/or about 10 minutes. In some embodiments, the sample preparation can occur within about 1 to about 5 minutes, e.g., within about 1, 2, 3, 4, to/or about 5 minutes.

In some embodiments, the nuclease and/or microbe inactivation can occur within about 1 to about 60 minutes, e.g. within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to/or about 60 minutes. In some embodiments, the nuclease and/or microbe inactivation can occur within about 1 to about 45 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or about 45 minutes. In some embodiments, the nuclease and/or microbe inactivation can occur within about 1 to about 60 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, to/or about 30 minutes. In some embodiments, the nuclease and/or microbe inactivation can occur within about 1 to about 25 minutes, e.g., within about 1, 2, 3, 4, 5, 6, 7, 8, 9, 10, 11, 12, 13, 14, 15, 16, 17, 18, 19, 20, 21, 22, 23, 24, to/or about 25 minutes. In some embodiments, the nuclease and/or microbe inactivation can occur within about 1 to about 20 minutes, e.g., within about 1, 2, 3, 4, 5, 6, 7, 8, 9, 10, 11, 12, 13, 14, 15, 16, 17, 18, 19, to/or about 20 minutes. In some embodiments, the nuclease and/or microbe inactivation can occur within about 1 to about 15 minutes, e.g., within about 1, 2, 3, 4, 5, 6, 7, 8, 9, 10, 11, 12, 13, to/or about 15 minutes. In some embodiments, the nuclease and/or microbe inactivation can occur within about 1 to about 10 minutes, e.g., within about 1, 2, 3, 4, 5, 6, 7, 8, 9, to/or about 10 minutes. In some embodiments, the nuclease and/or microbe inactivation can occur within about 1 to about 5 minutes, e.g., within about 1, 2, 3, 4, to/or about 5 minutes. In some embodiments, the nuclease inactivation step is of a duration selected from 5 minutes, 10 minutes, 15 minutes, and 20 minutes.

In some embodiments, one or more sample preparation steps can include one or more steps incubating the sample for a period of time at a temperature ranging from about 15-95 degrees C., 15-64 degrees C., 15-37 degrees C., 15-30 degrees C., 15-27 degrees C., 15-25 degrees C., 20-30 degrees C., 22-25 degrees C., −80 degrees C. to about 0 degrees C., −60 degrees C. to about 0 degrees C., −40 degrees C. to about 0 degrees C., −20 degrees C. to about 0 degrees C., −10 degrees C. to about 0 degrees C., −5 degrees C. to about 0 degrees C., or a combination thereof. In some embodiments, the period of time for each incubation can range from 0.5 min to about 60 minutes, such as about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, to/or 60 minutes. In some embodiments, the period of time for each incubation can range from about 1 hour to about 24 hours, such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, to/or about 24 hours.

In some embodiments, sample preparation can include one or more chemical inactivation steps. In some embodiments, nuclease inactivation can include one or more chemical inactivation steps. In some embodiments, viral inactivation can include one or more chemical inactivation steps. Chemical inactivation steps can include, but are not limited to, treatment with DEPC, 2-Mercaptoethanol, EDTA, EGTA, DTT, TCEP 2-nitro-5-thiocyanobenzoic acid, Ca²⁺, Sodium dodecyl sulfate, Carbodiimide and cholesterol sulfate, Iodoacetate, DNase inactivation reagent (Ambion Life Sciences), RNaseZap (Qiagen), SecurRIN advanced RNase inhibitor (e.g., cat no. RNI0301 from HghiQu GmbH), RNAse alert (Ambion), and combinations thereof.

In some embodiments, each of the chemicals in a step of the sample preparation reaction and/or solution can be included in the sample preparation solution or reaction at a concentration of 1 to 1000, e.g. 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 pM, nM, μM, mM, or M. In some embodiments, each of the chemicals a step of sample preparation reaction and/or solution can be included at 0.01 to about 100 w/v, v/v, or w/w percent of the reaction solution and/or sample preparation solution, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, 49, 49.1, 49.2, 49.3, 49.4, 49.5, 49.6, 49.7, 49.8, 49.9, 50, 50.1, 50.2, 50.3, 50.4, 50.5, 50.6, 50.7, 50.8, 50.9, 51, 51.1, 51.2, 51.3, 51.4, 51.5, 51.6, 51.7, 51.8, 51.9, 52, 52.1, 52.2, 52.3, 52.4, 52.5, 52.6, 52.7, 52.8, 52.9, 53, 53.1, 53.2, 53.3, 53.4, 53.5, 53.6, 53.7, 53.8, 53.9, 54, 54.1, 54.2, 54.3, 54.4, 54.5, 54.6, 54.7, 54.8, 54.9, 55, 55.1, 55.2, 55.3, 55.4, 55.5, 55.6, 55.7, 55.8, 55.9, 56, 56.1, 56.2, 56.3, 56.4, 56.5, 56.6, 56.7, 56.8, 56.9, 57, 57.1, 57.2, 57.3, 57.4, 57.5, 57.6, 57.7, 57.8, 57.9, 58, 58.1, 58.2, 58.3, 58.4, 58.5, 58.6, 58.7, 58.8, 58.9, 59, 59.1, 59.2, 59.3, 59.4, 59.5, 59.6, 59.7, 59.8, 59.9, 60, 60.1, 60.2, 60.3, 60.4, 60.5, 60.6, 60.7, 60.8, 60.9, 61, 61.1, 61.2, 61.3, 61.4, 61.5, 61.6, 61.7, 61.8, 61.9, 62, 62.1, 62.2, 62.3, 62.4, 62.5, 62.6, 62.7, 62.8, 62.9, 63, 63.1, 63.2, 63.3, 63.4, 63.5, 63.6, 63.7, 63.8, 63.9, 64, 64.1, 64.2, 64.3, 64.4, 64.5, 64.6, 64.7, 64.8, 64.9, 65, 65.1, 65.2, 65.3, 65.4, 65.5, 65.6, 65.7, 65.8, 65.9, 66, 66.1, 66.2, 66.3, 66.4, 66.5, 66.6, 66.7, 66.8, 66.9, 67, 67.1, 67.2, 67.3, 67.4, 67.5, 67.6, 67.7, 67.8, 67.9, 68, 68.1, 68.2, 68.3, 68.4, 68.5, 68.6, 68.7, 68.8, 68.9, 69, 69.1, 69.2, 69.3, 69.4, 69.5, 69.6, 69.7, 69.8, 69.9, 70, 70.1, 70.2, 70.3, 70.4, 70.5, 70.6, 70.7, 70.8, 70.9, 71, 71.1, 71.2, 71.3, 71.4, 71.5, 71.6, 71.7, 71.8, 71.9, 72, 72.1, 72.2, 72.3, 72.4, 72.5, 72.6, 72.7, 72.8, 72.9, 73, 73.1, 73.2, 73.3, 73.4, 73.5, 73.6, 73.7, 73.8, 73.9, 74, 74.1, 74.2, 74.3, 74.4, 74.5, 74.6, 74.7, 74.8, 74.9, 75, 75.1, 75.2, 75.3, 75.4, 75.5, 75.6, 75.7, 75.8, 75.9, 76, 76.1, 76.2, 76.3, 76.4, 76.5, 76.6, 76.7, 76.8, 76.9, 77, 77.1, 77.2, 77.3, 77.4, 77.5, 77.6, 77.7, 77.8, 77.9, 78, 78.1, 78.2, 78.3, 78.4, 78.5, 78.6, 78.7, 78.8, 78.9, 79, 79.1, 79.2, 79.3, 79.4, 79.5, 79.6, 79.7, 79.8, 79.9, 80, 80.1, 80.2, 80.3, 80.4, 80.5, 80.6, 80.7, 80.8, 80.9, 81, 81.1, 81.2, 81.3, 81.4, 81.5, 81.6, 81.7, 81.8, 81.9, 82, 82.1, 82.2, 82.3, 82.4, 82.5, 82.6, 82.7, 82.8, 82.9, 83, 83.1, 83.2, 83.3, 83.4, 83.5, 83.6, 83.7, 83.8, 83.9, 84, 84.1, 84.2, 84.3, 84.4, 84.5, 84.6, 84.7, 84.8, 84.9, 85, 85.1, 85.2, 85.3, 85.4, 85.5, 85.6, 85.7, 85.8, 85.9, 86, 86.1, 86.2, 86.3, 86.4, 86.5, 86.6, 86.7, 86.8, 86.9, 87, 87.1, 87.2, 87.3, 87.4, 87.5, 87.6, 87.7, 87.8, 87.9, 88, 88.1, 88.2, 88.3, 88.4, 88.5, 88.6, 88.7, 88.8, 88.9, 89, 89.1, 89.2, 89.3, 89.4, 89.5, 89.6, 89.7, 89.8, 89.9, 90, 90.1, 90.2, 90.3, 90.4, 90.5, 90.6, 90.7, 90.8, 90.9, 91, 91.1, 91.2, 91.3, 91.4, 91.5, 91.6, 91.7, 91.8, 91.9, 92, 92.1, 92.2, 92.3, 92.4, 92.5, 92.6, 92.7, 92.8, 92.9, 93, 93.1, 93.2, 93.3, 93.4, 93.5, 93.6, 93.7, 93.8, 93.9, 94, 94.1, 94.2, 94.3, 94.4, 94.5, 94.6, 94.7, 94.8, 94.9, 95, 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, 99.9, 100 w/v, v/v, or w/w percent of the reaction and/or sample preparation solution.

In some embodiments, sample preparation can include one or more biological inactivation steps. In some embodiments, nuclease inactivation can include one or more biological activation steps. In some embodiments, microbe inactivation can include one or more biological inactivation steps. In some embodiments, the biological inactivation step can include exposing the sample to an enzyme or other biological molecule. In some embodiments, the enzyme or biological molecule can inactivate one or more enzymes or other molecules in the sample, such as but not limited to, one or more nucleases. In some embodiments, the enzyme or other biological molecule can bind one or more components the sample (such as a binding protein like albumin etc.) such that the bound components are inactive. In some embodiments, the enzyme or other biological molecule included in a biologic inactivation step can include, but not limited to, a DNAse inhibitor enzyme (see e.g., Eur J Med Chem. 2014 Dec. 17; 88:101-11.doi: 10.1016/j.ejmech.2014.07.040.Epub 2014 Jul. 15.), an RNAse inhibitor enzyme (e.g. QIAGEN RNase Inhibitor (Cat. No. 129916 QIAGEN, human placental RNAse inhibitor), proteinase K, and combinations thereof.

The biological molecule can be included in the sample preparation solution or reaction at a concentration of 1 to 1000, e.g., 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 pM, nM, sM, mM, or M. The biological molecule can be included in the sample preparation solution or reaction at a concentration of 1 to 1000, e.g., 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 units per pL, nL, μL, mL, or L.

In some embodiments, sample preparation can include one or more physiological inactivation steps. In some embodiments, nuclease inactivation can include one or more physiological inactivation steps. In some embodiments, viral inactivation can include one or more physiological inactivation steps. The phrase “physiological inactivation” refers to conditions that deviate from the normal working physiological conditions (e.g., pH, osmolarity, temperature, salinity, etc.) necessary for causing or maintaining the activation of a component (e.g., an enzyme) present in a sample that result in the inactivation or inhibition of the function or activity of the component. In some embodiments, the pH of the sample can be altered by 1, 2, 3, 4, 5, 6, 7, 8, 9,10, 11, 12, 13, or 14 pH units away from normal physiological conditions for the sample and/or a component thereof within it. In some embodiments, the pH of the sample is adjusted to less than about 7, such as pH about 1, 2, 3, 4, 5, or about 6. In some embodiments, the pH of the sample is adjusted to greater than about 7, such as pH about 8, 9, 10, 11, 12, 13, or 14. In some embodiments, the pH is adjusted to about 7. It will be appreciated that some enzymes are active in an acidic or basic environment, and thus even a neutral pH (about 7) can serve, in some embodiments, to inactivate or inhibit such an enzyme or component of the sample. In some embodiments, the osmolarity and/or salinity of the sample can be altered outside of a normal physiological state with any suitable buffers or reagents.

In some embodiments, sample preparation can include one or more physical inactivation steps. In some embodiments, nuclease inactivation can include one or more physical inactivation steps. In some embodiments, microbe inactivation can include one or more physical inactivation steps. In some embodiments, physical inactivation can include, without limitation, mechanical methods (shaking, vibrations (including resonant vibrations, acoustic vibrations, mechanical vibrations), centrifugation, electromagnetic waves, sounds waves, light waves, magnetic fields, thermal shifts (heat-cold transitions and cycles), physical bombardment, and combinations thereof.

Where the sample preparation step includes one or more reagents, active agents, buffers, and the like, these can be contained in a sample preparation solution or microbe polynucleotide preparation solution, in the context of microbe detection. In some embodiments, the reagents of the sample preparation solution can be contained in a reaction vessel, reaction location, and/or device in solid or liquid form and the sample can be added to the reagents. In some embodiments, one or more reactions involved in sample preparation can begin once the sample is contacted and/or mixed with the sample preparation solution. In some embodiments, the sample preparation solution and/or microbe polynucleotide preparation solution is shelf-stable. In some embodiments, the sample preparation solution and/or microbe polynucleotide preparation solution is shelf-stable at ambient temperature. In some embodiments, the sample preparation solution and/or microbe polynucleotide preparation solution is shelf-stable at a temperature ranging from about 15 to about 30 degrees C. In some embodiments, the sample preparation solution and/or microbe polynucleotide preparation solution or one or more components thereof are lyophilized.

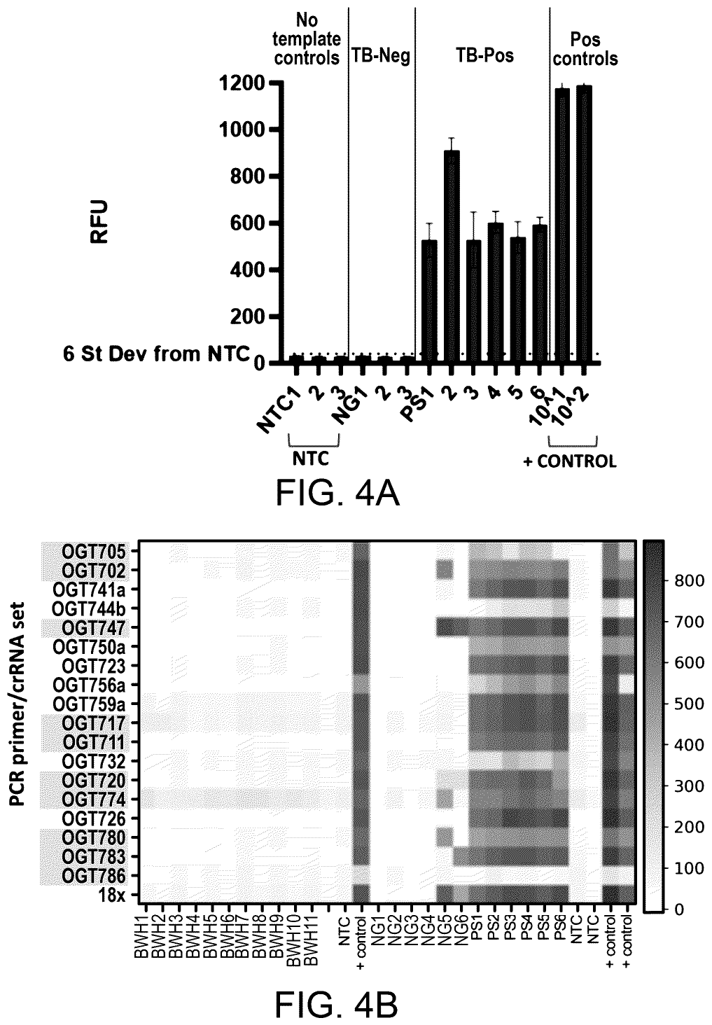

In some embodiments, the sample preparation can include one or more serial dilutions. In some embodiments, the dilution series is conducted such that one or more contaminants are decreased. In some embodiments, the one or more contaminants are non-cell free nucleic acids. In some embodiments, the dilution(s) are performed as described in WO 2019178157, particularly at paragraphs [98]-[112] and FIGS. 4a-4b therein. Dilution can refer to the process of adding additional solvent, liquid, or buffer to a solution or a sample to decrease its concentration. This process can keep the amount of solute constant, but increases the total amount of solution, thereby decreasing its final concentration. Dilution can also refer to taking different quantities of a sample (such as progressively smaller or larger quantities) and mixing such quantities with a fixed or variable level of a diluent or no added diluent, solvent, liquid or buffer. Dilution can also be achieved by mixing a solution of higher concentration with an identical solution of lesser concentration. In some aspects, a solvent in regards to dilution can be a buffer, a positive control, a negative control, culture medium, or a sample. In some aspects, a solute in regards to dilution can be a target nucleic acid, a buffer, a positive control, a negative control, culture medium, or a sample. In some aspects, a solute in regard to dilution can be a target nucleic acid. In some aspects, dilution can refer to decreasing a final concentration of a target nucleic acid.

The terms “serial dilution” or “dilution series” are used interchangeably herein. The terms refer to a dilution series generated in any manner. Dilution series can refer to the dilution of mass, volume, or a specific quantity, for example cell count. Therefore, to dilute a sample, the quantity of diluent to be added can be based on the mass, volume, or cell count of a sample. Further, a quantity or amount of diluent can refer to a mass, volume, or number of cells. In some instances, a dilution series or serial dilution can refer to a succession of step dilutions, (often, each with the same dilution factor), where the diluted material of the previous step is used to make the subsequent dilution. In other cases, each member of the dilution series is directly obtained from the undiluted sample. For example, the undiluted sample is used to make a two-fold dilution by adding an equal volume of diluent to an equal volume of the undiluted sample. In other instances, a four fold dilution in the series can be produced by adding diluent directly to a portion of the undiluted sample in a 3:1 ratio (e.g., 3 μl of diluent added to 1 μl of sample yields a four fold dilution). In still other instances, a dilution series can consist of a series of changes in quantity, based on the mass, volume, or number of cells of the sample.

In some instances, a first dilution to a second dilution can be less or greater than about 1:1, 1:2, 1:3, 1:4, 1:5, 1:6, 1;7, 1:8, 1:9, 1:10, 1:11, 1:12, 1:13, 1:14, 1:15, 1:20, 1:130, 1:50, 1:100; or 1:1000. In some instances a first dilution to a second dilution can be less or greater than about 1000:1, 100:1, 50:1, 30:1, 20:1, 15:1, 14; 1, 13:1, 12:1, 11:1, 10:1, 9:1, 8:1, 7:1, 6:1, 5:1, 4:1, 3:1, 2:1; or 1:1.

In some embodiments, a diluent used to dilute a sample or target nucleic acids described herein can be a reagent. In some embodiments a diluent used to dilute a sample or target nucleic acids described herein can be a buffer, medium such as growth media, a positive control, a negative control, culture medium, a sample, a synthetic molecule, or synthetic plasma. In some aspects, a buffer can be basic, acidic, neutral, or isotonic. A buffer can be a solution buffer that prevents a change in pH. A buffer can be an extraction buffer, a suspension buffer or a lysis buffer. A buffer can be phosphate-buffered saline (PBS). A buffer may comprise potassium chloride, sodium thioglycollate, dodecylamine, a sugar, fructose, glucose, mannitol, maltose, glycerol, alanine, arginine, histidine, lysine, proline, asparagine, aspartic acid, pheylalanine, inosine, insulin EDTA, NAOH, NaCl and/or Tris-HCl in any combination. In some embodiments a diluent used to dilute a sample or target nucleic acids described herein is pretreated to uniquely label the nucleic acids in the diluent before using to prepare members of the dilution series. In some embodiments all or a fraction of nucleic acids in the diluent are biotinylated. In some embodiments diluent-specific barcodes are attached to all or a fraction of nucleic acids in the diluent. In some embodiments diluent-specific barcodes are attached by the addition of nucleotides to the 3′ and/or 5′ terminus of all or a fraction of nucleic acids in the diluent. In some embodiments the diluent-specific barcodes attached to the nucleic acids sequences present originally in the diluent are sequenced which allows for determination of the contribution of Cs to Ctotai (see symbol definitions above) during analysis of the dilution series sequencing data.

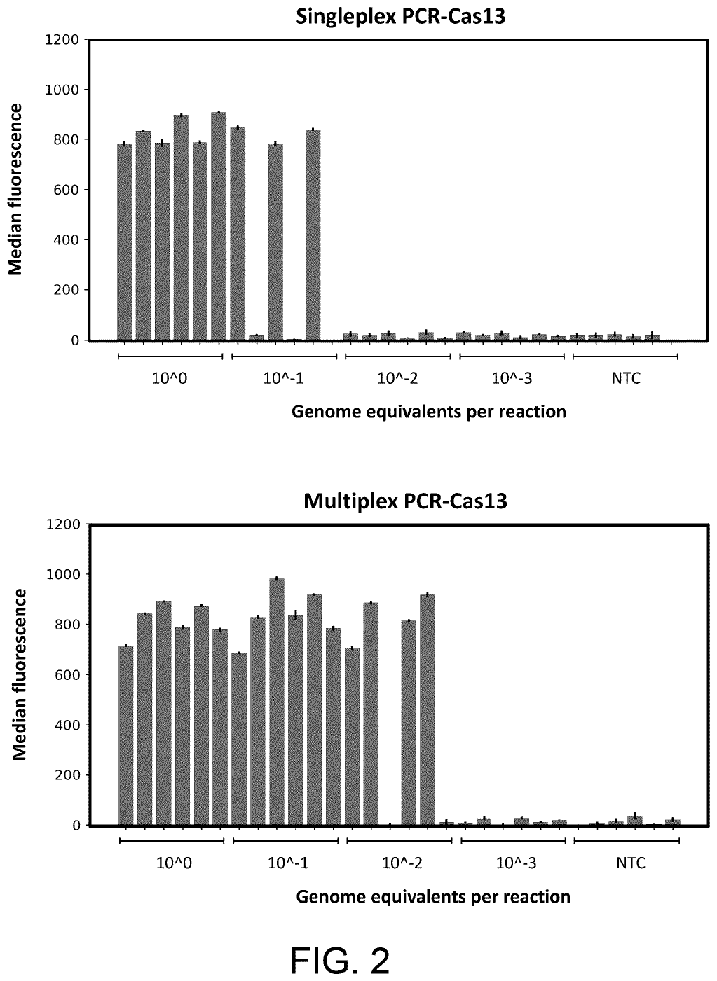

In some embodiments contaminants (such as host or other contaminating nucleic acids or molecules) can be removed and/or inactivated according to a method and/or using one or more reagents as described in WO 2018031486, particularly at paragraphs [0006]-[0032], [0041]-[0046], [0060]-[0070], [0079]-[0112], and [0116], and FIGS. 1-2.

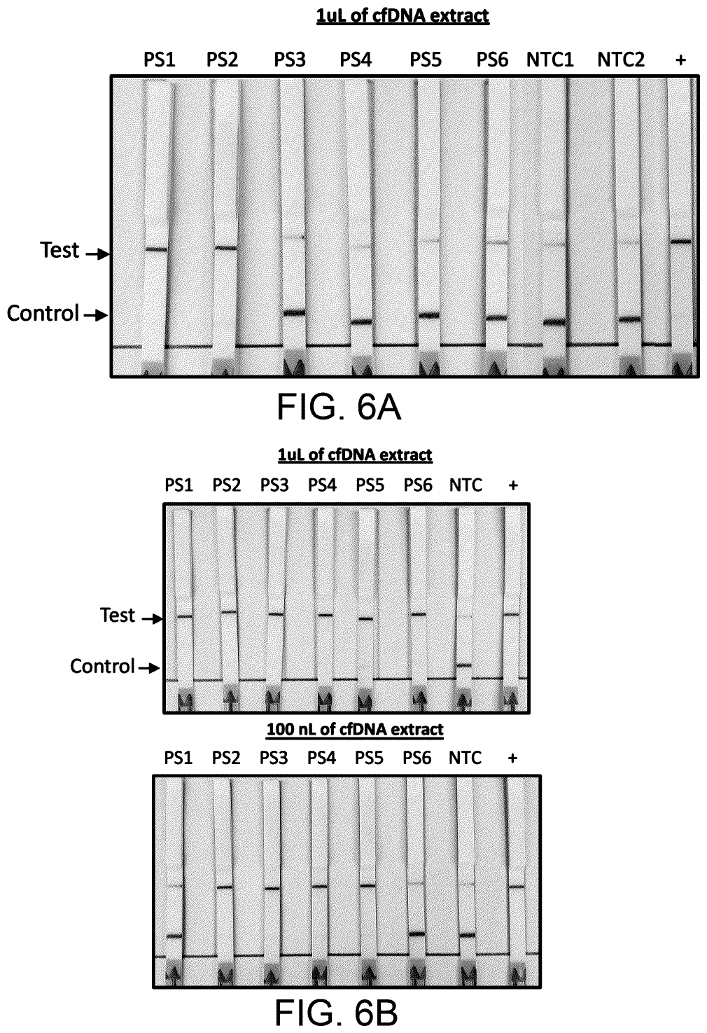

In some embodiments, one or more synthetic components or “spike-ins” can be added to a sample or dilution thereof to serve as reference or provide other information regarding a sample that can be detected and/or processed in a downstream step or analysis. In some cases, the spike-ins are nucleic acids. In some embodiments, the spike-in nucleic acid(s) added are as described in e.g., WO 2017/165864 (also published as US20170275691), particularly at para. [0003]-[0061], [0092]-[0013], [00131]-[0189], [0216]-[0239], Table 1, , SEQ ID NO: 1-110, and FIGS. 1-15.

In some embodiments, the sample is processed such that concurrent processing of different and multiple forms of nucleic acids can occur, such as concurrent processing of (e.g., single-stranded DNA, double-stranded DNA, single-stranded RNA, and/or double-stranded RNA). In some embodiments, concurrent processing of different and multiple forms of nucleic acids can occur via a method and/or using reagents described in U.S. Pat. No. 11,180,800 (a divisional of U.S. Pat. No. 10,697,008), particularly at Col. 1: In. 40-67, 2:1-67; 3:1-65; 4:1-67, 5:1-67, 6:1-67, 7:1-39, 10:13-67, 11:1-67, 12:1-67, 13:1-67, 14:1-3, Col 15-38 and 39:1-20, and FIGS. 1-9.

Due to the sensitivity of said systems, a number of applications that require from the rapid and sensitive detection may benefit from the embodiments disclosed herein and are contemplated to be within the scope of the invention.

Pooled and/or Tiled Amplification

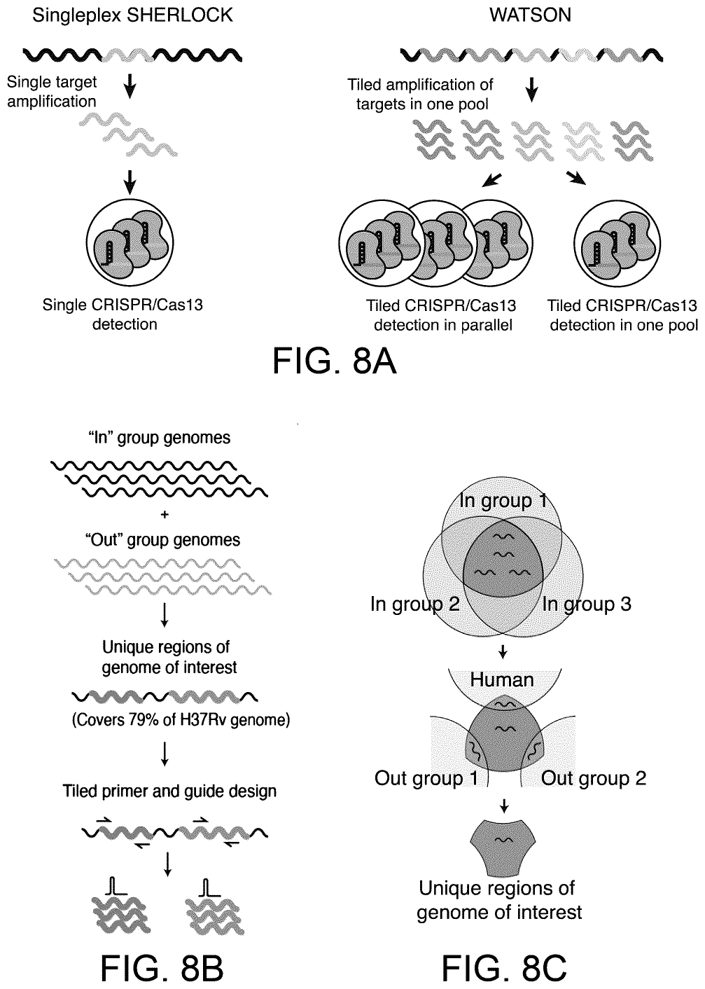

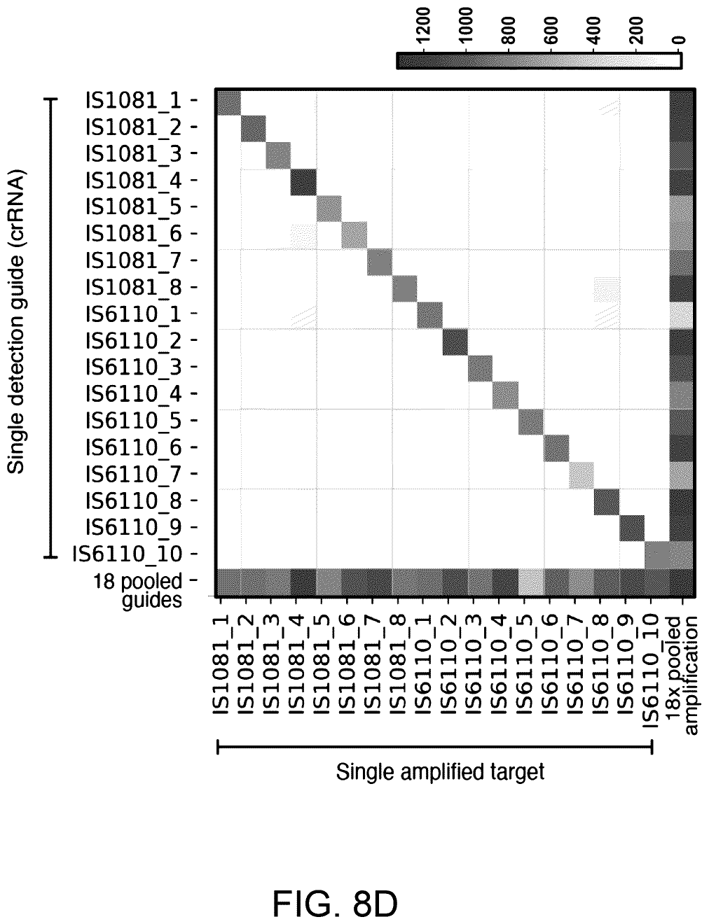

The amplification can be pooled and/or tiled as previously described. In some embodiments, target nucleic acids, and more particularly target regions in target elements across a genome can be tiled and amplified in a pooled amplification reaction as is shown and described in connection with FIG. 8A. In some embodiments, target amplification is pooled such that multiple amplification target regions and optionally target elements are amplified in the same reaction. In some embodiments, one or more of the target elements are repetitive target elements (i.e., they exist two or more times in the genome). As is also described elsewhere herein, tiling of target elements in a genome can increase the sensitivity of the assay, particularly for low abundance targets and for targets where the genome or nucleic acid may be fragmented, which is often the case for ccfDNA and cfDNA.

Target region amplification can be performed using any suitable method. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM). In certain embodiments, the amplification can utilize a transposase-based isothermal amplification method (see e.g. WO 2020/006049, which is incorporated by reference herein as if expressed in its entirety), nickase-based isothermal amplification method (see e.g. WO 2020/006067, which is incorporated by reference herein as if expressed in its entirety), or a helicase-based amplification method (see e.g., WO 2020/006036, which is incorporated by reference herein as if expressed in its entirety). In some embodiments, amplification is via LAMP. In some embodiments, amplification is via RPA.

In certain example embodiments, the RNA or DNA amplification is nucleic acid sequence-based amplification is NASBA, which is initiated with reverse transcription of target RNA by a sequence-specific reverse primer to create an RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected by the guide RNAs thus further enhancing the sensitivity of the assay. Binding of the target RNAs by the guide RNAs then leads to activation of the CRISPR effector protein and the methods proceed as outlined above. The NASBA reaction has the additional advantage of being able to proceed under moderate isothermal conditions, for example at approximately 41° C., making it suitable for systems and devices deployed for early and direct detection in the field and far from clinical laboratories.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, an RNA polymerase promoter, such as a T7 promoter, is added to one of the primers. This results in an amplified double-stranded DNA product comprising the target sequence and an RNA polymerase promoter. After, or during, the RPA reaction, an RNA polymerase is added that will produce RNA from the double-stranded DNA templates. The amplified target RNA can then in turn be detected by the CRISPR effector system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

RPA primers suitable for use with the present invention can be designed and optimized so as to amplify a target to achieve a desired sensitivity, optionally in conjunction with a CRISPR-Cas based nucleic acid detection method described elsewhere herein. A computational approach to primer and/or guideRNA design may be employed to design suitable RPA primers for a desired target and/or guideRNA(s) used. The Working examples herein demonstrate a computational pipeline for primer and guideRNA sets to uniquely recognize the Mycobacterium tuberculosis genome and its experimental validation for the development of primers and guideRNA sets for target amplification and CRISPR-Cas based nucleic acid detection. Such an approach can be applied for the design and optimization of RPA primers and guideRNA(s) for detection of a desired target.

In some embodiments, RPA primers are designed and optimized for use in a single or multiplexed amplification of a target, such as a single repetitive genetic element in a genome. In some embodiments, an assay can include sets of RPA primers designed and optimized for use in a multiplexed reaction where each set is specific for a single genetic element and where the collective sets are specific to targets tiled across multiple loci (or genetic elements) in the genome.

In some embodiments, optimization of RPA primers includes applying a trained algorithm to empirically screen and test potential primers in silico to obtain primer sets and in multiplexed amplification reactions.

In some embodiments, length of each RPA primer is about 25 to about 35, such as about 25, 26, 27, 28, 29, 30, 31, 32, 33, to/or 34, to/or 35 nucleotides in length.

In some embodiments, RPA primers in a given set for a multiplexed reaction produce an amplicon of about the same size.

In some embodiments, the RPA primers each have a GC content ranging from about 40 percent to about 60 percent. In some embodiments, the RPA primers each have a GC content of about 40, 41, 42, 43, 44, 45, 46, 47, 48, 49, 50, 51, 52, 53, 54, 55, 56, 57, 58, 59, to/or about 60 percent.

In some embodiments, the primers produce an amplicon of about 100 base pairs or less, about 90 base pairs or less, about 85 base pairs or less, about 80 base pairs or less, about 75 base pairs or less, about 70 base pairs or less, about 65 base pairs or less, about 60 base pairs or less, about 55 base pairs or less, about 50 base pairs or less, about 45 base pairs or less, about 40 base pairs or less, about 35 base pairs or less, about 30 base pairs or less, about 25 base pairs or less, about 20 base pairs or less, or about 15 base pairs or less.

In some embodiments, a primer pair as measured by a single-plex amplification provides a limit of detection of less than about 10, less than about 5, less than about 4, less than about 3, less than about 2, less than about 1, less than about 0.5, less than about 0.01, or less than about 0.001 genome equivalents per reaction.

In some embodiments, a pooled set of primer pairs as measured by a multi-plex reaction amplification provides a limit of detection of less than about 10, less than about 5, less than about 4, less than about 3, less than about 2, less than about 1, less than about 0.5, less than about 0.01, less than about 0.001, or less than about 0.0001 genome equivalents per reaction.

In some embodiments, the primer pairs in a multiplexed set (or pool) of primer pairs all have about the same performance (as measured by LOD of genome equivalents) when evaluated in a single-plex reaction.

### Amplification Reagents

The methods herein can utilize one or more amplification reagents. The amplification reagents can also be continued in kits and/or devices described herein. The amplification reagents can be part of the nucleic acid detection systems described herein. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl₂), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH₄)₂SO₄], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application and may be specific to the reaction in some cases. Amplification reactions may include dNTPs and/or nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or apatamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In certain embodiments, detection of DNA with the methods or systems of the invention requires transcription of the (amplified) DNA into RNA prior to detection.

In some embodiments, the amplification reagent or component thereof is shelf-stable. In some embodiments, the amplification reagent or component thereof is shelf-stable at ambient temperature. In some embodiments, the amplification reagent or component thereof is shelf-stable at 15-30 degrees C.

### Target Polynucleotide Enrichment

In certain example embodiments, target RNA or DNA may first be enriched prior to detection or amplification of the target RNA or DNA. In certain example embodiments, this enrichment may be achieved by binding of the target nucleic acids by a CRISPR effector system.

Current target-specific enrichment protocols require single-stranded nucleic acid prior to hybridization with probes. In some embodiments this step is skipped, which allows direct targeting to double-stranded DNA (either partly or completely double-stranded). In addition, the embodiments disclosed herein are enzyme-driven targeting methods that offer faster kinetics and easier workflow allowing for isothermal enrichment. In certain example embodiments enrichment may take place between 20-37° C.

In some embodiments, non-host nucleic acids in a sample, such as cfDNA, can be enriched as compared to an original sample. In some embodiments, enrichment is accomplished by removal of nucleic acids of a particular size or length that does not correspond to the desired nucleic acid target population. In some embodiments, enrichment is accomplished by removing or otherwise excluding nucleic acids whose length is larger than cfDNA fragment lengths. See e.g., and US20170016048 (also published as WO 2016187234 and U.S. Pat. No. 11,111,520), particularly at paragraphs [0006]-[0007], [0012], [0014], [0016], [0017], [0019], [0024]-[0033], [0050]-[0204], and FIGS. 1-3 therein. Amplification Primer Set and Amplified Target Element/Region Selection and Design

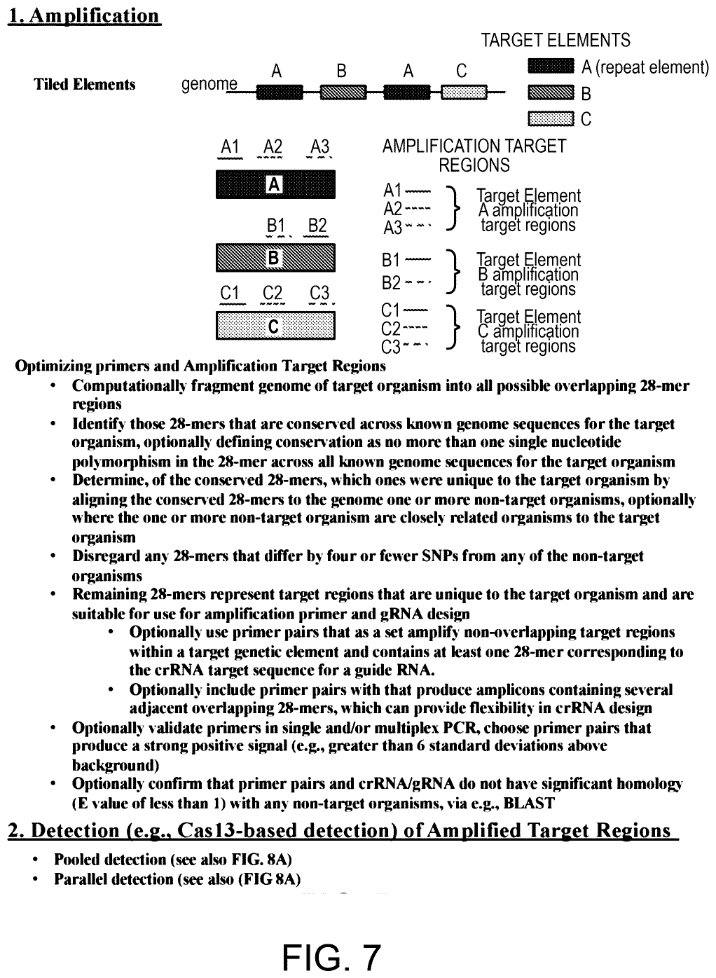

As shown in e.g., FIGS. 7 and 8A and in the Working Examples herein, the set of amplification primers and amplified target region/elements can be selected such that the assay can detect and discern between closely related microorganisms and viruses. In some embodiments the amplification target regions/target elements are optimized by computationally fragment genome of target organism into all possible overlapping 28-mer regions, identifying those 28-mers that are conserved across known genome sequences for the target organism, optionally defining conservation as no more than one single nucleotide polymorphism in the 28-mer across all known genome sequences for the target organism; determining, of the conserved 28-mers, which ones were unique to the target organism by aligning the conserved 28-mers to the genome one or more non-target organisms, optionally where the one or more non-target organism are closely related organisms to the target organism, and disregarding any 28-mers that differ by four or fewer SNPs from any of the non-target organisms (as they are not considered unique to the target organism). The remaining 28-mers represent target regions that are unique to the target organism and are suitable for use for amplification primer and crRNA/gRNA design. In some embodiments, primer pairs are chosen that, as a set, amplify non-overlapping target regions within a target genetic element and contains at least one 28-mer corresponding to the crRNA target sequence for a guide RNA. In some embodiments, primer pairs are included in the set of amplification primers that produce amplicons containing several adjacent overlapping 28-mers, which can provide flexibility in crRNA design. As mentioned elsewhere herein, target elements can be chosen based on number of times they are repeated in a genome (i.e., such elements are repeat elements). In some embodiments, one or more the target elements are each individually repeated 1-50 or more times in a genome. In some embodiments, selected primer pairs are further validated by singleplex and multiplex PCR. In some embodiments, the primers included in the amplification primer set can produce a strong positive signal when validated by singleplex and/or multiplex PCR, such as a greater than 6, 7, 8, 9 or 10 standard deviations above background. In some embodiments, primer pairs and/or crRNA sequences are confirmed to not have significant homology (E value of less than 1) with any non-target genome. This can be accomplished, e.g., by alignment/comparative genomic analysis (such as BLAST) against the known related or close genomes or across any suitable database such as NCBI and others that will be known and available to one of ordinary skill in the art.

### Amplification and Enrichment of Detection Targets and Signals

### CRISPR-Cas Detection

Embodiments of the set of guide polynucleotides includes a guide polynucleotide for each amplified target region. In some embodiments, the CRISPR-Cas detection can be pooled or run in parallel as described in association with FIG. 8A. CRISPR-Cas detection systems and methods are described in greater detail elsewhere herein.

### Enrichment of a CRISPR-Cas Target

In some embodiments, the amplified targets can be further enriched prior to detection with a CRISPR-Cas system. In certain example embodiments, a dead CRISPR effector protein may bind the amplified target nucleic acid in solution and then subsequently be isolated from said solution. For example, the dead CRISPR effector protein bound to the amplified target nucleic acid, may be isolated from the solution using an antibody or other molecule, such as an aptamer, that specifically binds the dead CRISPR effector protein.

In other example embodiments, the dead CRISPR effector protein may bound to a solid substrate. A fixed substrate may refer to any material that is appropriate for or can be modified to be appropriate for the attachment of a polypeptide or a polynucleotide. Possible substrates include, but are not limited to, glass and modified functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. In some embodiments, the solid support comprises a patterned surface suitable for immobilization of molecules in an ordered pattern. In certain embodiments a patterned surface refers to an arrangement of different regions in or on an exposed layer of a solid support. In some embodiments, the solid support comprises an array of wells or depressions in a surface. The composition and geometry of the solid support can vary with its use. In some embodiments, the solids support is a planar structure such as a slide, chip, microchip and/or array. As such, the surface of the substrate can be in the form of a planar layer. In some embodiments, the solid support comprises one or more surfaces of a flowcell. The term “flowcell” as used herein refers to a chamber comprising a solid surface across which one or more fluid reagent can be flowed. Example flowcells and related fluidic systems and detection platforms that can be readily used in the methods of the present disclosure are described, for example, in Bentley et al. Nature 456:53-59 (2008), WO 04/0918497, U.S. Pat. No. 7,057,026; WO 91/06678; WO 07/123744; U.S. Pat. Nos. 7,329,492; 7,211,414; 7,315,019; 7,405,281, and US 2008/0108082. In some embodiments, the solid support or its surface is non-planar, such as the inner or outer surface of a tube or vessel. In some embodiments, the solid support comprise microspheres or beads. “Microspheres,” “bead,” “particles,” are intended to mean within the context of a solid substrate to mean small discrete particles made of various material including, but not limited to, plastics, ceramics, glass, and polystyrene. In certain embodiments, the microspheres are magnetic microspheres or beads. Alternatively or additionally, the beads may be porous. The bead sizes range from nanometers, e.g., 100 nm, to millimeters, e.g., 1 mm.

The amplified target nucleic acids may then be exposed to the substrate to allow binding of the amplified target nucleic acids to the bound dead CRISPR effector protein. Non-target molecules may then be washed away. In certain example embodiments, the amplified target nucleic acids may then be released from the CRISPR effector protein/guide RNA complex for further detection using the methods disclosed herein. In certain example embodiments, the target nucleic acids may first be amplified as described herein.

In certain example embodiments, the CRISPR effector may be labeled with a binding tag. In certain example embodiments the CRISPR effector may be chemically tagged. For example, the CRISPR effector may be chemically biotinylated. In another example embodiment, a fusion may be created by adding additional sequence encoding a fusion to the CRISPR effector. One example of such a fusion is an AviTag™, which employs a highly targeted enzymatic conjugation of a single biotin on a unique 15 amino acid peptide tag. In certain embodiments, the CRISPR effector may be labeled with a capture tag such as, but not limited to, GST, Myc, hemagglutinin (HA), green fluorescent protein (GFP), flag, His tag, TAP tag, and Fc tag. The binding tag, whether a fusion, chemical tag, or capture tag, may be used to either pull down the CRISPR effector system once it has bound a target nucleic acid or to fix the CRISPR effector system on the solid substrate.

In certain example embodiments, the guide RNA may be labeled with a binding tag. In certain example embodiments, the entire guide RNA may be labeled using in vitro transcription (IVT) incorporating one or more biotinylated nucleotides, such as, biotinylated uracil. In some embodiments, biotin can be chemically or enzymatically added to the guide RNA, such as, the addition of one or more biotin groups to the 3′ end of the guide RNA. The binding tag may be used to pull down the guide RNA/target nucleic acid complex after binding has occurred, for example, by exposing the guide RNA/target nucleic acid to a streptavidin coated solid substrate.

Accordingly, in certain example embodiments, an engineered or non-naturally-occurring CRISPR effector may be used for enrichment purposes. In an embodiment, the modification may comprise mutation of one or more amino acid residues of the effector protein. The one or more mutations may be in one or more catalytically active domains of the effector protein. The effector protein may have reduced or abolished nuclease activity compared with an effector protein lacking said one or more mutations. The effector protein may not direct cleavage of the RNA strand at the target locus of interest. In a preferred embodiment, the one or more mutations may comprise two mutations. In a preferred embodiment, the one or more amino acid residues are modified in a C2c2 effector protein, e.g., an engineered or non-naturally-occurring effector protein or C2c2. In particular embodiments, the one or more modified of mutated amino acid residues are one or more of those in C2c2 corresponding to R597, H602, R1278 and H1283 (referenced to Lsh C2c2 amino acids), such as mutations R597A, H602A, R1278A and H1283A, or the corresponding amino acid residues in Lsh C2c2 orthologues.

In particular embodiments, the one or more modified of mutated amino acid residues are one or more of those in C2c2 corresponding to K2, K39, V40, E479, L514, V518, N524, G534, K535, E580, L597, V602, D630, F676, L709, I713, R717 (HEPN), N718, H722 (HEPN), E773, P823, V828, I879, Y880, F884, Y997, L1001, F1009, L1013, Y1093, L1099, L1111, Y1114, L1203, D1222, Y1244, L1250, L1253, K1261, I1334, L1355, L1359, R1362, Y1366, E1371, R1372, D1373, R1509 (HEPN), H1514 (HEPN), Y1543, D1544, K1546, K1548, V1551, I1558, according to C2c2 consensus numbering. In certain embodiments, the one or more modified of mutated amino acid residues are one or more of those in C2c2 corresponding to R717 and R1509. In certain embodiments, the one or more modified of mutated amino acid residues are one or more of those in C2c2 corresponding to K2, K39, K535, K1261, R1362, R1372, K1546 and K1548. In certain embodiments, said mutations result in a protein having an altered or modified activity. In certain embodiments, said mutations result in a protein having a reduced activity, such as reduced specificity. In certain embodiments, said mutations result in a protein having no catalytic activity (i.e., “dead” C2c2). In an embodiment, said amino acid residues correspond to Lsh C2c2 amino acid residues, or the corresponding amino acid residues of a C2c2 protein from a different species.

The above enrichment systems may also be used to deplete a sample of certain nucleic acids. For example, guide RNAs may be designed to bind non-target RNAs to remove the non-target RNAs from the sample. In one example embodiment, the guide RNAs may be designed to bind nucleic acids that do carry a particular nucleic acid variation. For example, in a given sample a higher copy number of non-variant nucleic acids may be expected. Accordingly, the embodiments disclosed herein may be used to remove the non-variant nucleic acids from a sample, to increase the efficiency with which the detection CRISPR effector system can detect the target variant sequences in a given sample.

Amplification and/or Enhancement of Detectable Signal of CRISPR-Cas Detection

In certain example embodiments, further modification may be introduced that further amplify the detectable positive signal. For example, activated CRISPR effector protein collateral activation may be used to generate a secondary target or additional guide sequence, or both. In one example embodiment, the reaction solution would contain a secondary target that is spiked in at high concentration. The secondary target may be distinct from the primary target (i.e., the target for which the assay is designed to detect) and in certain instances may be common across all reaction volumes. A secondary guide sequence for the secondary target may be protected, e.g., by a secondary structural feature such as a hairpin with a RNA loop, and unable to bind the second target or the CRISPR effector protein. Cleavage of the protecting group by an activated CRISPR effector protein (i.e., after activation by formation of complex with the primary target(s) in solution) and formation of a complex with free CRISPR effector protein in solution and activation from the spiked in secondary target. In certain other example embodiments, a similar concept is used with a second guide sequence to a secondary target sequence. The secondary target sequence may be protected a structural feature or protecting group on the secondary target. Cleavage of a protecting group off the secondary target then allows additional CRISPR effector protein/second guide sequence/secondary target complex to form. In yet another example embodiment, activation of CRISPR effector protein by the primary target(s) may be used to cleave a protected or circularized primer, which is then released to perform an isothermal amplification reaction, such as those disclosed herein, on a template that encodes a secondary guide sequence, secondary target sequence, or both. Subsequent transcription of this amplified template would produce more secondary guide sequence and/or secondary target sequence, followed by additional CRISPR effector protein collateral activation.

In some embodiments another CRISPR system can be used to enrich or amplify the detectable signal. In some embodiments the first CRISPR system(s) that is/are activated upon target binding can produce, such as via collateral activity, species that can activate (or be targets of) a second CRISPR system thus amplifying the signal for detection. In some embodiments a CRISPR type-III effector can be used as the signal amplifying system. In some embodiments, the type III effector is Csm6, which is which is activated by cyclic adenylate molecules or linear adenine homopolymers terminated with a 2′,3′-cyclic phosphate. In some embodiments, the first CRISPR system includes a Cas13 (e.g., Cas 13a, 13b, 13c, or 13d) and/or a Cas 12a effector(s) and the amplification system or molecule is or includes Csm6. See also Gootenberg et al. 2018. Science. 360:439-44 and WO 2019/051318, which are incorporated by reference herein as if expressed in their entireties.

### Sample Types

Appropriate samples for use in the methods disclosed herein include any conventional biological sample obtained from an organism or a part thereof, such as a plant, animal, bacteria, and the like. In particular embodiments, the biological sample is obtained from an animal subject, such as a human subject. A biological sample is any solid or fluid sample obtained from, excreted by or secreted by any living organism, including, without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as an infection with a pathogenic microorganism, such as a pathogenic bacteria or virus). For example, a biological sample can be a biological fluid obtained from, for example, blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface. In some embodiments, the sample contains or is believed to contain cfDNA and/or ccfDNA.

A sample can also be a sample obtained from any organ or tissue (including a biopsy or autopsy specimen, such as a tumor biopsy) or can include a cell (whether a primary cell or cultured cell) or medium conditioned by any cell, tissue or organ. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). In other examples, the sample includes circulating tumor cells (which can be identified by cell surface markers). In particular examples, samples are used directly (e.g., fresh or frozen), or can be manipulated prior to use, for example, by fixation (e.g., using formalin) and/or embedding in wax (such as formalin-fixed paraffin-embedded (FFPE) tissue samples). It will be appreciated that any method of obtaining tissue from a subject can be utilized, and that the selection of the method used will depend upon various factors such as the type of tissue, age of the subject, or procedures available to the practitioner. Standard techniques for acquisition of such samples are available in the art. See, for example Schluger et al., J. Exp. Med. 176:1327-33 (1992); Bigby et al., Am. Rev. Respir. Dis. 133:515-18 (1986); Kovacs et al., NFJM 318:589-93 (1988); and Ognibene et al., Am. Rev. Respir. Dis. 129:929-32 (1984).

The tissue sample can advantageously be sourced from any organism e.g., plant, animal, bacterial or fungal. Samples may be a tissue sample, which can optionally be cultured, dead or living tissue. The array of the invention allows the capture of any nucleic acid, e.g., mRNA molecules, which are present in cells that are capable of transcription and/or translation. The arrays and methods of the invention are particularly suitable for isolating and analyzing the transcriptome or genome of cells within a sample, wherein spatial resolution of the transcriptomes or genomes is desirable, e.g., where the cells are interconnected or in contact directly with adjacent cells. However, it will be apparent to a person of skill in the art that the methods of the invention may also be useful for the analysis of the transcriptome or genome of different cells or cell types within a sample even if said cells do not interact directly, e.g., a blood sample. In other words, the cells do not need to present in the context of a tissue and can be applied to the array as single cells (e.g., cells isolated from a non-fixed tissue). Such single cells, whilst not necessarily fixed to a certain position in a tissue, are nonetheless applied to a certain position on the array and can be individually identified. Thus, in the context of analyzing cells that do not interact directly, or are not present in a tissue context, the spatial properties of the described methods may be applied to obtaining or retrieving unique or independent transcriptome or genome information from individual cells. Additionally, the simultaneous sensing of proteome and transcriptome can be performed on different cells or cell types within a sample utilizing the methods described herein.

In some embodiments, the sample volume ranges from about 1 microliter to about 1 mL, or more. In some embodiments, the sample volume ranges from about 1-500 microliters, such as about 1, to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 microliters.

### Organisms of Interest

The nucleic acid detection systems can be configured through various combinations of amplification reagents (e.g., amplification primers and/or probes) and/or CRISPR-Cas detection reagents (e.g., guides) and be used to specifically detect the presence of an organism or organisms of interest in a sample and/or a subject from which the sample was obtained. In some embodiments, the organism is pathogenetic. In some embodiments, the organism is non-pathogenic (e.g., commensal bacteria, microbiome species and strains, etc.).

Depending on the particular application, “pathogens of interest” or “organisms of interest” may encompass all strains within a species, or include just a single strain. To accommodate varying levels of resolution, “in” and “out” groups are defined. The “in” group encompasses all genomes of interest. The “out” group comprises of all genomes that are not desired to detect as signal (theoretically all other genomes). Once the “in” and “out” groups are defined, a reference genome within the “in” group is chosen. This reference genome is used to generate a list of all possible genomic targets of a pre-defined size. Next, a sequence alignment tool, for example, Bowtie57, is used to identify matching sequences with all other genomes in the “in” and “out” groups. A candidate list of possible genomic targets comprises of those sequences that match with all genomes in the “in” group, and do not match with any of the genomes in the “out” group. Because there is some evidence that suggests that species-specific targets are likely to cover a large fraction of the genome, pooled CRISPR RNA (crRNA)guides for these targets can be computationally selected and can be empirically tested for efficiency using microbial genomic DNA (gDNA) samples that mimic the size profile of cfDNA and/or ccfDNA fragments.

In particular embodiments, the guide RNAs can be selected by one or more of sequence orthogonality, melting temperature and/or genomic distribution. In some embodiments, the guide RNAs are 28 nucleotides in length and contain one or no mismatch with the target nucleic acid, e.g., contain a mismatch tolerance of one nucleotide.

### Exemplary Organisms of Interest

The systems, devices, and methods described herein can be used to detect one or more organisms of interest, such as microbes. The term “microbe” as used herein includes bacteria, fungus, protozoa, parasites and viruses.

### Bacteria

The following provides an example list of the types of microbes that might be detected using the embodiments disclosed herein. In certain example embodiments, the microbe is a bacterium. Examples of bacteria that can be detected in accordance with the disclosed methods include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veroni biovar sobria (Aeromonas sobria), and Aeromonas caviae), Anaplasma phagocytophilum, Anaplasma marginale Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, B. fidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Microsporum canis, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Trichophyton rubrum, T. mentagrophytes, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others. In some embodiments, the bacteria detected are pathogenic. In some embodiments, the bacteria detected are non-pathogenic.

### Fungi

In certain example embodiments, the microbe is a fungus or a fungal species. Examples of fungi that can be detected in accordance with the disclosed methods include without limitation any one or more of (or any combination of), Aspergillus, Blastomyces, Candidiasis, Coccidiodomycosis, Cryptococcus neoformans, Cryptococcus gatti, sp. Histoplasma sp. (such as Histoplasma capsulatum), Pneumocystis sp. (such as Pneumocystis jirovecii), Stachybotrys (such as Stachybotrys chartarum), Mucroymcosis, Sporothrix, fungal eye infections ringworm, Eserohilum, Cladosporium.

In certain example embodiments, the fungus is a yeast. Examples of yeast that can be detected in accordance with disclosed methods include without limitation one or more of (or any combination of), Aspergillus species (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), a Geotrichum species, a Saccharomyces species, a Hansenula species, a Candida species (such as Candida albicans), a Kluyveromyces species, a Debaryomyces species, a Pichia species, or combination thereof. In certain example embodiments, the fungi is a mold. Example molds include, but are not limited to, a Penicillium species, a Cladosporium species, a Byssochlamys species, or a combination thereof.

### Protozoa

In certain example embodiments, the microbe is a protozoa. Examples of protozoa that can be detected in accordance with the disclosed methods and devices include without limitation any one or more of (or any combination of), Euglenozoa, Heterolobosea, Diplomonadida, Amoebozoa, Blastocystic, and Apicomplexa. Example Euglenoza include, but are not limited to, Trypanosoma cruzi (Chagas disease), T. brucei gambiense, T. brucei rhodesiense, Leishmania braziliensis, L. infantum, L. mexicana, L. major, L. tropica, and L. donovani. Example Heterolobosea include, but are not limited to, Naegleria fowleri. Example Diplomonadids include, but are not limited to, Giardia intestinalis (G. lamblia, G. duodenalis). Example Amoebozoa include, but are not limited to, Acanthamoeba castellanii, Balamuthia madrillaris, Entamoeba histolytica. Example Blastocysts include, but are not limited to, Blastocystic hominis. Example Apicomplexa include, but are not limited to, Babesia microti, Cryptosporidium parvum, Cyclospora cayetanensis, Plasmodium falciparum, P. vivax, P. ovale, P. malariae, and Toxoplasma gondii.

### Parasites

In some embodiments, the organism of interest is a parasite. Exemplary parasites include without limitation, Acanthamoeba spp., Balamuthia mandrillaris, Babesiosis spp. (e.g., Babesia B. divergens, B. bigemina, B. equi, B. microfti, B. duncani), Balantidiasis spp. (e.g., Balantidium coli), Blastocystis spp., Cryptosporidium spp., Cyclosporiasis spp. (e.g., Cyclospora cayetanensis), Dientamoebiasis spp. (e.g., Dientamoeba fragilis), Amoebiasis spp. (e.g., Entamoeba histolytica), Giardiasis spp. (e.g., Giardia lamblia), Isosporiasis spp. (e.g., Isospora belli), Leishmania spp., Naegleria spp. (e.g., Naegleria fowleri), Plasmodium spp. (e.g., Plasmodium falciparum, Plasmodium vivax, Plasmodium ovale curtisi, Plasmodium ovale wallikeri, Plasmodium malariae, Plasmodium knowlesi), Rhinosporidiosis spp. (e.g., Rhinosporidium seeberi), Sarcocystosis spp. (e.g., Sarcocystis bovihominis, Sarcocystis suihominis), Toxoplasma spp. (e.g., Toxoplasma gondii), Trichomonas spp. (e.g., Trichomonas vaginalis), Trypanosoma spp. (e.g., Trypanosoma brucei), Trypanosoma spp. (e.g., Trypanosoma cruzi), Tapeworm (e.g., Cestoda, Taenia multiceps, Taenia saginata, Taenia solium), Diphyllobothrium latum spp., Echinococcus spp. (e.g., Echinococcus granulosus, Echinococcus multilocularis, E. vogeli, E. oligarthrus), Hymenolepis spp. (e.g., Hymenolepis nana, Hymenolepis diminuta), Bertiella spp. (e.g., Bertiella mucronata, Bertiella studeri), Spirometra (e.g. Spirometra erinaceieuropaei), Clonorchis spp. (e.g., Clonorchis sinensis; Clonorchis viverrini), Dicrocoelium spp. (e.g., Dicrocoelium dendriticum), Fasciola spp. (e.g., Fasciola hepatica, Fasciola gigantica), Fasciolopsis spp. (e.g., Fasciolopsis buski), Metagonimus spp. (e.g., Metagonimus yokogawai), Metorchis spp. (e.g., Metorchis conjunctus), Opisthorchis spp. (e.g., Opisthorchis viverrini, Opisthorchis felineus), Clonorchis spp. (e.g., Clonorchis sinensis), Paragonimus spp. (e.g., Paragonimus westermani; Paragonimus africanus; Paragonimus caliensis; Paragonimus kellicotti; Paragonimus skrjabini; Paragonimus uterobilateralis), Schistosoma sp., Schistosoma spp. (e.g., Schistosoma mansoni, Schistosoma haematobium, Schistosoma japonicum, Schistosoma mekongi, and Schistosoma intercalatum), Echinostoma spp. (e.g., E. echinatum), Trichobilharzia spp. (e.g., Trichobilharzia regent), Ancylostoma spp. (e.g., Ancylostoma duodenale), Necator spp. (e.g., Necator americanus), Angiostrongylus spp., Anisakis spp., Ascaris spp. (e.g., Ascaris lumbricoides), Baylisascaris spp. (e.g., Baylisascaris procyonis), Brugia spp. (e.g. Brugia malayi, Brugia timori), Dioctophyme spp. (e.g., Dioctophyme renale), Dracunculus spp. (e.g., Dracunculus medinensis), Enterobius spp. (e.g., Enterobius vermicularis, Enterobius gregorii), Gnathostoma spp. (e.g., Gnathostoma spinigerum, Gnathostoma hispidum), Halicephalobus spp. (e.g., Halicephalobus gingivalis), Loa loa spp. (e.g., Loa loa filaria), Mansonella spp. (e.g., Mansonella streptocerca), Onchocerca spp. (e.g., Onchocerca volvulus), Strongyloides spp. (e.g., Strongyloides stercoralis), Thelazia spp. (e.g., Thelazia californiensis, Thelazia callipaeda), Toxocara spp. (e.g., Toxocara canis, Toxocara cati, Toxascaris leonine), Trichinella spp. (e.g., Trichinella spiralis, Trichinella britovi, Trichinella nelsoni, Trichinella nativa), Trichuris spp. (e.g., Trichuris trichiura, Trichuris vulpis), Wuchereria spp. (e.g., Wuchereria bancrofti), Dermatobia spp. (e.g., Dermatobia hominis), Tunga spp. (e.g., Tunga penetrans), Cochliomyia spp. (e.g., Cochliomyia hominivorax), Linguatula spp. (e.g., Linguatula serrata), Archiacanthocephala sp., Moniliformis sp. (e.g. Moniliformis moniliformis), Pediculus spp. (e.g., Pediculus humanus capitis, Pediculus humanus humanus), Pthirus spp. (e.g., Pthirus pubis), Arachnida spp. (e.g., Trombiculidae, Ixodidae, Argaside), Siphonaptera spp (e.g., Siphonaptera: Pulicinae), Cimicidae spp. (e.g., Cimex lectularius and Cimex hemipterus), Diptera spp., Demodex spp. (e.g., Demodex folliculorum/brevis/canis), Sarcoptes spp. (e.g., Sarcoptes scabiei), Dermanyssus spp. (e.g., Dermanyssus gallinae), Ornithonyssus spp. (e.g., Ornithonyssus sylviarum, Ornithonyssus bursa, Ornithonyssus bacoti), Laelaps spp. (e.g., Laelaps echidnina), Liponyssoides spp. (e.g., Liponyssoides sanguineus), and/or any combination thereof.

### Viruses

In certain example embodiments, the systems, devices, and methods, disclosed herein are directed to detecting viruses in a sample. The embodiments disclosed herein may be used to detect viral infection (e.g., of a subject or plant), or determination of a viral strain, including viral strains that differ by a single nucleotide polymorphism. The virus may be a DNA virus, an RNA virus, or a retrovirus. Non-limiting example of viruses useful with the present invention include, but are not limited to Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In certain example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human immunodeficiency virus 1/2, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In certain example embodiments, the virus may be a plant virus selected from the group comprising Tobacco mosaic virus (TMV), Tomato spotted wilt virus (TSWV), Cucumber mosaic virus (CMV), Potato virus Y (PVY), the RT virus Cauliflower mosaic virus (CaMV), Plum pox virus (PPV), Brome mosaic virus (BMV), Potato virus X (PVX), Citrus tristeza virus (CTV), Barley yellow dwarf virus (BYDV), Potato leafroll virus (PLRV), Tomato bushy stunt virus (TBSV), rice tungro spherical virus (RTSV), rice yellow mottle virus (RYMV), rice hoja blanca virus (RHBV), maize rayado fino virus (MRFV), maize dwarf mosaic virus (MDMV), sugarcane mosaic virus (SCMV), Sweet potato feathery mottle virus (SPFMV), sweet potato sunken vein closterovirus (SPSVV), Grapevine fanleaf virus (GFLV), Grapevine virus A (GVA), Grapevine virus B (GVB), Grapevine fleck virus (GFkV), Grapevine leafroll-associated virus-1, -2, and -3, (GLRaV-1, -2, and -3), Arabis mosaic virus (ArMV), or Rupestris stem pitting-associated virus (RSPaV). In a preferred embodiment, the target RNA molecule is part of said pathogen or transcribed from a DNA molecule of said pathogen. For example, the target sequence may be comprised in the genome of an RNA virus. It is further preferred that CRISPR effector protein hydrolyzes said target RNA molecule of said pathogen in said plant if said pathogen infects or has infected said plant. It is thus preferred that the CRISPR system is capable of cleaving the target RNA molecule from the plant pathogen both when the CRISPR system (or parts needed for its completion) is applied therapeutically, i.e., after infection has occurred or prophylactically, i.e., before infection has occurred.

In certain example embodiments, the virus may be a retrovirus. Example retroviruses that may be detected using the embodiments disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

In certain example embodiments, the virus is a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zozter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, among others. In some embodiments, a method of diagnosing a species-specific bacterial infection in a subject suspected of having a bacterial infection is described as obtaining a sample comprising bacterial ribosomal ribonucleic acid from the subject; contacting the sample with one or more of the probes described, and detecting hybridization between the bacterial ribosomal ribonucleic acid sequence present in the sample and the probe, wherein the detection of hybridization indicates that the subject is infected with Escherichia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabilis, Staphylococcus agalactiae, or Staphylococcus maltophilia or a combination thereof. The virus may also be a virus of the genus/family as described in Tables 8 and 9 of International Patent Publication WO2018/170340, incorporated herein by reference.

In certain embodiments, the virus is a drug resistant virus. By means of example, and without limitation, the virus may be a ribavirin resistant virus. Ribavirin is a very effective antiviral that hits a number of RNA viruses. Below are a few important viruses that have evolved ribavirin resistance. Foot and Mouth Disease Virus: doi:10.1128/JVI.03594-13. Polio virus: www.pnas.org/content/100/12/7289.full.pdf. Hepatitis C Virus: jvi.asm.org/content/79/4/2346.full. A number of other persistent RNA viruses, such as hepatitis and HIV, have evolved resistance to existing antiviral drugs. Hepatitis B Virus (lamivudine, tenofovir, entecavir): doi:10.1002/hep.22900. Hepatitis C Virus (Telaprevir, BILN2061, ITMN-191, SCH6, Boceprevir, AG-021541, ACH-806): doi:10.1002/hep.22549. HIV has many drug resistant mutations, see hivdb.stanford.edu/ for more information. Aside from drug resistance, there are a number of clinically relevant mutations that could be targeted with the CRISPR systems according to the invention as described herein. For instance, persistent versus acute infection in LCMV: doi:10.1073/pnas.1019304108; or increased infectivity of Ebola: doi:10.1016/j.cell.2016.10.014 and doi:10.1016/j.cell.2016.10.013.

In certain embodiments, the target sequences are diagnostic for monitoring drug resistance to treatment against malaria or other infectious diseases. Plasmodium, notably Plasmodia species affecting humans such as Plasmodium falciparum, Plasmodium vivax, Plasmodium ovale, Plasmodium malariae, and Plasmodium knowlesi are exemplary.

### Diseases of Interest

In some embodiments, the systems and methods described herein can be used to detect nucleic acids, such as cfDNA and/or ccfDNA from any cell or tissue that can be indicative of a disease (or normal) or disease state (or non-disease state). In some embodiments, the cell or tissue is normal and/or healthy tissue. In some embodiments, the cell or tissue is abnormal, pathologic, and/or diseased. In some embodiments, the cell or tissue is a cancer cell or tissue. The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The cancer may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumor including serous tumor, endometrioid tumor and mucinous cystadenocarcinoma, sex-cord-stromal tumor), prostate cancer, liver and bile duct carcinoma (e.g., hepatocellular carcinoma, cholangiocarcinoma, hemangioma), choriocarcinoma, seminoma, embryonal carcinoma, kidney cancer (e.g., renal cell carcinoma, clear cell carcinoma, Wilms tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, astrocytoma, medulloblastoma, craniopharyngioma, ependymoma, pinealoma, hemangioblastoma, acoustic neuroma, oligodendroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

Further embodiments are illustrated in the following Examples which are given for illustrative purposes only and are not intended to limit the scope of the invention.

### EXAMPLES

### Example 1—Detection of a Single Locus in the IS6110 Element by PCR and RPA (Single Plex)

It was first sought to apply SHERLOCK detection to detect a single sequence element, albeit on that is highly repetitive in the TB genome. After several iterations of primer and CRISPR guide (crRNA) design, Applicant used a working combination to detect TB DNA that Applicant had fragmented into 180 bp to simulate the size of cfDNA in a After several iterations of primer and CRISPR guide (crRNA) design, Applicant used a working combination to detect TB DNA that Applicant had fragmented into 180 bp to simulate the size of cfDNA in a patient sample. Using PCR amplification coupled with Cas13 detection, the sensitivity was one genome equivalent per reaction (limit of detection; LOD=1) using fluorescence detection. PCR is used here only to defining limits of detection and assay sensitivity. Other methods can be used in the assay here in place of PCR. Importantly, a similar sensitivity of LOD=1 was achieved using RPA, an isothermal amplification method that is more compatible with a final, field deployable format. See e.g., FIG. 1.

### Example 2—Detection of Multiple Loci in a Single Element by PCR (Multiplex)

It was next tested the increased sensitivity that could be achieved through detecting multiple loci across a single, albeit repetitive element. In principle, amplifying numerous loci serves to not only increase sensitivity by virtue of having multiple amplicons contributing to the downstream signal, but also by increasing the likelihood of detecting any of the possible loci when only a fraction of a genome is present in a sample, as is believed to be the case for cfDNA. To test this method, multiplexed PCR amplification was performed using a pool containing 10 sets of primers targeting different regions of the IS6110 element (16 copies per genome in H37Rv) and 8 sets targeting different regions of the IS1081 element (6 copies per genome in H37Rv). (A computational algorithm was developed that enables selection of PCR primers that are compatible in multiplexed amplification.) The output from this single PCR reaction was taken and multiplexed Cas13 detection in a single pool involving 18 corresponding, optimized crRNAs using a fluorescence read-out was performed.

It was found that this multiplexing increased sensitivity by 10-fold relative to the single-plex format, achieving LOD=10-1 genome equivalents per reaction. See e.g., FIG. 2.

### Example 3—Specificity of Multiplexed SHERLOCK Detection

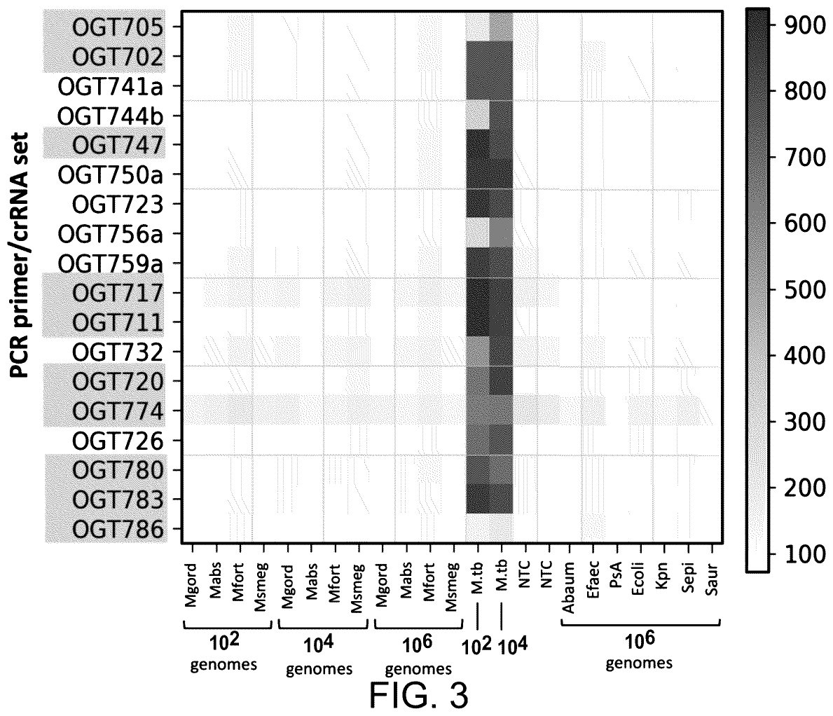

The computational pipeline to design primer and crRNA sets allows for unique recognition of a specific organism genome as opposed to the genomes of other organism, including related species. For example, in the context of detecting TB, the computational pipeline to design primer and crRNA sets allows for unique recognition of the Mtb genome and not the genomes of a number of other common bacterial pathogens, including mycobacterial species. Importantly, the desired specificity was confirmed experimentally when the assay was evaluated against other bacterial nucleic acids. See e.g., FIG. 3.

### Example 4—Assay Performance on Clinical Samples

To evaluate the clinical relevance of the analytical LODs, Applicant tested both the single-plex RPA-Cas3 and multiplexed PCR-Cas3 workflows on cfDNA that was extracted from clinical blood samples. The twelve cfDNA samples that were provided were obtained from individuals with suspected T—six were confirmed to be TB-positive by GeneXpert or culture and an internal qPCR experiment that targeted the IS6110 element (Samples PS1-PS6); and six were negative by GeneXpert, culture, and qPCR (Samples NG1-NG6). There were also 12 presumed negative samples from patients in Boston in which there was no reason to suspect TB (BWH1-BWH 12). See e.g., Table 8. As shown in Table 8, twelve samples were obtained from individuals with suspected TB. Samples PS1-PS6 were confirmed to be TB-positive by GeneXpert or culture and an internal qPCR experiment performed. The Ct values from the GPCR experiments are shown. Samples NG1-NG6 were negative by GeneXpert, culture, and qPCR. Twelve samples were obtained from patients in Boston and in which there was no reason to suspect TB.

TABLE 8
Cell-free DNA extracts from clinical blood samples as
evaluated in the SHERLOCK assay workflow

| Sample ID | GeneXpert | qPCR Ct value |
|---|---|---|
| PS1 | Positive | 36.1 |
| PS2 | Positive | 33.9 |
| PS3 | Positive | 36.1 |
| PS4 | Positive | 39.0 |
| PS5 | Positive | 35.8 |
| PS6 | Positive | 38.5 |
| NG1 | Negative | — |
| NG2 | Negative | — |
| NG3 | Negative | — |
| NG4 | Negative | — |
| NG5 | Negative | — |
| NG6 | Negative | — |

| | |
|---|---|
| BWH1-BWH12 | These 12 samples represent the presumed |
| | TB-negative cohort |

All cfDNA samples were extracted from 4 mL of blood from each individual using the Promega Maxwell RSC ccfDNA plasma kit and eluted in 100 uL buffer, of which 10 uL was used for qPCR. To benchmark the SHERLOCK assay results with the qPCR results, 10 uL of cfDNA extract as a starting concentration, as well as 1 uL and 100 nL were used to determine the limits of sensitivity. With a sample input of 10 μL of extracted cfDNA—which was extracted from the equivalent of 400 μL of whole blood—both multiplexed PCR-Cas13 and single-plex RPA-Cas13 workflows produced a detectable signal above that of the no template controls (NTC) using the fluorescent-based readout in all 6 confirmed-positive samples. See e.g., FIGS. 4A-4B. Interestingly, two of the 6 samples from symptomatic individuals but were negative by GeneXpert, culture and qPCR (i.e., NG5, NG6) produced a signal in the multiplexed PCR-Cas13 workflow; these were not tested in the RPA-Cas13 workflow due to limited volumes of the samples.

### Example 5—Performance in Point-of-Care, Lateral Flow Format

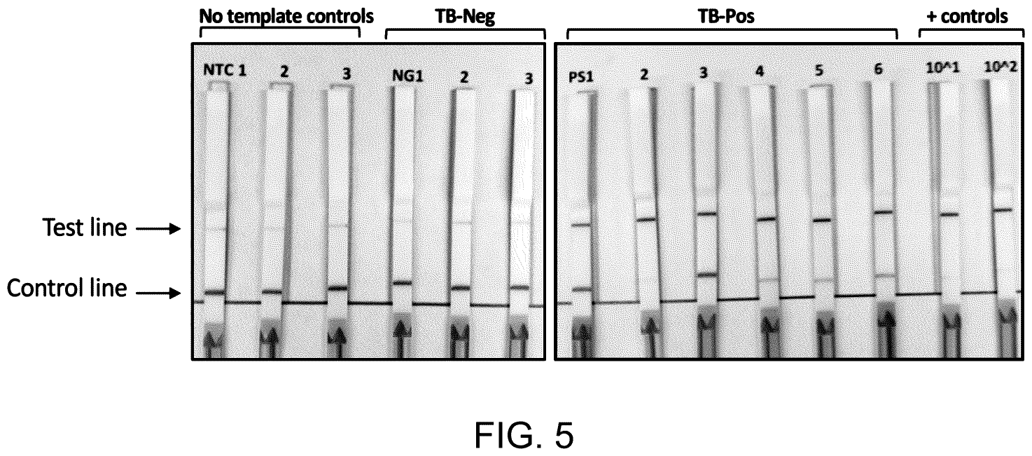

The performance of the workflow using an LFS as a test readout was compared to a microplate-based fluorescent readout. Using the single-plex RPA-Cas13 workflow on 10 μL of cfDNA extract, a 100% concordance between the two detection methods was observed. See e.g., FIG. 5. All six clinical samples that were confirmed by GeneXpert or culture and qPCR produced a positive result on the LFS; the three samples that were negative by GeneXpert, culture and qPCR produced a negative result on the LFS. Due to limited volumes of the clinical samples, NG4, 5, and 6 were not tested in this experiment).

### Example 6—Multiplexed RPA for Increased Sensitivity of Mtb Nucleic Acid Detection

As previously demonstrated, the single-plex RPA-Cas13 and PCR-Cas13 workflows currently achieve an analytical LOD of 1 genome equivalents per reaction and enables accurate detection in an LFS format of nucleic acids extracted from 40 μL of patient blood. Meanwhile, using a multiplexed PCR-Cas workflow, a 10-fold increased sensitivity with the ability to detect a positive signal from 4 μL of blood can be achieved. In order to allow the RPA-Cas13 method to achieve this same level of enhanced sensitivity, multiplexed amplification using RPA in a single repetitive element can be performed as was demonstrated with the multiplexed PCR-Cas13 workflow; and then be tiled across more loci in the Mtb genome to further increase sensitivity.

### Example 7—Genome-Wide Tiled Detection of Circulating Mycobacterium tuberculosis Cell-Free DNA Using Cas13

Infectious diseases remain a tremendous burden on the global health system. In 2019, an estimated 7.7 million people died from an infection, accounting for 14% of all deaths globally that year¹. Respiratory and diarrheal infections, along with tuberculosis, HIV/AIDS and malaria, continue to take their toll amidst the constant threat of emerging pathogens, such as SARS-CoV-2, which has claimed over 5 million lives since its inception². In response to the COVID-19 pandemic, highly sensitive diagnostic tests have been rapidly developed by targeting the SARS-CoV-2 genome, leveraging the programmability of nucleic acid detection. However, unlike SARS-CoV-2, which can be reliably detected using a non-invasive nasopharyngeal swab, many infections can require more challenging, even potentially onerous invasive sample collection, and yet still may elude diagnosis altogether. The need for sensitive, specific, and simple diagnostics for infectious diseases has never been clearer. Recently, biological and technical advances have enabled the use of liquid biopsies, wherein biomarkers of disease are detected in readily-accessible bodily fluids, like blood and urine, to obtain critical diagnostic information for a range of human conditions.

Cell-free human DNA in plasma is proving to be an invaluable biomarker in prenatal screening, oncology, toxicology and transplant medicine, where its detection and characterization in the peripheral circulation provides critical information on processes occurring in deeper, harder to access tissues. Along these lines, circulating microbial DNA may also be an easily accessible biomarker that enables the diagnosis of infections deep within the body, bypassing the need for biopsies or other difficult or invasive sample collection techniques. This modality of sample collection and patho-gen biomarker detection—ideally performed at the point-of-care—could transform infectious disease management. Blood-based nucleic acid tests (NATs) are well established for detecting viral infections such as HIV³ and hepatitis C⁴. Historically, such tests have played little role in the diagnosis of non-viral infections such as bacteria, in large part because of the relative scarcity of pathogen nucleic acids in blood for other infection types. Recently, however, polymerase chain reaction (PCR) or next generation sequencing (NGS) of cell-free DNA (cfDNA) in patient blood has been applied to detect pathogen nucleic acids as a means to identify an infecting agents”.

cfDNA in humans is predominantly derived from human cells. It circulates in biological fluids (e.g., blood, urine) as a result of cellular apoptosis and necrosis^{8,9}, with variable abundance (mean of ˜10 pg/μL, range of 1-1000 pg/μL)¹⁰. It is also highly fragmented with the fragmentation size determined by histone organization in chromosomes (peak fragment size of ˜160 bp)¹⁰⁻¹². A much smaller fraction of cfDNA is microbial in origin (<1%), and its fragmentation size is much less well characterized but likely smaller than host nuclear cfDNA, since it lacks the same chromosomal organization^{13,14} Nevertheless, fragments of cfDNA originating from pathogens at various body sites have been detected in purified plasma, prompting explorations of detecting these fragments for diagnosis¹⁵.

To achieve sensitive detection of low abundance, highly fragmented nucleic acids as would be required for bacterial cfDNA-based diagnostics, Applicant took advantage of the attomolar sensitivity of the SHERLOCK assay (Specific High Sensitivity Enzymatic Reporter UnLOCKing), a recently reported method applying nucleic acid detection to diagnostics. SHERLOCK assay combines traditional amplification with CRISPR-Cas13 detection, wherein amplified DNA is transcribed into RNA, and recognized by a complementary guide RNA (crRNA) complexed with the Cas13 enzyme; this interaction triggers collateral, non-specific Cas13 ribonuclease activity that is leveraged to generate a detectable reporter signal^{16,17}. The amplification and detection steps provide two independent recognition steps to ensure high specificity. Its sensitivity and specificity, as well as its requirement of only a relatively short recognition sequence of 28 nucleotides (nt) for the second recognition and detection step¹⁸, thus make it ideal for this application.

SHERLOCK assay was recently adapted to a microfluidic platform (Dro-pArray) that enables the detection of comprehensive panels of viral pathogens (in a system called CARMEN; Combinatorial Arrayed Reactions for Multiplexed Evaluation of Nucleic acids) and bacterial pathogens (in a system called bCARMEN) in thousands of parallel nanodroplets, each containing the reagents for the unique detection of a single genetic locus of each pathogen19,20. However, given that the abundance of bacterial nucleic acids in blood cfDNA during infection may be well below one genome equivalent per reasonably collected volume of sample (50-5000 μL), the sensitivity of any given assay will be limited by the frequency with which the target sequence is present in a queried sample. Splitting the initial collected sample into numerous parallel sub-samples for individual amplification or detection of different targets would necessarily reduce overall assay sensitivity. Meanwhile, detection of a single target alone could also contribute to suboptimal sensitivity by failing to take advantage of the fact that detection of any unique part of the bacterial genome would be sufficient for diagnosis. Detection of multiple targets would also ensure against mutations in any single target that could cause the assay to fail.

This Example demonstrates embodiments of a method, which Applicant termed WATSON (Whole-genome Assay using Tiled Surveillance Of Nucleic acids) assay to maximize the sensitivity of SHERLOCK assay for detecting cfDNA. Applicant adapted SHERLOCK assay to perform pooled amplification followed by simultaneous detection of many target sequences tiled across the pathogen genome. (FIG. 8A) When a pathogen genome is present at concentrations less than one genome equivalent, some genomic loci may be present and others absent. By going after multiple genomic targets, we increase the odds of detecting at least one target in a sample, such that a pathogen can be deemed to be present as long as any one of those sequences is detected in a sample. This enables pathogen detection even far below the limit of a single genome equivalent per sample. The tiled detection step can be performed either by numerous parallel CRISPR-Cas13 detection reactions or by a single pooled detection reaction (FIG. 8A). Applicant applied the WATSON assay to detect cfDNA from Mycobacterium tuberculosis (Mtb) the causative agent of tuberculosis, a disease for which current diagnostic tests are highly dependent on the acquisition of pathogen-containing sputum from patients who are often unable to produce a high quality sample. As such, the WHO has prioritized the development of a rapid biomarker-based, non-sputum-based test to detect all forms of tuberculosis²¹. Applicant demonstrates that the WATSON assay not only has higher sensitivity than singleplex SHERLOCK assay (targeting a single locus) in engineered samples, but also importantly, that the tiling amplification and detection strategy can detect pathogen cfDNA in patients with active pulmonary tuberculosis. Finally, Applicant also shows the potential for translating the WATSON assay to a field deployable, lateral flow platform, given the real-world requirements for diagnostics against infectious diseases such as tuberculosis.

### Results

Computational Design of Tiled Assay for Detection of M. tuberculosis (Mtb) Genomic Sequences

Applicant began by defining all possible sequence targets in the Mtb genome that are conserved across sequenced isolates of Mtb but are absent from related pathogens and the human genome (FIG. 8B-8C). Because the SHERLOCK assay requires a 28 nucleotide target sequence, Applicant first computationally fragmented the Mtb strain H37Rv reference genome into all possible overlapping 28-mers, and then identified those 28-mers that were conserved across 267 whole, closed genome sequences of the Mycobacterium tuberculosis complex (MTBC) in the NCBI database, spanning its 7 human-adapted phylogenetic lineages²². Conservation was defined as no more than one single nucleotide polymorphism (SNP) in the 28-mer across all MTBC genomes. Applicant found that 77% of all possible 28-mers in the reference genome were conserved across all 267 genomes. To then determine which of these conserved 28-mers were unique to MTBC, Applicant aligned them to the genomes of 88 non-tuberculous mycobacterial (NTM) isolates covering over 20 species (Table 9), reasoning that a MTBC-specific cfDNA assay was most likely to cross react with a closely related species. Applicant also aligned the 28-mers to a reference human genome (GRCh38.p11 [https://www.ncbi.nlm.nih.gov/assembly/GCF_000001405.40]). Any 28-mers that differed by fewer than four SNPs from any NTM, human, or bacterial genome were considered as not unique to MTBC and were excluded. The resulting 28-mers (65%) were considered to be both conserved across and unique to MTBC; they collectively spanned 79% of the reference genome. This conserved and unique part of the reference genome was used for primer and crRNA design.

To maximize assay sensitivity for proof of principle, Applicant restricted our initial design to regions that fell within the repetitive insertion elements IS6110 (1-25 copies per genome”, 16 copies in H37Rv) and IS1081 (5-7 copies per genome²⁴, 6 copies in H37Rv). Targeting multicopy genetic elements further increases the likelihood that any particular target is present in a sample containing highly fragmented, lowly abundant cfDNA. Unlike other TB NATs that also target IS6110 and/or IS1081, WATSON assay achieves a level of coverage across both repeat elements that is significantly greater than these other assays by virtue of tiling (Applicant's18-plex assay covers 59% of the repeat elements, whereas other targeted assays cover only about 5%). Importantly, Applicant's computational workflow for primer and crRNA design is generalizable to tile across entire pathogen genomes, beyond targets like the IS6110 and IS1081 elements, which Applicant demonstrates in this example as proof of principle. To facilitate the pooling of primers in the first step amplification, primers were designed to minimize 3′-3′ interactions by ensuring that the 5 nucleotides at the 3′ terminal end of each primer did not complement any part of any of the other primers in the pool (see Methods). A T7 promoter sequence was appended on the 5′-end of one primer of each pair to allow the amplified product to be transcribed into RNA for CRISPR-Cas13 detection. Applicant identified 18 primer pairs that spanned 18 non-overlapping regions within these elements and contained at least one 28-mer corresponding to the crRNA target sequence; some amplicons contained several adjacent overlapping 28-mers, which provides flexibility in crRNA design. The amplicons ranged in size from 70-97 bp, with the space between the forward and reverse primers ranging from 28-48 bps, and collectively spanned 58% of the IS6110 and ®S1081 sequences.

Applicant then generated crRNA for the Cas13-based detection step corresponding to the 28-mers contained within each of the 18 amplicons. If an amplicon allowed for adjacent overlapping 28-mers, Applicant designed and tested up to three crRNA for each target amplicon. crRNA were tested with singleplex and 18-plex PCR amplified Mtb gDNA using the previously reported DropArray microfluidic platform^{20,25}. From among 29 crRNA tested, Applicant chose the best 18 crRNA corresponding to the 18 primer pairs to be included in a WATSON assay based on their ability to produce a strong positive signal (>6 standard deviations above background) when tested against Mtb genomic DNA both in singleplex and 18-plex pooled format (FIG. 13A-13B, FIG. 8D). Finally, Applicant performed a BLAST search of all 18 pairs of primers and crRNA against all prokaryotes in NCBI and confirmed that they did not have significant homology (E value <1) with any non-MTBC pathogens. Primer and crRNA sequences are listed in Table 10.

| | |
|---|---|
| NCBI | |
| Accession | |
| Number | Genome Header |

“″In Grou”″ Genomes of Mycobacterium tuberculosis complex

| | |
|---|---|
| CP013475.1 | Mycobacterium tuberculosis 1458, complete genome |
| CP009198.1 | Mycobacterium tuberculosis 1821ADB35 genome |
| CP009199.1 | Mycobacterium tuberculosis 1821ADB36 genome |
| CP009200.1 | Mycobacterium tuberculosis 1821ADB37 genome |
| CP009201.1 | Mycobacterium tuberculosis 1821ADB38 genome |
| CP009202.1 | Mycobacterium tuberculosis 1821ADB40 genome |
| CP009203.1 | Mycobacterium tuberculosis 1821ADB41 genome |
| CP009204.1 | Mycobacterium tuberculosis 1821ADB42 genome |
| CP009205.1 | Mycobacterium tuberculosis 1821ADB44 genome |
| CP009206.1 | Mycobacterium tuberculosis 1821ADB45 genome |
| CP007299.1 | Mycobacterium tuberculosis 18b genome |
| CP010339.1 | Mycobacterium tuberculosis 22103, complete genome |
| CP010337.1 | Mycobacterium tuberculosis 22115, complete genome |
| HG813240.1 | Mycobacterium tuberculosis 49-02 complete genome |
| NC_020089.1 | Mycobacterium tuberculosis 7199-99 complete genome |
| CP009426.1 | Mycobacterium tuberculosis 96075, complete genome |
| CP009427.1 | Mycobacterium tuberculosis 96121, complete genome |
| CP017598.1 | Mycobacterium tuberculosis Beijing-like/1104 |
| | chromosome, complete genome |
| CP017593.1 | Mycobacterium tuberculosis Beijing-like/35049 |
| | chromosome, complete genome |
| CP017594.1 | Mycobacterium tuberculosis Beijing-like/36918 |
| | chromosome, complete genome |
| CP017595.1 | Mycobacterium tuberculosis Beijing-like/38774 |
| | chromosome, complete genome |
| CP017597.1 | Mycobacterium tuberculosis Beijing-like/50148 |
| | chromosome, complete genome |
| CP011510.1 | Mycobacterium tuberculosis Beijing, complete genome |
| CP017596.1 | Mycobacterium tuberculosis Beijing/391 chromosome, |
| | complete genome |
| NC_021054.1 | Mycobacterium tuberculosis Beijing/NITR203, complete genome |
| CP002883.1 | Mycobacterium tuberculosis BT1, complete genome |
| CP002882.1 | Mycobacterium tuberculosis BT2, complete genome |
| CM001226.1 | Mycobacterium tuberculosis BTB05-552 chromosome, |
| | whole genome shotgun sequence |
| CM001227.1 | Mycobacterium tuberculosis BTB05-559 chromosome, |
| | whole genome shotgun sequence |
| CP023170.1 | Mycobacterium tuberculosis C3 chromosome |
| NC_021251.1 | Mycobacterium tuberculosis CCDC5079, complete genome |
| NC_017522.1 | Mycobacterium tuberculosis CCDC5180, complete genome |
| CP002885.1 | Mycobacterium tuberculosis CCDC5180, complete genome |
| NC_002755.2 | Mycobacterium tuberculosis CDC1551, complete genome |
| CP023576.1 | Mycobacterium tuberculosis CSV10399 chromosome, |
| | complete genome |
| CP023577.1 | Mycobacterium tuberculosis CSV11678 chromosome, complete |
| | genome |
| CP023600.1 | Mycobacterium tuberculosis CSV3611 chromosome, complete genome |
| CP023599.1 | Mycobacterium tuberculosis CSV383 chromosome, complete genome |
| CP023573.1 | Mycobacterium tuberculosis CSV4519 chromosome, complete genome |
| CP023574.1 | Mycobacterium tuberculosis CSV4644 chromosome, complete genome |
| CP023575.1 | Mycobacterium tuberculosis CSV5769 chromosome, complete genome |
| CP023601.1 | Mycobacterium tuberculosis CSV9577 chromosome, complete genome |
| NC_017524.1 | Mycobacterium tuberculosis CTRI-2, complete genome |
| CP018778.1 | Mycobacterium tuberculosis DK9897, complete genome |
| NC_021740.1 | Mycobacterium tuberculosis EAI5, complete genome |
| NC_021194.1 | Mycobacterium tuberculosis EAI5/NITR206, complete genome |
| NC_020559.1 | Mycobacterium tuberculosis Erdman = ATCC 35801 |
| | DNA, complete genome |
| NC_009565.1 | Mycobacterium tuberculosis F11, complete genome |
| CP010330.1 | Mycobacterium tuberculosis F28, complete genome |
| CP016972.1 | Mycobacterium tuberculosis H37Ra chromosome, complete genome |
| NC_009525.1 | Mycobacterium tuberculosis H37Ra, complete genome |
| NC_000962.3 | Mycobacterium tuberculosis H37Rv, complete genome |
| NC_018143.2 | Mycobacterium tuberculosis H37Rv, complete genome |
| CP009480.1 | Mycobacterium tuberculosis H37Rv, complete genome |
| CM002882.1 | Mycobacterium tuberculosis H37RvAE chromosome, whole genome |
| | shotgun sequence |
| CM001515.1 | Mycobacterium tuberculosis H37RvCO chromosome, whole genome |
| | shotgun sequence |
| CM002883.1 | Mycobacterium tuberculosis H37RvLP chromosome, whole genome |
| | shotgun sequence |
| CM002884.1 | Mycobacterium tuberculosis H37RvMA chromosome, whole genome |
| | shotgun sequence |
| CP007027.1 | Mycobacterium tuberculosis H37RvSiena, complete genome |
| NC_022350.1 | Mycobacterium tuberculosis Haarlem, complete genome |
| CP002871.1 | Mycobacterium tuberculosis HKBS1, complete genome |
| AP018033.1 | Mycobacterium tuberculosis HN-024 |
| AP018034.1 | Mycobacterium tuberculosis HN-205 |
| AP018035.1 | Mycobacterium tuberculosis HN-321 |
| AP018036.1 | Mycobacterium tuberculosis HN-506 |
| CM001043.1 | Mycobacterium tuberculosis HN878 chromosome, whole genome |
| | shotgun sequence |
| CP018300.1 | Mycobacterium tuberculosis I0002353-6, complete genome |
| CP018301.1 | Mycobacterium tuberculosis I0002801-4, complete genome |
| CP018302.1 | Mycobacterium tuberculosis I0004000-1, complete genome |
| CP018303.1 | Mycobacterium tuberculosis I0004241-1, complete genome |
| CP007803.1 | Mycobacterium tuberculosis K, complete genome |
| CP007809.1 | Mycobacterium tuberculosis KIT87190, complete genome |
| AP014573.1 | Mycobacterium tuberculosis Kurono DNA, complete genome |
| NC_012943.1 | Mycobacterium tuberculosis KZN 1435, complete genome |
| CM000787.2 | Mycobacterium tuberculosis KZN 4207 chromosome, whole genome |
| | shotgun sequence |
| NC_016768.1 | Mycobacterium tuberculosis KZN 4207, complete genome |
| NC_018078.1 | Mycobacterium tuberculosis KZN 605, complete genome |
| CM000789.2 | Mycobacterium tuberculosis KZN R506 chromosome, whole genome |
| | shotgun sequence |
| CM000788.2 | Mycobacterium tuberculosis KZN V2475 chromosome, whole genome |
| | shotgun sequence |
| CP023606.1 | Mycobacterium tuberculosis LE103 chromosome, complete genome |
| CP023602.1 | Mycobacterium tuberculosis LE13 chromosome, complete genome |
| CP023607.1 | Mycobacterium tuberculosis LE371 chromosome, complete genome |
| CP023608.1 | Mycobacterium tuberculosis LE410 chromosome, complete genome |
| CP023578.1 | Mycobacterium tuberculosis LE486 chromosome, complete genome |
| CP023579.1 | Mycobacterium tuberculosis LE492 chromosome, complete genome |
| CP023603.1 | Mycobacterium tuberculosis LE63 chromosome, complete genome |
| CP023604.1 | Mycobacterium tuberculosis LE76 chromosome, complete genome |
| CP023605.1 | Mycobacterium tuberculosis LE79 chromosome, complete genome |
| CP023619.1 | Mycobacterium tuberculosis LN1100 chromosome, complete genome |
| CP023580.1 | Mycobacterium tuberculosis LN180 chromosome, complete genome |
| CP023620.1 | Mycobacterium tuberculosis LN1856 chromosome, complete genome |
| CP023581.1 | Mycobacterium tuberculosis LN2358 chromosome, complete genome |
| CP023621.1 | Mycobacterium tuberculosis LN2900 chromosome, complete genome |
| CP023612.1 | Mycobacterium tuberculosis LN2978 chromosome, complete genome |
| CP023610.1 | Mycobacterium tuberculosis LN317 chromosome, complete genome |
| CP023613.1 | Mycobacterium tuberculosis LN3584 chromosome, complete genome |
| CP023614.1 | Mycobacterium tuberculosis LN3588 chromosome, complete genome |
| CP023615.1 | Mycobacterium tuberculosis LN3589 chromosome, complete genome |
| CP023616.1 | Mycobacterium tuberculosis LN3668 chromosome, complete genome |
| CP023617.1 | Mycobacterium tuberculosis LN3672 chromosome, complete genome |
| CP023618.1 | Mycobacterium tuberculosis LN3695 chromosome, complete genome |
| CP023582.1 | Mycobacterium tuberculosis LN3756 chromosome, complete genome |
| CP023609.1 | Mycobacterium tuberculosis LN55 chromosome, complete genome |
| CP023611.1 | Mycobacterium tuberculosis LN763 chromosome, complete genome |
| CP018304.1 | Mycobacterium tuberculosis M0002959-6, complete genome |
| CP018305.1 | Mycobacterium tuberculosis M0018684-2, complete genome |
| CP023585.1 | Mycobacterium tuberculosis MDRDM1098 chromosome, complete |
| | genome |
| CP023583.1 | Mycobacterium tuberculosis MDRDM260 chromosome, complete |
| | genome |
| CP023584.1 | Mycobacterium tuberculosis MDRDM627 chromosome, complete |
| | genome |
| CP023622.1 | Mycobacterium tuberculosis MDRDM827 chromosome, complete |
| | genome |
| CP023626.1 | Mycobacterium tuberculosis MDRMA1565 chromosome, complete |
| | genome |
| CP023627.1 | Mycobacterium tuberculosis MDRMA2019 chromosome, complete |
| | genome |
| CP023623.1 | Mycobacterium tuberculosis MDRMA203 chromosome, complete |
| | genome |
| CP023628.1 | Mycobacterium tuberculosis MDRMA2082 chromosome, complete |
| | genome |
| CP023629.1 | Mycobacterium tuberculosis MDRMA2260 chromosome, complete |
| | genome |
| CP023630.1 | Mycobacterium tuberculosis MDRMA2441 chromosome, complete |
| | genome |
| CP023586.1 | Mycobacterium tuberculosis MDRMA2491 chromosome, complete |
| | genome |
| CP023624.1 | Mycobacterium tuberculosis MDRMA 701 chromosome, complete |
| | genome |
| CP023625.1 | Mycobacterium tuberculosis MDRMA863 chromosome, complete |
| | genome |
| CP023587.1 | Mycobacterium tuberculosis ME1473 chromosome, complete genome |
| CP020381.2 | Mycobacterium tuberculosis MTB1, complete genome |
| CP022014.1 | Mycobacterium tuberculosis MTB2 chromosome, complete genome |
| AP017901.1 | Mycobacterium tuberculosis NCGM946K2 |
| CM002049.1 | Mycobacterium tuberculosis PanR0201 chromosome, whole genome |
| | shotgun sequence |
| CM002022.1 | Mycobacterium tuberculosis PanR0202 chromosome, whole genome |
| | shotgun sequence |
| CM002052.1 | Mycobacterium tuberculosis PanR0203 chromosome, whole genome |
| | shotgun sequence |
| CM002053.1 | Mycobacterium tuberculosis PanR0205 chromosome, whole genome |
| | shotgun sequence |
| CM002054.1 | Mycobacterium tuberculosis PanR0206 chromosome, whole genome |
| | shotgun sequence |
| CM002056.1 | Mycobacterium tuberculosis PanR0207 chromosome, whole genome |
| | shotgun sequence |
| CM002055.1 | Mycobacterium tuberculosis PanR0208 chromosome, whole genome |
| | shotgun sequence |
| CM002057.1 | Mycobacterium tuberculosis PanR0209 chromosome, whole genome |
| | shotgun sequence |
| CM002075.1 | Mycobacterium tuberculosis PanR0301 chromosome, whole genome |
| | shotgun sequence |
| CM002074.1 | Mycobacterium tuberculosis PanR0304 chromosome, whole genome |
| | shotgun sequence |
| CM002078.1 | Mycobacterium tuberculosis PanR0305 chromosome, whole genome |
| | shotgun sequence |
| CM002079.1 | Mycobacterium tuberculosis PanR0306 chromosome, whole genome |
| | shotgun sequence |
| CM002077.1 | Mycobacterium tuberculosis PanR0307 chromosome, whole genome |
| | shotgun sequence |
| CM002076.1 | Mycobacterium tuberculosis PanR0308 chromosome, whole genome |
| | shotgun sequence |
| CM002071.1 | Mycobacterium tuberculosis PanR0309 chromosome, whole genome |
| | shotgun sequence |
| CM002080.1 | Mycobacterium tuberculosis PanR0311 chromosome, whole genome |
| | shotgun sequence |
| CM002072.1 | Mycobacterium tuberculosis PanR0313 chromosome, whole genome |
| | shotgun sequence |
| CM002073.1 | Mycobacterium tuberculosis PanR0314 chromosome, whole genome |
| | shotgun sequence |
| CM002070.1 | Mycobacterium tuberculosis PanR0315 chromosome, whole genome |
| | shotgun sequence |
| CM002069.1 | Mycobacterium tuberculosis PanR0316 chromosome, whole genome |
| | shotgun sequence |
| CM002068.1 | Mycobacterium tuberculosis PanR0317 chromosome, whole genome |
| | shotgun sequence |
| CM002067.1 | Mycobacterium tuberculosis PanR0401 chromosome, whole genome |
| | shotgun sequence |
| CM002066.1 | Mycobacterium tuberculosis PanR0402 chromosome, whole genome |
| | shotgun sequence |
| CM002065.1 | Mycobacterium tuberculosis PanR0403 chromosome, whole genome |
| | shotgun sequence |
| CM002064.1 | Mycobacterium tuberculosis PanR0404 chromosome, whole genome |
| | shotgun sequence |
| CM002063.1 | Mycobacterium tuberculosis PanR0405 chromosome, whole genome |
| | shotgun sequence |
| CM002062.1 | Mycobacterium tuberculosis PanR0407 chromosome, whole genome |
| | shotgun sequence |
| CM002061.1 | Mycobacterium tuberculosis PanR0409 chromosome, whole genome |
| | shotgun sequence |
| CM002059.1 | Mycobacterium tuberculosis PanR0410 chromosome, whole genome |
| | shotgun sequence |
| CM002060.1 | Mycobacterium tuberculosis PanR0411 chromosome, whole genome |
| | shotgun sequence |
| CM002058.1 | Mycobacterium tuberculosis PanR0412 chromosome, whole genome |
| | shotgun sequence |
| CM002127.1 | Mycobacterium tuberculosis PanR0501 chromosome, whole genome |
| | shotgun sequence |
| CM002126.1 | Mycobacterium tuberculosis PanR0503 chromosome, whole genome |
| | shotgun sequence |
| CM002125.1 | Mycobacterium tuberculosis PanR0505 chromosome, whole genome |
| | shotgun sequence |
| CM002124.1 | Mycobacterium tuberculosis PanR0601 chromosome, whole genome |
| | shotgun sequence |
| CM002122.1 | Mycobacterium tuberculosis PanR0602 chromosome, whole genome |
| | shotgun sequence |
| CM002121.1 | Mycobacterium tuberculosis PanR0603 chromosome, whole genome |
| | shotgun sequence |
| CM002120.1 | Mycobacterium tuberculosis PanR0604 chromosome, whole genome |
| | shotgun sequence |
| CM002123.1 | Mycobacterium tuberculosis PanR0605 chromosome, whole genome |
| | shotgun sequence |
| CM002119.1 | Mycobacterium tuberculosis PanR0606 chromosome, whole genome |
| | shotgun sequence |
| CM002114.1 | Mycobacterium tuberculosis PanR0607 chromosome, whole genome |
| | shotgun sequence |
| CM002116.1 | Mycobacterium tuberculosis PanR0609 chromosome, whole genome |
| | shotgun sequence |
| CM002118.1 | Mycobacterium tuberculosis PanR0610 chromosome, whole genome |
| | shotgun sequence |
| CM002115.1 | Mycobacterium tuberculosis PanR0611 chromosome, whole genome |
| | shotgun sequence |
| CM002117.1 | Mycobacterium tuberculosis PanR0702 chromosome, whole genome |
| | shotgun sequence |
| CM002113.1 | Mycobacterium tuberculosis PanR0703 chromosome, whole genome |
| | shotgun sequence |
| CM002048.1 | Mycobacterium tuberculosis PanR0704 chromosome, whole genome |
| | shotgun sequence |
| CM002111.1 | Mycobacterium tuberculosis PanR0707 chromosome, whole genome |
| | shotgun sequence |
| CM002109.1 | Mycobacterium tuberculosis PanR0708 chromosome, whole genome |
| | shotgun sequence |
| CM002112.1 | Mycobacterium tuberculosis PanR0801 chromosome, whole genome |
| | shotgun sequence |
| CM002050.1 | Mycobacterium tuberculosis PanR0802 chromosome, whole genome |
| | shotgun sequence |
| CM002108.1 | Mycobacterium tuberculosis PanR0803 chromosome, whole genome |
| | shotgun sequence |
| CM002110.1 | Mycobacterium tuberculosis PanR0804 chromosome, whole genome |
| | shotgun sequence |
| CM002107.1 | Mycobacterium tuberculosis PanR0805 chromosome, whole genome |
| | shotgun sequence |
| CM002106.1 | Mycobacterium tuberculosis PanR0902 chromosome, whole genome |
| | shotgun sequence |
| CM002105.1 | Mycobacterium tuberculosis PanR0903 chromosome, whole genome |
| | shotgun sequence |
| CM002104.1 | Mycobacterium tuberculosis PanR0904 chromosome, whole genome |
| | shotgun sequence |
| CM002102.1 | Mycobacterium tuberculosis PanR0906 chromosome, whole genome |
| | shotgun sequence |
| CM002103.1 | Mycobacterium tuberculosis PanR0907 chromosome, whole genome |
| | shotgun sequence |
| CM002101.1 | Mycobacterium tuberculosis PanR0908 chromosome, whole genome |
| | shotgun sequence |
| CM002100.1 | Mycobacterium tuberculosis PanR0909 chromosome, whole genome |
| | shotgun sequence |
| CM002051.1 | Mycobacterium tuberculosis PanR1005 chromosome, whole genome |
| | shotgun sequence |
| CM002097.1 | Mycobacterium tuberculosis PanR1006 chromosome, whole genome |
| | shotgun sequence |
| CM002098.1 | Mycobacterium tuberculosis PanR1007 chromosome, whole genome |
| | shotgun sequence |
| CM002099.1 | Mycobacterium tuberculosis PanR1101 chromosome, whole genome |
| | shotgun sequence |
| CP010895.1 | Mycobacterium tuberculosis PR08 genome |
| CP010968.1 | Mycobacterium tuberculosis PR10 genome |
| CM001045.1 | Mycobacterium tuberculosis R1207 chromosome, whole genome |
| | shotgun sequence |
| CP023169.1 | Mycobacterium tuberculosis S3 chromosome |
| CM001225.1 | Mycobacterium tuberculosis S96-129 chromosome, whole genome |
| | shotgun sequence |
| CP012506.2 | Mycobacterium tuberculosis SCAID 187.0 chromosome, complete |
| | genome |
| CP016888.1 | Mycobacterium tuberculosis SCAID 252.0 chromosome, complete |
| | genome |
| CP016794.1 | Mycobacterium tuberculosis SCAID 320.0 chromosome, complete |
| | genome |
| CP023592.1 | Mycobacterium tuberculosis SLM036 chromosome, complete genome |
| CP023593.1 | Mycobacterium tuberculosis SLM040 chromosome, complete genome |
| CP023594.1 | Mycobacterium tuberculosis SLM056 chromosome, complete genome |
| CP023595.1 | Mycobacterium tuberculosis SLM060 chromosome, complete genome |
| CP023596.1 | Mycobacterium tuberculosis SLM063 chromosome, complete genome |
| CP023597.1 | Mycobacterium tuberculosis SLM088 chromosome, complete genome |
| CP023598.1 | Mycobacterium tuberculosis SLM100 chromosome, complete genome |
| CP017920.1 | Mycobacterium tuberculosis TB282 chromosome, complete genome |
| CP023631.1 | Mycobacterium tuberculosis TBDM1506 chromosome, complete |
| | genome |
| CP023632.1 | Mycobacterium tuberculosis TBDM2189 chromosome, complete |
| | genome |
| CP023633.1 | Mycobacterium tuberculosis TBDM2444 chromosome, complete |
| | genome |
| CP023634.1 | Mycobacterium tuberculosis TBDM2487 chromosome, complete |
| | genome |
| CP023635.1 | Mycobacterium tuberculosis TBDM2489 chromosome, complete |
| | genome |
| CP023636.1 | Mycobacterium tuberculosis TBDM2699 chromosome, complete |
| | genome |
| CP023637.1 | Mycobacterium tuberculosis TBDM2717 chromosome, complete |
| | genome |
| CP023588.1 | Mycobacterium tuberculosis TBDM425 chromosome, complete genome |
| CP023638.1 | Mycobacterium tuberculosis TBV4766 chromosome, complete genome |
| CP023639.1 | Mycobacterium tuberculosis TBV4768 chromosome, complete genome |
| CP023640.1 | Mycobacterium tuberculosis TBV4952 chromosome, complete genome |
| CP023589.1 | Mycobacterium tuberculosis TBV5000 chromosome, complete genome |
| CP023590.1 | Mycobacterium tuberculosis TBV5362 chromosome, complete genome |
| CP023591.1 | Mycobacterium tuberculosis TBV5365 chromosome, complete genome |
| CP009207.1 | Mycobacterium tuberculosis TRS1 genome |
| CP009195.1 | Mycobacterium tuberculosis TRS10 genome |
| CP009197.1 | Mycobacterium tuberculosis TRS11 genome |
| CP009182.1 | Mycobacterium tuberculosis TRS12 genome |
| CP009175.1 | Mycobacterium tuberculosis TRS13 genome |
| CP009181.1 | Mycobacterium tuberculosis TRS14 genome |
| CP009184.1 | Mycobacterium tuberculosis TRS15 genome |
| CP009172.1 | Mycobacterium tuberculosis TRS16 genome |
| CP009173.1 | Mycobacterium tuberculosis TRS17 genome |
| CP009185.1 | Mycobacterium tuberculosis TRS18 genome |
| CP009177.1 | Mycobacterium tuberculosis TRS19 genome |
| CP009186.1 | Mycobacterium tuberculosis TRS2 genome |
| CP009174.1 | Mycobacterium tuberculosis TRS20 genome |
| CP009176.1 | Mycobacterium tuberculosis TRS21 genome |
| CP009183.1 | Mycobacterium tuberculosis TRS22 genome |
| CP009180.1 | Mycobacterium tuberculosis TRS23 genome |
| CP009191.1 | Mycobacterium tuberculosis TRS24 genome |
| CP009192.1 | Mycobacterium tuberculosis TRS25 genome |
| CP009193.1 | Mycobacterium tuberculosis TRS26 genome |
| CP009190.1 | Mycobacterium tuberculosis TRS27 genome |
| CP009194.1 | Mycobacterium tuberculosis TRS28 genome |
| CP009187.1 | Mycobacterium tuberculosis TRS29 genome |
| CP009189.1 | Mycobacterium tuberculosis TRS4 genome |
| CP009179.1 | Mycobacterium tuberculosis TRS5 genome |
| CP009188.1 | Mycobacterium tuberculosis TRS6 genome |
| CP009178.1 | Mycobacterium tuberculosis TRS7 genome |
| CP009196.1 | Mycobacterium tuberculosis TRS8 genome |
| CP008702.1 | Mycobacterium tuberculosis TRS9 genome |
| NC_016934.1 | Mycobacterium tuberculosis UT205 complete genome |
| CP012090.1 | Mycobacterium tuberculosis W-148, complete genome |
| CM001044.1 | Mycobacterium tuberculosis X122 chromosome, whole genome |
| | shotgun sequence |
| CP009100.1 | Mycobacterium tuberculosis ZMC13-264, complete genome |
| CP009101.1 | Mycobacterium tuberculosis ZMC13-88, complete genome |
| NC_015758.1 | Mycobacterium africanum GM041182 complete genome |
| NC_002945.4 | Mycobacterium bovis AF2122/97 genome assembly, chromosome: |
| | Mycobacterium_bovis_AF2122/97 |
| NC_008769.1 | Mycobacterium bovis BCG Pasteur 1173P2, complete genome |
| CP003494.1 | Mycobacterium bovis BCG str. ATCC 35743, complete genome |
| NC_020245.2 | Mycobacterium bovis BCG str. Korea 1168P, complete genome |
| NC_016804.1 | Mycobacterium bovis BCG str. Mexico, complete genome |
| AM412059.1 | Mycobacterium bovis BCG str. Moreau RDJ complete genome |
| NC_012207.1 | Mycobacterium bovis BCG str. Tokyo 172 DNA, complete genome |
| CP014566.1 | Mycobacterium bovis BCG str. Tokyo 172 substrain TRCS, complete |
| | genome |
| CP008744.1 | Mycobacterium bovis BCG strain 3281, complete genome |
| CP009243.1 | Mycobacterium bovis BCG strain Russia 368, complete genome |
| CP012095.1 | Mycobacterium bovis strain 1595, complete genome |
| CP009449.1 | Mycobacterium bovis strain ATCC BAA-935, complete genome |
| CP013741.1 | Mycobacterium bovis strain BCG-1 (Russia), complete genome |
| CP015773.2 | Mycobacterium bovis strain SP38, complete genome |

“″Out Grou”″ Genomes of Non-tuberculous Mycobacteria

| | |
|---|---|
| CP009616.1 | Mycobacterium abscessus strain 4529, complete genome |
| CP009615.1 | Mycobacterium abscessus strain DJO-44274, complete genome |
| CP014950.1 | Mycobacterium abscessus strain FLAC003 chromosome, complete |
| | genome |
| CP014951.1 | Mycobacterium abscessus strain FLAC004 chromosome, complete |
| | genome |
| CP014952.1 | Mycobacterium abscessus strain FLAC005 chromosome, complete |
| | genome |
| CP016188.1 | Mycobacterium abscessus strain FLAC006 chromosome, complete |
| | genome |
| CP014953.1 | Mycobacterium abscessus strain FLAC007 chromosome, complete |
| | genome |
| CP014954.1 | Mycobacterium abscessus strain FLAC008 chromosome, complete |
| | genome |
| CP014955.1 | Mycobacterium abscessus strain FLAC013 chromosome, complete |
| | genome |
| CP016190.1 | Mycobacterium abscessus strain FLAC028 chromosome, complete |
| | genome |
| CP014956.1 | Mycobacterium abscessus strain FLAC029 chromosome, complete |
| | genome |
| CP016191.1 | Mycobacterium abscessus strain FLAC030 chromosome, complete |
| | genome |
| CP014957.1 | Mycobacterium abscessus strain FLAC031 chromosome, complete |
| | genome |
| CP014958.1 | Mycobacterium abscessus strain FLAC045 chromosome, complete |
| | genome |
| CP016192.1 | Mycobacterium abscessus strain FLAC046 chromosome, complete |
| | genome |
| CP014959.1 | Mycobacterium abscessus strain FLAC048 chromosome, complete |
| | genome |
| CP014960.1 | Mycobacterium abscessus strain FLAC049 chromosome, complete |
| | genome |
| CP014961.1 | Mycobacterium abscessus strain FLAC054 chromosome, complete |
| | genome |
| CP016193.1 | Mycobacterium abscessus strain FLAC055 chromosome, complete |
| | genome |
| CP013049.1 | Mycobacterium abscessus strain NOV0213, complete genome |
| CP009407.1 | Mycobacterium abscessus subsp. bolletii 103, complete genome |
| AP014547.1 | Mycobacterium abscessus subsp. bolletii CCUG 48898 = JCM 15300 |
| | DNA, complete genome |
| CP009408.1 | Mycobacterium abscessus subsp. bolletii strain MA 1948, complete |
| | genome |
| CP009613.1 | Mycobacterium abscessus subsp. bolletii strain MC1518, complete |
| | genome |
| CP009447.1 | Mycobacterium abscessus subsp. bolletii strain MM1513, complete |
| | genome |
| NC_018150.2 | Mycobacterium abscessus subsp. massiliense str. GO 06, complete |
| | genome |
| CP021122.1 | Mycobacterium abscessus subsp. massiliense strain FLAC047 |
| | chromosome, complete genome |
| CP012044.1 | Mycobacterium abscessus UC22, complete genome |
| LT549889.1 | Mycobacterium aurum isolate liquid genome assembly, chromosome: I |
| NC_008595.1 | Mycobacterium avium 104, complete genome |
| CP016396.1 | Mycobacterium avium strain RCAD0278, complete genome |
| CP009493.1 | Mycobacterium avium subsp. avium 2285 ®), complete genome |
| CP009482.1 | Mycobacterium avium subsp. avium 2285 (S), complete genome |
| CP009614.1 | avium subsp. avium strain DJO-44271, complete genome |
| CP018363.1 | Mycobacterium avium subsp. hominissuis strain H87 chromosome, |
| | complete genome |
| CP016818.1 | Mycobacterium avium subsp. hominissuis strain HP17 chromosome, |
| | complete genome |
| CP018020.1 | Mycobacterium avium subsp. hominissuis strain OCU873s_P7_4s |
| | chromosome, complete genome |
| CP018014.1 | Mycobacterium avium subsp. hominissuis strain OCU901s_S2_2s |
| | chromosome, complete genome |
| NC_021200.1 | Mycobacterium avium subsp. paratuberculosis MAP4, complete |
| | genome |
| NC_002944.2 | Mycobacterium avium subsp. paratuberculosis str. k10, complete |
| | genome |
| CP010113.1 | Mycobacterium avium subsp. paratuberculosis strain E1, complete |
| | genome |
| CP010114.1 | Mycobacterium avium subsp. paratuberculosis strain E93, complete |
| | genome |
| CP022095.1 | Mycobacterium avium subsp. paratuberculosis strain FDAARGOS_305 |
| | chromosome, complete genome |
| CP015495.1 | Mycobacterium avium subsp. paratuberculosis strain |
| | MAP/TANUVAS/TN/India/2008, complete genome |
| CP007220.1 | Mycobacterium chelonae CCUG 47445, complete genome |
| CP020821.1 | Mycobacterium colombiense CECT 3035, complete genome |
| CP011269.1 | Mycobacterium fortuitum strain CT6, complete genome |
| CP012150.1 | Mycobacter164ittorodii strain X7B, complete genome |
| CP011883.2 | Mycobacterium haemophilus DSM 44634 strain ATCC 29548, |
| | complete genome |
| CP011530.1 | Mycobacterium immunogenum strain CCUG 47286, complete genome |
| CP016189.1 | Mycobacterium immunogenum strain FLAC016 chromosome, complete |
| | genome |
| NC_018612.1 | Mycobacterium indicus pranii MTCC 9506, complete genome |
| CP009499.1 | Mycobacterium intracellulare 1956, complete genome |
| NC_016946.1 | Mycobacterium intracellulare ATCC 13950, complete genome |
| NC_016947.1 | Mycobacterium intracellulare MOTT-02, complete genome |
| NC_016948.1 | Mycobacterium intracellulare MOTT-64, complete genome |
| CP023146.1 | Mycobacterium intracellulare strain FLAC0133 chromosome, complete |
| | genome |
| CP009483.1 | Mycobacterium kansasii 824, complete genome |
| NC_011896.1 | Mycobacterium leprae Br4923, complete genome sequence |
| NC_002677.1 | Mycobacterium leprae TN chromosome, complete genome |
| CP019882.1 | Mycobacter164ittoralerale strain F4 chromosome, complete genome |
| NC_023036.2 | Mycobacterium neoaurum VKM Ac-1815D, complete genome |
| CP014475.1 | Mycobacterium phlei strain CCUG 21000, complete genome |
| NC_016604.1 | Mycobacterium rhodesiae NBB3, complete genome |
| AP018164.1 | Mycobacterium shigaense DNA, complete genome, strain: UN-152 |
| NC_015576.1 | Mycobacterium sinense strain JDM601, complete genome |
| LN831039.1 | Mycobacterium smegmatis genome assembly NCTC8159, |
| | chromosome: 1 |
| NC_008596.1 | Mycobacterium smegmatis str. MC2 155 chromosome, complete |
| | genome |
| NC_018289.1 | Mycobacterium smegmatis str. MC2 155, complete genome |
| CP009494.1 | Mycobacterium smegmatis str. MC2 155, complete genome |
| CP009495.1 | Mycobacterium smegmatis strain INHR1, complete genome |
| CP009496.1 | Mycobacterium smegmatis strain INHR2, complete genome |
| CP011773.1 | Mycobacterium sp. EPa45, complete genome |
| CP010271.1 | Mycobacterium sp. EPM10906, complete genome |
| NC_009077.1 | Mycobacterium sp. JLS, complete genome |
| NC_017904.1 | Mycobacterium sp. MOTT36Y, complete genome |
| CP011022.1 | Mycobacterium sp. NRRL B-3805, complete genome |
| CP023435.1 | Mycobacterium sp. PYR15 chromosome, complete genome |
| CP010071.1 | Mycobacterium sp. QIA-37, complete genome |
| CP009914.1 | Mycobacterium sp. VKM Ac-1817D, complete genome |
| CP018043.1 | Mycobacterium sp. WY10, complete genome |
| AP018165.1 | Mycobacterium stephanolepidis DNA, complete genome |
| LT906469.1 | Mycobacterium terrae strain NCTC10856 genome assembly, |
| | chromosome: 1 |
| LT906483.1 | Mycobacterium thermoresistibile strain NCTC10409 genome assembly, |
| | chromosome: 1 |
| CP011491.1 | Mycobacterium vaccae 95051, complete genome |
| NC_008726.1 | Mycobacterium vanbaalenii PYR-1, complete genome |
| CP015965.1 | Mycobacterium yongonense strain Asan 36527, complete genome |
| CP015964.1 | Mycobacterium yongonense strain Asan 36912, complete genome |

TABLE 10
Primer and guide sequences for the exemplary WATSON assay, singleplex
SHERLOCK assay, and singleplex RPA*

| | Forward Primer Nucleotide | Reverse | | |
|---|---|---|---|---|
| Primer | Sequence (Underlined | Primer | | |
| Pair | Nucleotides are | Nucleotide | Guide RNA template | |
| Name | T7 promoter) | Sequence | ssDNA Nucleotide Sequence | Description |
| IS1081-1 | GAAATTAATACGACTCACTAT | GGCGAGG | GCTCGACGAAGCCGTAGAGGCGTTTCGGGTT | WATSON |
| | AGGGCAAGTCGCAAGTGTCG | AAGGTAT | TTAGTCCCCTTCGTTTTTGGGGTAGTCTAAAT | Primer Pair |
| | ATCATGGCCAAAGA (SEQ ID | ACGGGCC | CCCCTATAGTGAGTCGTATTAATTTC (SEQ ID | (In 18x) |
| | NO: 2) | G (SEQ ID | NO: 4) | |
| | | NO: 3) | | |
| IS1081-2 | GAAATTAATACGACTCACTAT | GTCGGTG | AAACTCCCCGCGGTGGCCGAGCACCTCGGTT | WATSON |
| | AGGGATCAGTTGTTGCCCAAT | CGGGCGG | TTAGTCCCCTTCGTTTTTGGGGTAGTCTAAAT | Primer Pair |
| | ATGATCGGGTACT (SEQ ID | TGT (SEQ | CCCCTATAGTGAGTCGTATTAATTTC (SEQ ID | (In 18x) |
| | NO: 5) | ID NO: 6) | NO: 7) | |
| IS1081-3 | GAAATTAATACGACTCACTAT | CGAGAGC | TTCTGGCTGACCAACTCGCACAGGCGAGGTT | WATSON |
| | AGGGCTCTTCTCATCTTATCG | AGCCCGC | TTAGTCCCCTTCGTTTTTGGGGTAGTCTAAAT | Primer Pair |
| | ACGCCGAGCAGC (SEQ ID NO: | GCAGC | CCCCTATAGTGAGTCGTATTAATTTC (SEQ ID | (In 18x) |
| | 8) | (SEQ ID | NO: 10) | |
| | | NO: 9) | | |
| IS1081-4 | GAAATTAATACGACTCACTAT | TCGATGG | CGATGAGCGGTCCAATCAGCGCAACGGCGTT | WATSON |
| | AGGGCGGGCTACCGCGAACG | TTGCGGC | TTAGTCCCCTTCGTTTTTGGGGTAGTCTAAAT | Primer Pair |
| | CAGC (SEQ ID NO: 11) | ACGGGTG | CCCCTATAGTGAGTCGTATTAATTTC (SEQ ID | (In 18x) |
| | | T (SEQ ID | NO: 13) | |
| | | NO: 12) | | |
| IS1081-5 | GAAATTAATACGACTCACTAT | TCGGGCT | CACCCCGAAGCCCTCCTGGCCGTGGGTGGTT | WATSON |
| | AGGGAACCCACTACGCAGCC | GGTCGTA | TTAGTCCCCTTCGTTTTTGGGGTAGTCTAAAT | Primer Pair |
| | AATCTGATGGCAGC (SEQ ID | GATGGAG | CCCCTATAGTGAGTCGTATTAATTTC (SEQ ID | (In 18x) |
| | NO: 14) | TGCAGC | NO: 16) | |
| | | (SEQ ID | | |
| | | NO: 15) | | |
| IS1081-6 | GAAATTAATACGACTCACTAT | CAGTCTA | ACTGACCAGCACCGAAGAACCCGCCAAGGT | WATSON |
| | AGGGCTCACCCGAGCCCGAG | GGTGGTC | TTTAGTCCCCTTCGTTTTTGGGGTAGTCTAAA | Primer Pair |
| | CAGC (SEQ ID NO: 17) | AGTGCTG | TCCCCTATAGTGAGTCGTATTAATTTC (SEQ | (In 18x) |
| | | GGGTGT | ID NO: 19) | |
| | | (SEQ ID | | |
| | | NO: 18) | | |
| IS1081-7 | GAAATTAATACGACTCACTAT | GCGACCC | CTGGCTGGCGTTCTTCCGCGACCTGGTCGTTT | WATSON |
| | AGGGCCGCCGAGGACGGGGC | CGGACAG | TAGTCCCCTTCGTTTTTGGGGTAGTCTAAATC | Primer Pair |
| | CG (SEQ ID NO: 20) | GCCG | CCCTATAGTGAGTCGTATTAATTTC (SEQ ID | (In 18x) |
| | | (SEQ ID | NO: 22) | |
| | | NO: 21) | | |
| IS1081-8 | GAAATTAATACGACTCACTAT | CGCCGCA | GGTGCGCGAGGCAGGCCGCGTCGTCGGGGT | WATSON |
| | AGGGGCGCCAGGCGCAGGTC | AGCGCTT | TTTAGTCCCCTTCGTTTTTGGGGTAGTCTAAA | Primer Pair |
| | GATG (SEQ ID NO: 23) | CAGCTCA | TCCCCTATAGTGAGTCGTATTAATTTC (SEQ | (In 18x) |
| | | (SEQ ID | ID NO: 25) | |
| | | NO: 24) | | |
| IS6110-1 | GAAATTAATACGACTCACTAT | ATGAACC | CCGCGTCGGCTTTCTTCGCGGCCGAGCTGTT | WATSON |
| | AGGGAATTGCGAAGGGCGAA | GGGTAAT | TTAGTCCCCTTCGTTTTTGGGGTAGTCTAAAT | Primer Pair |
| | CGCGATTTTAAAGA (SEQ ID | TAGCGTG | CCCCTATAGTGAGTCGTATTAATTTC (SEQ ID | (In 18x) |
| | NO: 26) | CTGGCCG | NO: 28) | |
| | | (SEQ ID | | |
| | | NO: 27) | | |
| IS6110-2 | GAAATTAATACGACTCACTAT | CGCTCGC | GTTATCCACCATACGGATAGGGGATCTCGTT | WATSON |
| | AGGGCCAACAAGAAGGCGTA | TGAACCG | TTAGTCCCCTTCGTTTTTGGGGTAGTCTAAAT | Primer Pair |
| | CTCGACCTGAAAGA (SEQ ID | GATCGAT | CCCCTATAGTGAGTCGTATTAATTTC (SEQ ID | (in 18x |
| | NO: 29) | GTGTACT | NO: 31) | Singleplex |
| | | (SEQ ID | | PCR) |
| | | NO: 30) | | |
| IS6110-3 | GAAATTAATACGACTCACTAT | CGCACCG | TCAGTGAGGTCGCCCGTCTACTTGGTGTGTT | WATSON |
| | AGGGTCAGCACGATTCGGAG | TCTCCGC | TTAGTCCCCTTCGTTTTTGGGGTAGTCTAAAT | Primer Pair |
| | TGGGCAGC (SEQ ID NO: 32) | GCAGC | CCCCTATAGTGAGTCGTATTAATTTC (SEQ ID | (In 18x) |
| | | (SEQ ID | NO: 34) | |
| | | NO: 33) | | |
| IS6110-4 | GAAATTAATACGACTCACTAT | CCGGTTG | CACAGCTGACCGAGCTGGGTGTGCCGATGTT | WATSON |
| | AGGGCCGATGGTTTGCGGTG | ATGTGGT | TTAGTCCCCTTCGTTTTTGGGGTAGTCTAAAT | Primer Pair |
| | GGGTGT (SEQ ID NO: 35) | CGTAGTA | CCCCTATAGTGAGTCGTATTAATTTC (SEQ ID | (In 18x) |
| | | GGTCGAT | NO: 37) | |
| | | G (SEQ ID | | |
| | | NO: 36) | | |
| IS6110-5 | GAAATTAATACGACTCACTAT | GACGGTG | ACGGTGTTTACGGTGCCCGCAAAGTGTGGTT | WATSON |
| | AGGGTCCACGCCGCCAACTA | CATCTGG | TTAGTCCCCTTCGTTTTTGGGGTAGTCTAAAT | Primer Pair |
| | CGGTGT (SEQ ID NO: 38) | CCACCTC | CCCCTATAGTGAGTCGTATTAATTTC (SEQ ID | (In 18x) |
| | | GATG | NO: 40) | |
| | | (SEQ ID | | |
| | | NO: 39) | | |
| IS6110-6 | GAAATTAATACGACTCACTAT | CGCCGCA | GTCGATGCCGGCGCACGGCCCGGGACCAGTT | WATSON |
| | AGGGGCGCCAGGCGCAGGTC | AGCGCTT | TTAGTCCCCTTCGTTTTTGGGGTAGTCTAAAT | Primer Pair |
| | GATG (SEQ ID NO: 41) | CAGCTCA | CCCCTATAGTGAGTCGTATTAATTTC (SEQ ID | (In 18x) |
| | | (SEQ ID | NO: 43) | |
| | | NO: 42) | | |
| IS6110-7 | GAAATTAATACGACTCACTAT | TCTTGTT | CCACCTCCATGGTCCTCGACGCGATCGAGTT | WATSON |
| | AGGGTGGCGGGTCGCTTCCAC | GGCGGGT | TTAGTCCCCTTCGTTTTTGGGGTAGTCTAAAT | Primer Pair |
| | GATG (SEQ ID NO: 44) | CCAGATG | CCCCTATAGTGAGTCGTATTAATTTC (SEQ ID | (In 18x) |
| | | GCTT | NO: 46) | |
| | | (SEQ ID | | |
| | | NO: 45) | | |
| IS6110-8 | GAAATTAATACGACTCACTAT | TTGATCA | TATGACAATGCACTAGCCGAGACGATCAGTT | WATSON |
| | AGGGCGGTCGGAGCGGTCGG | GCTCGGT | TTAGTCCCCTTCGTTTTTGGGGTAGTCTAAAT | Primer Pair |
| | AAGCTC (SEQ ID NO: 47) | CTTGTAT | CCCCTATAGTGAGTCGTATTAATTTC (SEQ ID | (In 18x) |
| | | AGGCCG | NO: 49) | |
| | | (SEQ ID | | |
| | | NO: 48) | | |
| IS6110-9 | GAAATTAATACGACTCACTAT | AGCGATC | CTCGGCCTGTCCGGGACCACCCGCGGCAGTT | WATSON |
| | AGGGATGCACCGTCGAACGG | GTGGTCC | TTAGTCCCCTTCGTTTTTGGGGTAGTCTAAAT | Primer Pair |
| | CTGATGACCAA (SEQ ID NO: | TGCGGGC | CCCCTATAGTGAGTCGTATTAATTTC (SEQ ID | (In 18x) |
| | 50) | TT (SEQ ID | NO: 52) | |
| | | NO: 51) | | |
| IS6110-10 | GAAATTAATACGACTCACTAT | CATCCGC | TCATCGAGGAGGTACCCGCCGGAGCTGCGTT | WATSON |
| | AGGGTGGAAAGGATGGGGTC | ACCGCCC | TTAGTCCCCTTCGTTTTTGGGGTAGTCTAAAT | Primer Pair |
| | ATGTCAGGTGGT (SEQ ID NO: | GCTCA | CCCCTATAGTGAGTCGTATTAATTTC (SEQ ID | (In 18x) |
| | 53) | (SEQ ID | NO: 55) | |
| | | NO: 54) | | |
| RPA- | GAAATTAATACGACTCACTAT | GCTCGCT | GTTATCCACCATACGGATAGGGGATCTCGTT | RPA Singleplex |
| IS6110-2 | AGGGAACAAGAAGGCGTACT | GAACCGG | TTAGTCCCCTTCGTTTTTGGGGTAGTCTAAAT | Primer Pair |
| | CGACCTGAAAGA (SEQ ID NO: | ATCGATG | CCCCTATAGTGAGTCGTATTAATTTC (SEQ ID | |
| | 56) | TGTACT | NO: 58) | |
| | | (SEQ ID | | |
| | | NO: 57) | | |

*Underlined nucleotides represent the T7 promoter sequence necessary for Cas13 Detection

### Evaluation of WATSON Assay on Engineered Samples

Applicant first evaluated the performance of the WATSON assay using fragmented gDNA as input material. Purified gDNA from Mtb H37Rv was enzymatically fragmented to a median size of 180 bp^{5,26,27} (FIG. 14A-14B). Applicant created a dilution series of the fragmented Mtb gDNA amidst a constant background of 1 genome equivalent (GE) per μL of purified, fragmented human gDNA, to mimic what has been reported physiologically for human cfDNA^{10, 12} Applicant performed pooled 18-plex amplifi-cation followed by CRISPR/Cas13 detection in nanodroplets using the DropArray platform^{19,20}, either in parallel with each individual crRNA, or in a single pool of all 18 crRNAs together.

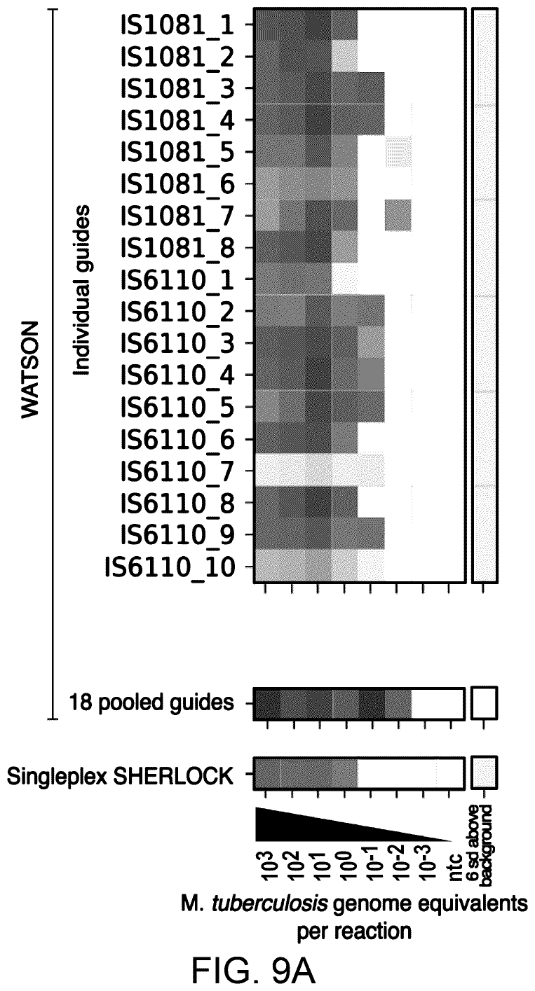

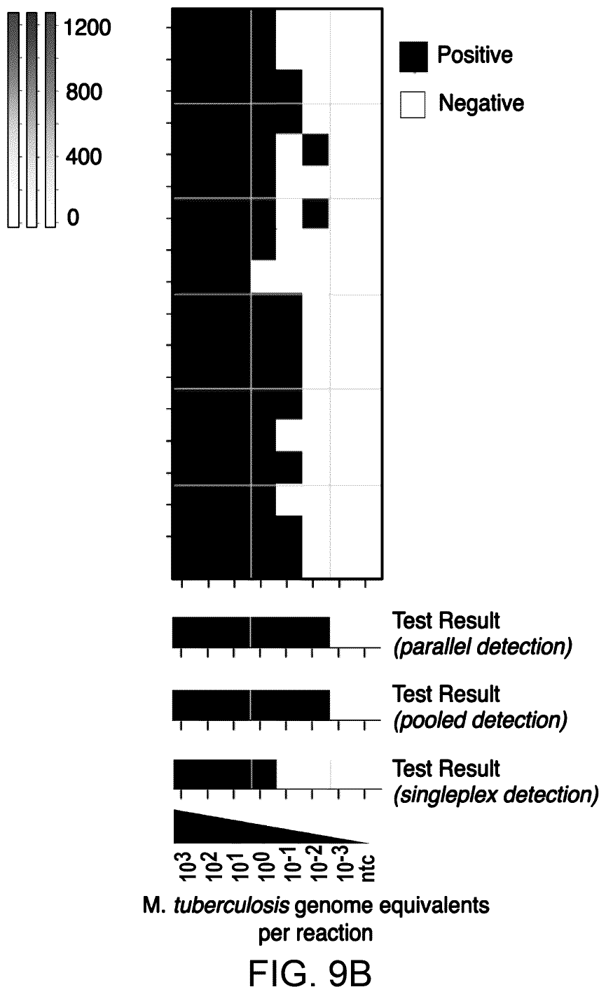

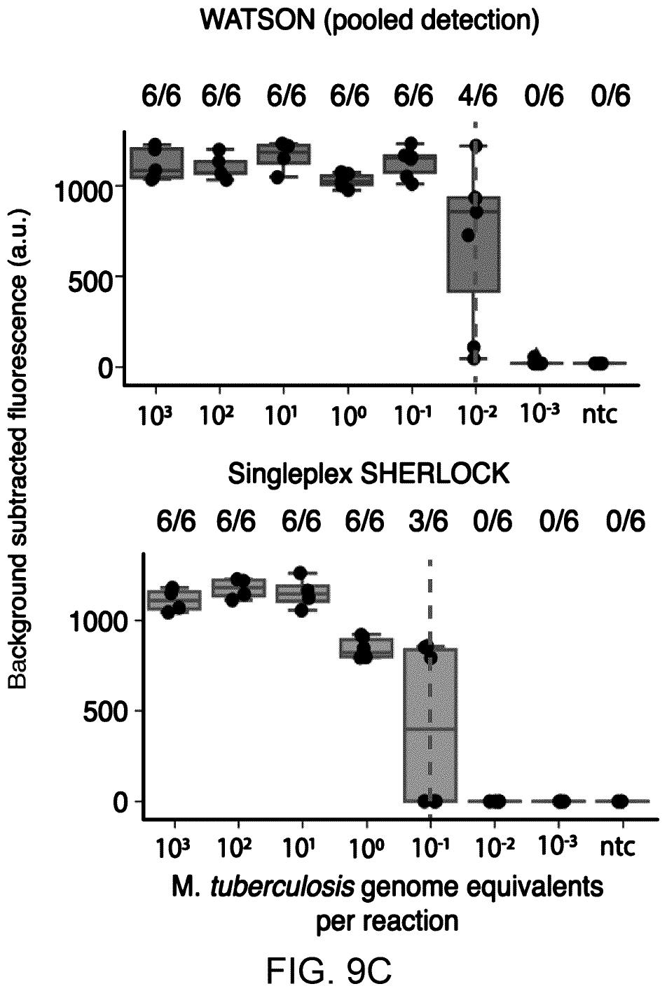

A heatmap for an exemplary dilution series shows fluorescent signals from each CRISPR/Cas13 detection reaction, generated by CRISPR/Cas13 collateral cleavage of a fluorescent reporter, for each individual crRNA tested in parallel and provides information on which of the individual tiled targets is present in the sample (FIG. 9A). The heatmap signals can be converted to a binary call of positivity based on a value >6 standard deviations above the average fluorescence of the no template control sample (FIG. 2b). A positive signal from any one of these 18 independent readouts is sufficient to yield a positive test result determination, reflecting the principle that the detection of any one target sequence is sufficient for a positive result (FIG. 9B). Similarly, the fluorescent signal from a single, pooled CRISPR/Cas13 detection reaction can be converted to a binary test result based on a similar threshold of >6 standard deviations above the no template control. The test results from parallel and pooled detection were the same, detecting Mtb down to an input of 0.01 GE per reaction (FIG. 9B). While parallel detection provides detailed information on the performance of individual guides, pooled detection makes the assay as technically simple as possible.

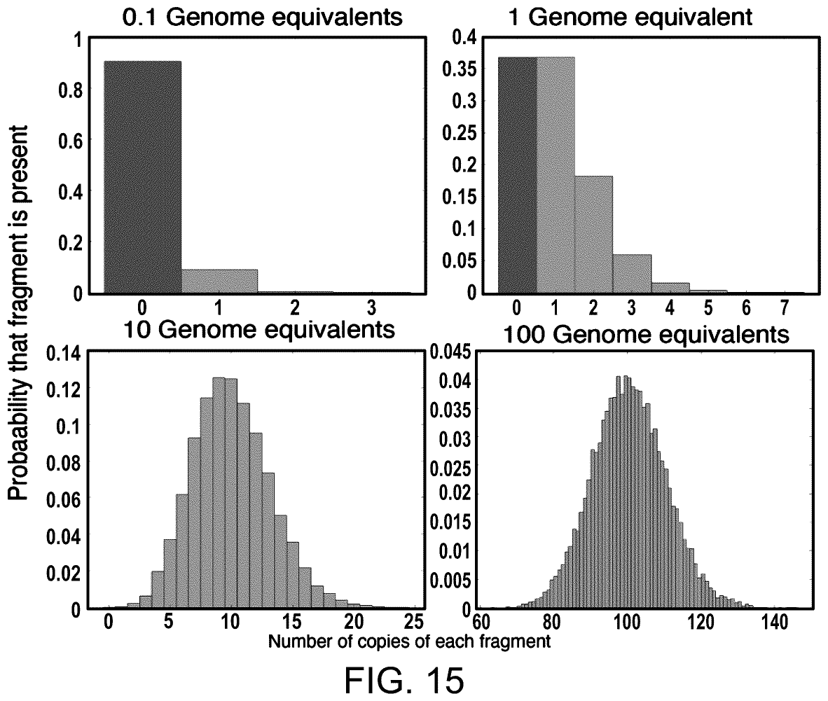

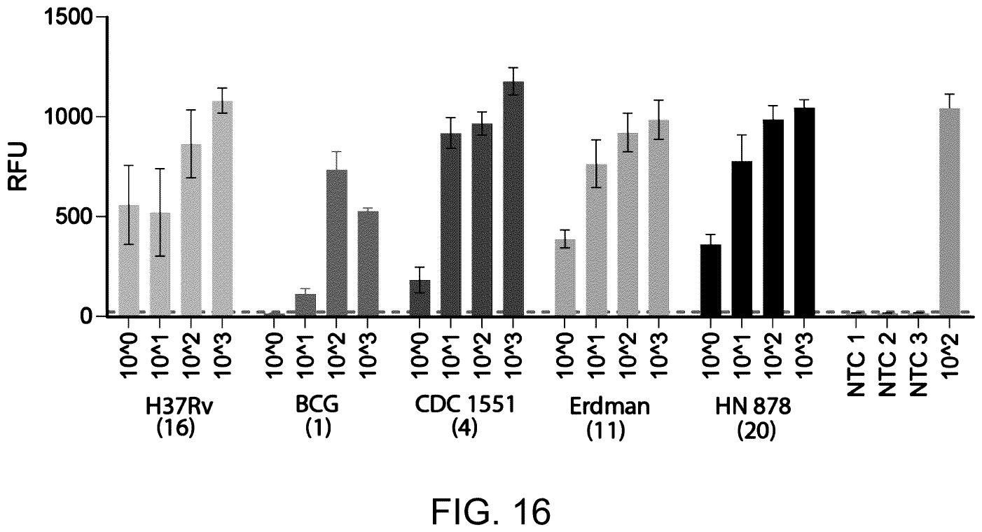

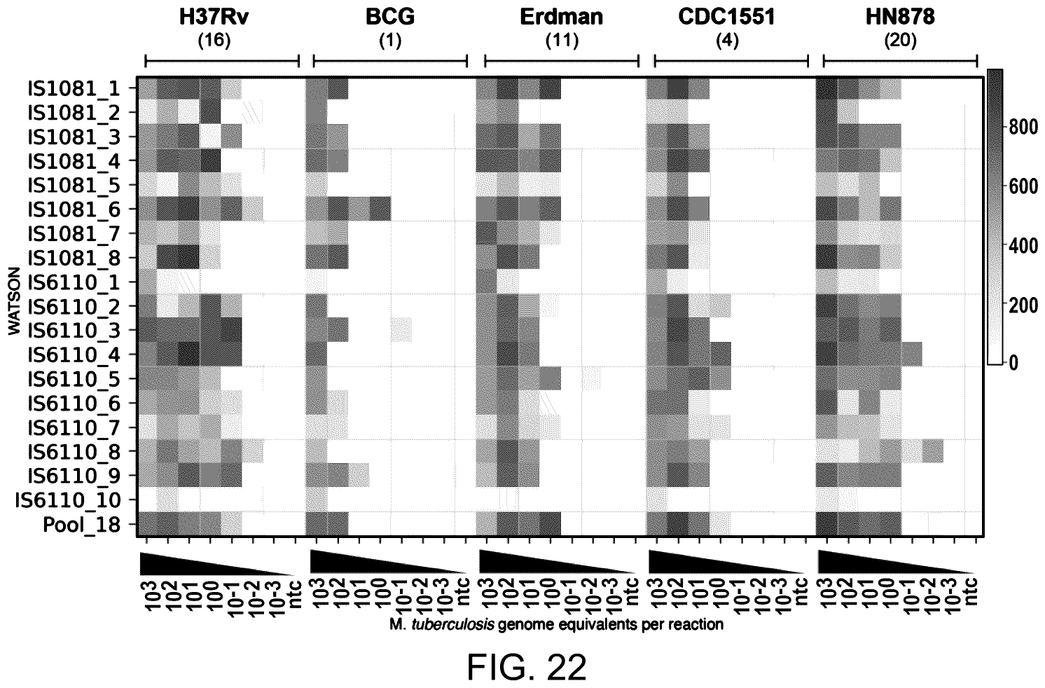

To compare the limit of detection (LoD) of the WATSON assay (pooled amplification and pooled detection) to singleplex SHERLOCK (single-plex amplification and detection) assay, Applicant performed six replicates of the dilution series with each assay. For singleplex SHERLOCK assay, the single best primer pair and crRNA from this set (IS6110_2) was used. Single-plex SHERLOCK assay had a LoD between 0.1 and 1 Mtb GE/reaction (0.5-5fg of fragmented DNA), while WATSON assay using pooled detection showed an improvement in LoD by 10- to 100-fold (0.01-0.1 GE/reaction, FIG. 9C). Variable signal across replicates at the LoD is consistent with a stochastic distribution of fragments in the samples at low copy numbers (FIG. 15). WATSON assay showed a comparable detection signal when testing DNA from Mtb strains across the phylogenetic tree, although LoD varied depending on the expected number of copies of IS6110 in the strain (FIG. 16, FIG. 22). WATSON assay did not produce a detectable signal when DNA from other NTMs and other clinically relevant bacteria were tested (FIG. 17A-17B).

### Evaluation of WATSON Assay on Clinical Samples

Having determined the sensitivity and specificity of WATSON assay in engineered samples of the reference strain, Applicant then sought to address WATSON assay's ability to detect Mtb nucleic acids in cfDNA of patients with tuberculosis, and compare tiling to detection of a single locus. Applicant compared the 18-plex version of a WATSON assay to singleplex SHERLOCK assay on clinical plasma samples obtained from patients with active pulmonary tuberculosis as confirmed by sputum-based culture and/or the Cep-heid GeneXpert qPCR diagnostic test.

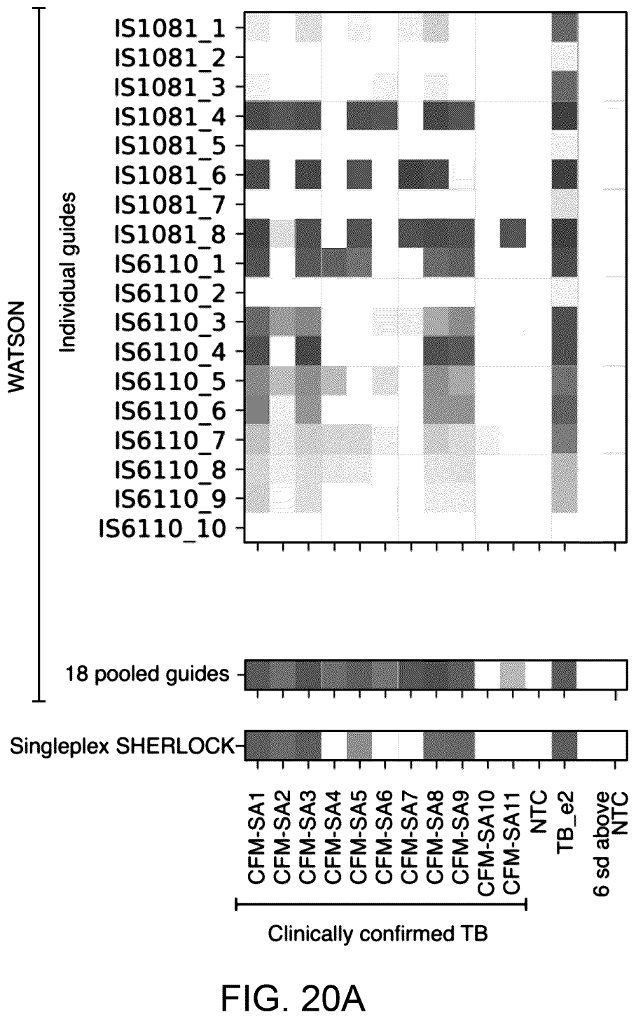

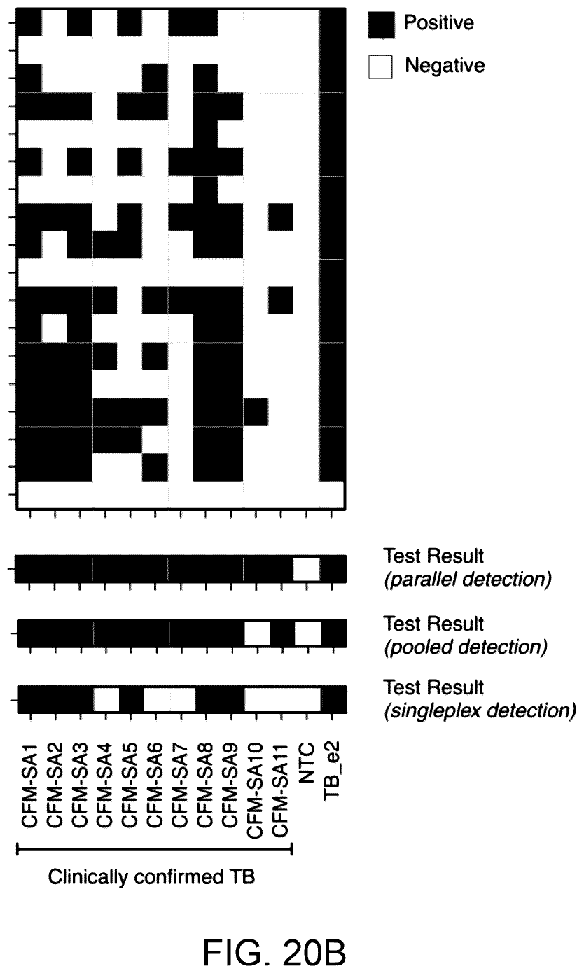

Applicant started with clinical samples from South Africa that were also positive for blood cfDNA as confirmed by a cfDNA-based qPCR assay that targets a single 72 bp region of IS611028. (FIG. 10) cfDNA was extracted from the equivalent of 400 μL of plasma from 11 patients. WATSON assay detected a positive signal in 10 of the 11 samples, positively identifying 91% on this small set of samples while singleplex SHERLOCK assay was positive in only 6 of the 11 samples (55%), highlighting the improved performance and thus value of tiling over single locus targeting. (FIG. 20A-20B) The single difference observed between singleplex SHERLOCK assay and the qPCR assay for some samples was likely due to differences in numbers of thermal cycles used for amplification for the various assays (see Methods).

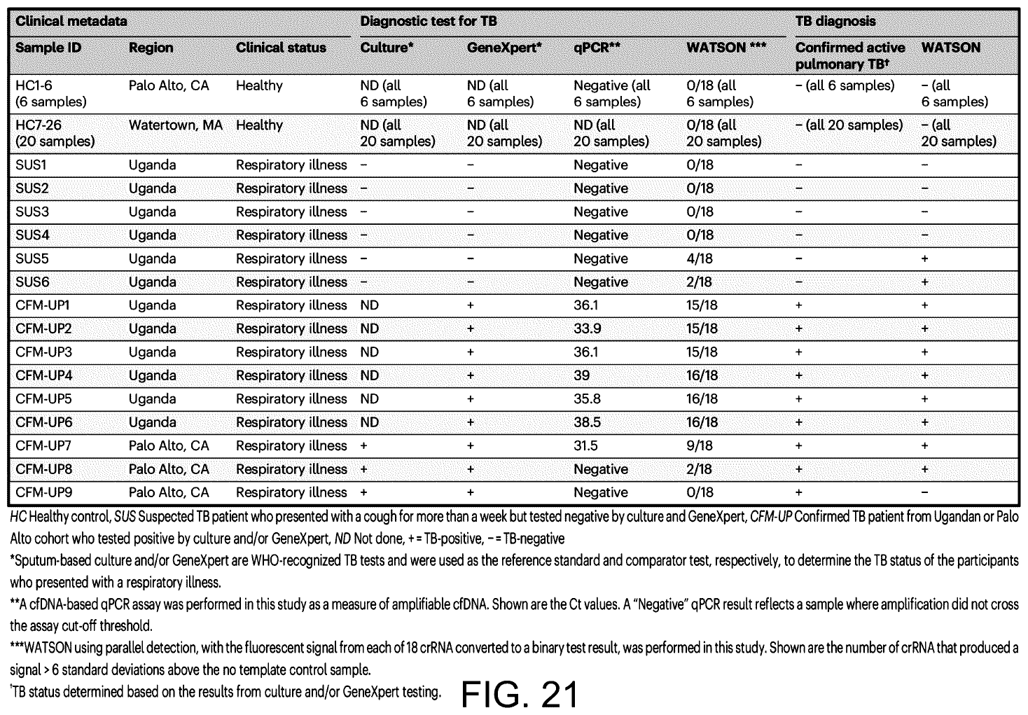

Applicant next tested WATSON assay on a broader set of clinical plasma samples from Uganda that included 9 patients with active pulmonary tuberculosis, as confirmed by sputum culture and/or GeneXpert but which had not been pre-screened by qPCR, 6 clinically suspected but sputum culture- and GeneXpert-negative cases, and 26 healthy controls. We thus also performed qPCR targeting the single region of IS6110 on the same cfDNA extracted from the samples to compare methods and evaluate the benefits of tiling28 (FIG. 21).

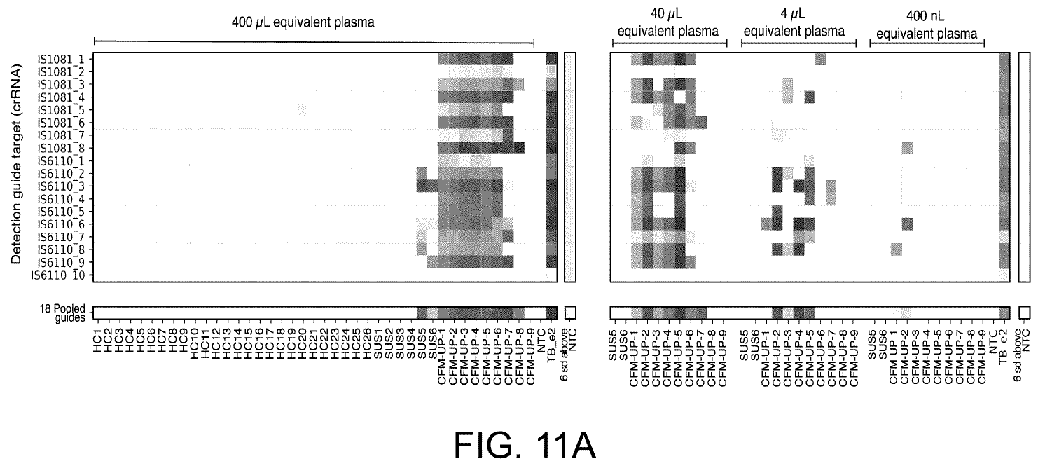

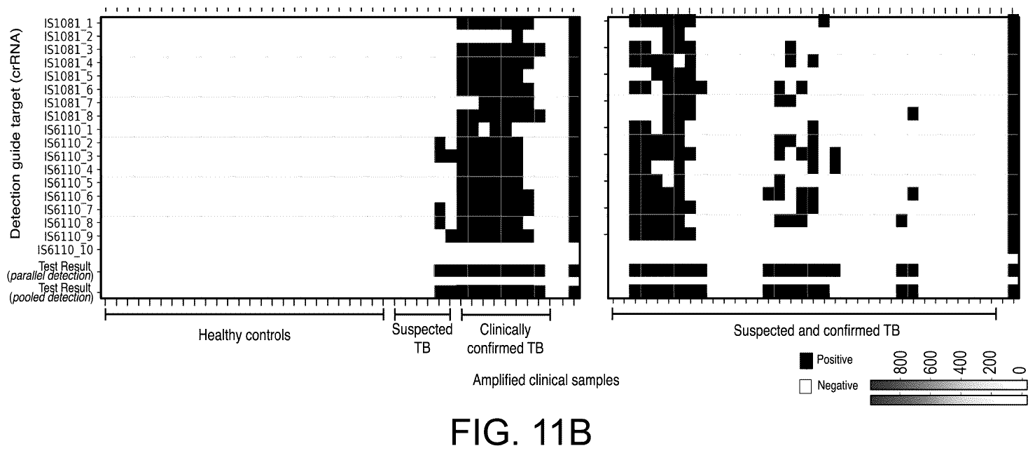

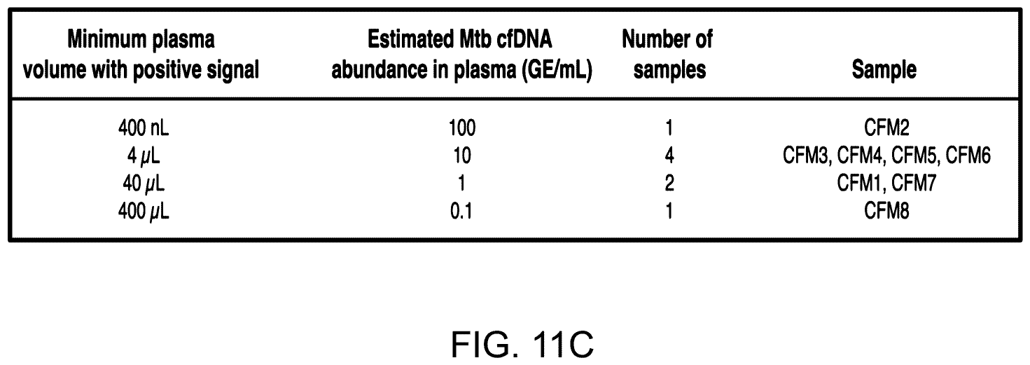

Applicant performed WATSON assay on cfDNA extracted from the equivalent of 400 μL of patient plasma and detected a positive signal in 8 of 9 cfDNA samples (89%) from confirmed active tuberculosis patients (CFM1-9) and in 0 of 26 healthy controls (HCl-26, FIG. 11A). In 6 of the 8 confirmed TB-positive samples (CFM1-6), nearly all (>14/18) crRNAs individually produced a signal. In one sample (CFM7), 9/18 crRNAs produced a signal. In the other positive sample (CFM8), only 2/18 crRNAs produced a positive signal, suggesting a very low abundance of Mtb cfDNA. Of note, 2 of the 9 WATSON-positive samples were qPCR-negative (CFM-UP8-9) highlighting WATSON assay's potential to detect Mtb cfDNA over the singleplex qPCR assay. Additionally, 400 μL samples from 6 sputum culture-, GeneXpert-, cfDNA qPCR-negative, but clinically suspected tuberculosis patients were tested (SUS1-6); all 6 were also negative by qPCR. However, interestingly, in 2 of the 6 samples from suspected tuberculosis cases that lacked laboratory confirmation by any other method, a positive signal was detected, albeit from a minority of targets (4/18 in SUS5 and 2/18 in SUS6). There was no clinical follow-up was available for the two patients from which these two samples were collected.

Applicant then retested all 9 samples from patients confirmed to have active pulmonary TB and the 2 samples from suspected TB patients that were WATSON assay-positive (CFM-UP1-9 and SUS5,6) using limiting amounts of sample input volume (cfDNA extracted from the equivalent of 40 μL, 4 μL and 400 nL of plasma). Unsurprisingly, the three positive samples in which only a minority of targets were detected from the equivalent of 400 μL (CFM8, SUS5 and SUS6) were negative when the input volume was decreased 10-fold (i.e., from 40 μL). In contrast, the remaining 7 positive samples were positive not only when 40 μL was used, but even when the equivalent of just 4 μL of plasma was used as the input, with two samples being positive even with an input equivalent to as little as 400 nL of plasma. Parallel detection and pooled detection were highly concordant, with 43/44 calls in agreement across the 4 input levels of these 11 samples, with a single discrepancy (CFM7 at 4 μL).

With a small input volume (the equivalent of 4 μL of plasma), WATSON assay detected a positive signal in 7 of the 9 confirmed positive samples (78%) (FIG. 11B, parallel detection). Importantly, this level of detection is the direct result of the increased opportunities for target detection provided by tiling, since no individual crRNA was detected in more than 44% of these samples. Additionally, and importantly, these results confirmed the presence of Mtb cfDNA fragment sizes that are compatible with WATSON assay's amplification and detection strategy. Intriguingly, the WATSON assay's detection of Mtb gDNA in two suspected tuberculosis cases that lacked laboratory confirmation evidence that the WATSON assay might be able to detect cases which current diagnostic approaches miss. Applicant sought to estimate the amount of Mtb cfDNA in the plasma samples of patients with active pulmonary tuberculosis using the WATSON assay. Since IS6110 copy number can vary widely across Mtb strains” (from 1 to 25), only signals produced by IS1081 were used for these estimates. Based on the data from the engineered samples, the WATSON assay's LoD using only IS1081-based amplicons is about 0.05 GEs/reaction. Using this LoD as a benchmark and the minimum sample volume producing a positive signal from a given patient plasma sample, Applicant back-calculated the estimated amount of Mtb cfDNA in the original sample. Efficiency of cfDNA extraction from plasma was assumed to be on the order of magnitude of 100%, based on previously reported data for the method used in this study²⁶. 1 of 9 positive samples (11%) had on the order of 10 GE/mL, 4 of 9 samples (44%) had 1 GE/mL, 2 of 9 samples (22%) had 0.1 GE/mL, 1 of 9 samples (11%) had 0.01 GE/mL, and 1 of 9 samples (11%) had <0.01 GE/mL. This suggests a very wide dynamic range (over 3 orders of magnitude) in Mtb cfDNA abundance in confirmed pulmonary tuberculosis patients (FIG. 11C).

### Point-of-Care Diagnostic Workflow

To evidence the WATSON assay's applicability as a point-of-care platform with progress towards addressing some of the most infrastructure-heavy aspects, Applicant demonstrated the ability to detect Mtb cfDNA using isothermal recombinase polymerization amplification (RPA) to replace PCR thermocycling, and a lateral flow strip to replace fluorescence Cas13 signal detection^{16,31}.

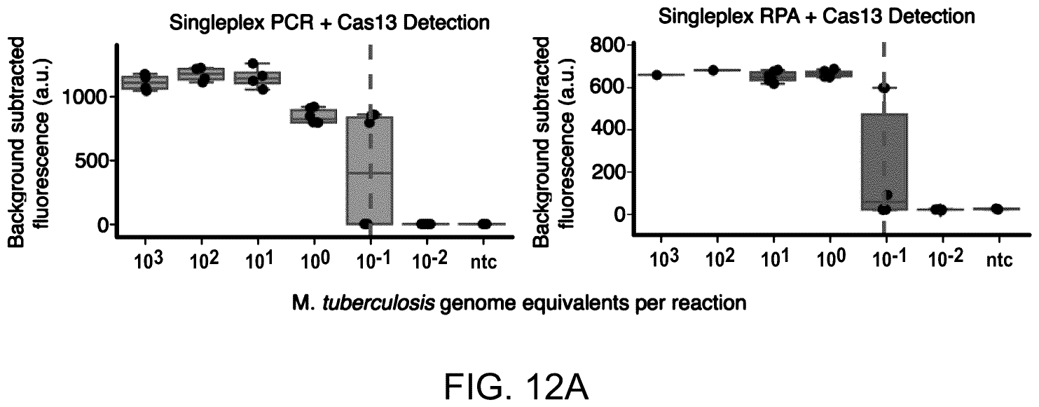

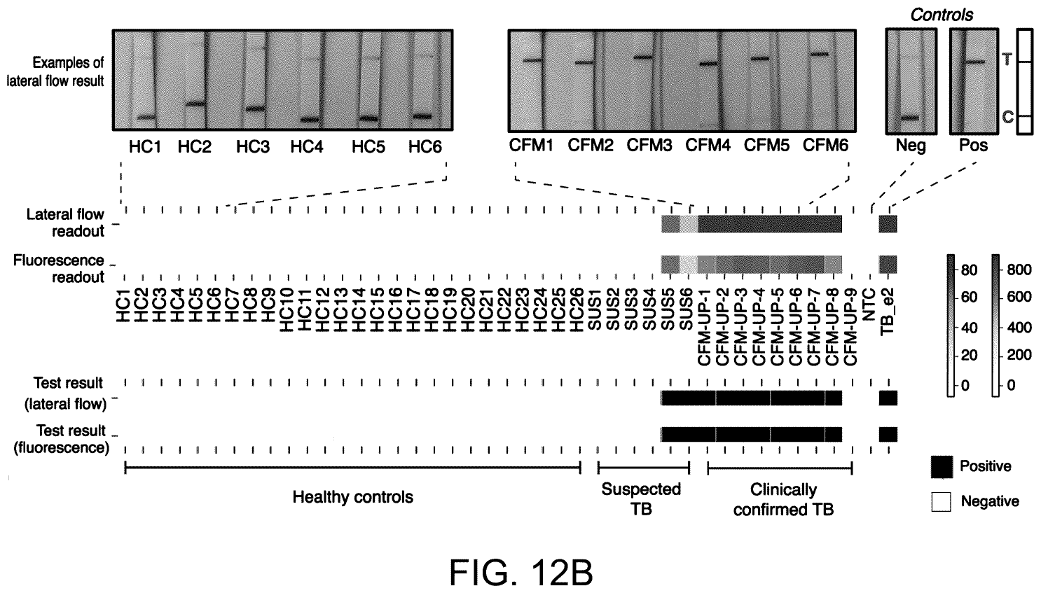

In the original description of the SHERLOCK assay, RPA was used as the amplification method, favored for its isothermal nature which dispenses with thermal cycling. Applicant screened over 20 RPA primer pairs and selected the primer/crRNA combination with the best LoD with engineered samples. Importantly, this pair, targeting a single 89-bp region of the multicopy IS6110 element, had a sensitivity comparable to that of singleplex PCR (Pearson's R=0.91), when coupled with CRISPR-Cas13 detection, suggesting that moving from PCR to RPA amplification will likely also ultimately have the needed sensitivity required for bacterial cfDNA detection. While RPA cannot currently be multiplexed as widely as PCR, it has several advantages for point-of-care deployment, most notably isothermal amplification at temperatures achievable without specialized equipment, thus motivating ongoing efforts to achieve highly multiplexed RPA (FIG. 12A).

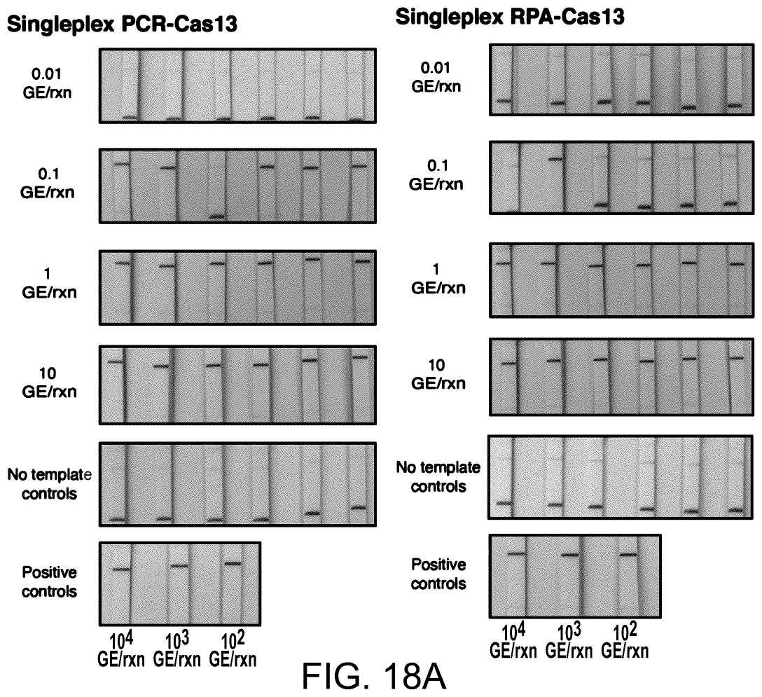

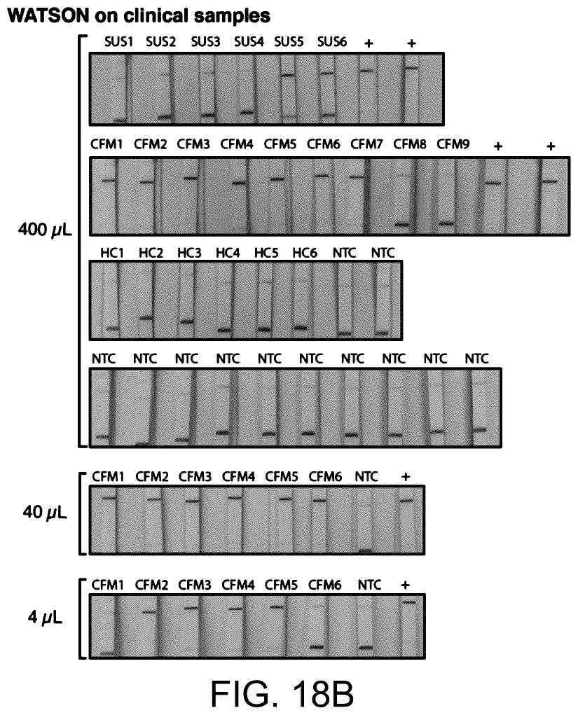

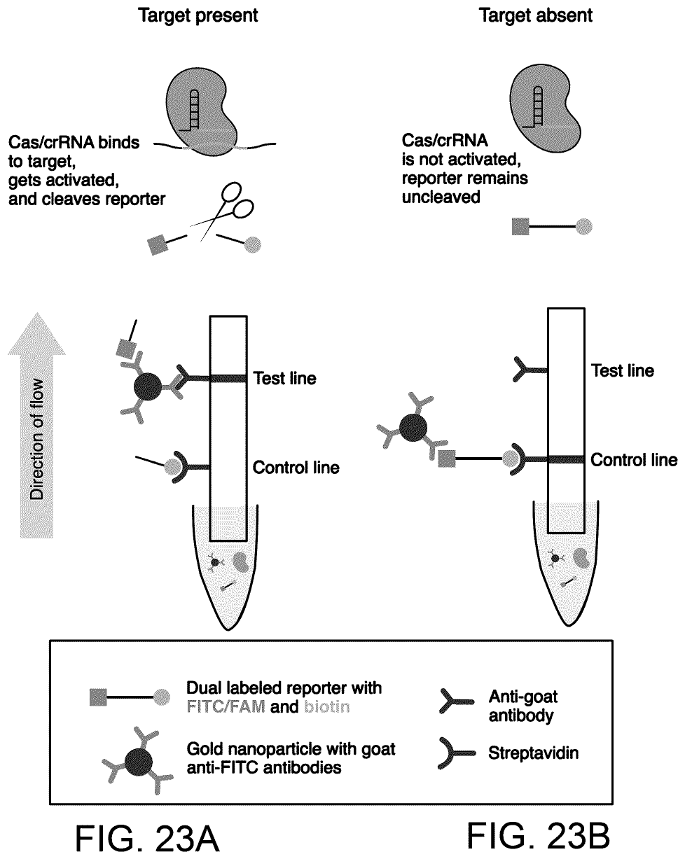

Another key aspect of point-of-care testing is ease of signal detection. The collateral RNAse activity of the activated Cas13 enzyme can be exploited in a variety of ways to provide an easily interpretable diagnostic result. By placing a Cas13-cleavable RNA linker between biotin and fluorescein amidite (FAM), Cas13 activation has been shown to be detectable in a lateral flow assay (LFA)³¹, a convenient format for field-deployable, point-of-care tests in resource-limited settings (FIG. 23A-23B). To evaluate LFA sensitivity for cfDNA from plasma of patients with active pulmonary tuberculosis, Applicant compared the detection of LFA signal, quantified by image analysis (see Methods), with fluorescence signal for the same set of 18-plex pooled PCR amplified clinical samples. Applicant found the two detection modalities to be concordant—affording the same sensitivity and specificity of assay for the equivalent of 400 μL patient plasma. Applicant thus demonstrates Mtb cfDNA detection in a LFA platform and a path forward for its application as a point-of-care test (FIG. 12B, FIG. 18A-18B).

### Discussion

Liquid biopsies are beginning to revolutionize disease diagnostics and management^{8,32,33}. In particular, detection of circulating microbial cfDNA has the potential to transform infectious disease diagnostics given its sensitivity, relatively non-invasive nature, applicability to many different infection types, and potential robustness to prior, recent antibiotic treatment. Indeed, there is growing clinical evidence on the efficacy of NGS-based microbial cfDNA tests^{32,34}. The advantage of sequencing microbial cfDNA is its sensitivity in detecting rare pathogen nucleic acids, since the detection of any fragment of the pathogen genome may be sufficient for diagnosis and de novo sequencing requires no hypothesis about organism identity, although in practice the presence of the pathogen in a reference database, a minimum genome coverage threshold or other metrics may be needed to improve specificity6. Offsetting this advantage is the potential loss in sensitivity due to complex library construction, wherein short and single stranded fragments may be lost¹². Additional key disadvantages with NGS is the significant infrastructure, cost, and time currently needed to process and sequence samples, and interpret results. As an alternative, PCR-based detection of cfDNA has been proposed, but is not yet clinically deployed due, in large part, to insufficient sensitivity of these assays as current PCR-based cfDNA assays typically only detect a single genomic sequence. For example, in the case of Mtb, reported sensitivity ranges from 45%-65%.³⁵⁻³⁷ Encouragingly, a recent study reported improved sensitivity (>90%) in a pooled adult and pediatric group using a singleplex CRISPR-based diagnostic, albeit on limited sample numbers from a single geo-graphical location7. Here Applicant demonstrates that tiling—pooled amplification of targets across the genome with pooled CRISPR/Cas13-based detection of these targets—affords improved sensitivity over single-plex assays, which will pave the way for improving diagnostic sensitivities even further and minimizing the volume of blood that is required from patients.

This Example demonstrates a WATSON assay, which is a highly sensitive and specific diagnostic strategy that combines tiled, pooled amplification and CRISPR/Cas-13 detection that is able to detect cfDNA in patients with acute pulmonary tuberculosis, with progress supporting a field-deployable, point-of-care platform. Applicant has created a comprehensive assay development workflow for a WATSON assay as a modular diagnostic, starting with computational design of tiled amplification primers and crRNA that takes into account pathogen genomic diversity, testing and validation of primer and crRNA pools, to a final implementation in a single pooled amplification and detection step. By taking a tiled approach that can detect relatively short genomic fragments, Applicant increases the likelihood of detecting any one target that is present in a sample, thereby enabling Applicant's approach to detect considerably less than 1 genome equivalent per sample. Additionally, Applicant demonstrates that this CRISPR/Cas-13 detection strategy is able to accommodate the very short fragments of pathogen nucleic acids present in patient blood as cfDNA. The WATSON assay thus has the potential to be applied to the detection of pathogen cfDNA as a new approach to infectious disease diagnostic testing and as a tool that enables the study of the microbial cfDNA landscape in the context of infection. As a liquid biopsy approach, it has the potential to obviate the need for more invasive sampling of some infections and make more uniform sample collection for all infection-types.

In this Example, Applicant found that the WATSON assay detected Mtb cfDNA in 10 of 11 samples (91%) obtained from confirmed TB patients from the South African cohort and 8 of 9 samples (89%) from confirmed TB patients from the Ugandan cohort (FIG. 10 and FIG. 21). This performance was achieved using cfDNA from 400 μL of plasma, a volume similar to or less than previous studies. Even when using 100-fold less input volume (equivalent of 4 μL plasma), the WATSON assay had a sensitivity of 78%. This Example reveals the wide range in abundance of Mtb cfDNA present in confirmed pulmonary tuberculosis patients. It also highlights the potential of the WATSON assay. If the need for higher sensitivity is recognized as more samples are tested, this can be achieved by the WATSON assay not only by collecting and testing larger volumes of blood, but importantly, by increasing tiling across more of the genome.. There are many more loci within the entire Mtb genome that could be additionally targeted beyond this initial proof of principle set of 18 primer pairs and crRNA that currently targets only the IS6110 and IS1081 regions of the Mtb genome. Conversely, if increased tiling across many more targets can drive the sensitivity to even lower LoDs, then only very small amounts of collected blood will be required. In this way, the WATSON assay even has the potential to be used with finger-stick sample collection, which can produce up to 50 μL of blood. To further address the need for a rapid, point-of-care diagnostic, Applicant demonstrated the potential for the workflow to be adapted to more point-of-care settings, using a lateral flow assay (LFA) and an isothermal method for amplification. Importantly, LFA sensitivity was comparable to fluorescence readout, and singleplex RPA was comparable to singleplex PCR. In sum this Example can at least demonstrate that the WATSON assay has the needed range of sensitivity to detect cfDNA in patients and a path to a fully-integrated test that can be deployed in resource-limited settings where infrastructure-heavy molecular diagnostics, such as aPCR and NGS are currently not feasible.

While WATSON showed a 10- to 100-fold improvement in analytical sensitivity over singleplex SHERLOCK assay, the potential for WATSON to improve in clinical testing is highlighted in the 2 of 6 clinically suspected tuberculosis samples, despite their being negative by sputum-based culture and GeneXpert testing as well as cfDNA-based qPCR testing. It is tantalizing to think that the assay may be able to detect infections that currently elude current assays.

Given the WATSON assay's ability to detect Mtb cfDNA in blood and without being bound by theory, Applicant envision it being used as a diagnostic for many different infectious syndromes, pathogens, conditions, physiological states and/or the like by detecting cfDNA of the infecting agent or other components in blood as well as other bodily fluids such as urine, which has been shown to contain microbial cfDNA³⁸⁻³⁹. The programmable nature of nucleic acid detection has no limits with regards to the range of pathogens to which it can be applied. In addition to its application as a diagnostic, given the technical ease and minimally invasive nature of the WATSON assay, it has the potential to be applied in other ways to impact patient care, including following circulating pathogen cfDNA levels as a biomarker to define the efficacy and even duration of antibiotic treatment” or as an alternative metric for efficacy in clinical trials of drugs. In the case of the exemplary disease, Mtb, shown in this Example, cfDNA levels could potentially supplement or even replace sputum-based measures of early bactericidal activity³⁷, thereby informing drug development and other interventional strategies.

In sum, this Example describes and demonstrates a WATSON assay, which is a nucleic acid detection method that builds on existing CRISPR-diagnostics and represents a unique strategy for designing and detecting multiple genomic targets while remaining substantially faster and easier to execute than current sequencing based tests. It leverages the two-step amplification and detection by Cas13, to ensure the unique capability of detecting very short nucleic fragments with high sensitivity and specificity and increases the clinical sensitivity and improves existing cfDNA strategies through tiling across the pathogen genome. When applied to clinical samples, the WATSON assay is able to sensitively detect an exemplary pathogen cfDNA in patients (those with active pulmonary tuberculosis) and demonstrates the potential to perform substantially better than previously reported amplification based methods for the same exemplary target pathogen. Importantly, this Example reveals that detectable DNA of pathogens such as Mtb can be found in the plasma of a high frequency of patients with pulmonary TB, the power of tiling to improve the ability to detect it, and the feasibility of the WATSON assay as a diagnostic test, such as a point-of-care diagnostic.

### Materials and Methods

### Identification of Unique MTBC Genomic Regions

Computational analysis was done using custom Python scripts. Complete, closed genome sequences without gaps were used to identify suitable targets. They were downloaded from NCBI and included 267 whole genome sequences of the Mycobacterium tuberculosis complex (MTBC), 88 sequences from non-tuberculous mycobacteria (NTMs), and the reference human genome (GRCh38.p11 [https://www.ncbi.nlm. nih.gov/assembly/GCF_000001405.40])) The reference MTB sequence (H37Rv, accession: NC_000962) was broken down into a sliding set of 28-mers. The total number of 28-mers generated was 4,411,504 (i.e., the size of the genome minus 28). These 28-mers were then tested for alignment with all other MTBC sequences (‘in-group’), as well as NTMs and human genome (‘out-group’), using a fast sequence alignment tool (Bowtie2.) 28-mers that were one or fewer SNPs apart across all MTBC sequences were defined as the conserved targets (77% of all targets). These 28-mers were then screened to exclude any that were less than 4 SNPs away from any part of the out-group genomes. The remaining targets (65% of original) were then mapped back to the reference genome (H37Rv) and these genomic regions were used for further analysis.

### Genome-Wide Tiled Primer Design

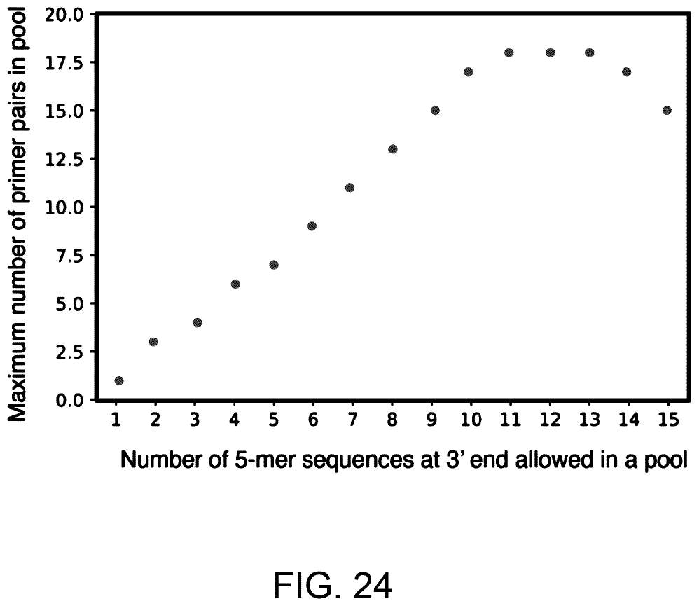

Effective multiplexing can be undermined by off-target interactions between primers. To minimize detrimental interactions among pooled primers, Applicant designed primers based on the principle of minimizing 3′-3′ interactions between primer pairs. All primers in a pool contained the same set of 5-mers at their 3′ ends, where the sequence of compatible 5-mers and corresponding primer pairs were determined by an iterative, heuristic search algorithm. First, all possible 5-mers (45=1024) were ranked based on how many times they were present in the top strand of the IS6110 and IS1081 regions. 5-mers that contained three or more repeated nucleotides were removed from this list of 5-mers. Of the remaining ranked 5-mers, Applicant selected the top 100 and identified the loci of potential primer pairs for each one based on whether that 5-mer and its reverse complement were separated by 28 to 48 base pairs to allow for crRNA binding. To create a pool of primers wherein all primers contained the same 5-mer sequence at the 3′ end, Applicant started with the top ranked 5-mer, generated 30 nucleotide-long primer pairs at all of the identified loci, and only included those in the pool if 1) amplicons were non-overlapping with any others in the pool, 2) neither forward or reverse primers contained a stretch of 5 bases complementary to the 5-mer and 3) they did not contain stretches of four or more homo-polymers. The 5′-end of primers were then extended or trimmed to ensure their melting temperatures were within 63-65 C. To increase the size of a pool, Applicant allowed primers to contain a second 5-mer sequence at their 3′ ends—in addition to the 5-mer from the first round that yielded the greatest number of primers in a pool—by generating another set of 30 nucleotide-long primer pairs, each containing one of the two “allowed” 5-mer sequences and only included those in a pool if 1) amplicons were non-overlapping with any others in the pool, 2) neither forward or reverse primers con-tained a stretch of 5 bases complementary to either of the two 5-mers in the pool, and 3) they did not contain stretches of four or more homopolymers. Applicant generated all possible pools for primers that contained up to 15 different 5-mer sequences at their 3′ ends; and identified a maximum of 18 primer pairs that could be pooled together, wherein each primer pair contained one of 11 different 5-mer sequences at their 3′ end. (FIG. 24).Primer and crRNA preparation

Individual primers were ordered from Integrated DNA Technologies and resuspended in nuclease-free water and stored at −20° C. crRNAs were ordered as complementary ssDNA sequences with a T7 promoter binding sequence attached to the 5′-end. Each crRNA was synthesized via in vitro transcription (IVT) using the HiScribe T7 High Yield RNA Synthesis Kit (New England Biolabs) by incubating a ssDNA template (at 1 μM final concentration) in reaction buffer at 37° C. with T7 promoter primer (1 μM final concentration) for 12 hours. In vitro transcribed product was then diluted down to a final concentration of 225 nM of crRNA and quantified using a Nanodrop instrument (Thermo Scientific). crRNAs were stored at −80° C. For one crRNA, (IS6110_2), in addition to IVT, synthetic RNA was also purchased from Synthego corporation. In this case, the RNA was rehydrated with nuclease free water, diluted to 225 nM, and stored at −80° C. For pooled crRNA detection, all 18 crRNAs were mixed to a total concentration of 225 nM (12.5 nM per crRNA) and stored at −80° C. until further use. The sequences of all primers and crRNAs are provided in Table 10.

### Bacterial Culture and Genomic DNA Preparation

Genomic DNA was isolated from Mycobacterium tuberculosis H37Rv grown in Middlebrook 7H9 medium supplemented with OADC using a cetrimide-based protocol as previously described⁴². Mtb genomic DNA (1 μg) was digested with NEBNext dsDNA Fragmentase (30 min for a median size of about 180 bp) using 2× the recommended concentration of Fragmentase enzyme. Fragmented gDNA was purified with AMPure XP DNA SPRI beads (2.5×) and eluted in 20 μL nuclease-free water. The concentration of purified fragmented DNA was quantified using Qubit (Life Technologies), and fragment size profiles were determined on the Agilent 4200 Tapestation (High sensitivity D1000 kit). Based on the mass of 1 genome of the TB reference strain H37Rv (4,411,532 bp=5 fg), we estimated the number of genome equivalents per μL (GE/μL) in the quantitated purified fragmented DNA. 1 GE is defined as the mass of fragmented Mtb DNA equal to 5 fg. Dilutions of fragmented DNA were prepared in nuclease-free water and stored at −20° C. in lo-bind plasticware.

Non-tuberculosis mycobacteria were cultured using the same methods as Mycobacterium tuberculosis. Extractions were carried out using the DNeasy Blood and Tissue Nucleic Acid Extraction Kit using the gram-negative bacteria sample preparation, “Purification of Total DNA from Animal Tissues (SpinColumn Protocol)”. For other bacteria, liquid cultures were grown overnight and extracted using the DNeasy kit as described above.

cfDNA Preparation from Clinical Samples

The cfDNA from clinical samples (CFM 1-9, SUS 1-6, and HC 1-6) were collected from study participants at Stanford University and in Uganda. Approval was obtained for the collection of samples CFM 1-9 and SUS 1-6 from the institutional review board (IRB) at the Uganda National Council for Science and Technology; and for samples HC 1-6, IRB approval was obtained from Stanford University. All participants were >18 years of age and provided written informed consent²⁸. Samples from patients with pulmonary TB were confirmed via sputum-based culture and/or the Xpert MTB/RIF assay (Cepheid, Sunnyvale, CA, USA); as well as a cfDNA-based real time PCR assay, which targeted a single 72-bp region of IS6110, as previously reported²⁸. Additional healthy control samples (HC 7-26) were obtained from Research Blood Components, LLC (Watertown, MA).

Blood from all samples were collected and cfDNA extracted per the optimized protocol identified and reported previously²⁸. Briefly, blood was collected in K2EDTA tubes (Becton, Dickinson, Franklin Lakes, NJ), centrifuged at 500×g for 10 minutes at room temperature, and the plasma was transferred to a new tube, stored at −80° C. and shipped to Stanford University. cfDNA was extracted from 4 mL of plasma using the Maxwell RSC system (Promega) and the Maxwell RSC large-volume ccfDNA kit. Samples were eluted in 100 μL, of which 10 μL was used in the cfDNA-based real time PCR assay and the experiments reported herein, unless otherwise specified.

Singleplex and genome-wide (multiplexed) PCR amplification Singleplex and multiplexed PCR amplification was carried out using the Multiplex PCR Plus kit (Qiagen). For singleplex amplification, the final concentration of the single primer pair was 2 AM, and 0.5×Q solution in 1× QIAGEN Multiplex PCR Master Mix. For multiplexed amplification, the final concentrations in each reaction were 400 nM per primer pair, with a total primer concentration of 7.2 μM and 0.5×Q solution in 1× QIAGEN Multiplex PCR Master Mix. For general experiments, 1/50 of the volume was DNA template. When using clinical cfDNA isolates, 1/5 of the volume was DNA template in the final reaction. Reactions were incubated at 95° C. for 5 minutes, followed by 40 cycles of 95° C. for 30 seconds, 60° C. for 90 seconds, and 72° C. for 30 seconds. And finally, 68° C. for 10 minutes and then held at 4° C.

### Recombinase Polymerase Amplification (RPA)

RPA Reactions were performed using TwistAmp Basic kits (TwistDx UK). 50 μL reactions were performed as directed by the manufacturer's protocol. To make other reaction volumes, single-use pellets were rehydrated and pooled to generate a master reaction mix that was distributed into 10-20 μL individual reactions. Final primer concentration was 500 nM of each primer (primer sequences are listed in Table 10). Magnesium acetate (280 mM) was added to the wall of the tube, so that reactions began simultaneously upon centrifugation at 3200×g. RPA reactions were incubated at 37° C. in a thermocycler for 20 minutes, with 10 minutes at 75° C. to inactivate polymerase unless otherwise specified.

### Cas13 Detection

Leptotrichia wadei Cas13a enzyme was purchased from a commercial vendor (Genscript) and aliquoted into 3 μL aliquots and flash frozen with liquid nitrogen and stored at −80 C until use. Each crRNA was either synthesized using in vitro transcription, or purchased from Synthego Corporation (IS6110_2). LwCas13 was kept on ice until rehydrating in 49 μL of Cas13 storage buffer (50 mM Tris-Hcl at pH 7.5, 600 mM NaCl, and 5% glycerol). The Cas13 detection mix contains Cas Cleavage Buffer (40 mM Tris, 6 mM MgCl2, 1 mM DTT), RNase inhibitor to 1 U/μL (New England BioLabs), T7 polymerase to 1.5 U/μL (New England BioLabs), rNTPs to 1 mM (New England BioLabs), MgCl2 to 9 mM, rehydrated Cas13 protein to 45 nM, RNase Alert v2 reporter to 125 nM (Life Technologies) or in the case of lateral flow readout FAM-Biotin reporter (Integrated DNA Technologies) to a final concentration of 1 μM. The final crRNA concentration in detection reactions was 22.5 nM (1.25 nM of each of 18 crRNA for pooled detection; 22.5 nM of a single crRNA for parallel detection). For the clinical samples that were detected by lateral flow, the concentration of each crRNA was 12.5 nM, resulting in a total crRNA concentration of 225 nM.

Fluorescent-based Cas13 detection was measured using a Spectramax M5 Plate Reader (Molecular Devices), using 490 nM for the excitation wavelength and 520 nM for the emission wavelength unless otherwise specified. RPA reactions were added to the Cas13 detection mix in a ratio of 1:19. SHERLOCK reactions were incubated for 2 hours at 37° C. unless otherwise specified.

### Droplet Experiment Protocol

Droplets experiments were performed as previously described²⁰ using the DropArray platform. Briefly, detection sets were prepared at 2.2×final concentration of 45 nM purified Leptotrichia wadei Cas13a, 22.5 nM total crRNA concentration (1.25 nM of each of 18 crRNA for pooled detection; 22.5 nM of one crRNA for individual detection), 500 nM quenched fluorescent RNA reporter (RNAse Alert v2, Thermo Scientific), 2 μl murine RNase inhibitor (New England Biolabs) in nuclease assay buffer (40 mM Tris-HCl, 60 mM NaCl, pH 7.3) with 1 mM NTPs and 0.6 μl T7 polymerase mix (New England Biolabs). Amplified samples were diluted 1:10 into nuclease-free water supplemented with 13.2 mM MgCl2 prior to barcoding with fluorescent dyes. 20 μL of each sample and detection mix were then emulsified into droplets using a BioRad QX200 droplet generator using fluorous oil (3M 7500, 70 μL) containing 2% 008-fluorosurfactant (RAN Biotechnologies.) Droplets were pooled and loaded into a DropArray chip, incubated at 37° C., and imaged for assay signal at 0, 1 hour, and 3 hour time points relative to the start of the incubation.

### Lateral Flow Assay

For lateral flow detection, Applicant used the commercially available lateral flow assay kit, Milenia Genline HybriDetect™ kit (TwistDx) (FIG. 23A-23B). In the Cas13 detection step, the RNase Alert v2 fluorescent reporter was replaced with a FAM-Biotin labeled poly-U reporter (5′-FAM-UUUUUUUUUUUUUU-Biotin-3′) (SEQ ID NO: 59) (Integrated DNA Technologies) in the Cas13 detection mix to a final concentration of 1 μM; then added to the Hybridetect™ assay buffer at a ratio of 1:4. Lateral flow strips were then inserted into the microtube containing the buffer and incubated for 10 minutes, after which results were quantified using ImageJ software. Normalized Signal was calculated as (Test Band Intensity)/(Test Band Intensity+Control Band Intensity). Statistics & reproducibility

Data are shown as original values or median with error bars depicting range and standard deviation. Technical and biological replicates of samples were tested for reproducibility (up to six replicates) and the variability was quantified and is discussed in the manuscript text. For clinical testing, sample size calculations were not performed as comprehensive clinical evaluation is not the goal of this study. Sample size of the experimental group (active and suspected TB) was based on availability of clinical samples. Sample size of the control group (healthy individuals) was chosen to be larger than the experimental group. Experiments were not randomized but the investigators were blinded to allocation during experiments and data analysis. No data were excluded from the analyses.

### REFERENCES FOR EXAMPLE 7

1. World Health Organization, Global Health Estimates 2019: Deaths by Cause, Age, Sex, by Country and by Region, 2000-2019. (2019).
2. Mathieu, E. et al. Coronavirus Pandemic (COVID-19). Our World in Data (2020).
3. Branson, B. M. et al. Laboratory testing for the diagnosis of HIV infection: updated recommendations. (Centers for Disease Control and Prevention, 2014).
4. Testing Recommendations for Hepatitis C Virus Infection | CDC. (2021).
5. Fernindez-Carballo, B. L., Broger, T., Wyss, R., Banaei, N. & Denkinger, C. M. Toward the development of a circulating free DNA-based in vitro diagnostic test for infectious diseases: A review of evidence for tuberculosis. J. Clin. Microbiol. 57, e01234-01218 (2019).
6. Blauwkamp, T. A. et al. Analytical and clinical validation of a microbial cell-free DNA sequencing test for infectious disease. Nat. Microbiol. 4, 663-674 (2019).
7. Huang, Z. et al. CRISPR detection of circulating cell-free Myco-bacterium tuberculosis DNA in adults and children, including chil-dren with HIV: a molecular diagnostics study. Lancet Microbe 3, e482-e492 (2022).
8. Wan, J. C. M. et al. Liquid biopsies come of age: towards imple-mentation of circulating tumour DNA. Nat. Rev. Cancer 17, 223-238 (2017).
9. Vorperian, S. K., Moufarrej, M. N., Tabula Sapiens, C. & Quake, S. R. Cell types of origin of the cell-free transcriptome. Nat. Biotechnol. 40, 855-861 (2022).
10. Breitbach, S. et al. Direct quantification of cell-free, circulating DNA from unpurified plasma. PLoS One 9, e87838 (2014).
11. Leary, R. J. et al. Development of personalized tumor biomarkers using massively parallel sequencing. Sci. Transl. Med. 2, 20ra14 (2010).
12. Burnham, P. et al. Single-stranded DNA library preparation uncovers the origin and diversity of ultrashort cell-free DNA in plasma. ScientificRep. 6, 27859 (2016).
13. Kowarsky, M. et al. Numerous uncharacterized and highly divergent microbes which colonize humans are revealed by circulating cell-free DNA. Proc. Natl Acad. Sci. U.S.A 114, 9623-9628 (2017).
14. Huang, Y.-F. et al. Analysis of microbial sequences in plasma cell-free DNA for early-onset breast cancer patients and healthy females. BMC Med. Genomics 11,16(2018).
15. Han, D. et al. Liquid biopsy for infectious diseases: a focus on microbial cell-free DNA sequencing. Theranostics 10, 5501-5513 (2020).
16. Gootenberg, J. S. et al. Nucleic acid detection with CRISPR-Cas13a/C2c2. Science 356, 438-442 (2017).
17. Gootenberg, J. S. et al. Multiplexed and portable nucleic acid detection platform with Cas13, Cas12a, and Csm6. Science 360, 439-444 (2018).
18. Abudayyeh, O. O. et al. C2c2 is a single-component programmable RNA-guided RNA-targeting CRISPR effector. Science 353, aaf5573 (2016).
19. Thakku, S. G. et al. Multiplexed detection of bacterial nucleic acids using Cas13 in droplet microarrays. PNAS Nexus 1, https://doi.org/10.1101/2021.11.12.468388 (2022).
20. Ackerman, C. M. et al. Massively multiplexed nucleic acid detection with Cas13. Nature 582, 277-282 (2020).
21. Denkinger, C. M. et al. Guidance for the evaluation of tuberculosis diagnostics that meet the World Health Organization (WHO) Target product profiles: An introduction to WHO process and study design principles. J. Infect. Dis. 220,S91-S98 (2019).
22. Coscolla, M. & Gagneux, S. Consequences of genomic diversity in Mycobacterium tuberculosis. Semin. Immunol. 26, 431-444 (2014).
23. Roychowdhury, T., Mandal, S. & Bhattacharya, A. Analysis of IS6110 insertion sites provide a glimpse into genome evolution of Myco-bacterium tuberculosis. Sci. Rep. 5,12567 (2015).
24. Collins, D. M. & Stephens, D. M. Identification of an insertion sequence, IS1081, in Mycobacterium bovis. FEMS Microbiol. Lett. 67, 11-15 (1991).
25. Kulesa, A., Kehe, J., Hurtado, J. E., Tawde, P. & Blainey, P. C. Com-binatorial drug discovery in nanoliter droplets. Proc. Natl Acad. Sci. U.S.A. 115, 6685-6690 (2018).
26. Green, C. et al. Rapid diagnosis of tuberculosis through the detection of mycobacterial DNA in urine by nucleic acid amplifi-cation methods. Lancet Infect. Dis. 9, 505-511 (2009).
27. Li, Y. et al. Size separation of circulatory DNA in maternal plasma permits ready detection of fetal DNA polymorphisms. Clin. Chem. 50, 1002-1011 (2004).
28. Murugesan, K. et al. Investigation of preanalytical variables impacting pathogen cell-free DNA in blood and urine. J. Clin. Microbiol. 57,e00782-00719 (2019).
29. Alonso, H., Samper, S., Martin, C. & Otal, I. Mapping IS6110 in high-copy number Mycobacterium tuberculosis strains shows specific insertion points in the Beijing genotype. BMC Genom. 14, 422 (2013).
30. Cook, L. et al. Does Size Matter? Comparison of extraction yields for different-sized DNA fragments by seven different routine and four new circulating cell-free extraction methods. J. Clin. Microbiol. 56, e01061-01018 (2018).
31. Myhrvold, C. et al. Field-deployable viral diagnostics using CRISPR-Cas13. Science 360, 444-448 (2018).
32. Cheng, A. P. et al. A cell-free DNA metagenomic sequencing assay that integrates the host injury response to infection. Proc. Natl Acad. Sci. 116, 18738-18744 (2019).
33. Ignatiadis, M., Sledge, G. W. & Jeffrey, S. S. Liquid biopsy enters the clinic-implementation issues and future challenges. Nat. Rev. Clin. Oncol. 18, 297-312 (2021).
34. Morales, M. The Next Big Thing? Next-generation sequencing of microbial cell-free DNA using the Karius Test. Clin. Microbiol. Newsl. 43, 69-79 (2021).
35. Click, E. S. et al. Detection of apparent cell-free M. tuberculosis DNA from plasma. Sci. Rep. 8,645(2018).
36. Ushio, R. et al. Digital PCR assay detection of circulating Myco-bacterium tuberculosis DNA in pulmonary tuberculosis patient plasma. Tuberculosis 99, 47-53 (2016).
37. Pan, S.-W. et al. Mycobacterium tuberculosis-derived circulating cell-free DNA in patients with pulmonary tuberculosis and persons with latent tuberculosis infection. PLoS One 16,e0253879 (2021).
38. Patel, K. et al. Evaluation of aurine-based rapid molecular diag-nostic test with potential to be used at point-of-care for pulmonary tuberculosis: Cape Town cohort. J. Mol. Diagn. 20, 215-224 (2018).
39. Labugger, I. et al. Detection of transrenal DNA for the diagnosis of pulmonary tuberculosis and treatment monitoring. Infection 45, 269-276 (2017).
40. Magombedze, G., Pasipanodya, J. G. & Gumbo, T. Bacterial load slopes represent biomarkers of tuberculosis therapy success, fail-ure, and relapse. Commun. Biol. 4, 1-13 (2021).
41. Jindani, A., Aber, V., Edwards, E. & Mitchison, D. The early bacter-icidal activity of drugs in patients with pulmonary tuberculosis. Am. Rev. Respir. Dis. 121, https://doi.org/10.1164/arrd.1980.121.6. 939 (1980).
42. Somerville, W., Thibert, L., Schwartzman, K. & Behr, M. A. Extraction of Mycobacterium tuberculosis DNA: a question of containment. J. Clin. Microbiol. 43, 2996-2997 (2005).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A nucleic acid detection system comprising:
a. a set of amplification primer pairs configured to amplify a set of target regions in one or more target elements, wherein primers of the set of amplification primer pairs are optimized for pooled amplification;
b. one or more Cas proteins having collateral activity;
c. a set of guide polynucleotides comprising a guide polynucleotide specific for each target region amplified by the set of amplification primer pairs, and wherein each of the guide polynucleotides in the set of guide polynucleotides is capable of forming a CRISPR-Cas complex with the one or more Cas proteins; and
d. an oligonucleotide-based detection construct comprising a non-target sequence, wherein the non-target sequence is configured to be cleaved by the collateral activity of the one or more Cas proteins.

2. The nucleic acid detection system of claim 1, wherein the set of amplification primer pairs optimized for pooled detection are configured to minimize 3′ to 3′ interactions between primers.

3. The nucleic acid detection system of claim 1, wherein
a. the guide polynucleotides of the set of guide polynucleotides are optimized for pooled detection;
b. the set of amplification primer pairs comprises 2-10 or more amplification primer pairs;
c. the set of guide polynucleotides comprises 2-50 or more guide polynucleotides;
d. the system is configured to detect 2-50 or more target elements;
e. the system is configured to detect 2-50 or more target regions; or
f. any combination of (a)-(e).

4. The nucleic acid detection system of claim 4, wherein:
the one or more target elements is specific to a microorganism or a virus; or
wherein the one or more target elements are elements in a genome of a microorganism or virus.

5. (canceled)

6. The nucleic acid detection system of claim 1, wherein the one or more target elements are elements in circulating cell free (ccfDNA).

7. The nucleic acid detection system of claim 1, wherein one or more of the one or more target elements is a repetitive target element.

8. The nucleic acid detection system of claim 1, wherein the set of amplification primer pairs comprises PCR primers, isothermal amplification primers, proximity dependent probes, or any combination thereof.

9-16. (canceled)

17. The nucleic acid detection system of claim 1, wherein one or more primers of the set of amplification primer pairs comprises an origin-specific barcode, a set-specific barcode, a unique molecular identifier (UMI), or any combination thereof.

18. The nucleic acid detection system of claim 1, wherein the set of amplification primer pairs is configured to amplify at least one or more target regions of a target element that cover at least 10 percent, at least 20 percent, at least 30 percent, at least 40 percent, at least 50 percent, or at least 60 percent of a target element.

19-20. (canceled)

21. The nucleic acid detection system of claim 1, wherein the nucleic acid detection system further comprises a nucleic acid enrichment reagent, wherein the nucleic acid enrichment reagent comprises a ccfDNA enrichment agent, DNA methylation enrichment agents, size selection reagents to enrich for a nucleic acid, a magnetic or paramagnetic particle configured to bind nucleic acids, or any combination thereof.

22. (canceled)

23. The nucleic acid detection system of claim 1, further comprising one or more nuclease inactivation reagents, microorganism or virus inactivation reagents, or both.

24. The nucleic acid detection system of claim 1, wherein the one or more Cas proteins comprise an RNA-targeting protein, a DNA-targeting protein, or a combination thereof.

25. The nucleic acid detection system of claim 1, wherein the one or more Cas proteins comprise a Cas13, a Cas12, or a combination thereof.

26-28. (canceled)

29. The nucleic acid detection system of claim 1, wherein the nucleic acid detection system comprises two or more CRISPR-Cas systems, wherein the two or more CRISPR-Cas systems comprise an RNA-targeting effector protein, a DNA-targeting effector protein, or a combination thereof.

30. (canceled)

31. The nucleic acid detection system of claim 1, wherein the one or more guide polynucleotides are each about 28 nucleotides in length and have a mismatch of one or less to the corresponding target sequence.

32. (canceled)

33. The nucleic acid detection system of claim 1, wherein the oligonucleotide-based detection construct comprises a masking construct configured to suppress generation of a detectable positive signal until the non-target sequence is cleaved by the collateral activity of the one or more Cas proteins.

34-47. (canceled)

48. A method of detecting one or more nucleic acids in a sample, the method comprising:
a. contacting one or more samples with a set of amplification primer pairs configured to amplify a set of target regions in one or more target elements, wherein primers of the set of amplification primer pairs are optimized for pooled amplification;
b. amplifying, by pooled amplification, two or more target regions in one or more target elements by the set of amplification primer pairs thereby producing one or more amplified target regions;
c. contacting the one or more amplified target regions with one or more Cas proteins having collateral activity, a set of guide polynucleotides comprising a guide polynucleotide specific for each of the amplified target regions, wherein each of the guide polynucleotides in the set of guide polynucleotides is capable of forming a CRISPR-Cas complex with the one or more Cas proteins;
d. contacting the one or more amplified target regions, the one or more Cas proteins having collateral activity, guide polynucleotides, or complexes thereof with one or more oligonucleotide-based detection constructs each comprising a non-target sequence to be cleaved by the collateral activity of the one or more of the one or more Cas proteins upon activation of the Cas proteins; and
e. detecting a signal produced from the one or more oligonucleotide-based detection constructs in response cleavage of one or more of the non-target sequences by collateral activity of an activated CRISPR-Cas protein, thereby detecting one or more target elements in the sample.

49-52. (canceled)

53. The method of claim 48, wherein the one or more samples comprises plasma, blood, urine, or saliva.

54-55. (canceled)

56. The method of claim 48, further comprising extracting cell free DNA, DNA, RNA, or other nucleic acids or any combination thereof from a sample prior to amplification.

57. The method of claim 48, wherein the one or more samples is/are obtained from one or more subjects, and wherein one or more of the one or more subjects has an active microorganism and/or viral infection, has been infected, or is suspected of being infected with a microorganism and/or virus.

58-99. (canceled)


SEQUENCE LISTING (59 sequences)

SEQ ID NO: 1 - protein, 1001 aa, synthetic construct
MKISKVXXXVXKKXXXGKLXKXVNERNRXAKRLSNXLBKYIXXIDKIXKKEXXKKFXAXEEITLKLNQXXXBXLXKAXXDLRKDNXYSXJKKILHNEDINXEEXELLINDXLEKLXKIESXKYSYQKXXXNYXMSVQEHSKKSIXRIXESAKRNKEALDKFLKEYAXLDPRMEXLAKLRKLLELYFYFKNDXIXXEEEXNVXXHKXLKENHPDFVEXXXNKENAELNXYAIEXKKJLKYYFPXKXAKNSNDKIFEKQELKKXWIHQJENAVERILLXXGKVXYKLQXGYLAELWKIRINEIFIKYIXVGKAVAXFALRNXXKBENDILGGKIXKKLNGITSFXYEKIKAEEILQREXAVEVAFAANXLYAXDLXXIRXSILQFFGGASNWDXFLFFHFATSXISDKKWNAELIXXKKJGLVIREKLYSNNVAMFYSKDDLEKLLNXLXXFXLRASQVPSFKKVYVRXBFPQNLLKKFNDEKDDEAYSAXYYLLKEIYYNXFLPYFSANNXFFFXVKNLVLKANKDKFXXAFXDIREMNXGSPIEYLXXTQXNXXNEGRKKEEKEXDFIKFLLQIFXKGFDDYLKNNXXFILKFIPEPTEXIEIXXELQAWYIVGKFLNARKXNLLGXFXSYLKLLDDIELRALRNENIKYQSSNXEKEVLEXCLELIGLLSLDLNDYFBDEXDFAXYJGKXLDFEKKXMKDLAELXPYDQNDGENPIVNRNIXLAKKYGTLNLLEKJXDKVSEKEIKEYYELKKEIEEYXXKGEELHEEWXQXKNRVEXRDILEYXEELXGQIINYNXLXNKVLLYFQLGLHYLLLDILGRLVGYTGIWERDAXLYQIAAMYXNGLPEYIXXKKNDKYKDGQIVGXKINXFKXDKKXLYNAGLELFENXNEHKNIXIRNYIAHFNYLSKAESSLLXYSENLRXLFSYDRKLKNAVXKSLINILLRHGMVLKFKFGTDKKSVXIRSXKKIXHLKSIAKKLYYPEVXVSKEYCKLVKXLLKYK

SEQ ID NO: 2 - DNA, 55 bp, synthetic construct
  source 1..55: mol_type = other DNA
gaaattaatacgactcactatagggcaagtcgcaagtgtcgatcatggccaaaga

SEQ ID NO: 3 - DNA, 22 bp, synthetic construct
  source 1..22: mol_type = other DNA
ggcgaggaaggtatacgggccg

SEQ ID NO: 4 - DNA, 89 bp, synthetic construct
  source 1..89: mol_type = other DNA
gctcgacgaagccgtagaggcgtttcgggttttagtccccttcgtttttggggtagtctaaatcccctatagtgagtcgtattaatttc

SEQ ID NO: 5 - DNA, 55 bp, synthetic construct
  source 1..55: mol_type = other DNA
gaaattaatacgactcactatagggatcagttgttgcccaatatgatcgggtact

SEQ ID NO: 6 - DNA, 17 bp, synthetic construct
  source 1..17: mol_type = other DNA
gtcggtgcgggcggtgt

SEQ ID NO: 7 - DNA, 89 bp, synthetic construct
  source 1..89: mol_type = other DNA
aaactccccgcggtggccgagcacctcggttttagtccccttcgtttttggggtagtctaaatcccctatagtgagtcgtattaatttc

SEQ ID NO: 8 - DNA, 54 bp, synthetic construct
  source 1..54: mol_type = other DNA
gaaattaatacgactcactatagggctcttctcatcttatcgacgccgagcagc

SEQ ID NO: 9 - DNA, 19 bp, synthetic construct
  source 1..19: mol_type = other DNA
cgagagcagcccgcgcagc

SEQ ID NO: 10 - DNA, 89 bp, synthetic construct
  source 1..89: mol_type = other DNA
ttctggctgaccaactcgcacaggcgaggttttagtccccttcgtttttggggtagtctaaatcccctatagtgagtcgtattaatttc

SEQ ID NO: 11 - DNA, 45 bp, synthetic construct
  source 1..45: mol_type = other DNA
gaaattaatacgactcactatagggcgggctaccgcgaacgcagc

SEQ ID NO: 12 - DNA, 22 bp, synthetic construct
  source 1..22: mol_type = other DNA
tcgatggttgcggcacgggtgt

SEQ ID NO: 13 - DNA, 89 bp, synthetic construct
  source 1..89: mol_type = other DNA
cgatgagcggtccaatcagcgcaacggcgttttagtccccttcgtttttggggtagtctaaatcccctatagtgagtcgtattaatttc

SEQ ID NO: 14 - DNA, 55 bp, synthetic construct
  source 1..55: mol_type = other DNA
gaaattaatacgactcactatagggaacccactacgcagccaatctgatggcagc

SEQ ID NO: 15 - DNA, 27 bp, synthetic construct
  source 1..27: mol_type = other DNA
tcgggctggtcgtagatggagtgcagc

SEQ ID NO: 16 - DNA, 89 bp, synthetic construct
  source 1..89: mol_type = other DNA
caccccgaagccctcctggccgtgggtggttttagtccccttcgtttttggggtagtctaaatcccctatagtgagtcgtattaatttc

SEQ ID NO: 17 - DNA, 45 bp, synthetic construct
  source 1..45: mol_type = other DNA
gaaattaatacgactcactatagggctcacccgagcccgagcagc

SEQ ID NO: 18 - DNA, 27 bp, synthetic construct
  source 1..27: mol_type = other DNA
cagtctaggtggtcagtgctggggtgt

SEQ ID NO: 19 - DNA, 89 bp, synthetic construct
  source 1..89: mol_type = other DNA
actgaccagcaccgaagaacccgccaaggttttagtccccttcgtttttggggtagtctaaatcccctatagtgagtcgtattaatttc

SEQ ID NO: 20 - DNA, 43 bp, synthetic construct
  source 1..43: mol_type = other DNA
gaaattaatacgactcactatagggccgccgaggacggggccg

SEQ ID NO: 21 - DNA, 18 bp, synthetic construct
  source 1..18: mol_type = other DNA
gcgaccccggacaggccg

SEQ ID NO: 22 - DNA, 89 bp, synthetic construct
  source 1..89: mol_type = other DNA
ctggctggcgttcttccgcgacctggtcgttttagtccccttcgtttttggggtagtctaaatcccctatagtgagtcgtattaatttc

SEQ ID NO: 23 - DNA, 45 bp, synthetic construct
  source 1..45: mol_type = other DNA
gaaattaatacgactcactataggggcgccaggcgcaggtcgatg

SEQ ID NO: 24 - DNA, 21 bp, synthetic construct
  source 1..21: mol_type = other DNA
cgccgcaagcgcttcagctca

SEQ ID NO: 25 - DNA, 89 bp, synthetic construct
  source 1..89: mol_type = other DNA
ggtgcgcgaggcaggccgcgtcgtcggggttttagtccccttcgtttttggggtagtctaaatcccctatagtgagtcgtattaatttc

SEQ ID NO: 26 - DNA, 55 bp, synthetic construct
  source 1..55: mol_type = other DNA
gaaattaatacgactcactatagggaattgcgaagggcgaacgcgattttaaaga

SEQ ID NO: 27 - DNA, 28 bp, synthetic construct
  source 1..28: mol_type = other DNA
atgaaccgggtaattagcgtgctggccg

SEQ ID NO: 28 - DNA, 89 bp, synthetic construct
  source 1..89: mol_type = other DNA
ccgcgtcggctttcttcgcggccgagctgttttagtccccttcgtttttggggtagtctaaatcccctatagtgagtcgtattaatttc

SEQ ID NO: 29 - DNA, 55 bp, synthetic construct
  source 1..55: mol_type = other DNA
gaaattaatacgactcactatagggccaacaagaaggcgtactcgacctgaaaga

SEQ ID NO: 30 - DNA, 28 bp, synthetic construct
  source 1..28: mol_type = other DNA
cgctcgctgaaccggatcgatgtgtact

SEQ ID NO: 31 - DNA, 89 bp, synthetic construct
  source 1..89: mol_type = other DNA
gttatccaccatacggataggggatctcgttttagtccccttcgtttttggggtagtctaaatcccctatagtgagtcgtattaatttc

SEQ ID NO: 32 - DNA, 49 bp, synthetic construct
  source 1..49: mol_type = other DNA
gaaattaatacgactcactatagggtcagcacgattcggagtgggcagc

SEQ ID NO: 33 - DNA, 19 bp, synthetic construct
  source 1..19: mol_type = other DNA
cgcaccgtctccgcgcagc

SEQ ID NO: 34 - DNA, 89 bp, synthetic construct
  source 1..89: mol_type = other DNA
tcagtgaggtcgcccgtctacttggtgtgttttagtccccttcgtttttggggtagtctaaatcccctatagtgagtcgtattaatttc

SEQ ID NO: 35 - DNA, 47 bp, synthetic construct
  source 1..47: mol_type = other DNA
gaaattaatacgactcactatagggccgatggtttgcggtggggtgt

SEQ ID NO: 36 - DNA, 29 bp, synthetic construct
  source 1..29: mol_type = other DNA
ccggttgatgtggtcgtagtaggtcgatg

SEQ ID NO: 37 - DNA, 89 bp, synthetic construct
  source 1..89: mol_type = other DNA
cacagctgaccgagctgggtgtgccgatgttttagtccccttcgtttttggggtagtctaaatcccctatagtgagtcgtattaatttc

SEQ ID NO: 38 - DNA, 47 bp, synthetic construct
  source 1..47: mol_type = other DNA
gaaattaatacgactcactatagggtccacgccgccaactacggtgt

SEQ ID NO: 39 - DNA, 25 bp, synthetic construct
  source 1..25: mol_type = other DNA
gacggtgcatctggccacctcgatg

SEQ ID NO: 40 - DNA, 89 bp, synthetic construct
  source 1..89: mol_type = other DNA
acggtgtttacggtgcccgcaaagtgtggttttagtccccttcgtttttggggtagtctaaatcccctatagtgagtcgtattaatttc

SEQ ID NO: 41 - DNA, 45 bp, synthetic construct; identical to SEQ ID NO: 23
  source 1..45: mol_type = other DNA

SEQ ID NO: 42 - DNA, 21 bp, synthetic construct
  source 1..21: mol_type = other DNA
cgccgcaagcgcttcagctca

SEQ ID NO: 43 - DNA, 89 bp, synthetic construct
  source 1..89: mol_type = other DNA
gtcgatgccggcgcacggcccgggaccagttttagtccccttcgtttttggggtagtctaaatcccctatagtgagtcgtattaatttc

SEQ ID NO: 44 - DNA, 46 bp, synthetic construct
  source 1..46: mol_type = other DNA
gaaattaatacgactcactatagggtggcgggtcgcttccacgatg

SEQ ID NO: 45 - DNA, 25 bp, synthetic construct
  source 1..25: mol_type = other DNA
tcttgttggcgggtccagatggctt

SEQ ID NO: 46 - DNA, 89 bp, synthetic construct
  source 1..89: mol_type = other DNA
ccacctccatggtcctcgacgcgatcgagttttagtccccttcgtttttggggtagtctaaatcccctatagtgagtcgtattaatttc

SEQ ID NO: 47 - DNA, 47 bp, synthetic construct
  source 1..47: mol_type = other DNA
gaaattaatacgactcactatagggcggtcggagcggtcggaagctc

SEQ ID NO: 48 - DNA, 27 bp, synthetic construct
  source 1..27: mol_type = other DNA
ttgatcagctcggtcttgtataggccg

SEQ ID NO: 49 - DNA, 89 bp, synthetic construct
  source 1..89: mol_type = other DNA
tatgacaatgcactagccgagacgatcagttttagtccccttcgtttttggggtagtctaaatcccctatagtgagtcgtattaatttc

SEQ ID NO: 50 - DNA, 52 bp, synthetic construct
  source 1..52: mol_type = other DNA
gaaattaatacgactcactatagggatgcaccgtcgaacggctgatgaccaa

SEQ ID NO: 51 - DNA, 23 bp, synthetic construct
  source 1..23: mol_type = other DNA
agcgatcgtggtcctgcgggctt

SEQ ID NO: 52 - DNA, 89 bp, synthetic construct
  source 1..89: mol_type = other DNA
ctcggcctgtccgggaccacccgcggcagttttagtccccttcgtttttggggtagtctaaatcccctatagtgagtcgtattaatttc

SEQ ID NO: 53 - DNA, 53 bp, synthetic construct
  source 1..53: mol_type = other DNA
gaaattaatacgactcactatagggtggaaaggatggggtcatgtcaggtggt

SEQ ID NO: 54 - DNA, 19 bp, synthetic construct
  source 1..19: mol_type = other DNA
catccgcaccgcccgctca

SEQ ID NO: 55 - DNA, 89 bp, synthetic construct
  source 1..89: mol_type = other DNA
tcatcgaggaggtacccgccggagctgcgttttagtccccttcgtttttggggtagtctaaatcccctatagtgagtcgtattaatttc

SEQ ID NO: 56 - DNA, 53 bp, synthetic construct
  source 1..53: mol_type = other DNA
gaaattaatacgactcactatagggaacaagaaggcgtactcgacctgaaaga

SEQ ID NO: 57 - DNA, 27 bp, synthetic construct
  source 1..27: mol_type = other DNA
gctcgctgaaccggatcgatgtgtact

SEQ ID NO: 58 - DNA, 89 bp, synthetic construct; identical to SEQ ID NO: 31
  source 1..89: mol_type = other DNA

SEQ ID NO: 59 - RNA, 14 nt, synthetic construct
  source 1..14: mol_type = other RNA
tttttttttttttt